(19) United States
(12) Patent Application Publication
(10) Pub. No.: US 2019/0345138 A1
(43) Pub. Date: Nov. 14, 2019

(54) HETEROCYCLIC AMIDES AS KINASE INHIBITORS

(71) Applicant: GlaxoSmithKline Intellectual Property Development Limited, Brentford, Middlesex (GB)

(72) Inventors: Alain Claude-Marie DAUGAN, Les Ulis (FR); Frederic G. DONCHE, Les Ulis (FR); Nicolas Eric FAUCHER, Les Ulis (FR); Nicolas S. GEORGE, Les Ulis (FR)

(21) Appl. No.: 16/461,410

(22) PCT Filed: Nov. 17, 2017
(86) PCT No.: PCT/IB2017/057225

§ 371 Date: May 16, 2019

Related U.S. Application Data
(60) Provisional application No. 62/585,267, filed on Nov. 13, 2017.
(60) Provisional application No. 62/424,047, filed on Nov. 18, 2016.

(51) Int. Cl. C07D 401/14; C07D 413/14; C07D 487/04; C07D 417/14; A61P 35/00; A61P 17/00

(52) U.S. Cl. CPC C07D 401/14; C07D 413/14; A61P 17/00; C07D 417/14; A61P 35/00; C07D 487/04

(57) ABSTRACT

Disclosed are compounds having the formula:

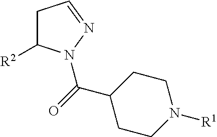

wherein R¹ and R² are as defined herein, and methods of making and using the same.

### FIELD OF THE INVENTION

The present invention relates to heterocyclic amides that inhibit RIP1 kinase and methods of making and using the same.

### BACKGROUND OF THE INVENTION

Receptor-interacting protein-1 (RIP1) kinase, originally referred to as RIP, is a TKL family serine/threonine protein kinase involved in innate immune signaling. RIP1 kinase is a RHIM domain containing protein, with an N-terminal kinase domain and a C-terminal death domain (Trends Biochem. Sci., 30, 151-159 (2005)). The death domain of RIP1 mediates interaction with other death domain containing proteins including Fas and TNFR-1 (Cell, 81 513-523 (1995)), TRAIL-R1 and TRAIL-R2 (Immunity, 7, 821-830 (1997)), and TRADD (Immunity, 4, 387-396 (1996)), while the RHIM domain is crucial for binding other RHIM domain containing proteins such as TRIF (Nat. Immunol., 5, 503-507 (2004)), DAI (EMBO Rep. 10, 916-922 (2009)) and RIP3 (J. Biol. Chem., 274, 16871-16875 (1999)); Curr. Biol., 9, 539-542 (1999)) and exerts many of its effects through these interactions. RIP1 is a central regulator of cell signaling, and is involved in mediating both pro-survival and programmed cell death pathways which will be discussed below.

The role for RIP1 in cell signaling has been assessed under various conditions [including TLR3 (Nat Immunol., 5, 503-507 (2004)), TLR4 (J. Biol. Chem., 280, 36560-6566 (2005)), TRAIL (Cell Signal., 27(2), 306-314 (2015)), FAS (J. Biol. Chem., 279, 7925-7933 (2004))], but is best understood in the context of mediating signals downstream of the death receptor TNFR1 (Cell, 114, 181-190 (2003)). Engagement of the TNFR by TNF leads to its oligomerization, and the recruitment of multiple proteins, including linear K63-linked polyubiquitinated RIP1 (Mol. Cell, 22, 245-257 (2006)), TRAF2/5 (J. Mol. Biol., 396, 528-539 (2010)), TRADD (Nat. Immunol., 9, 1037-1046 (2008)), and cIAPs (Proc. Natl. Acad. Sci. USA., 105, 11778-11783 (2008)), to the cytoplasmic tail of the receptor. This complex which is dependent on RIP1 as a scaffolding protein (i.e. kinase independent), termed complex I, provides a platform for pro-survival signaling through the activation of the NFκB and MAP kinases pathways (Sci. Signal., 115, re4 (2010)). Alternatively, binding of TNF to its receptor under conditions promoting the deubiquitination of RIP1 (by proteins such as A20 and CYLD or inhibition of the cIAPs) results in receptor internalization and the formation of complex II or DISC (death-inducing signaling complex) (Cell Death Dis., 2, e230 (2011)). Formation of the DISC, which contains RIP1, TRADD, FADD and caspase 8, results in the activation of caspase 8 and the onset of programmed apoptotic cell death also in a RIP1 kinase independent fashion (FEBS J, 278, 877-887 (2012)). Apoptosis is largely a quiescent form of cell death, and is involved in routine processes such as development and cellular homeostasis.

Under conditions where the DISC forms and RIP3 is expressed, but apoptosis is inhibited (such as FADD/caspase 8 deletion, caspase inhibition, or viral infection), a third RIP1 kinase-dependent possibility exists. RIP3 can now enter this complex, become phosphorylated by RIP1 and initiate a caspase-independent programmed necrotic cell death through the activation of MLKL and PGAM5 (Cell, 148, 213-227 (2012)); (Cell, 148, 228-243 (2012)); (Proc. Natl. Acad. Sci. USA., 109, 5322-5327 (2012)). As opposed to apoptosis, programmed necrosis (not to be confused with passive necrosis which is not programmed) results in the release of danger associated molecular patterns (DAMPs) from the cell. These DAMPs are capable of providing a “danger signal” to surrounding cells and tissues, eliciting proinflammatory responses including inflammasome activation, cytokine production and cellular recruitment (Nat. Rev. Immunol., 8, 279-289 (2008)).

Dysregulation of RIP1 kinase-mediated programmed cell death has been linked to various inflammatory diseases, as demonstrated by use of the RIP3 knockout mouse (where RIP1-mediated programmed necrosis is completely blocked) and by Necrostatin-1 (a tool inhibitor of RIP1 kinase activity with poor oral bioavailability). The RIP3 knockout mouse has been shown to be protective in inflammatory bowel disease (including ulcerative colitis and Crohn's disease) (Nature, 477, 330-334 (2011)), psoriasis (Immunity, 35, 572-582 (2011)), retinal-detachment-induced photoreceptor necrosis (PNAS, 107, 21695-21700, (2010)), retinitis pigmentosa (Proc. Natl. Acad. Sci., 109:36, 14598-14603 (2012)), cerulein-induced acute pancreatits (Cell, 137, 1100-1111 (2009)), and sepsis/systemic inflammatory response syndrome (SIRS) (Immunity, 35, 908-918 (2011)). Necrostatin-1 has been shown to be effective in alleviating ischemic brain injury (Nat. Chem. Biol., 1, 112-119 (2005)), retinal ischemia/reperfusion injury (J. Neurosci. Res., 88, 1569-1576 (2010)), Huntington's disease (Cell Death Dis., 2 e115 (2011)), renal ischemia reperfusion injury (Kidney Int., 81, 751-761 (2012)), cisplatin induced kidney injury (Ren. Fail., 34, 373-377 (2012)), and traumatic brain injury (Neurochem. Res., 37, 1849-1858 (2012)). Other diseases or disorders regulated at least in part by RIP1-dependent apoptosis, necrosis or cytokine production include hematological and solid organ malignancies (Genes Dev., 27, 1640-1649 (2013)), bacterial infections and viral infections (Cell Host & Microbe, 15, 23-35 (2014)) (including, but not limited to, tuberculosis and influenza (Cell, 153, 1-14 (2013)) and Lysosomal storage diseases (particularly, Gaucher Disease, Nature Medicine Advance Online Publication, 19 Jan. 2014, doi:10.1038/nm.3449).

A potent, selective, small molecule inhibitor of RIP1 kinase activity would block RIP1-dependent cellular necrosis and thereby provide a therapeutic benefit in diseases or events associated with DAMPs, cell death, and/or inflammation.

### SUMMARY OF THE INVENTION

The invention is directed to a compound according to Formula (I)

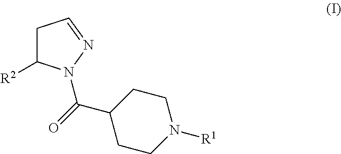

wherein:
R¹ is a substituted or unsubstituted 5-6 membered heteroaryl or 9-10 membered heteroaryl group,
wherein said substituted 5-6 membered heteroaryl or 9-10 membered heteroaryl group is substituted by 1 or 2 substituents independently selected from hydroxyl, cyano, halogen, (C₁-C₄)alkyl, halo(C₁-C₄)alkyl, hydroxy(C₁-C₄)alkyl, (C₂-C₄)alkynyl, optionally substituted (C₁-C₄)alkoxy, optionally substituted 5-6 membered heterocycloalkyl-CO—, fused 5-6 membered heterocycloalkyl, H₂N—, ((C₁-C₄)alkyl)-NH—, ((C₁-C₄)alkyl)((C₁-C₄)alkyl)N—, H₂NCO—, H₂NCO—(C₁-C₄)alkyl-, ((C₁-C₄)alkyl)NHCO—, (hydroxy-(C₁-C₄)alkyl)NHCO—, (C₃-C₆)cycloalkyl-NHCO—, optionally substituted 5-6 membered heterocycloalkyl-NHCO—, ((C₁-C₄)alkyl)((C₁-C₄)alkyl)N—CO—, (C₁-C₄)alkyl-CONH—, ((C₁-C₄)alkyl)((C₁-C₄)alkyl)N—NHCO—, —CO₂H, —CO₂(C₁-C₄)alkyl, (C₁-C₄)alkylthio-, phenyl-(C₁-C₄)alkylthio-, (C₁-C₄)alkyl-SO₂—, phenyl, optionally substituted 5-6 membered heterocycloalkyl, and optionally substituted 5-6 membered heteroaryl group,
   wherein said optionally substituted (C₁-C₄)alkoxy is optionally substituted by hydroxyl, —CO₂H, —CONH₂, 5-6 membered heterocycloalkyl, or 5-6 membered heteroaryl; or said optionally substituted 5-6 membered heterocycloalkyl-CO—, optionally substituted 5-6 membered heterocycloalkyl, or optionally substituted 5-6 membered heteroaryl group is optionally substituted by (C₁-C₄)alkyl or oxo; or said optionally substituted 5-6 membered heterocycloalkyl-NHCO— is optionally substituted by (C₁-C₄)alkyl-CO—; and
      R² is a substituted or unsubstituted phenyl or 5-6 membered heteroaryl group,
wherein said substituted phenyl or 5-6 membered heteroaryl group is substituted by 1 or 2 substituents independently selected from halogen, (C₁-C₄)alkyl, (C₁-C₄)alkoxy, and cyano;
   or a pharmaceutically acceptable salt thereof.

The compounds of Formula (II) that inhibit the activity and/or function of RIP1 kinase have the stereochemistry as designated in Formula (II)

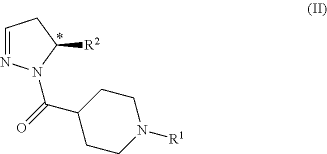

wherein R¹, and R² are defined in accordance with Formula (I). Generally, based on the definition of R² provided herein, the stereochemistry at the * chiral carbon center is (S).

Compounds of Formula (II) having the (R) stereochemistry at the * chiral carbon center (generally, as based on the definition of R² provided herein) may be useful tool compounds as negative controls to help confirm the on-target effects of the active (S) enantiomer.

The invention is further directed to a compound according to Formula (II) wherein:

R¹ is a substituted or unsubstituted 5-6 membered heteroaryl or 9-10 membered heteroaryl group,
wherein said substituted 5-6 membered heteroaryl or 9-10 membered heteroaryl group is substituted by 1 or 2 substituents independently selected from hydroxyl, cyano, halogen, (C₁-C₄)alkyl, halo(C₁-C₄)alkyl, hydroxy(C₁-C₄)alkyl, (C₂-C₄)alkynyl, optionally substituted (C₁-C₄)alkoxy, optionally substituted 5-6 membered heterocycloalkyl-CO—, fused 5-6 membered heterocycloalkyl, H₂N—, ((C₁-C₄)alkyl)-NH—, ((C₁-C₄)alkyl)((C₁-C₄)alkyl)N—, H₂NCO—, H₂NCO—(C₁-C₄)alkyl-, ((C₁-C₄)alkyl)NHCO—, (hydroxy-(C₁-C₄)alkyl)NHCO—, (C₃-C₆)cycloalkyl-NHCO—, optionally substituted 5-6 membered heterocycloalkyl-NHCO—, ((C₁-C₄)alkyl)((C₁-C₄)alkyl)N—CO—, (C₁-C₄)alkyl-CONH—, ((C₁-C₄)alkyl)((C₁-C₄)alkyl)N—NHCO—, —CO₂H, —CO₂(C₁-C₄)alkyl, (C₁-C₄)alkylthio-, phenyl-(C₁-C₄)alkylthio-, (C₁-C₄)alkyl-SO₂—, phenyl, optionally substituted 5-6 membered heterocycloalkyl, and optionally substituted 5-6 membered heteroaryl group,
   wherein said optionally substituted (C₁-C₄)alkoxy is optionally substituted by hydroxyl, —CO₂H, —CONH₂, 5-6 membered heterocycloalkyl, or 5-6 membered heteroaryl; or said optionally substituted 5-6 membered heterocycloalkyl-CO—, optionally substituted 5-6 membered heterocycloalkyl, or optionally substituted 5-6 membered heteroaryl group is optionally substituted by (C₁-C₄)alkyl or oxo; or said optionally substituted 5-6 membered heterocycloalkyl-NHCO— is optionally substituted by (C₁-C₄)alkyl-CO—; and
      R² is a substituted or unsubstituted phenyl or 5-6 membered heteroaryl group,
wherein said substituted phenyl or 5-6 membered heteroaryl group is substituted by 1 or 2 substituents independently selected from halogen, (C₁-C₄)alkyl, (C₁-C₄)alkoxy, and cyano;
   or a pharmaceutically acceptable salt thereof.

The invention is further directed to a compound according to Formula (II) wherein: R¹ is a substituted or unsubstituted 5-6 membered heteroaryl group, wherein said substituted or unsubstituted 5-6 membered heteroaryl group is a substituted or unsubstituted pyrimidinyl or oxadiazolyl group,
wherein said substituted pyrimidinyl group is substituted by 1 or 2 substituents independently selected from cyano, halogen, (C₁-C₄)alkyl, H₂N—, H₂NCO—, and —CO₂H; or said substituted oxadiazolyl group is substituted by (C₁-C₄)alkyl; and
   R² is a substituted or unsubstituted phenyl or pyridyl group,

wherein said substituted phenyl or pyridyl group is substituted by 1 or 2 fluoro groups; or a pharmaceutically acceptable salt thereof.

The compounds according to Formulas (I) and (II), or pharmaceutically acceptable salts thereof, inhibit the activity and/or function of RIP1 kinase. Accordingly, these compounds may be particularly useful for the treatment of RIP1 kinase-mediated diseases or disorders. Such RIP1 kinase-mediated diseases or disorders are diseases or disorders that are mediated by activation of RIP1 kinase, and as such, are diseases or disorders where inhibition of RIP1 kinase would provide benefit.

### BRIEF DESCRIPTION OF THE DRAWINGS

FIG. 1A shows the temperature loss over time in mice after oral pre-dosing with the compound of Examples 20, 32, 78, 131, or vehicle followed by simultaneous i.v. administration of mouse TNF and zVAD.

FIG. 1B shows the temperature loss in mice 3 hours after oral pre-dosing with the compound of Examples 20, 32, 78, 131, or vehicle followed by simultaneous i.v. administration of mouse TNF and zVAD.

FIG. 2A shows the temperature loss over time in mice after oral pre-dosing with the compound of Examples 71, 85, 108, 109, or vehicle followed by simultaneous i.v. administration of mouse TNF and zVAD.

FIG. 2B shows the temperature loss in mice 3 hours after oral pre-dosing with the compound of Examples 71, 85, 108, 109, or vehicle followed by simultaneous i.v. administration of mouse TNF and zVAD.

FIG. 3A shows the temperature loss over time in mice after oral pre-dosing with the compound of Example 78 or vehicle followed by simultaneous i.v. administration of mouse TNF and zVAD.

FIG. 3B shows the temperature loss in mice 3 hours after oral pre-dosing with the compound of Example 78 or vehicle followed by simultaneous i.v. administration of mouse TNF and zVAD.

FIG. 4A shows the temperature loss over time in mice after oral pre-dosing with the compound of Example 108 or vehicle followed by simultaneous i.v. administration of mouse TNF and zVAD.

FIG. 4B shows the temperature loss in mice 2 hours after oral pre-dosing with the compound of Example 108 or vehicle followed by simultaneous i.v. administration of mouse TNF and zVAD.

FIG. 5A shows the temperature loss over time in mice after oral pre-dosing with the compound of Example 78 or vehicle followed by simultaneous i.v. administration of mouse TNF.

FIG. 5B shows the temperature loss in mice 7.5 hours after oral pre-dosing with the compound of Example 78 or vehicle followed by simultaneous i.v. administration of mouse TNF.

FIG. 6A shows the scotopic B-wave electroretinography recordings at P39 and P46 in Rd10 mice after start of daily in-diet dosing with compound of Example 78 or control diet at P28 followed by switch from dark rearing to 12-hour light/dark cycle at P30.

FIG. 6B shows the photopic B-wave electroretinography recordings at P39 and P46 in Rd10 mice after start of daily in-diet dosing with compound of Example 78 or control diet at P28 followed by switch from dark rearing to 12-hour light/dark cycle at P30.

FIG. 6C shows the measurement of the thickness of the Outer Nuclear Cell (ONL) layers at various distances from the Optic Nerve Head (ONH) in hematoxylin and eosin stained retinal tissue sections collected at P46 in Rd10 mice after start of daily in-diet dosing with compound of Example 78 or control diet at P28 followed by switch from dark rearing to 12-hour light/dark cycle at P30.

FIG. 7 shows the clinical scores over time in mice after daily in-diet dosing with compound of Example 78 or control diet followed by induction of experimental autoimmune encephalomyelitis with MOG₃₅₋₅₅, heat inactivated Mycobacterium tuberculosis, and pertussis toxin.

FIG. 8A shows compound of Example 78 improves fed blood glucose over time without altering body weight in db/db mice.

(* p<0.05)

FIG. 8B shows compound of Example 78 improves fed blood glucose over time without altering body weight in db/db mice.

FIG. 9A shows compound of Example 78 improves fasted blood glucose without altering body weight in db/db mice at 8 weeks of dosing. (* p<0.05)

FIG. 9B shows compound of Example 78 improves fasted blood glucose without altering body weight in db/db mice at 8 weeks of dosing.

FIG. 10A shows effect of compound of Example 78 on food intake and body weight in obese, high fat diet-fed mice. (* p<0.05; ** p<0.001)

FIG. 10B shows effect of compound of Example 78 on food intake and body weight in obese, high fat diet-fed mice. (**p<0.001)

FIG. 11A shows subcutaneous pancreatic tumor model with Example 78 alone or in combination with anti-PD1.

FIG. 11B shows subcutaneous bladder tumor model with Example 78 alone or in combination with anti-PD1.

FIG. 12A shows the percentage of mice without severe dermatitis over time. After weaning mice received daily in-diet dosing with compound of Example 78 or control diet as indicated and were monitored for development of dermatitis.

FIG. 12B shows the percentage of mice without severe dermatitis over time. Once mice developed clinical signs of dermatitis (about 6 weeks of age), mice received daily in-diet dosing with compound of Example 78 or control diet as indicated and were monitored for development of severe dermatitis.

FIG. 13 shows an X-ray powder diffraction pattern of Compound A—Form 1.

FIG. 14 shows a differential scanning calorimetry trace of Compound A—Form 1.

FIG. 15 shows an X-ray powder diffraction pattern of Compound A—Form 2.

FIG. 16 shows a differential scanning calorimetry trace of Compound A—Form 2.

### DETAILED DESCRIPTION OF THE INVENTION

This invention relates to compounds of Formulas (I) and (II) as defined above or pharmaceutically acceptable salts thereof.

In one embodiment, the invention is directed to a compound according to Formula (I)

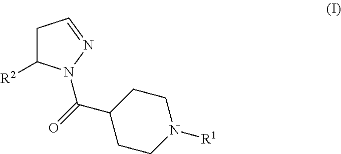

wherein:
R¹ is a substituted or unsubstituted 5-6 membered heteroaryl or 9-10 membered heteroaryl group,
wherein said substituted 5-6 membered heteroaryl or 9-10 membered heteroaryl group is substituted by 1 or 2 substituents independently selected from hydroxyl, cyano, halogen, (C₁-C₄)alkyl, halo(C₁-C₄)alkyl, hydroxy(C₁-C₄)alkyl, (C₂-C₄)alkynyl, optionally substituted (C₁-C₄)alkoxy, optionally substituted 5-6 membered heterocycloalkyl-CO—, fused 5-6 membered heterocycloalkyl, H₂N—, ((C₁-C₄)alkyl)-NH—, ((C₁-C₄)alkyl)((C₁-C₄)alkyl)N—, H₂NCO—, H₂NCO—(C₁-C₄)alkyl-, ((C₁-C₄)alkyl)NHCO—, (hydroxy-(C₁-C₄)alkyl)NHCO—, (C₃-C₆)cycloalkyl-NHCO—, optionally substituted 5-6 membered heterocycloalkyl-NHCO—, ((C₁-C₄)alkyl)((C₁-C₄)alkyl)N—CO—, (C₁-C₄)alkyl-CONH—, ((C₁-C₄)alkyl)((C₁-C₄)alkyl)N—NHCO—, —CO₂H, —CO₂(C₁-C₄)alkyl, (C₁-C₄)alkylthio-, phenyl-(C₁-C₄)alkylthio-, (C₁-C₄)alkyl-SO₂—, phenyl, optionally substituted 5-6 membered heterocycloalkyl, and optionally substituted 5-6 membered heteroaryl group,
   wherein said optionally substituted (C₁-C₄)alkoxy is optionally substituted by hydroxyl, —CO₂H, —CONH₂, 5-6 membered heterocycloalkyl, or 5-6 membered heteroaryl; or said optionally substituted 5-6 membered heterocycloalkyl-CO—, optionally substituted 5-6 membered heterocycloalkyl, or optionally substituted 5-6 membered heteroaryl group is optionally substituted by (C₁-C₄)alkyl or oxo; or said optionally substituted 5-6 membered heterocycloalkyl-NHCO— is optionally substituted by (C₁-C₄)alkyl-CO—; and
      R² is a substituted or unsubstituted phenyl or 5-6 membered heteroaryl group,
wherein said substituted phenyl or 5-6 membered heteroaryl group is substituted by 1 or 2 substituents independently selected from halogen, (C₁-C₄)alkyl, (C₁-C₄)alkoxy, and cyano;
   or a pharmaceutically acceptable salt thereof,
wherein said compound or pharmaceutically acceptable salt thereof is not:
(5-(5-fluoropyridin-3-yl)-4,5-dihydro-1H-pyrazol-1-yl)(1-(5-methylpyrimidin-2-yl)piperidin-4-yl)methanone;
(1-(5-fluoropyridin-2-yl)piperidin-4-yl)(5-phenyl-4,5-dihydro-1H-pyrazol-1-yl)methanone;
(1-(5-methylpyridin-2-yl)piperidin-4-yl)(5-phenyl-4,5-dihydro-1H-pyrazol-1-yl)methanone;
(1-(5-methylpyrimidin-2-yl)piperidin-4-yl)(5-phenyl-4,5-dihydro-1H-pyrazol-1-yl)methanone;
(1-(5-fluoropyrimidin-2-yl)piperidin-4-yl)(5-phenyl-4,5-dihydro-1H-pyrazol-1-yl)methanone;
(5-(5-fluoropyridin-3-yl)-4,5-dihydro-1H-pyrazol-1-yl)(1-(5-fluoropyrimidin-2-yl)piperidin-4-yl)methanone;
(5-(5-fluoropyridin-3-yl)-4,5-dihydro-1H-pyrazol-1-yl)(1-(pyrimidin-2-yl)piperidin-4-yl)methanone;
(1H-indol-2-yl)(5-phenyl-4,5-dihydro-1H-pyrazol-1-yl)methanone;
(5-(pyridin-3-yl)-4,5-dihydro-1H-pyrazol-1-yl)(1-(pyrimidin-2-yl)piperidin-4-yl)methanone;
(5-(6-methylpyridin-3-yl)-4,5-dihydro-1H-pyrazol-1-yl)(1-(pyrimidin-2-yl)piperidin-4-yl)methanone;
(5-(6-methylpyridin-3-yl)-4,5-dihydro-1H-pyrazol-1-yl)(1-(pyridin-2-yl)piperidin-4-yl)methanone;
(1-(pyridin-2-yl)piperidin-4-yl)(5-(pyridin-3-yl)-4,5-dihydro-1H-pyrazol-1-yl)methanone;
(5-(5-methylpyrazin-2-yl)-4,5-dihydro-1H-pyrazol-1-yl)(1-(pyridin-2-yl)piperidin-4-yl)methanone;
(1-(benzo[d]oxazol-2-yl)piperidin-4-yl)(5-(pyridin-3-yl)-4,5-dihydro-1H-pyrazol-1-yl)methanone;
(5-phenyl-4,5-dihydro-1H-pyrazol-1-yl)(1-(pyrimidin-2-yl)piperidin-4-yl)methanone;
(5-phenyl-4,5-dihydro-1H-pyrazol-1-yl)(1-(pyridin-2-yl)piperidin-4-yl)methanone 2,2,2-trifluoroacetate;
(5-phenyl-4,5-dihydro-1H-pyrazol-1-yl)(1-(5-(trifluoromethyl)pyridin-2-yl)piperidin-4-yl)methanone 2,2,2-trifluoroacetate;
(1-(benzo[d]oxazol-2-yl)piperidin-4-yl)(5-phenyl-4,5-dihydro-1H-pyrazol-1-yl)methanone 2,2,2-trifluoroacetate;
(1-(5-fluoropyrimidin-2-yl)piperidin-4-yl)(5-(6-methylpyridin-3-yl)-4,5-dihydro-1H-pyrazol-1-yl)methanone; or
4-(1-(1-(5-fluoropyrimidin-2-yl)piperidine-4-carbonyl)-4,5-dihydro-1H-pyrazol-5-yl)benzonitrile.

In another embodiment, the invention is directed to a compound according to Formula (I) wherein:

R¹ is a substituted or unsubstituted 5-6 membered heteroaryl or 9-10 membered heteroaryl group,
wherein said substituted 5-6 membered heteroaryl or 9-10 membered heteroaryl group is substituted by 1 or 2 substituents independently selected from hydroxyl, cyano, halogen, (C₁-C₄)alkyl, halo(C₁-C₄)alkyl, hydroxy(C₁-C₄)alkyl, (C₂-C₄)alkynyl, optionally substituted (C₁-C₄)alkoxy, optionally substituted 5-6 membered heterocycloalkyl-CO—, fused 5-6 membered heterocycloalkyl, H₂N—, ((C₁-C₄)alkyl)-NH—, ((C₁-C₄)alkyl)((C₁-C₄)alkyl)N—, H₂NCO—, H₂NCO—(C₁-C₄)alkyl-, ((C₁-C₄)alkyl)NHCO—, (hydroxy-(C₁-C₄)alkyl)NHCO—, (C₃-C₆)cycloalkyl-NHCO—, optionally substituted 5-6 membered heterocycloalkyl-NHCO—, ((C₁-C₄)alkyl)((C₁-C₄)alkyl)N—CO—, (C₁-C₄)alkyl-CONH—, ((C₁-C₄)alkyl)((C₁-C₄)alkyl)N—NHCO—, —CO₂H, —CO₂(C₁-C₄)alkyl, (C₁-C₄)alkylthio-, phenyl-(C₁-C₄)alkylthio-, (C₁-C₄)alkyl-SO₂—, phenyl, optionally substituted 5-6 membered heterocycloalkyl, and optionally substituted 5-6 membered heteroaryl group,
   wherein said optionally substituted (C₁-C₄)alkoxy is optionally substituted by hydroxyl, —CO₂H, —CONH₂, 5-6 membered heterocycloalkyl, or 5-6 membered heteroaryl; or said optionally substituted 5-6 membered heterocycloalkyl-CO—, optionally substituted 5-6 membered heterocycloalkyl, or optionally substituted 5-6 membered heteroaryl group is optionally substituted by (C₁-C₄)alkyl or oxo; or said optionally substituted 5-6 membered heterocycloalkyl-NHCO— is optionally substituted by (C₁-C₄)alkyl-CO—; and
      R² is a substituted or unsubstituted phenyl or 5-6 membered heteroaryl group,
wherein said substituted phenyl or 5-6 membered heteroaryl group is substituted by 1 or 2 substituents independently selected from halogen, (C₁-C₄)alkyl, (C₁-C₄)alkoxy, and cyano;
   or a pharmaceutically acceptable salt thereof,
wherein said compound or pharmaceutically acceptable salt thereof is not:
(5-(5-fluoropyridin-3-yl)-4,5-dihydro-1H-pyrazol-1-yl)(1-(5-methylpyrimidin-2-yl)piperidin-4-yl)methanone;
(1-(5-fluoropyridin-2-yl)piperidin-4-yl)(5-phenyl-4,5-dihydro-1H-pyrazol-1-yl)methanone;
(1-(5-methylpyridin-2-yl)piperidin-4-yl)(5-phenyl-4,5-dihydro-1H-pyrazol-1-yl)methanone;
(1-(5-methylpyrimidin-2-yl)piperidin-4-yl)(5-phenyl-4,5-dihydro-1H-pyrazol-1-yl)methanone;
(1-(5-fluoropyrimidin-2-yl)piperidin-4-yl)(5-phenyl-4,5-dihydro-1H-pyrazol-1-yl)methanone;
(5-(5-fluoropyridin-3-yl)-4,5-dihydro-1H-pyrazol-1-yl)(1-(5-fluoropyrimidin-2-yl)piperidin-4-yl)methanone;
(5-(5-fluoropyridin-3-yl)-4,5-dihydro-1H-pyrazol-1-yl)(1-(pyrimidin-2-yl)piperidin-4-yl)methanone;
(1H-indol-2-yl)(5-phenyl-4,5-dihydro-1H-pyrazol-1-yl)methanone;
(5-(pyridin-3-yl)-4,5-dihydro-1H-pyrazol-1-yl)(1-(pyrimidin-2-yl)piperidin-4-yl)methanone;
(5-(6-methylpyridin-3-yl)-4,5-dihydro-1H-pyrazol-1-yl)(1-(pyrimidin-2-yl)piperidin-4-yl)methanone;
(5-(6-methylpyridin-3-yl)-4,5-dihydro-1H-pyrazol-1-yl)(1-(pyridin-2-yl)piperidin-4-yl)methanone;
(1-(pyridin-2-yl)piperidin-4-yl)(5-(pyridin-3-yl)-4,5-dihydro-1H-pyrazol-1-yl)methanone;
(5-(5-methylpyrazin-2-yl)-4,5-dihydro-1H-pyrazol-1-yl)(1-(pyridin-2-yl)piperidin-4-yl)methanone;
(1-(benzo[d]oxazol-2-yl)piperidin-4-yl)(5-(pyridin-3-yl)-4,5-dihydro-1H-pyrazol-1-yl)methanone;
(5-phenyl-4,5-dihydro-1H-pyrazol-1-yl)(1-(pyrimidin-2-yl)piperidin-4-yl)methanone;
(5-phenyl-4,5-dihydro-1H-pyrazol-1-yl)(1-(pyridin-2-yl)piperidin-4-yl)methanone;
(5-phenyl-4,5-dihydro-1H-pyrazol-1-yl)(1-(5-(trifluoromethyl)pyridin-2-yl)piperidin-4-yl)methanone;
(1-(benzo[d]oxazol-2-yl)piperidin-4-yl)(5-phenyl-4,5-dihydro-1H-pyrazol-1-yl)methanone;
(1-(5-fluoropyrimidin-2-yl)piperidin-4-yl)(5-(6-methylpyridin-3-yl)-4,5-dihydro-1H-pyrazol-1-yl)methanone; or
4-(1-(1-(5-fluoropyrimidin-2-yl)piperidine-4-carbonyl)-4,5-dihydro-1H-pyrazol-5-yl)benzonitrile.

In one embodiment, the invention is directed to compounds of Formula (II)

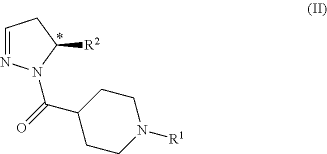

wherein:
R¹ is a substituted or unsubstituted 5-6 membered heteroaryl or 9-10 membered heteroaryl group,
wherein said substituted 5-6 membered heteroaryl or 9-10 membered heteroaryl group is substituted by 1 or 2 substituents independently selected from hydroxyl, cyano, halogen, (C₁-C₄)alkyl, halo(C₁-C₄)alkyl, hydroxy(C₁-C₄)alkyl, (C₂-C₄)alkynyl, optionally substituted (C₁-C₄)alkoxy, optionally substituted 5-6 membered heterocycloalkyl-CO—, fused 5-6 membered heterocycloalkyl, H₂N—, ((C₁-C₄)alkyl)-NH—, ((C₁-C₄)alkyl)((C₁-C₄)alkyl)N—, H₂NCO—, H₂NCO—(C₁-C₄)alkyl-, ((C₁-C₄)alkyl)NHCO—, (hydroxy-(C₁-C₄)alkyl)NHCO—, (C₃-C₆)cycloalkyl-NHCO—, optionally substituted 5-6 membered heterocycloalkyl-NHCO—, ((C₁-C₄)alkyl)((C₁-C₄)alkyl)N—CO—, (C₁-C₄)alkyl-CONH—, ((C₁-C₄)alkyl)((C₁-C₄)alkyl)N—NHCO—, —CO₂H, —CO₂(C₁-C₄)alkyl, (C₁-C₄)alkylthio-, phenyl-(C₁-C₄)alkylthio-, (C₁-C₄)alkyl-SO₂—, phenyl, optionally substituted 5-6 membered heterocycloalkyl, and optionally substituted 5-6 membered heteroaryl group,
   wherein said optionally substituted (C₁-C₄)alkoxy is optionally substituted by hydroxyl, —CO₂H, —CONH₂, 5-6 membered heterocycloalkyl, or 5-6 membered heteroaryl; or said optionally substituted 5-6 membered heterocycloalkyl-CO—, optionally substituted 5-6 membered heterocycloalkyl, or optionally substituted 5-6 membered heteroaryl group is optionally substituted by (C₁-C₄)alkyl or oxo; or said optionally substituted 5-6 membered heterocycloalkyl-NHCO— is optionally substituted by (C₁-C₄)alkyl-CO—; and
      R² is a substituted or unsubstituted phenyl or 5-6 membered heteroaryl group,
wherein said substituted phenyl or 5-6 membered heteroaryl group is substituted by 1 or 2 substituents independently selected from halogen, (C₁-C₄)alkyl, (C₁-C₄)alkoxy, and cyano;
   or a pharmaceutically acceptable salt thereof,

wherein the said compound or pharmaceutically acceptable salt thereof is not:
(S)-(1-(5-fluoropyridin-2-yl)piperidin-4-yl)(5-phenyl-4,5-dihydro-1H-pyrazol-1-yl)methanone;
(S)-(1-(5-fluoropyrimidin-2-yl)piperidin-4-yl)(5-phenyl-4,5-dihydro-1H-pyrazol-1-yl)methanone;
(S)-(5-(5-fluoropyridin-3-yl)-4,5-dihydro-1H-pyrazol-1-yl)(1-(5-fluoropyrimidin-2-yl)piperidin-4-yl)methanone;
(S)-(5-(pyridin-3-yl)-4,5-dihydro-1H-pyrazol-1-yl)(1-(pyrimidin-2-yl)piperidin-4-yl)methanone;
(S)-(1-(pyridin-2-yl)piperidin-4-yl)(5-(pyridin-3-yl)-4,5-dihydro-1H-pyrazol-1-yl)methanone;
(S)-(5-phenyl-4,5-dihydro-1H-pyrazol-1-yl)(1-(pyrimidin-2-yl)piperidin-4-yl)methanone; or
(S)-(5-phenyl-4,5-dihydro-1H-pyrazol-1-yl)(1-(pyridin-2-yl)piperidin-4-yl)methanone.

In another embodiment, the invention is further directed to a compound according to Formula (II) wherein:

R¹ is a substituted or unsubstituted 5-6 membered heteroaryl group, wherein said substituted or unsubstituted 5-6 membered heteroaryl group is a substituted or unsubstituted pyrimidinyl or oxadiazolyl group,
wherein said substituted pyrimidinyl group is substituted by 1 or 2 substituents independently selected from cyano, halogen, (C₁-C₄)alkyl, H₂N—, H₂NCO—, and —CO₂H; or said substituted oxadiazolyl group is substituted by (C₁-C₄)alkyl; and
   R² is a substituted or unsubstituted phenyl or pyridyl group,
wherein said substituted phenyl or pyridyl group is substituted by 1 or 2 fluoro groups;
   or a pharmaceutically acceptable salt thereof,

wherein the said compound or pharmaceutically acceptable salt thereof is not:
(S)-(1-(5-fluoropyrimidin-2-yl)piperidin-4-yl)(5-phenyl-4,5-dihydro-1H-pyrazol-1-yl)methanone,
(S)-(5-(5-fluoropyridin-3-yl)-4,5-dihydro-1H-pyrazol-1-yl)(1-(5-fluoropyrimidin-2-yl)piperidin-4-yl)methanone,
(S)-(5-(pyridin-3-yl)-4,5-dihydro-1H-pyrazol-1-yl)(1-(pyrimidin-2-yl)piperidin-4-yl)methanone, or
(S)-(5-phenyl-4,5-dihydro-1H-pyrazol-1-yl)(1-(pyrimidin-2-yl)piperidin-4-yl)methanone.

In one embodiment, the invention is also directed to a compound according to Formula (I) or Formula (II) wherein:

R¹ is a substituted or unsubstituted 5-6 heteroaryl group;

wherein said substituted 5-6 heteroaryl group is substituted by 1 or 2 substituents independently selected from hydroxyl, cyano, halogen, (C₁-C₄)alkyl, halo(C₁-C₄)alkyl, hydroxy(C₁-C₄)alkyl, (C₂-C₄)alkynyl, optionally substituted (C₁-C₄)alkoxy, optionally substituted 5-6 membered heterocycloalkyl-CO—, fused 5-6 membered heterocycloalkyl; H₂N—, ((C₁-C₄)alkyl)-NH—, ((C₁-C₄)alkyl)((C₁-C₄)alkyl)N—, H₂NCO—, H₂NCO—(C₁-C₄)alkyl-, ((C₁-C₄)alkyl)NHCO—, (hydroxy-(C₁-C₄)alkyl)NHCO—, (C₃-C₆)cycloalkyl-NHCO—, optionally substituted 5-6 membered heterocycloalkyl-NHCO—, ((C₁-C₄)alkyl)((C₁-C₄)alkyl)-NCO—, (C₁-C₄)alkyl-CONH—, ((C₁-C₄)alkyl)((C₁-C₄)alkyl)N—NHCO—, —CO₂H, —CO₂(C₁-C₄)alkyl, (C₁-C₄)alkylthio-, phenyl-(C₁-C₄)alkylthio-, (C₁-C₄)alkyl-SO₂—, phenyl, optionally substituted 5-6 membered heterocycloalkyl, and optionally substituted 5-6 membered heteroaryl group,

wherein said optionally substituted (C₁-C₄)alkoxy is optionally substituted by hydroxyl, —CO₂H, —CONH₂, 5-6 membered heterocycloalkyl, or 5-6 membered heteroaryl; or said optionally substituted 5-6 membered heterocycloalkyl-CO—, optionally substituted 5-6 membered heterocycloalkyl, or optionally substituted 5-6 membered heteroaryl group is optionally substituted by (C₁-C₄)alkyl or oxo; or said optionally substituted 5-6 membered heterocycloalkyl-NHCO— is optionally substituted by (C₁-C₄)alkyl-CO—; and

R² is a substituted or unsubstituted phenyl or 5-6 membered heteroaryl group,

wherein said substituted phenyl or 5-6 membered heteroaryl group is substituted by 1 or 2 substituents independently selected from halogen, (C₁-C₄)alkyl, (C₁-C₄)alkoxy, and cyano;

or a pharmaceutically acceptable salt thereof.

In another embodiment of Formula (I) or Formula (II), R¹ is a substituted or unsubstituted 5-6 membered heteroaryl group;

wherein said substituted 5-6 membered heteroaryl group is substituted by 1 or 2 substituents independently selected from cyano, halogen, (C₁-C₄)alkyl, H₂NCO—, and —CO₂H; and

R² is a substituted or unsubstituted phenyl or 5-6 membered heteroaryl group,

wherein said substituted phenyl or 5-6 membered heteroaryl group is substituted by 1 or 2 substituents independently selected from halogen, (C₁-C₄)alkyl, (C₁-C₄)alkoxy, and cyano;

or a pharmaceutically acceptable salt thereof.

In another embodiment of Formula (I) or Formula (II), R¹ is a substituted or unsubstituted 5-6 membered heteroaryl group,

wherein said substituted or unsubstituted 5-6 membered heteroaryl group is a substituted or unsubstituted pyrimidinyl, pyrazinyl, pyridazinyl, pyridyl, oxazolyl, thiazolyl, oxadiazolyl, tetrazolyl, or thiadiazolyl,

wherein said substituted pyrimidinyl, pyrazinyl, pyridazinyl, pyridyl, oxazolyl, thiazolyl, or oxadiazolyl is substituted by 1 or 2 substituents independently selected from hydroxyl, cyano, halogen, (C₁-C₄)alkyl, halo(C₁-C₄)alkyl, hydroxy(C₁-C₄)alkyl, (C₂-C₄)alkynyl, (C₁-C₄)alkoxy, optionally substituted (C₁-C₄)alkoxy, optionally substituted 5-6 membered heterocycloalkyl-CO—, fused 5-6 membered heterocycloalkyl; ((C₁-C₄)alkyl)-NH—, ((C₁-C₄)alkyl)((C₁-C₄)alkyl)N—, H₂NCO—, H₂NCO—(C₁-C₄)alkyl-, ((C₁-C₄)alkyl)NHCO—, (hydroxy-(C₁-C₄)alkyl)NHCO—, (C₃-C₆)cycloalkyl-NHCO—, optionally substituted 5-6 membered heterocycloalkyl-NHCO—, ((C₁-C₄)alkyl)((C₁-C₄)alkyl)-NCO—, (C₁-C₄)alkyl-CONH—, ((C₁-C₄)alkyl)((C₁-C₄)alkyl)N—NHCO—, —CO₂H, —CO₂(C₁-C₄)alkyl, (C₁-C₄)alkylthio-, phenyl-(C₁-C₄)alkylthio-, (C₁-C₄)alkyl-SO₂—, phenyl, optionally substituted 5-6 membered heterocycloalkyl, and optionally substituted 5-6 membered heteroaryl group,

wherein said optionally substituted (C₁-C₄)alkoxy is optionally substituted by hydroxyl, —CO₂H, —CONH₂, 5-6 membered heterocycloalkyl, or 5-6 membered heteroaryl; or said optionally substituted 5-6 membered heterocycloalkyl-CO—, optionally substituted 5-6 membered heterocycloalkyl, or optionally substituted 5-6 membered heteroaryl group is optionally substituted by (C₁-C₄)alkyl or oxo; or said optionally substituted 5-6 membered heterocycloalkyl-NHCO— is optionally substituted by (C₁-C₄)alkyl-CO—.

In another embodiment of Formula (I) or Formula (II), R¹ is a substituted or unsubstituted 5-6 membered heteroaryl group,

wherein said substituted or unsubstituted 5-6 membered heteroaryl group is a substituted or unsubstituted tetrazolyl or thiadiazolyl,

wherein said substituted tetrazolyl or thiadiazolyl is substituted by 1 or 2 substituents independently selected from hydroxyl, cyano, halogen, (C₁-C₄)alkyl, halo(C₁-C₄)alkyl, hydroxy(C₁-C₄)alkyl, (C₂-C₄)alkynyl, (C₁-C₄)alkoxy, optionally substituted (C₁-C₄)alkoxy, optionally substituted 5-6 membered heterocycloalkyl-CO—, fused 5-6 membered heterocycloalkyl; H₂N—, ((C₁-C₄)alkyl)-NH—, ((C₁-C₄)alkyl)((C₁-C₄)alkyl)N—, H₂NCO—, H₂NCO—(C₁-C₄)alkyl-, ((C₁-C₄)alkyl)NHCO—, (hydroxy-(C₁-C₄)alkyl)NHCO—, (C₃-C₆)cycloalkyl-NHCO—, optionally substituted 5-6 membered heterocycloalkyl-NHCO—, ((C₁-C₄)alkyl)((C₁-C₄)alkyl)-NCO—, (C₁-C₄)alkyl-CONH—, ((C₁-C₄)alkyl)((C₁-C₄)alkyl)N—NHCO—, —CO₂H, —CO₂(C₁-C₄)alkyl, (C₁-C₄)alkylthio-, phenyl-(C₁-C₄)alkylthio-, (C₁-C₄)alkyl-SO₂—, phenyl, optionally substituted 5-6 membered heterocycloalkyl, and optionally substituted 5-6 membered heteroaryl group,

wherein said optionally substituted (C₁-C₄)alkoxy is optionally substituted by hydroxyl, —CO₂H, —CONH₂, 5-6 membered heterocycloalkyl, or 5-6 membered heteroaryl; or said optionally substituted 5-6 membered heterocycloalkyl-CO—, optionally substituted 5-6 membered heterocycloalkyl, or optionally substituted 5-6 membered heteroaryl group is optionally substituted by (C₁-C₄)alkyl or oxo; or said optionally substituted 5-6 membered heterocycloalkyl-NHCO— is optionally substituted by (C₁-C₄)alkyl-CO—.

In another embodiment of Formula (I) or Formula (II), R¹ is a substituted or unsubstituted 5-6 membered heteroaryl group, wherein said substituted or unsubstituted 5-6 membered heteroaryl group is a substituted or unsubstituted pyrimidinyl, pyrazinyl, pyridazinyl, or pyridyl,

wherein said substituted pyrimidinyl, pyrazinyl, pyridazinyl, or pyridyl is substituted by 1 or 2 substituents independently selected from hydroxyl, cyano, halogen, (C₁-C₄)alkyl, halo(C₁-C₄)alkyl, hydroxy(C₁-C₄)alkyl, (C₂-C₄)alkynyl, optionally substituted (C₁-C₄)alkoxy, optionally substituted 5-6 membered heterocycloalkyl-CO—, fused 5-6 membered heterocycloalkyl; ((C₁-C₄)alkyl)-NH—, ((C₁-C₄)alkyl)((C₁-C₄)alkyl)N—, H₂NCO—, H₂NCO—(C₁-C₄)alkyl-, ((C₁-C₄)alkyl)NHCO—, (hydroxy-(C₁-C₄)alkyl)NHCO—, (C₃-C₆)cycloalkyl-NHCO—, optionally substituted 5-6 membered heterocycloalkyl-NHCO—, ((C₁-C₄)alkyl)((C₁-C₄)alkyl)-NCO—, (C₁-C₄)alkyl-CONH—, ((C₁-C₄)alkyl)((C₁-C₄)alkyl)N—NHCO—, —CO₂H, —CO₂(C₁-C₄)alkyl, (C₁-C₄)alkylthio-, phenyl-(C₁-C₄)alkylthio-, (C₁-C₄)alkyl-SO₂—, phenyl, optionally substituted 5-6 membered heterocycloalkyl, and optionally substituted 5-6 membered heteroaryl group,

wherein said optionally substituted (C₁-C₄)alkoxy is optionally substituted by hydroxyl, —CO₂H, —CONH₂, 5-6 membered heterocycloalkyl, or 5-6 membered heteroaryl; or said optionally substituted 5-6 membered heterocycloalkyl-CO—, optionally substituted 5-6 membered heterocycloalkyl, or optionally substituted 5-6 membered heteroaryl group is optionally substituted by (C₁-C₄)alkyl or oxo; or said optionally substituted 5-6 membered heterocycloalkyl-NHCO— is optionally substituted by (C₁-C₄)alkyl-CO—;

or a pharmaceutically acceptable salt thereof.

In another embodiment of Formula (I) or Formula (II), R¹ is a substituted or unsubstituted 5-6 membered heteroaryl group, wherein said substituted or unsubstituted 5-6 membered heteroaryl group is a substituted or unsubstituted pyrimidinyl, pyrazinyl, pyridazinyl, or pyridyl,

wherein said substituted pyrimidinyl, pyrazinyl, pyridazinyl, or pyridyl is substituted by 1 or 2 substituents independently selected from cyano, halogen, (C₁-C₄)alkyl, H₂N—, H₂NCO—, and —CO₂H; or a pharmaceutically acceptable salt thereof.

In another embodiment, R¹ is a substituted or unsubstituted 5-6 membered heteroaryl group, wherein said substituted or unsubstituted 5-6 membered heteroaryl group is a substituted or unsubstituted pyrimidinyl,

wherein said substituted pyrimidinyl is substituted by 1 or 2 substituents independently selected from cyano, halogen, (C₁-C₄)alkyl, H₂NCO—, and —CO₂H; or a pharmaceutically acceptable salt thereof.

In one embodiment, R¹ is substituted or unsubstituted 5-6 membered heteroaryl group, wherein said substituted or unsubstituted 5-6 membered heteroaryl group is a pyrimidinyl. In specific embodiments, R¹ is a substituted 2,4-disubstituted pyrimidinyl, 4,6-disubstituted pyrimidinyl, 2,5-disubstituted pyrimidinyl, 4,5-disubstituted pyrimidinyl, 2,4,5-trisubstituted pyrimidinyl, 2,4,6-trisubstituted pyrimidinyl, or 4,5,6-trisubstituted pyrimidinyl.

More specifically, when R¹ is a 2,4-disubstituted pyrimidinyl, R¹ is a 2-substituted pyrimidin-4-yl or a 4-substituted pyrimidin-2-yl; when R¹ is a 4,6-disubstituted pyrimidinyl, R¹ is a 4-substituted pyrimidin-6-yl or a 6-substituted pyrimidin-4-yl; when R¹ is a 2,5-disubstituted pyrimidinyl, R¹ is a 2-substituted pyrimidin-5-yl or a 5-substituted pyrimidin-2-yl; when R¹ is a 4,5-disubstituted pyrimidinyl, R¹ is a 4-substituted pyrimidin-5-yl or a 5-substituted pyrimidin-4-yl; when R¹ is a 2,4,5-trisubstituted pyrimidinyl, R¹ is a 2,4-disubstituted pyrimidin-5-yl, a 2,5-disubstituted pyrimidin-4-yl, or a 4,5-disubstituted pyrimidin-2-yl; when R¹ is a 2,4,6-trisubstituted pyrimidinyl, R¹ is a 2,4-disubstituted pyrimidin-6-yl, a 2,6-disubstituted pyrimidin-4-yl, or a 4,6-disubstituted pyrimidin-2-yl; and when R¹ is a 4,5,6-trisubstituted pyrimidinyl, R¹ is a 4,5-disubstituted pyrimidin-6-yl, a 4,6-disubstituted pyrimidin-5-yl, or a 5,6-disubstituted pyrimidin-4-yl.

In one embodiment of Formula (I) or Formula (II), R¹ is substituted or unsubstituted 5-6 membered heteroaryl group, wherein said substituted or unsubstituted 5-6 membered heteroaryl group is a pyrimidinyl. In specific embodiments, R¹ is a substituted or unsubstituted pyimidin-2-yl, pyrimidin-4-yl, pyrimidin-5-yl, or pyrimidin-6-yl. In specific embodiments, R¹ is a 4-substituted pyimidin-2-yl, 2-substituted pyrimidin-4-yl, 2-substituted pyrimidin-5-yl, 5-substituted pyrimidin-2-yl, 4-substituted pyrimidin-6-yl, 6-substituted pyrimidin-4-yl, 4-substituted pyrimidin-5-yl, 5-substituted pyrimidin-4-yl, 2,4-disubstituted pyrimidin-5-yl, 2,5-disubstituted pyrimidin-4-yl, 4,5-disubstituted pyrimidin-2-yl, 2,4-disubstituted pyrimidin-6-yl, 2,6-disubstituted pyrimidin-4-yl, 4,6-disubstituted pyrimidin-2-yl, 2,4-disubstituted pyrimidin-6-yl, 2,6-disubstituted pyrimidin-4-yl, 4,6-disubstituted pyrimidin-2-yl, 4,5-disubstituted pyrimidin-6-yl, 4,6-disubstituted pyrimidin-5-yl, or 5,6-disubstituted pyrimidin-4-yl.

In other embodiments of the compounds of Formula (I) and Formula (II), R¹ is a substituted or unsubstituted 5-6 membered heteroaryl group, wherein said substituted or unsubstituted 5-6 membered heteroaryl group is a pyrimidinyl group,

wherein said substituted pyrimidinyl group is a 2,4-disubstituted pyrimidinyl group, substituted by hydroxyl, cyano, halogen, (C₁-C₄)alkyl, halo(C₁-C₄)alkyl, hydroxy(C₁-C₄)alkyl, (C₂-C₄)alkynyl, (C₁-C₄)alkoxy, optionally substituted (C₁-C₄)alkoxy, optionally substituted 5-6 membered heterocycloalkyl-CO—, fused 5-6 membered heterocycloalkyl; H₂N—, ((C₁-C₄)alkyl)-NH—, ((C₁-C₄)alkyl)((C₁-C₄)alkyl)N—, H₂NCO—, H₂NCO—(C₁-C₄)alkyl-, ((C₁-C₄)alkyl)NHCO—, (hydroxy-(C₁-C₄)alkyl)NHCO—, (C₃-C₆)cycloalkyl-NHCO—, optionally substituted 5-6 membered heterocycloalkyl-NHCO—, ((C₁-C₄)alkyl)((C₁-C₄)alkyl)-NCO—, (C₁-C₄)alkyl-CONH—, ((C₁-C₄)alkyl)((C₁-C₄)alkyl)N—NHCO—, —CO₂H, —CO₂(C₁-C₄)alkyl, (C₁-C₄)alkylthio-, phenyl-(C₁-C₄)alkylthio-, (C₁-C₄)alkyl-SO₂—, phenyl, optionally substituted 5-6 membered heterocycloalkyl, and optionally substituted 5-6 membered heteroaryl group,

wherein said optionally substituted (C₁-C₄)alkoxy is optionally substituted by hydroxyl, —CO₂H, —CONH₂, 5-6 membered heterocycloalkyl, or 5-6 membered heteroaryl; or said optionally substituted 5-6 membered heterocycloalkyl-CO—, optionally substituted 5-6 membered heterocycloalkyl, or optionally substituted 5-6 membered heteroaryl group is optionally substituted by (C₁-C₄)alkyl or oxo; or said optionally substituted 5-6 membered heterocycloalkyl-NHCO— is optionally substituted by (C₁-C₄)alkyl-CO—.

In other embodiments of the compounds of Formula (I) or Formula (II), R¹ is a substituted or unsubstituted 5-6 membered heteroaryl group, wherein said substituted or unsubstituted 5-6 membered heteroaryl group is a pyrimidinyl,

wherein said substituted pyrimidinyl is a pyrimidin-2-yl substituted at the 4-position or a pyrimidin-4-yl substituted at the 2-position by hydroxyl, cyano, halogen, (C₁-C₄)alkyl, halo(C₁-C₄)alkyl, hydroxy(C₁-C₄)alkyl, (C₂-C₄)alkynyl, (C₁-C₄)alkoxy, optionally substituted (C₁-C₄)alkoxy, optionally substituted 5-6 membered heterocycloalkyl-CO—, fused 5-6 membered heterocycloalkyl; H₂N—, ((C₁-C₄)alkyl)-NH—, ((C₁-C₄)alkyl)((C₁-C₄)alkyl)N—, H₂NCO—, H₂NCO—(C₁-C₄)alkyl-, ((C₁-C₄)alkyl)NHCO—, (hydroxy-(C₁-C₄)alkyl)NHCO—, (C₃-C₆)cycloalkyl-NHCO—, optionally substituted 5-6 membered heterocycloalkyl-NHCO—, ((C₁-C₄)alkyl)((C₁-C₄)alkyl)-NCO—, (C₁-C₄)alkyl-CONH—, ((C₁-C₄)alkyl)((C₁-C₄)alkyl)N—NHCO—, —CO₂H, —CO₂(C₁-C₄)alkyl, (C₁-C₄)alkylthio-, phenyl-(C₁-C₄)alkylthio-, (C₁-C₄)alkyl-SO₂—, phenyl, optionally substituted 5-6 membered heterocycloalkyl, or optionally substituted 5-6 membered heteroaryl group,

wherein said optionally substituted (C₁-C₄)alkoxy is optionally substituted by hydroxyl, —CO₂H, —CONH₂, 5-6 membered heterocycloalkyl, or 5-6 membered heteroaryl; or said optionally substituted 5-6 membered heterocycloalkyl-CO—, optionally substituted 5-6 membered heterocycloalkyl, or optionally substituted 5-6 membered heteroaryl group is optionally substituted by (C₁-C₄)alkyl or oxo; or said optionally substituted 5-6 membered heterocycloalkyl-NHCO— is optionally substituted by (C₁-C₄)alkyl-CO—.

In another embodiment of the compounds of Formula (I) or Formula (II), R¹ is a substituted 5-6 membered heteroaryl group, wherein said substituted 5-6 membered heteroaryl group is substituted pyrimidinyl, wherein said substituted pyrimidinyl group is a 2,4-disubstituted pyrimidinyl group substituted at the 2-position of the pyrimidinyl by cyano, (C₁-C₄)alkoxy, optionally substituted (C₁-C₄)alkoxy, ((C₁-C₄)alkyl)((C₁-C₄)alkyl)N—, or (C₁-C₄)alkylthio-, wherein said optionally substituted (C₁-C₄)alkoxy is optionally substituted by —CO₂H.

In another embodiment of the compounds of Formula (I) or Formula (II), R¹ is a substituted 5-6 membered heteroaryl group, wherein said substituted 5-6 membered heteroaryl group is a substituted pyrimidinyl, wherein said substituted pyrimidinyl is a pyrimidin-4-yl substituted at the 2-position by cyano, (C₁-C₄)alkoxy, optionally substituted (C₁-C₄)alkoxy, ((C₁-C₄)alkyl)((C₁-C₄)alkyl)N—, H₂NCO—, or (C₁-C₄)alkylthio-, wherein said optionally substituted (C₁-C₄)alkoxy is optionally substituted by —CO₂H.

In more specific embodiments, R¹ is a substituted 5-6 membered heteroaryl group, wherein said substituted 5-6 membered heteroaryl group is a substituted pyrimidinyl, wherein said substituted pyrimidinyl is a 2,4-disubstituted pyrimidinyl substituted at the 2-position of the pyrimidinyl by cyano, methoxy, HOC₂CH₂O—, dimethylamine, or CH₃S—.

In more specific embodiments, R¹ is a substituted 5-6 membered heteroaryl group, wherein said substituted 5-6 membered heteroaryl group is a substituted pyrimidinyl, wherein said substituted pyrimidinyl is a pyrimidin-4-yl substituted at the 2-position by cyano, methoxy, HOC₂CH₂O—, dimethylamine, H₂NCO—, or CH₃S—.

In another embodiment, R¹ is substituted or unsubstituted 5-6 membered heteroaryl group, wherein said substituted or unsubstituted 5-6 membered heteroaryl group is a pyrimidinyl, wherein said substituted pyrimidinyl group is a 2,4-disubstituted pyrimidinyl group. In another embodiment, R¹ is a substituted 5-6 membered heteroaryl group, wherein said substituted 5-6 membered heteroaryl group is a pyrimidinyl group, wherein said substituted pyrimidinyl group is a 2,4-disubstituted pyrimidinyl group substituted at the 4-position of the pyrimidinyl ring by H₂NCO—.

In another embodiment, R¹ is substituted or unsubstituted 5-6 membered heteroaryl group, wherein said substituted or unsubstituted 5-6 membered heteroaryl group is a pyrimidinyl, wherein said substituted pyrimidinyl is a pyrimidin-4-yl substituted at the 2-position by H₂NCO—. In another embodiment, R¹ is substituted or unsubstituted 5-6 membered heteroaryl group, wherein said substituted or unsubstituted 5-6 membered heteroaryl group is a pyrimidinyl, wherein said substituted pyrimidinyl is a pyrimidin-2-yl substituted at the 4-position by H₂NCO—.

In another embodiment of the compounds of Formula (I) and Formula (II), R¹ is a substituted 5-6 membered heteroaryl group, wherein said substituted 5-6 membered heteroaryl group is a substituted pyrimidinyl, wherein said substituted pyrimidinyl is a 2,4-disubstituted pyrimidinyl substituted at the 4-position of the pyrimidinyl by hydroxyl, cyano, hydroxy(C₁-C₄)alkyl, (C₁-C₄)alkoxy, optionally substituted (C₁-C₄)alkoxy, optionally substituted 5-6 membered heterocycloalkyl-CO—, H₂N—, ((C₁-C₄)alkyl)-NH—, H₂NCO—, (C₁-C₄)alkyl-CONH—, (hydroxy-(C₁-C₄)alkyl)NHCO—, (C₃-C₆)cycloalkyl-NHCO—, optionally substituted 5-6 membered heterocycloalkyl-NHCO—, ((C₁-C₄)alkyl)((C₁-C₄)alkyl)-NCO—, ((C₁-C₄)alkyl)((C₁-C₄)alkyl)N—NHCO—, —CO₂H, phenyl-(C₁-C₄)alkylthio-, phenyl, optionally substituted 5-6 membered heterocycloalkyl, or optionally substituted 5-6 membered heteroaryl group.

In another embodiment of the compounds of Formula (I) or Formula (II), R¹ is a substituted 5-6 membered heteroaryl group, wherein said substituted 5-6 membered heteroaryl group is a substituted pyrimidinyl, wherein said substituted pyrimidinyl is a pyrimidin-2-yl substituted at the 4-position by hydroxyl, cyano, hydroxy(C₁-C₄)alkyl, (C₁-C₄)alkoxy, optionally substituted (C₁-C₄)alkoxy, optionally substituted 5-6 membered heterocycloalkyl-CO—, H₂N—, ((C₁-C₄)alkyl)-NH—, H₂NCO—, (C₁-C₄)alkyl-CONH—, (hydroxy-(C₁-C₄)alkyl)NHCO—, (C₃-C₆)cycloalkyl-NHCO—, optionally substituted 5-6 membered heterocycloalkyl-NHCO—, ((C₁-C₄)alkyl)((C₁-C₄)alkyl)-NCO—, ((C₁-C₄)alkyl)((C₁-C₄)alkyl)N—NHCO—, —CO₂H, phenyl-(C₁-C₄)alkylthio-, phenyl, optionally substituted 5-6 membered heterocycloalkyl, or optionally substituted 5-6 membered heteroaryl group.

In more specific embodiments, R¹ is a substituted 5-6 membered heteroaryl group, wherein said substituted 5-6 membered heteroaryl group is a substituted pyrimidinyl, wherein said substituted pyrimidinyl is a 2,4-disubstituted pyrimidinyl substituted at the 4-position of the pyrimidinyl by hydroxyl, cyano, HO—CH₂—, methoxy, ethoxy, HO₂CCH₂O—, morpholine-CO—, piperazine-CO—, N-methylpiperazine-CO, H₂N—, CH₃NH—, H₂NCO—, HO—CH₂CH₂—NHCO—, cyclopropyl-NHCO, H₂NCO—, CH₃CONH—, N-acetyl-piperidine-NHCO—, (CH₃CH₂)(CH₃CH₂)N—CO—, N′,N′-dimethylhydrazine-CO—, —CO₂H, benzyl-SH—, phenyl, dihydroimidazole, morpholine, or tetrazole.

In more specific embodiments, R¹ is a substituted 5-6 membered heteroaryl group, wherein said substituted 5-6 membered heteroaryl group is a substituted pyrimidinyl, wherein said substituted pyrimidinyl is a pyrimidin-2-yl substituted at the 4-position by hydroxyl, cyano, HO—CH₂—, methoxy, ethoxy, HO₂CCH₂O—, morpholine-CO—, piperazine-CO—, N-methylpiperazine-CO, H₂N—, CH₃NH—, H₂NCO—, HO—CH₂CH₂—NHCO—, cyclopropyl-NHCO, H₂NCO—, CH₃CONH—, N-acetyl-piperidine-NHCO—, (CH₃CH₂)(CH₃CH₂)N—CO—, N′,N′-dimethylhydrazine-CO—, —CO₂H, benzyl-SH—, phenyl, dihydroimidazole, morpholine, or tetrazole.

In more specific embodiments, R¹ is a substituted 5-6 membered heteroaryl group, wherein said substituted 5-6 membered heteroaryl group is a substituted pyrimidinyl, wherein said substituted pyrimidinyl is a 2,4-disubstituted pyrimidinyl substituted at the 4-position of the pyrimidinyl by H₂NCO—.

In more specific embodiments, R¹ is a substituted 5-6 membered heteroaryl group, wherein said substituted 5-6 membered heteroaryl group is a pyrimidinyl, wherein said substituted pyrimidinyl is a pyrimidin-2-yl substituted at the 4-position by H₂NCO—.

In other embodiments of the compounds of Formula (I) or Formula (II), R¹ is substituted or unsubstituted 5-6 membered heteroaryl group, wherein said substituted or unsubstituted 5-6 membered heteroaryl group is a pyrimidinyl,

wherein said substituted pyrimidinyl is a 4,6-disubstituted pyrimidinyl, substituted by hydroxyl, cyano, halo(C₁-C₄)alkyl, (C₂-C₄)alkynyl, (C₁-C₄)alkoxy, H₂N—, H₂NCO—, (C₁-C₄)alkyl-CONH—, (C₁-C₄)alkylthio-, and optionally substituted 5-6 membered heteroaryl group, wherein said optionally substituted 5-6 membered heteroaryl group is optionally substituted by (C₁-C₄)alkyl.

In other embodiments of the compounds of Formula (I) or Formula (II), R¹ is substituted or unsubstituted 5-6 membered heteroaryl group, wherein said substituted or unsubstituted 5-6 membered heteroaryl group is a pyrimidinyl,

wherein said substituted pyrimidinyl is a pyrimidin-4-yl substituted at the 6-position or a pyrimidin-6-yl substituted at the 4-position by hydroxyl, cyano, halo(C₁-C₄)alkyl, (C₂-C₄)alkynyl, (C₁-C₄)alkoxy, H₂N—, H₂NCO—, (C₁-C₄)alkyl-CONH—, (C₁-C₄)alkylthio-, and optionally substituted 5-6 membered heteroaryl group, wherein said optionally substituted 5-6 membered heteroaryl group is optionally substituted by (C₁-C₄)alkyl.

In another embodiment, R¹ is substituted or unsubstituted 5-6 membered heteroaryl group, wherein said substituted or unsubstituted 5-6 membered heteroaryl group is a pyrimidinyl, wherein said substituted pyrimidinyl is a 4,6-disubstituted pyrimidinyl group substituted by cyano, H₂N—, or H₂NCO—.

In another embodiment, R¹ is substituted or unsubstituted 5-6 membered heteroaryl group, wherein said substituted or unsubstituted 5-6 membered heteroaryl group is a pyrimidinyl, wherein said substituted pyrimidinyl is a pyrimidin-4-yl substituted at the 6-position or a pyrimidin-6-yl substituted at the 4-position by cyano, H₂N—, or H₂NCO—.

In other embodiments of the compounds of Formula (I) and Formula (II), R¹ is substituted or unsubstituted 5-6 membered heteroaryl group, wherein said substituted or unsubstituted 5-6 membered heteroaryl group is a pyrimidinyl, wherein said substituted pyrimidinyl is a 2,5-disubstituted pyrimidinyl, substituted by hydroxyl, cyano, halogen, (C₁-C₄)alkoxy, H₂NCO—, —CO₂(C₁-C₄)alkyl, or (C₁-C₄)alkyl-SO₂—.

In other embodiments of the compounds of Formula (I) or Formula (II), R¹ is substituted or unsubstituted 5-6 membered heteroaryl group, wherein said substituted or unsubstituted 5-6 membered heteroaryl group is a pyrimidinyl, wherein said substituted pyrimidinyl is a pyrimidin-2-yl substituted at the 5-position or a pyrimidin-5-yl substituted at the 2-position by hydroxyl, cyano, halogen, (C₁-C₄)alkoxy, H₂NCO—, —CO₂(C₁-C₄)alkyl, or (C₁-C₄)alkyl-SO₂—.

In one embodiment, R¹ is substituted or unsubstituted 5-6 membered heteroaryl group, wherein said substituted or unsubstituted 5-6 membered heteroaryl group is a pyrimidinyl, wherein the said substituted pyrimidinyl is a 2,5-disubstituted pyrimidinyl substituted by H₂NCO—.

In one embodiment, R¹ is substituted or unsubstituted 5-6 membered heteroaryl group, wherein said substituted or unsubstituted 5-6 membered heteroaryl group is a pyrimidinyl, wherein the said substituted pyrimidinyl is a pyrimidin-2-yl substituted at the 5-position or a pyrimidin-5-yl substituted at the 2-position by H₂NCO—.

In another embodiment of the compounds of Formula (I) or Formula (II), R¹ is a substituted 5-6 membered heteroaryl group, wherein said substituted 5-6 membered heteroaryl group is a pyrimidinyl, wherein said substituted pyrimidinyl is a 2,5-disubstituted pyrimidinyl substituted at the 5-position of the pyrimidinyl ring by hydroxyl, cyano, halogen, (C₁-C₄)alkoxy, H₂NCO—, —CO₂(C₁-C₄)alkyl, or (C₁-C₄)alkyl-SO₂—.

In another embodiment of the compounds of Formula (I) or Formula (II), R¹ is a substituted 5-6 membered heteroaryl group, wherein said substituted 5-6 membered heteroaryl group is a pyrimidinyl, wherein said substituted pyrimidinyl is a pyrimidin-2-yl substituted at the 5-position by hydroxyl, cyano, halogen, (C₁-C₄)alkoxy, H₂NCO—, —CO₂(C₁-C₄)alkyl, or (C₁-C₄)alkyl-SO₂—.

In specific embodiments, R¹ is a substituted 5-6 membered heteroaryl group, wherein said substituted 5-6 membered heteroaryl group is a substituted pyrimidinyl, wherein said substituted pyrimidinyl is a 2,5-disubstituted pyrimidinyl substituted at the 5-position of the pyrimidinyl by hydroxyl, cyano, fluoro, methoxy, H₂NCO—, —CO₂CH₃, or CH₃—SO₂—.

In specific embodiments, R¹ is a substituted 5-6 membered heteroaryl group, wherein said substituted 5-6 membered heteroaryl group is a pyrimidinyl, wherein said substituted pyrimidinyl is a pyrimidin-2-yl substituted at the 5-position by hydroxyl, cyano, fluoro, methoxy, H₂NCO—, —CO₂CH₃, or CH₃—SO₂—.

In one embodiment, R¹ is substituted or unsubstituted 5-6 membered heteroaryl group, wherein said substituted or unsubstituted 5-6 membered heteroaryl group is a pyrimidinyl, wherein the said substituted pyrimidinyl is a pyrimidin-2-yl substituted at the 5-position by H₂NCO—.

In one embodiment of the compounds of Formula (I) or Formula (II), R¹ is substituted or unsubstituted 5-6 membered heteroaryl group, wherein said substituted or unsubstituted 5-6 membered heteroaryl group is a pyrimidinyl, wherein said substituted pyrimidinyl is a 4,5-disubstituted pyrimidinyl group substituted by cyano.

In one embodiment of the compounds of Formula (I) or Formula (II), R¹ is substituted or unsubstituted 5-6 membered heteroaryl group, wherein said substituted or unsubstituted 5-6 membered heteroaryl group is a pyrimidinyl, wherein said substituted pyrimidinyl is a pyrimidin-4-yl substituted at the 5-position or a pyrimidin-5-yl substituted at the 4-position by cyano.

In other embodiments of the compounds of Formula (I) or Formula (II), R¹ is substituted or unsubstituted 5-6 membered heteroaryl group, wherein said substituted or unsubstituted 5-6 membered heteroaryl group is a pyrimidinyl,

wherein said substituted pyrimidinyl is a 2,4,5-trisubstituted pyrimidinyl, wherein said 2,4,5-trisubstituted pyrimidinyl is substituted by 2 substituents independently selected from halogen, optionally substituted (C₁-C₄)alkoxy, H₂N—, H₂NCO—, H₂NCO—(C₁-C₄)alkyl-, and optionally substituted 5-6 membered heterocycloalkyl group,

wherein said optionally substituted (C₁-C₄)alkoxy is optionally substituted by hydroxyl, —CONH₂, 5-6 membered heterocycloalkyl, or 5-6 membered heteroaryl group, or optionally substituted 5-6 membered heterocycloalkyl is optionally substituted by oxo.

In other embodiments of the compounds of Formula (I) or Formula (II), R¹ is substituted or unsubstituted 5-6 membered heteroaryl group, wherein said substituted or unsubstituted 5-6 membered heteroaryl group is a pyrimidinyl,

wherein said substituted pyrimidinyl is a pyrimidin-2-yl substituted at the 4-position and 5-position by substituents independently selected from halogen, optionally substituted (C₁-C₄)alkoxy, H₂N—, H₂NCO—, H₂NCO—(C₁-C₄)alkyl-, and optionally substituted 5-6 membered heterocycloalkyl group,

wherein said optionally substituted (C₁-C₄)alkoxy is optionally substituted by hydroxyl, —CONH₂, 5-6 membered heterocycloalkyl, or 5-6 membered heteroaryl group, or optionally substituted 5-6 membered heterocycloalkyl is optionally substituted by oxo.

In other embodiments of the compounds of Formula (I) or (II), R¹ is substituted or unsubstituted 5-6 membered heteroaryl group, wherein said substituted or unsubstituted 5-6 membered heteroaryl group is a pyrimidinyl,

wherein said substituted pyrimidinyl is a 2,4,5-trisubstituted pyrimidinyl,

wherein said 2,4,5-trisubstituted pyrimidinyl is substituted by a substituent in the 4-position of the pyrimidinyl and a substitutent in the 5-position of the pyrimidinyl,

wherein the 4-position of the pyrimidinyl is substituted by H₂N—, H₂NCO—, H₂NCO—(C₁-C₄)alkyl-, optionally substituted (C₁-C₄)alkoxy, or optionally substituted 5-6 membered heterocycloalkyl,

wherein optionally substituted (C₁-C₄)alkoxy is substituted by hydroxyl, —CONH₂, 5-6 membered heterocycloalkyl, or 5-6 membered heteroaryl, or optionally substituted 5-6 membered heterocycloalkyl is optionally substituted by oxo.

wherein the 5-position is substituted by halogen.

In other embodiments of the compounds of Formula (I) or Formula (II), R¹ is substituted or unsubstituted 5-6 membered heteroaryl group, wherein said substituted or unsubstituted 5-6 membered heteroaryl group is a pyrimidinyl,

wherein said substituted pyrimidinyl is a pyrimidin-2-yl substituted at the 4-position and 5-position,

wherein the 4-position of the pyrimidin-2-yl is substituted by H₂N—, H₂NCO—, H₂NCO—(C₁-C₄)alkyl-, optionally substituted (C₁-C₄)alkoxy, or optionally substituted 5-6 membered heterocycloalkyl,

wherein optionally substituted (C₁-C₄)alkoxy is substituted by hydroxyl, —CONH₂, 5-6 membered heterocycloalkyl, or 5-6 membered heteroaryl, or optionally substituted 5-6 membered heterocycloalkyl is optionally substituted by oxo; and

wherein the 5-position of the pyrimidin-2-yl is substituted by halogen.

In specific embodiments, R¹ is a substituted or unsubstituted 5-6 membered heteroaryl group, wherein said substituted or unsubstituted 5-6 membered heteroaryl group is a pyrimidinyl,

wherein said substituted pyrimidinyl group is a 2,4,5-trisubstituted pyrimidinyl,

wherein said 2,4,5-trisubstituted pyrimidinyl is substituted by a substituent in the 4-position of the pyrimidinyl and a substitutent in the 5-position of the pyrimidinyl,

wherein the 4-position of the pyrimidinyl is substituted by H₂N—, H₂NCO—, H₂N—CO—CH₂—, pyrrolidinone, N-methylpiperazine, HO—CH₂CH₂—O—, H₂NCO—CH₂—O—, morpholine-CH₂CH₂—O—, tetrazolyl-CH₂—O—, or pyrrolidin-2-one; and

wherein the 5-position of the pyrimidinyl is substituted by fluoro or chloro.

In specific embodiments, R¹ is a substituted or unsubstituted 5-6 membered heteroaryl group, wherein said substituted or unsubstituted 5-6 membered heteroaryl group is a pyrimidinyl,

wherein said substituted pyrimidinyl is a pyrimidin-2-yl substituted at the 4-position and 5-position,

wherein the 4-position of the pyrimidin-2-yl is substituted by H₂N—, H₂NCO—, H₂N—CO—CH₂—, pyrrolidinone, N-methylpiperazine, HO—CH₂CH₂—O—, H₂NCO—CH₂—O—, morpholine-CH₂CH₂—O—, tetrazolyl-CH₂—O—, or pyrrolidin-2-one; and

wherein the 5-position of the pyrimidin-2-yl is substituted by fluoro or chloro.

In other embodiments of the compounds of Formulas (I) or Formula (II), R¹ is substituted or unsubstituted 5-6 membered heteroaryl group, wherein said substituted or unsubstituted 5-6 membered heteroaryl group is a pyrimidinyl,

wherein said substituted pyrimidinyl is a 2,4,6-trisubstituted pyrimidinyl, whereby said 2,4,6-trisubstituted pyrimidinyl is substituted by 2 substituents independently selected from (C₁-C₄)alkyl, (C₁-C₄)alkoxy, H₂N—, or (C₁-C₄)alkylthio-.

In other embodiments of the compounds of Formula (I) or Formula (II), R¹ is substituted or unsubstituted 5-6 membered heteroaryl group, wherein said substituted or unsubstituted 5-6 membered heteroaryl group is a pyrimidinyl,

wherein said substituted pyrimidinyl is a pyrimidin-2-yl, pyrimidin-4-yl, or pyrimidin-6-yl substituted by 2 substituents independently selected from halogen, (C₁-C₄)alkyl, (C₁-C₄)alkoxy, H₂N—, and (C₁-C₄)alkylthio-.

In other embodiments of the compounds of Formulas (I), (II), and (III), R¹ is substituted or unsubstituted 5-6 membered heteroaryl group, wherein said substituted or unsubstituted 5-6 membered heteroaryl group is a pyrimidinyl, wherein said substituted pyrimidinyl is a 4,5,6-trisubstituted pyrimidinyl,

wherein said 4,5,6-trisubstituted pyrimidinyl is substituted by 2 substituents independently selected from halogen, H₂N—, H₂NCO—, ((C₁-C₄)alkyl)((C₁-C₄)alkyl)N—CO—, —CO₂H.

In other embodiments of the compounds of Formula (I) or Formula (II), R¹ is substituted or unsubstituted 5-6 membered heteroaryl group, wherein said substituted or unsubstituted 5-6 membered heteroaryl group is a pyrimidinyl, wherein said substituted pyrimidinyl is a pyrimidin-4-yl, pyrimidin-5-yl, or pyrimidin-6-yl substituted by 2 substituents independently selected from halogen, H₂N—, H₂NCO—, ((C₁-C₄)alkyl)((C₁-C₄)alkyl)N—CO—, and —CO₂H.

In other embodiments, R¹ is substituted or unsubstituted 5-6 membered heteroaryl group, wherein said substituted or unsubstituted 5-6 membered heteroaryl group is a pyrimidinyl, wherein said substituted pyrimidinyl is a 4,5,6-trisubstituted pyrimidinyl, wherein said 4,5,6-trisubstituted pyrimidinyl is substituted by 2 substituents selected from halogen and —CO₂H.

In other embodiments, R¹ is substituted or unsubstituted 5-6 membered heteroaryl group, wherein said substituted or unsubstituted 5-6 membered heteroaryl group is a pyrimidinyl, wherein said substituted pyrimidinyl is a pyrimidin-4-yl, pyrimidin-5-yl, or pyrimidin-6-yl substituted by 2 substituents independently selected from halogen and —CO₂H.

In other embodiments, R¹ is a substituted or unsubstituted 5-6 membered heteroaryl group, wherein said substituted or unsubstituted 5-6 membered heteroaryl group is a pyrimidinyl, wherein said substituted pyrimidinyl is a 4,5,6-trisubstituted pyrimidinyl, wherein the 4-position or 6-position of the pyrimidinyl ring is substituted by H₂N, H₂NCO—, ((C₁-C₄)alkyl)((C₁-C₄)alkyl)N—CO—, or —CO₂H; and wherein the 5-position of the pyrimidinyl ring is substituted by halogen.

In other embodiments, R¹ is a substituted or unsubstituted 5-6 membered heteroaryl group, wherein said substituted or unsubstituted 5-6 membered heteroaryl group is a pyrimidinyl, wherein said substituted pyrimidinyl is a pyrimin-4-yl substituted at the 6-position or a pyrimin-6-yl substituted at the 4-position by H₂N, H₂NCO—, ((C₁-C₄)alkyl)((C₁-C₄)alkyl)N—CO—, or —CO₂H; and wherein the 5-position of the pyrimin-4-yl or pyrimin-6-yl is substituted by halogen.

In other embodiments, R¹ is a substituted or unsubstituted 5-6 membered heteroaryl group, wherein said substituted or unsubstituted 5-6 membered heteroaryl group is a pyrimidinyl, wherein said substituted pyrimidinyl is a 4,5,6-trisubstituted pyrimidinyl, wherein the 4-position or the 6-position of the pyrimidinyl is substituted by —CO₂H; and wherein the 5-position of the pyrimidinyl is substituted by halogen.

In other embodiments, R¹ is a substituted or unsubstituted 5-6 membered heteroaryl group, wherein said substituted or unsubstituted 5-6 membered heteroaryl group is a pyrimidinyl, wherein said substituted pyrimidinyl is a pyrimin-4-yl substituted at the 6-position or a pyrimin-6-yl substituted at the 4-position by —CO₂H; and wherein the 5-position of the pyrimin-4-yl or pyrimin-6-yl is substituted by halogen.

In specific embodiments, R¹ is a substituted or unsubstituted 5-6 membered heteroaryl group, wherein said substituted or unsubstituted 5-6 membered heteroaryl group is a pyrimidinyl, wherein said substituted pyrimidinyl is a 4,5,6-trisubstituted pyrimidinyl, wherein the 4-position or the 6-position of the pyrimidinyl is substituted by H₂N, H₂NCO—, (CH₃CH₂)(CH₃CH₂)N—CO—, or —CO₂H; and wherein the 5-position of the pyrimidinyl is substituted by fluoro.

In specific embodiments, R¹ is a substituted or unsubstituted 5-6 membered heteroaryl group, wherein said substituted or unsubstituted 5-6 membered heteroaryl group is a pyrimidinyl, wherein said substituted pyrimidinyl is a pyrimin-4-yl substituted at the 6-position or a pyrimin-6-yl substituted at the 4-position by H₂N, H₂NCO—, (CH₃CH₂)(CH₃CH₂)N—CO—, or —CO₂H; and wherein the 5-position of the pyrimin-4-yl or pyrimin-6-yl is substituted by fluoro.

In specific embodiments, R¹ is a substituted or unsubstituted 5-6 membered heteroaryl group, wherein said substituted or unsubstituted 5-6 membered heteroaryl group is a pyrimidinyl group, wherein said substituted pyrimidinyl is a 4,5,6-trisubstituted pyrimidinyl, wherein the 4-position or the 6-position of the pyrimidinyl is substituted by —CO₂H; and wherein the 5-position of the pyrimidinyl is substituted by fluoro.

In specific embodiments, R¹ is a substituted or unsubstituted 5-6 membered heteroaryl group, wherein said substituted or unsubstituted 5-6 membered heteroaryl group is a pyrimidinyl, wherein said substituted pyrimidinyl is a pyrimin-4-yl substituted at the 6-position or a pyrimin-6-yl substituted at the 4-position by —CO₂H; and wherein the 5-position of the pyrimin-4-yl or pyrimin-6-yl is substituted by fluoro.

In other embodiments of the compounds of Formulas (I) and (II), R¹ is substituted or unsubstituted 5-6 membered heteroaryl group, wherein said substituted or unsubstituted 5-6 membered heteroaryl group is a pyrazinyl, wherein said substituted pyrazinyl is substituted by cyano, (C₁-C₄)alkyl, (C₁-C₄)alkoxy, H₂NCO—, (C₁-C₄)alkyl-CONH, or phenyl.

In other embodiments, R¹ is substituted or unsubstituted 5-6 membered heteroaryl group, wherein said substituted or unsubstituted 5-6 membered heteroaryl group is a pyrizinyl, wherein said pyrizinyl is a 2,6-disubstituted pyrazinyl, wherein said 2,6-pyrazinyl is substituted in the 2-position of the pyrizinyl and the 6-position of the pyrazinyl, wherein the 2-position of the pyrazinyl or the 6-position of the pyrazinyl is substituted by cyano, H₂NCO—, or (C₁-C₄)alkyl-CONH—, or phenyl.

In other embodiments, R¹ is substituted or unsubstituted 5-6 membered heteroaryl group, wherein said substituted or unsubstituted 5-6 membered heteroaryl group is a pyrazinyl, wherein said pyrazinyl is a pyrazin-2-yl substituted at the 6-position or pyrazin-6-yl substituted at the 2-position by cyano, H₂NCO—, or (C₁-C₄)alkyl-CONH—, or phenyl.

In specific embodiments, R¹ is substituted or unsubstituted 5-6 membered heteroaryl group, wherein said substituted or unsubstituted 5-6 membered heteroaryl group is a pyrizinyl, wherein said pyrizinyl is a 2,6-disubstituted pyrazinyl, wherein said substituted pyrizinyl is substituted in the 2-position of the pyrizinyl and the 6-position of the pyrazinyl group, wherein the 2-position of the pyrazinyl or the 6-position of the pyrazinyl is substituted by cyano, H₂NCO—, CH₃CONH—, or phenyl.

In specific embodiments, R¹ is substituted or unsubstituted 5-6 membered heteroaryl group, wherein said substituted or unsubstituted 5-6 membered heteroaryl group is a pyrazinyl, wherein said pyrazinyl is a pyrazin-2-yl substituted at the 6-position or pyrazin-6-yl substituted at the 2-position by cyano, H₂NCO—, CH₃CONH—, or phenyl.

In other embodiments, R¹ is substituted or unsubstituted 5-6 membered heteroaryl group, wherein said substituted or unsubstituted 5-6 membered heteroaryl group is a pyrizinyl, wherein said substituted pyrizine group is a 2,5-disubstituted pyrizinyl, wherein the 2-position of the 2,5-disubstituted pyrazinyl group or the 5-position of the 2,5-disubstituted pyrazinyl group is substituted by cyano or H₂NCO—.

In other embodiments, R¹ is substituted or unsubstituted 5-6 membered heteroaryl group, wherein said substituted or unsubstituted 5-6 membered heteroaryl group is a pyrazinyl, wherein said substituted pyrazinyl is a pyrazin-2-yl substituted at the 5-position or pyrazin-5-yl substituted at the 2-position by cyano or H₂NCO—.

In other embodiments, R¹ is substituted or unsubstituted 5-6 membered heteroaryl group, wherein said substituted or unsubstituted 5-6 membered heteroaryl group is a pyrizinyl, wherein said pyrizinyl is 2,3-disubstituted pyrazinyl, wherein said substituted pyrizinyl group is substituted in the 2-position of the pyrizinyl and the 3-position of the pyrazinyl group, wherein the 2-position of the pyrazinyl group or the 3-position of the pyrazinyl group is substituted by cyano.

In other embodiments, R¹ is substituted or unsubstituted 5-6 membered heteroaryl group, wherein said substituted or unsubstituted 5-6 membered heteroaryl group is a pyrazinyl, wherein said pyrazinyl is a pyrazin-2-yl substituted at the 3-position or pyrazin-3-yl substituted at the 2-position by cyano.

In other embodiments of the compounds of Formula (I) or Formula (II), R¹ is a substituted or unsubstituted 5-6 membered heteroaryl group, wherein said substituted or unsubstituted 5-6 membered heteroaryl group is a pyridazinyl, wherein said substituted pyridazinyl is substituted by cyano, halogen, halo(C₁-C₄)alkyl, (C₁-C₄)alkoxy, or H₂NCO—.

In other embodiments, R¹ is a substituted or unsubstituted 5-6 membered heteroaryl group, wherein said substituted or unsubstituted 5-6 membered heteroaryl group is a pyridazinyl, wherein said pyridazinyl is 3,6-disubstituted pyridazinyl, wherein said 3,6-disubstituted pyridazinyl is substituted at the 3-position of the pyridazinyl and the 6-position of the pyridazinyl, wherein the 3-position of the pyridazinyl or the 6-position of the pyridazinyl is substituted by cyano, halogen, halo(C₁-C₄)alkyl, (C₁-C₄)alkoxy, or H₂NCO—.

In other embodiments, R¹ is a substituted or unsubstituted 5-6 membered heteroaryl group, wherein said substituted or unsubstituted 5-6 membered heteroaryl group is a pyridazinyl, wherein said pyridazinyl is pyridazin-3-yl substituted at the 6-position or pyridazin-6-yl substituted at the 3-position by cyano, halogen, halo(C₁-C₄)alkyl, (C₁-C₄)alkoxy, or H₂NCO—.

In specific embodiments, R¹ is a substituted or unsubstituted 5-6 membered heteroaryl group, wherein said substituted or unsubstituted 5-6 membered heteroaryl group is a pyridazinyl, wherein said substituted pyridazinyl is a 3,6-disubstituted pyridazinyl, wherein said 3,6-disubstituted pyridazinyl is substituted at the 3-position of the pyridazinyl and the 6-position of the pyridazinyl, wherein the 3-position of the pyridazinyl or the 6-position of the pyridazinyl is substituted by cyano, fluoro, chloro, trifluoromethyl, methoxy, or H₂NCO—.

In specific embodiments, R¹ is a substituted or unsubstituted 5-6 membered heteroaryl group, wherein said substituted or unsubstituted 5-6 membered heteroaryl group is a pyridazinyl group, wherein said pyridazinyl group is a pyridazin-3-yl substituted at the 6-position or a pyridazin-6-yl substituted at the 3-position by cyano, fluoro, chloro, trifluoromethyl, methoxy, or H₂NCO—.

In another embodiment of the compounds of Formula (I) or Formula (II), R¹ is a substituted or unsubstituted 5-6 membered heteroaryl group, wherein said substituted or unsubstituted 5-6 membered heteroaryl group is a pyridyl, wherein said substituted pyridyl is substituted by H₂NCO—.

In another embodiment, R¹ is a substituted or unsubstituted 5-6 membered heteroaryl group, wherein said substituted or unsubstituted 5-6 membered heteroaryl group is a substituted or unsubstituted oxazolyl, oxadiazolyl, thiazolyl, or tetrazolyl,

wherein said substituted oxazolyl, oxadiazolyl, thiazolyl, or tetrazolyl is substituted by 1 or 2 substituents independently selected from hydroxyl, cyano, halogen, (C₁-C₄)alkyl, halo(C₁-C₄)alkyl, hydroxy(C₁-C₄)alkyl, (C₂-C₄)alkynyl, optionally substituted (C₁-C₄)alkoxy, optionally substituted 5-6 membered heterocycloalkyl-CO—, fused 5-6 membered heterocycloalkyl; H₂N—, ((C₁-C₄)alkyl)-NH—, ((C₁-C₄)alkyl)((C₁-C₄)alkyl)N—, H₂NCO—, ((C₁-C₄)alkyl)NHCO—, (hydroxy-(C₁-C₄)alkyl)NHCO—, (C₃-C₆)cycloalkyl-NHCO—, optionally substituted 5-6 membered heterocycloalkyl-NHCO—, ((C₁-C₄)alkyl)((C₁-C₄)alkyl)-NCO—, (C₁-C₄)alkyl-CONH—, ((C₁-C₄)alkyl)((C₁-C₄)alkyl)N—NHCO—, —CO₂H, —CO₂(C₁-C₄)alkyl, (C₁-C₄)alkylthio-, phenyl-(C₁-C₄)alkylthio-, (C₁-C₄)alkyl-SO₂—, phenyl, optionally substituted 5-6 membered heterocycloalkyl, and optionally substituted 5-6 membered heteroaryl group,

wherein said optionally substituted (C₁-C₄)alkoxy is optionally substituted by hydroxyl, —CO₂H, 5-6 membered heterocycloalkyl, or 5-6 membered heteroaryl; or said optionally substituted 5-6 membered heterocycloalkyl-CO—, optionally substituted 5-6 membered heterocycloalkyl, or optionally substituted 5-6 membered heteroaryl group is optionally substituted by (C₁-C₄)alkyl or oxo; or said optionally substituted 5-6 membered heterocycloalkyl-NHCO— is optionally substituted by (C₁-C₄)alkyl-CO—; or a pharmaceutically acceptable salt thereof.

In another embodiment, R¹ is a substituted or unsubstituted 5-6 membered heteroaryl group, wherein said substituted or unsubstituted 5-6 membered heteroaryl group is a substituted or unsubstituted thiadiazolyl,

wherein said substituted or thiadiazolyl is substituted by 1 or 2 substituents independently selected from hydroxyl, cyano, halogen, (C₁-C₄)alkyl, halo(C₁-C₄)alkyl, hydroxy(C₁-C₄)alkyl, (C₂-C₄)alkynyl, optionally substituted (C₁-C₄)alkoxy, optionally substituted 5-6 membered heterocycloalkyl-CO—, fused 5-6 membered heterocycloalkyl; H₂N—, ((C₁-C₄)alkyl)-NH—, ((C₁-C₄)alkyl)((C₁-C₄)alkyl)N—, H₂NCO—, ((C₁-C₄)alkyl)NHCO—, (hydroxy-(C₁-C₄)alkyl)NHCO—, (C₃-C₆)cycloalkyl-NHCO—, optionally substituted 5-6 membered heterocycloalkyl-NHCO—, ((C₁-C₄)alkyl)((C₁-C₄)alkyl)-NCO—, (C₁-C₄)alkyl-CONH—, ((C₁-C₄)alkyl)((C₁-C₄)alkyl)N—NHCO—, —CO₂H, —CO₂(C₁-C₄)alkyl, (C₁-C₄)alkylthio-, phenyl-(C₁-C₄)alkylthio-, (C₁-C₄)alkyl-SO₂—, phenyl, optionally substituted 5-6 membered heterocycloalkyl, and optionally substituted 5-6 membered heteroaryl group,

wherein said optionally substituted (C₁-C₄)alkoxy is optionally substituted by hydroxyl, —CO₂H, 5-6 membered heterocycloalkyl, or 5-6 membered heteroaryl; or said optionally substituted 5-6 membered heterocycloalkyl-CO—, optionally substituted 5-6 membered heterocycloalkyl, or optionally substituted 5-6 membered heteroaryl group is optionally substituted by (C₁-C₄)alkyl or oxo; or said optionally substituted 5-6 membered heterocycloalkyl-NHCO— is optionally substituted by (C₁-C₄)alkyl-CO—; or a pharmaceutically acceptable salt thereof.

In yet another embodiment, R¹ is a substituted or unsubstituted 5-6 membered heteroaryl group, wherein said substituted or unsubstituted 5-6 membered heteroaryl group is a substituted or unsubstituted oxazolyl, oxadiazolyl, thiazolyl, or tetrazolyl,

wherein said substituted oxazolyl, oxadiazolyl, thiazolyl, or tetrazolyl is substituted by 1 or 2 substituents independently selected from cyano, (C₁-C₄)alkyl, H₂NCO—, ((C₁-C₄)alkyl)NHCO—, —CO₂(C₁-C₄)alkyl, and phenyl; or a pharmaceutically acceptable salt thereof.

In yet another embodiment, R¹ is a substituted or unsubstituted 5-6 membered heteroaryl group, wherein said substituted or unsubstituted 5-6 membered heteroaryl group is a substituted or unsubstituted thiadiazolyl,

wherein said substituted thiadiazolyl is substituted by 1 or 2 substituents independently selected from cyano, (C₁-C₄)alkyl, H₂NCO—, ((C₁-C₄)alkyl)NHCO—, —CO₂(C₁-C₄)alkyl, and phenyl; or a pharmaceutically acceptable salt thereof.

In another embodiment of the compounds of Formula (I) or Formula (II), R¹ is a substituted or unsubstituted 5-6 membered heteroaryl group, wherein said substituted or unsubstituted 5-6 membered heteroaryl group is an oxazolyl, wherein said substituted oxazolyl is substituted by cyano, H₂NCO—, —CO₂(C₁-C₄)alkyl, or phenyl.

In other embodiments, R¹ is a substituted or unsubstituted 5-6 membered heteroaryl group, wherein said substituted or unsubstituted 5-6 membered heteroaryl group is an oxazolyl, wherein said substituted oxazolyl is substituted a 2,4-disubstituted oxazolyl, wherein the 4-position of the oxazolyl is substituted by cyano, H₂NCO—, or —CO₂(C₁-C₄)alkyl.

In other embodiments, R¹ is a substituted or unsubstituted 5-6 membered heteroaryl group, wherein said substituted or unsubstituted 5-6 membered heteroaryl group is an oxazolyl, wherein said substituted oxazolyl is an oxazol-2-yl substituted at the 4-position by cyano, H₂NCO—, or —CO₂(C₁-C₄)alkyl.

In specific embodiments, R¹ is a substituted or unsubstituted 5-6 membered heteroaryl group, wherein said substituted or unsubstituted 5-6 membered heteroaryl group is an oxazolyl, wherein said substituted oxazolyl is a 2,4-disubstituted oxazolyl group, wherein the 4-position of the oxazolyl is substituted by cyano, H₂NCO—, or —CO₂(CH₂CH₃).

In specific embodiments, R¹ is a substituted or unsubstituted 5-6 membered heteroaryl group, wherein said substituted or unsubstituted 5-6 membered heteroaryl group is an oxazolyl, wherein said substituted oxazolyl is an oxazol-2-yl substituted at the 4-position by cyano, H₂NCO—, or —CO₂(CH₂CH₃).

In other embodiments, R¹ is a substituted or unsubstituted 5-6 membered heteroaryl group, wherein said substituted or unsubstituted 5-6 membered heteroaryl group is an oxazolyl, wherein said oxazolyl is substituted a 2,5-disubstituted oxazolyl group, wherein the 5-position of the 2,5-disubstituted oxazolyl group is substituted by cyano, H₂NCO—, —CO₂(C₁-C₄)alkyl, or phenyl.

In other embodiments, R¹ is a substituted or unsubstituted 5-6 membered heteroaryl group, wherein said substituted or unsubstituted 5-6 membered heteroaryl group is an oxazolyl, wherein said substituted oxazolyl is an oxazol-2-yl substituted at the 5-position by cyano, H₂NCO—, —CO₂(C₁-C₄)alkyl, or phenyl.

In specific embodiments, R¹ is a substituted or unsubstituted 5-6 membered heteroaryl group, wherein said substituted or unsubstituted 5-6 membered heteroaryl group is an oxazolyl, wherein said oxazolyl is substituted a 2,5-disubstituted oxazolyl group, wherein the 5-position of the 2,5-oxazolyl group is substituted by cyano, H₂NCO—, —CO₂(CH₂CH₃), or phenyl.

In specific embodiments, R¹ is a substituted or unsubstituted 5-6 membered heteroaryl group, wherein said substituted or unsubstituted 5-6 membered heteroaryl group is an oxazolyl, wherein said substituted oxazolyl is an oxazol-2-yl substituted at the 5-position by cyano, H₂NCO—, —CO₂(CH₂CH₃), or phenyl.

In another embodiment, R¹ is a substituted or unsubstituted 5-6 membered heteroaryl group, wherein said substituted or unsubstituted 5-6 membered heteroaryl group is a substituted or unsubstituted oxadiazolyl, wherein said substituted oxadiazolyl is substituted by (C₁-C₄)alkyl; or a pharmaceutically acceptable salt thereof.

In other embodiments, R¹ is substituted or unsubstituted 5-6 membered heteroaryl group, wherein said substituted or unsubstituted 5-6 membered heteroaryl group is a 1,3,4-oxadiazol-2-yl, wherein said substituted 1,3,4-oxadiazol-2-yl is substituted at the 5-position by (C₁-C₄)alkyl.

In specific embodiments, R¹ is substituted or unsubstituted 5-6 membered heteroaryl group, wherein said substituted or unsubstituted 5-6 membered heteroaryl group is an oxadiazolyl, wherein said substituted oxadiazolyl is substituted by methyl.

In specific embodiments, R¹ is substituted or unsubstituted 5-6 membered heteroaryl group, wherein said substituted or unsubstituted 5-6 membered heteroaryl group is a 1,3,4-oxadiazol-2-yl, wherein said substituted 1,3,4-oxadiazol-2-yl is substituted at the 5-position by methyl.

In other embodiments of the compounds of Formula (I) or Formula (II), R¹ is substituted or unsubstituted 5-6 membered heteroaryl group, wherein said substituted or unsubstituted 5-6 membered heteroaryl group is a thiazolyl, wherein said substituted thiazolyl is substituted by cyano, (C₁-C₄)alkyl, H₂NCO—, or ((C₁-C₄)alkyl)NHCO—.

In specific embodiments, R¹ is substituted or unsubstituted 5-6 membered heteroaryl group, wherein said substituted or unsubstituted 5-6 membered heteroaryl group is a thiazolyl, wherein said substituted thiazolyl is substituted by cyano, methyl, H₂NCO, or (CH₃)NHCO—.

In other embodiments of the compounds of Formula (I) or Formula (II), R¹ is substituted or unsubstituted 5-6 membered heteroaryl group, wherein said substituted or unsubstituted 5-6 membered heteroaryl group is an oxadiazolyl or a thiadiazolyl, wherein said substituted oxadiazolyl or thiadiazolyl is substituted by (C₁-C₄)alkyl or phenyl.

In other embodiments of the compounds of Formula (I) or Formula (II), R¹ is a substituted or unsubstituted 9-10 membered heteroaryl group, wherein said substituted or unsubstituted 9-10 membered heteroaryl group is a substituted or unsubstituted purinyl, quinoxalinyl, pyrazolopyrimidinyl, or imidazopyridazinyl, wherein said substituted purinyl, quinoxalinyl, pyrazolopyrimidinyl, or imidazopyridazinyl is substituted by hydroxyl, or (C₁-C₄)alkyl.

In specific embodiments, R¹ is a substituted or unsubstituted 9-10 membered heteroaryl group, wherein said substituted or unsubstituted 9-10 membered heteroaryl group is a substituted or unsubstituted 7H-purinyl or 1H-pyrazolo[3,4-d]pyrimidinyl, wherein said substituted 7H-purinyl or 1H-pyrazolo[3,4-d]pyrimidinyl is substituted by hydroxyl or methyl.

In other embodiments, R¹ is an unsubstituted 9-10 membered heteroaryl group, wherein said unsubstituted 9-10 membered heteroaryl group is a purinyl, quinoxalinyl, pyrazolopyrimidinyl, or imidazopyridazinyl.

In specific embodiments, R¹ is an unsubstituted 9-10 membered heteroaryl group, wherein said unsubstituted 9-10 membered heteroaryl group is 7H-purinyl, 9H-purinyl, quinozaline, pyrazolo[1,5-a]pyrimidinyl, 1H-pyrazolo[3,4-d]pyrimidinyl, or imidazo[1,2-b]pyridazinyl.

In embodiments of Formula (I) or Formula (II), R¹ is a substituted or unsubstituted 5-6 membered heteroaryl group, wherein said substituted or unsubstituted 5-6 membered heteroaryl group is a substituted or unsubstituted pyrimidinyl or oxadiazolyl, wherein said substituted pyrimidinyl is substituted by 1 or 2 substituents independently selected from cyano, halogen, (C₁-C₄)alkyl, H₂N—, H₂NCO—, and —CO₂H, or said substituted oxadiazolyl is optionally substituted by (C₁-C₄)alkyl. In another embodiment of the compounds of Formula (II), R¹ is a substituted or unsubstituted 5-6 membered heteroaryl group, wherein said substituted or unsubstituted 5-6 membered heteroaryl group is a pyrimidinyl, wherein said substituted pyrimidinyl is a pyrimin-4-yl substituted at the 6-position or a pyrimin-6-yl substituted at the 4-position by H₂NCO—.

In other embodiments of the compounds of Formula (I) or Formula (II), R¹ is a substituted or unsubstituted 5-6 membered heteroaryl group, wherein said substituted or unsubstituted 5-6 membered heteroaryl group is a pyrimidinyl, wherein said substituted pyrimidinyl is pyrimidin-4-yl, pyrimidin-5-yl, or pyrimidin-6-yl substituted by 2 substituents independently selected from halogen and —CO₂H. In specific embodiments, R¹ is a substituted 5-6 membered heteroaryl group, wherein said substituted 5-6 membered heteroaryl group is a pyrimidinyl, wherein said substituted pyrimidinyl is a substituted pyrimidinyl is pyrimidin-4-yl substituted by 2 substituents independently selected from fluoro and —CO₂H.

In other embodiments of the compounds of Formula (I) or Formula (II), R¹ is a substituted or unsubstituted 5-6 membered heteroaryl group, wherein said substituted or unsubstituted 5-6 membered heteroaryl group is an oxadiazolyl optionally substituted by (C₁-C₄)alkyl.

In one embodiment of the compounds of Formula (I) or Formula (II), R² is a substituted or unsubstituted phenyl or 5-6 membered heteroaryl group, wherein said substituted phenyl or 5-6 membered heteroaryl group is substituted by 1 or 2 substituents independently selected from halogen, (C₁-C₄)alkyl, (C₁-C₄)alkoxy, and cyano.

In another embodiment, R² is a substituted or unsubstituted phenyl or 5-6 membered heteroaryl group, wherein said substituted phenyl or 5-6 membered heteroaryl group is optionally substituted by 1 or 2 substituents independently selected from halogen and cyano;

or a pharmaceutically acceptable salt thereof.

In other embodiments, R² is a substituted or unsubstituted phenyl or 5-6 membered heteroaryl group, wherein said substituted or unsubstituted 5-6 membered heteroaryl group is pyridyl, thiazolyl, or isothiazolyl wherein said substituted phenyl, pyridyl, thiazolyl, or isothiazolyl is substituted by 1 or 2 substituents independently selected from halogen, cyano, (C₁-C₄)alkyl, and (C₁-C₄)alkoxy.

In one embodiment of the compounds of Formula (I) or Formula (II), R² is a substituted or unsubstituted phenyl or 5-6 membered heteroaryl group, wherein said substituted or unsubstituted 5-6 membered heteroaryl group is a pyridyl, wherein said substituted phenyl or pyridyl is substituted by one or two substituents independently selected from halogen and cyano.

In another embodiment, R² is substituted 5-6 membered heteroaryl group, wherein said substituted 5-6 membered heteroaryl group is a substituted or unsubstituted pyridyl group, wherein said substituted pyridyl group is substituted by one or two fluoro groups; or a pharmaceutically acceptable salt thereof.

In specific embodiments, R² is a substituted or unsubstituted 5-6 membered heteroaryl group, wherein said substituted or unsubstituted 5-6 membered heteroaryl group is a pyridyl, wherein said substituted pyridyl is substituted by one halogen, wherein the halogen is fluoro.

In other embodiments, R² is a substituted or unsubstituted phenyl group, wherein said substituted phenyl is substituted by one or two substituents independently selected from halogen and cyano.

In specific embodiments, R² is a substituted or unsubstituted phenyl group, wherein said substituted phenyl is substituted by cyano.

In other embodiments, R² is a substituted or unsubstituted phenyl group, wherein said substituted phenyl group is substituted by one or two halogens, wherein the halogen is fluoro.

In specific embodiments, R² is a substituted or unsubstituted phenyl group, wherein said substituted phenyl is substituted by two halogens, wherein the halogen is fluoro.

In one embodiment of the compounds of Formulas (I) and (II), R² is unsubstituted phenyl.

In another embodiment, the invention is directed to compounds according to Formula (I) or Formula (II), wherein R¹ is a substituted 5-6 membered heteroaryl group, wherein said substituted 5-6 membered heteroaryl group is substituted by 1 or 2 substituents independently selected from cyano, halogen, (C₁-C₄)alkyl, H₂N—, H₂NCO—, and —CO₂H; and R² is a substituted or unsubstituted phenyl or pyridyl, wherein the substituted phenyl or pyridyl is substituted by 1 or 2 substituents independently selected from halogen and cyano; or a pharmaceutically acceptable salt thereof. In another embodiment, the invention is directed to a compound according to Formulas (I) and (II), wherein R¹ is a substituted 5-6 membered heteroaryl group, wherein said substituted 5-6 membered heteroaryl group is a pyrimidinyl or oxadiazolyl, wherein the substituted pyrimidinyl or oxadiazolyl is substituted by 1 or 2 substituents independently selected from cyano, halogen, (C₁-C₄)alkyl, H₂N—, H₂NCO—, and —CO₂H; and R² is a substituted or unsubstituted phenyl or pyridyl, wherein the substituted phenyl or pyridyl is substituted by 1 or 2 substituents independently selected from cyano and halogen; or a pharmaceutically acceptable salt thereof. In another embodiment, the invention is directed to a compound according to Formulas (I) and (II), wherein R¹ is a substituted 5-6 membered heteroaryl group, wherein said substituted 5-6 membered heteroaryl group is a pyrimidinyl or oxadiazolyl, wherein the substituted pyrimidinyl or oxadiazolyl is substituted by 1 or 2 substituents independently selected from cyano, fluoro, methyl, H₂N—, H₂NCO—, and —CO₂H; and R² is a substituted or unsubstituted phenyl or pyridyl, wherein the substituted phenyl or pyridyl is substituted by 1 or 2 substituents independently selected from cyano and fluoro; or a pharmaceutically acceptable salt thereof.

In another embodiment, the invention is directed to a compound according to Formulas (I) and (II), wherein R¹ is a substituted 5-6 membered heteroaryl group, wherein said substituted 5-6 membered heteroaryl group is a pyrimidinyl, wherein the substituted pyrimidinyl is substituted by H₂NCO—; and R² is a substituted or unsubstituted 5-6 heteroaryl group, wherein the substituted 5-6 heteroaryl group is substituted by a halogen; or a pharmaceutically acceptable salt thereof. In another embodiment, the invention is directed to a compound according to Formulas (I) and (II), wherein R¹ is a substituted 5-6 membered heteroaryl group, wherein said substituted 5-6 membered heteroaryl group is a pyrimidinyl, wherein the substituted pyrimidinyl is substituted by H₂NCO—; and R² is a substituted or unsubstituted 5-6 heteroaryl group, wherein the substituted 5-6 heteroaryl group is a pyridyl, wherein the pyridyl is substituted by fluoro; or a pharmaceutically acceptable salt thereof.

In a further embodiment, the invention is directed to a compound according to Formula (I) or Formula (II), wherein R¹ is a substituted 5-6 membered heteroaryl group, wherein the substituted 5-6 membered heteroaryl group is substituted by cyano; and R² is a substituted or unsubstituted phenyl, wherein the substituted phenyl is substituted by 1 or 2 halogens; or a pharmaceutically acceptable salt, thereof. In a specific embodiment, the invention is directed to a compound according to Formula (I) or Formula (II), wherein R¹ is pyrimidyl, wherein the pyrimidyl is substituted by cyano; and R² is a substituted or unsubstituted phenyl, wherein the substituted phenyl is substituted by one or two fluoro; or a pharmaceutically acceptable salt thereof. In a specific embodiment, the invention is directed to a compound according to Formula (I) or Formula (II), wherein R¹ is pyrimidin-6-yl-4-carbonitrile; and R² is 3,5-difluorophenyl; or a pharmaceutically acceptable salt thereof.

In a further embodiment, the invention is directed to a compound according to Formula (I) or Formula (II), wherein R¹ is a substituted 5-6 membered heteroaryl group, wherein said substituted 5-6 membered heteroaryl group is substituted by H₂NCO—; and R² is a substituted or unsubstituted 5-6 heteroaryl group, wherein the substituted 5-6 heteroaryl group is substituted by 1 or 2 halogens; or a pharmaceutically acceptable salt thereof. In a specific embodiment, the invention is directed to a compound according to Formula (I) or Formula (II), wherein R¹ is a substituted 5-6 membered heteroaryl group, wherein said substituted 5-6 membered heteroaryl group is a pyrimidinyl, wherein substituted pyrimidinyl is substituted by H₂NCO—; and R² is a substituted or unsubstituted 5-6 heteroaryl group, wherein the substituted 5-6 heteroaryl group is pyridyl, wherein the substituted pyridyl is substituted one fluoro; or a pharmaceutically acceptable salt thereof. In a specific embodiment, the invention is directed to a compound according to Formula (I) or Formula (II), wherein R¹ is pyrimidin-6-yl-4-carboxamide; and R² is 5-fluoropyridin-3-yl; or a pharmaceutically acceptable salt thereof.

In a further embodiment, the invention is directed to a compound according to Formula (I) or Formula (II), wherein R¹ is a substituted 5-6 membered heteroaryl group, wherein said substituted 5-6 membered heteroaryl group is substituted by 1 or 2 substituents independently selected from halogen and —CO₂H; and R² is an unsubstituted phenyl, wherein the substituted phenyl is substituted by 1 or 2 halogens; or a pharmaceutically acceptable salt thereof. In a specific embodiment, the invention is directed to a compound according to Formula (I) or Formula (II), wherein R¹ is a substituted 5-6 membered heteroaryl group, wherein said substituted 5-6 membered heteroaryl group is a pyrimidinyl, wherein the substituted pyrimidinyl is substituted by one fluoro and one —CO₂H; and R² is an unsubstituted phenyl; or a pharmaceutically acceptable salt thereof. In a specific embodiment, the invention is directed to a compound according to Formula (I) or Formula (II), wherein R¹ is 5-fluoropyrimidin-6-yl-4-carboxylic acid; and R² is phenyl; or a pharmaceutically acceptable salt thereof.

In a further embodiment, the invention is directed to a compound according to Formulas (I) and (II), wherein R¹ is a substituted 5-6 membered heteroaryl group, wherein said substituted 5-6 membered heteroaryl group is substituted by (C₁-C₄)alkyl; and R² is a substituted or unsubstituted phenyl, wherein the substituted phenyl is substituted by 1 or 2 halogens; or a pharmaceutically acceptable salt thereof. In a specific embodiment, the invention is directed to a compound according to Formula (I) or Formula (II), wherein R¹ is a substituted 5-6 membered heteroaryl group, wherein said substituted 5-6 membered heteroaryl group is a oxadiazolyl, wherein the substituted oxadiazolyl is substituted by methyl; and R² is a substituted phenyl, wherein the substituted phenyl is substituted by two halogens; or a pharmaceutically acceptable salt thereof. In a specific embodiment, the invention is directed to a compound according to Formulas (I) and (II), wherein R¹ is 5-methyl-1,3,4-oxadiazol-2-yl; and R² is 3,5-difluorophenyl; or a pharmaceutically acceptable salt thereof.

In another embodiment, a compound of Formula (I) excludes the following compounds:
(5-(5-fluoropyridin-3-yl)-4,5-dihydro-1H-pyrazol-1-yl)(1-(5-methylpyrimidin-2-yl)piperidin-4-yl)methanone;
(1-(5-fluoropyridin-2-yl)piperidin-4-yl)(5-phenyl-4,5-dihydro-1H-pyrazol-1-yl)methanone;
(1-(5-methylpyridin-2-yl)piperidin-4-yl)(5-phenyl-4,5-dihydro-1H-pyrazol-1-yl)methanone;
(1-(5-methylpyrimidin-2-yl)piperidin-4-yl)(5-phenyl-4,5-dihydro-1H-pyrazol-1-yl)methanone;
(1-(5-fluoropyrimidin-2-yl)piperidin-4-yl)(5-phenyl-4,5-dihydro-1H-pyrazol-1-yl)methanone;
(5-(5-fluoropyridin-3-yl)-4,5-dihydro-1H-pyrazol-1-yl)(1-(5-fluoropyrimidin-2-yl)piperidin-4-yl)methanone;
(5-(5-fluoropyridin-3-yl)-4,5-dihydro-1H-pyrazol-1-yl)(1-(pyrimidin-2-yl)piperidin-4-yl)methanone;
(1H-indol-2-yl)(5-phenyl-4,5-dihydro-1H-pyrazol-1-yl)methanone;
(5-(pyridin-3-yl)-4,5-dihydro-1H-pyrazol-1-yl)(1-(pyrimidin-2-yl)piperidin-4-yl)methanone;
(5-(6-methylpyridin-3-yl)-4,5-dihydro-1H-pyrazol-1-yl)(1-(pyrimidin-2-yl)piperidin-4-yl)methanone;
(5-(6-methylpyridin-3-yl)-4,5-dihydro-1H-pyrazol-1-yl)(1-(pyridin-2-yl)piperidin-4-yl)methanone;
(1-(pyridin-2-yl)piperidin-4-yl)(5-(pyridin-3-yl)-4,5-dihydro-1H-pyrazol-1-yl)methanone;
(5-(5-methylpyrazin-2-yl)-4,5-dihydro-1H-pyrazol-1-yl)(1-(pyridin-2-yl)piperidin-4-yl)methanone;
(1-(benzo[d]oxazol-2-yl)piperidin-4-yl)(5-(pyridin-3-yl)-4,5-dihydro-1H-pyrazol-1-yl)methanone;
(5-phenyl-4,5-dihydro-1H-pyrazol-1-yl)(1-(pyrimidin-2-yl)piperidin-4-yl)methanone;
(5-phenyl-4,5-dihydro-1H-pyrazol-1-yl)(1-(pyridin-2-yl)piperidin-4-yl)methanone 2,2,2-trifluoroacetate;
(5-phenyl-4,5-dihydro-1H-pyrazol-1-yl)(1-(5-(trifluoromethyl)pyridin-2-yl)piperidin-4-yl)methanone 2,2,2-trifluoroacetate;
(1-(benzo[d]oxazol-2-yl)piperidin-4-yl)(5-phenyl-4,5-dihydro-1H-pyrazol-1-yl)methanone 2,2,2-trifluoroacetate;
(1-(5-fluoropyrimidin-2-yl)piperidin-4-yl)(5-(6-methylpyridin-3-yl)-4,5-dihydro-1H-pyrazol-1-yl)methanone; and
4-(1-(1-(5-fluoropyrimidin-2-yl)piperidine-4-carbonyl)-4,5-dihydro-1H-pyrazol-5-yl)benzonitrile.

In another embodiment, a compound of Formula (II) excludes the following compounds:
(S)-(1-(5-fluoropyridin-2-yl)piperidin-4-yl)(5-phenyl-4,5-dihydro-1H-pyrazol-1-yl)methanone;
(S)-(1-(5-fluoropyrimidin-2-yl)piperidin-4-yl)(5-phenyl-4,5-dihydro-1H-pyrazol-1-yl)methanone;
(S)-(5-(5-fluoropyridin-3-yl)-4,5-dihydro-1H-pyrazol-1-yl)(1-(5-fluoropyrimidin-2-yl)piperidin-4-yl)methanone;
(S)-(5-(pyridin-3-yl)-4,5-dihydro-1H-pyrazol-1-yl)(1-(pyrimidin-2-yl)piperidin-4-yl)methanone;
(S)-(1-(pyridin-2-yl)piperidin-4-yl)(5-(pyridin-3-yl)-4,5-dihydro-1H-pyrazol-1-yl)methanone;
(S)-(5-phenyl-4,5-dihydro-1H-pyrazol-1-yl)(1-(pyrimidin-2-yl)piperidin-4-yl)methanone; and
(S)-(5-phenyl-4,5-dihydro-1H-pyrazol-1-yl)(1-(pyridin-2-yl)piperidin-4-yl)methanone.

It will be appreciated that the present invention encompasses compounds of Formula (I) and Formula (II) as the free base or free acid and as pharmaceutically acceptable salts thereof. In one embodiment, the invention relates to compounds of Formula (I) and Formula (II) in the form of a free base. In one embodiment, the invention relates to compounds of Formula (I) and Formula (II) in the form of a free acid. In another embodiment, the invention relates to compounds of Formulas (I) and (II) in the form of a pharmaceutically acceptable salt. It will be further appreciated that, in one embodiment, the invention relates to compounds of the Examples in the form of a free base. In another embodiment, the invention relates to compounds of the Examples in the form of a a pharmaceutically acceptable salt.

The compounds of this invention include the following compounds described herein:
(S)-(1-(4-(benzylthio)pyrimidin-2-yl)piperidin-4-yl)(5-phenyl-4,5-dihydro-1H-pyrazol-1-yl)methanone;
2-(4-(5-phenyl-4,5-dihydro-1H-pyrazole-1-carbonyl)piperidin-1-yl)pyrimidine-5-carbonitrile;
(1-(4-methoxypyrimidin-2-yl)piperidin-4-yl)(5-phenyl-4,5-dihydro-1H-pyrazol-1-yl)methanone;
(5-phenyl-4,5-dihydro-1H-pyrazol-1-yl)(1-(4-phenylpyrimidin-2-yl)piperidin-4-yl)methanone;
2-(4-(5-phenyl-4,5-dihydro-1H-pyrazole-1-carbonyl)piperidin-1-yl)pyrimidine-4-carbonitrile;
(1-(4-aminopyrimidin-2-yl)piperidin-4-yl)(5-phenyl-4,5-dihydro-1H-pyrazol-1-yl)methanone;
(1-(5-methoxypyrimidin-2-yl)piperidin-4-yl)(5-phenyl-4,5-dihydro-1H-pyrazol-1-yl)methanone;
(S)-(1-(5-(methylsulfonyl)pyrimidin-2-yl)piperidin-4-yl)(5-phenyl-4,5-dihydro-1H-pyrazol-1-yl)methanone;
(S)-(1-(7H-purin-2-yl)piperidin-4-yl)(5-phenyl-4,5-dihydro-1H-pyrazol-1-yl)methanone;
(S)-methyl 2-(4-(5-phenyl-4,5-dihydro-1H-pyrazole-1-carbonyl)piperidin-1-yl)pyrimidine-5-carboxylate;
(S)-(1-(2-aminopyrimidin-4-yl)piperidin-4-yl)(5-phenyl-4,5-dihydro-1H-pyrazol-1-yl)methanone;
(S)-(1-(6-methoxypyrimidin-4-yl)piperidin-4-yl)(5-phenyl-4,5-dihydro-1H-pyrazol-1-yl)methanone;
(S)-(1-(5-methoxypyrimidin-2-yl)piperidin-4-yl)(5-phenyl-4,5-dihydro-1H-pyrazol-1-yl)methanone;
(S)-6-(4-(5-phenyl-4,5-dihydro-1H-pyrazole-1-carbonyl)piperidin-1-yl)pyrimidine-4-carbonitrile;
(S)-(1-(2-(methylamino)pyrimidin-4-yl)piperidin-4-yl)(5-phenyl-4,5-dihydro-1H-pyrazol-1-yl)methanone;
(S)-(1-(4-(methylamino)pyrimidin-2-yl)piperidin-4-yl)(5-phenyl-4,5-dihydro-1H-pyrazol-1-yl)methanone;
(S)-(1-(2-methoxypyrimidin-4-yl)piperidin-4-yl)(5-phenyl-4,5-dihydro-1H-pyrazol-1-yl)methanone;
(S)-4-(4-(5-phenyl-4,5-dihydro-1H-pyrazole-1-carbonyl)piperidin-1-yl)pyrimidine-2-carbonitrile;
(S)-2-(4-(5-phenyl-4,5-dihydro-1H-pyrazole-1-carbonyl)piperidin-1-yl)pyrimidin-4(3H)-one;
(S)-(1-(6-aminopyrimidin-4-yl)piperidin-4-yl)(5-phenyl-4,5-dihydro-1H-pyrazol-1-yl)methanone;
(S)-(5-phenyl-4,5-dihydro-1H-pyrazol-1-yl)(1-(pyrazolo[1,5-a]pyrimidin-5-yl)piperidin-4-yl)methanone;
(S)-(1-(imidazo[1,2-b]pyridazin-6-yl)piperidin-4-yl)(5-phenyl-4,5-dihydro-1H-pyrazol-1-yl)methanone;
(S)-(1-(9-methyl-9H-purin-2-yl)piperidin-4-yl)(5-phenyl-4,5-dihydro-1H-pyrazol-1-yl)methanone;
(S)-2-(4-(5-phenyl-4,5-dihydro-1H-pyrazole-1-carbonyl)piperidin-1-yl)-5H-pyrrolo[2,3-d]pyrimidin-6(7H)-one;
(S)-6-(4-(5-phenyl-4,5-dihydro-1H-pyrazole-1-carbonyl)piperidin-1-yl)pyridazine-3-carboxamide;
(S)-(5-phenyl-4,5-dihydro-1H-pyrazol-1-yl)(1-(6-(trifluoromethyl)pyridazin-3-yl)piperidin-4-yl)methanone;
(S)-(1-(4-amino-5-fluoropyrimidin-2-yl)piperidin-4-yl)(5-phenyl-4,5-dihydro-1H-pyrazol-1-yl)methanone;
(S)-2-(4-(5-phenyl-4,5-dihydro-1H-pyrazole-1-carbonyl)piperidin-1-yl)pyrimidine-4-carboxamide;
(S)-2-(4-(5-phenyl-4,5-dihydro-1H-pyrazole-1-carbonyl)piperidin-1-yl)pyrimidine-4-carboxylic acid;
(S)-6-(4-(5-phenyl-4,5-dihydro-1H-pyrazole-1-carbonyl)piperidin-1-yl)nicotinamide;
(S)-6-(4-(5-phenyl-4,5-dihydro-1H-pyrazole-1-carbonyl)piperidin-1-yl)pyrazine-2-carboxamide;
(S)-6-(4-(5-phenyl-4,5-dihydro-1H-pyrazole-1-carbonyl)piperidin-1-yl)pyrimidine-4-carboxamide;
(S)-(1-(6-amino-2-methylpyrimidin-4-yl)piperidin-4-yl)(5-phenyl-4,5-dihydro-1H-pyrazol-1-yl)methanone;
(S)-(1-(2-amino-6-methoxypyrimidin-4-yl)piperidin-4-yl)(5-phenyl-4,5-dihydro-1H-pyrazol-1-yl)methanone;
(S)-(1-(6-amino-2-methoxypyrimidin-4-yl)piperidin-4-yl)(5-phenyl-4,5-dihydro-1H-pyrazol-1-yl)methanone;
(S)—N-(2-(4-(5-phenyl-4,5-dihydro-1H-pyrazole-1-carbonyl)piperidin-1-yl)pyrimidin-4-yl)acetamide;
(S)-6-(4-(5-phenyl-4,5-dihydro-1H-pyrazole-1-carbonyl)piperidin-1-yl)-1H-pyrazolo[3,4-d]pyrimidin-4(7H)-one;
(S)-(5-phenyl-4,5-dihydro-1H-pyrazol-1-yl)(1-(6-phenylpyrazin-2-yl)piperidin-4-yl)methanone;
(S)-(5-phenyl-4,5-dihydro-1H-pyrazol-1-yl)(1-(quinoxalin-2-yl)piperidin-4-yl)methanone;
(S)-5-(4-(5-phenyl-4,5-dihydro-1H-pyrazole-1-carbonyl)piperidin-1-yl)pyrazine-2-carbonitrile;
(S)-(1-(6-aminopyrazin-2-yl)piperidin-4-yl)(5-phenyl-4,5-dihydro-1H-pyrazol-1-yl)methanone;
(S)-6-(4-(5-phenyl-4,5-dihydro-1H-pyrazole-1-carbonyl)piperidin-1-yl)pyridazine-3-carbonitrile;
(S)-(1-(6-hydroxypyrimidin-4-yl)piperidin-4-yl)(5-phenyl-4,5-dihydro-1H-pyrazol-1-yl)methanone;
(S)-3-(4-(5-phenyl-4,5-dihydro-1H-pyrazole-1-carbonyl)piperidin-1-yl)pyrazine-2-carbonitrile;
(S)-(1-(2-(methylthio)pyrimidin-4-yl)piperidin-4-yl)(5-phenyl-4,5-dihydro-1H-pyrazol-1-yl)methanone;
(S)-(5-phenyl-4,5-dihydro-1H-pyrazol-1-yl)(1-(2-(trifluoromethyl)pyrimidin-4-yl)piperidin-4-yl)methanone;
(S)-6-(4-(5-phenyl-4,5-dihydro-1H-pyrazole-1-carbonyl)piperidin-1-yl)pyrazine-2-carbonitrile;
(S)-(1-(6-methoxypyrazin-2-yl)piperidin-4-yl)(5-phenyl-4,5-dihydro-1H-pyrazol-1-yl)methanone;
(S)-(1-(6-methoxypyridazin-3-yl)piperidin-4-yl)(5-phenyl-4,5-dihydro-1H-pyrazol-1-yl)methanone;
(S)-4-(4-(5-phenyl-4,5-dihydro-1H-pyrazole-1-carbonyl)piperidin-1-yl)pyrimidine-5-carbonitrile;
(S)-(5-phenyl-4,5-dihydro-1H-pyrazol-1-yl)(1-(6-(trifluoromethyl)pyrimidin-4-yl)piperidin-4-yl)methanone;
(S)-(1-(1H-pyrazolo[3,4-d]pyrimidin-6-yl)piperidin-4-yl)(5-phenyl-4,5-dihydro-1H-pyrazol-1-yl)methanone;
(S)-2-(4-(5-phenyl-4,5-dihydro-1H-pyrazole-1-carbonyl)piperidin-1-yl)thiazole-4-carbonitrile;
(S)—N-methyl-2-(4-(5-phenyl-4,5-dihydro-1H-pyrazole-1-carbonyl)piperidin-1-yl)thiazole-4-carboxamide;
(S)-2-(4-(5-phenyl-4,5-dihydro-1H-pyrazole-1-carbonyl)piperidin-1-yl)thiazole-5-carboxamide;
(S)-2-(4-(5-phenyl-4,5-dihydro-1H-pyrazole-1-carbonyl)piperidin-1-yl) thiazole-4-carboxamide;
(S)-(1-(5-phenyl-1,3,4-oxadiazol-2-yl)piperidin-4-yl)(5-phenyl-4,5-dihydro-1H-pyrazol-1-yl)methanone;
(S)-(1-(4-ethoxypyrimidin-2-yl)piperidin-4-yl)(5-phenyl-4,5-dihydro-1H-pyrazol-1-yl)methanone;
(S)-(1-(6-(methylthio)pyrimidin-4-yl)piperidin-4-yl)(5-phenyl-4,5-dihydro-1H-pyrazol-1-yl)methanone;
(S)-(1-(6-amino-2-(methylthio)pyrimidin-4-yl)piperidin-4-yl)(5-phenyl-4,5-dihydro-1H-pyrazol-1-yl)methanone;
(S)-(1-(6-amino-5-fluoropyrimidin-4-yl)piperidin-4-yl)(5-phenyl-4,5-dihydro-1H-pyrazol-1-yl)methanone;
(S)-3-(1-(1-(pyrazolo[1,5-a]pyrimidin-5-yl)piperidine-4-carbonyl)-4,5-dihydro-1H-pyrazol-5-yl)benzonitrile;
(S)-5-(4-(5-phenyl-4,5-dihydro-1H-pyrazole-1-carbonyl)piperidin-1-yl)pyrazine-2-carboxamide;
(S)—N-(6-(4-(5-phenyl-4,5-dihydro-1H-pyrazole-1-carbonyl)piperidin-1-yl)pyrazin-2-yl)acetamide;
(S)-ethyl 2-(4-(5-phenyl-4,5-dihydro-1H-pyrazole-1-carbonyl)piperidin-1-yl)oxazole-4-carboxylate;
(S)-ethyl 2-(4-(5-phenyl-4,5-dihydro-1H-pyrazole-1-carbonyl)piperidin-1-yl)oxazole-5-carboxylate;
(S)-(5-phenyl-4,5-dihydro-1H-pyrazol-1-yl)(1-(5-phenyloxazol-2-yl)piperidin-4-yl)methanone;
(S)-6-(4-(5-(3-cyanophenyl)-4,5-dihydro-1H-pyrazole-1-carbonyl)piperidin-1-yl)pyrimidine-4-carbonitrile;
(S)-3-(1-(1-(4-methoxypyrimidin-2-yl)piperidine-4-carbonyl)-4,5-dihydro-1H-pyrazol-5-yl)benzonitrile;
(S)-6-(4-(5-(3-cyanophenyl)-4,5-dihydro-1H-pyrazole-1-carbonyl)piperidin-1-yl)pyrimidine-4-carboxamide;
(S)-2-(4-(5-(3-cyanophenyl)-4,5-dihydro-1H-pyrazole-1-carbonyl)piperidin-1-yl)pyrimidine-4-carboxamide;
(S)-3-(1-(1-(4-amino-5-fluoropyrimidin-2-yl)piperidine-4-carbonyl)-4,5-dihydro-1H-pyrazol-5-yl)benzonitrile;
(S)-3-(1-(1-(imidazo[1,2-b]pyridazin-6-yl)piperidine-4-carbonyl)-4,5-dihydro-1H-pyrazol-5-yl)benzonitrile;
(S)-4-(4-(5-(3-cyanophenyl)-4,5-dihydro-1H-pyrazole-1-carbonyl)piperidin-1-yl)pyrimidine-2-carbonitrile;
(S)-3-(1-(1-(2-methoxypyrimidin-4-yl)piperidine-4-carbonyl)-4,5-dihydro-1H-pyrazol-5-yl)benzonitrile;
(S)-3-(1-(1-(1H-pyrazolo[3,4-d]pyrimidin-6-yl)piperidine-4-carbonyl)-4,5-dihydro-1H-pyrazol-5-yl)benzonitrile;
(S)-(5-(3,5-difluorophenyl)-4,5-dihydro-1H-pyrazol-1-yl)(1-(5-methyl-1,3,4-oxadiazol-2-yl)piperidin-4-yl)methanone;
(S)-6-(4-(5-(3,5-difluorophenyl)-4,5-dihydro-1H-pyrazole-1-carbonyl)piperidin-1-yl)pyrimidine-4-carbonitrile;
(S)-(5-(3,5-difluorophenyl)-4,5-dihydro-1H-pyrazol-1-yl)(1-(4-methoxypyrimidin-2-yl)piperidin-4-yl)methanone;
(S)-2-(4-(5-(3,5-difluorophenyl)-4,5-dihydro-1H-pyrazole-1-carbonyl)piperidin-1-yl)-5H-pyrrolo[2,3-d]pyrimidin-6(7H)-one;
(S)-(1-(4-amino-5-fluoropyrimidin-2-yl)piperidin-4-yl)(5-(3,5-difluorophenyl)-4,5-dihydro-1H-pyrazol-1-yl)methanone;
(S)-(5-(3,5-difluorophenyl)-4,5-dihydro-1H-pyrazol-1-yl)(1-(2-(methylthio)pyrimidin-4-yl)piperidin-4-yl)methanone;
(S)-2-(4-(5-(3,5-difluorophenyl)-4,5-dihydro-1H-pyrazole-1-carbonyl)piperidin-1-yl)pyrimidine-4-carboxamide;
(S)-(1-(2-aminopyrimidin-4-yl)piperidin-4-yl)(5-(3,5-difluorophenyl)-4,5-dihydro-1H-pyrazol-1-yl)methanone;
(S)-6-(4-(5-(3,5-difluorophenyl)-4,5-dihydro-1H-pyrazole-1-carbonyl)piperidin-1-yl)pyrimidine-4-carboxamide;
(S)-(1-(1H-pyrazolo[3,4-d]pyrimidin-6-yl)piperidin-4-yl)(5-(3,5-difluorophenyl)-4,5-dihydro-1H-pyrazol-1-yl)methanone;
(S)-(5-(3,5-difluorophenyl)-4,5-dihydro-1H-pyrazol-1-yl)(1-(imidazo[1,2-b]pyridazin-6-yl)piperidin-4-yl)methanone;
(S)-4-(4-(5-(3,5-difluorophenyl)-4,5-dihydro-1H-pyrazole-1-carbonyl)piperidin-1-yl)pyrimidine-5-carbonitrile;
(S)-4-(4-(5-(3,5-difluorophenyl)-4,5-dihydro-1H-pyrazole-1-carbonyl)piperidin-1-yl)pyrimidine-2-carbonitrile;
(S)-(5-(3,5-difluorophenyl)-4,5-dihydro-1H-pyrazol-1-yl)(1-(pyrazolo[1,5-a]pyrimidin-5-yl)piperidin-4-yl)methanone;
(S)-(5-(3,5-difluorophenyl)-4,5-dihydro-1H-pyrazol-1-yl)(1-(6-methoxypyrimidin-4-yl)piperidin-4-yl)methanone;
(S)-2-(4-(5-(3,5-difluorophenyl)-4,5-dihydro-1H-pyrazole-1-carbonyl)piperidin-1-yl)pyrimidine-5-carbonitrile;
(S)-(1-(4-aminopyrimidin-2-yl)piperidin-4-yl)(5-(3,5-difluorophenyl)-4,5-dihydro-1H-pyrazol-1-yl)methanone;
(S)-6-(4-(5-(3,5-difluorophenyl)-4,5-dihydro-1H-pyrazole-1-carbonyl)piperidin-1-yl)pyridazine-3-carbonitrile;
(S)-5-(4-(5-(3,5-difluorophenyl)-4,5-dihydro-1H-pyrazole-1-carbonyl)piperidin-1-yl)pyrazine-2-carbonitrile;
(S)-2-(4-(5-(3,5-difluorophenyl)-4,5-dihydro-1H-pyrazole-1-carbonyl)piperidin-1-yl)pyrimidine-5-carboxamide;
(S)-2-(4-(5-(3,5-difluorophenyl)-4,5-dihydro-1H-pyrazole-1-carbonyl)piperidin-1-yl)pyrimidin-4(3H)-one;
(S)-(1-(6-aminopyrimidin-4-yl)piperidin-4-yl)(5-(3,5-difluorophenyl)-4,5-dihydro-1H-pyrazol-1-yl)methanone;
(S)-(5-(3,5-difluorophenyl)-4,5-dihydro-1H-pyrazol-1-yl)(1-(2-methoxypyrimidin-4-yl)piperidin-4-yl)methanone;
(S)-(5-(3,5-difluorophenyl)-4,5-dihydro-1H-pyrazol-1-yl)(1-(2-(methylamino)pyrimidin-4-yl)piperidin-4-yl)methanone;
(S)-(5-(2,5-difluorophenyl)-4,5-dihydro-1H-pyrazol-1-yl)(1-(5-methoxypyrimidin-2-yl)piperidin-4-yl)methanone,
(S)-2-(4-(5-(2,5-difluorophenyl)-4,5-dihydro-1H-pyrazole-1-carbonyl)piperidin-1-yl)pyrimidine-4-carboxamide;
(S)-5-(4-(5-(2,5-difluorophenyl)-4,5-dihydro-1H-pyrazole-1-carbonyl)piperidin-1-yl)pyrazine-2-carbonitrile;
(S)-6-(4-(5-(2,5-difluorophenyl)-4,5-dihydro-1H-pyrazole-1-carbonyl)piperidin-1-yl)pyrimidine-4-carbonitrile;
(S)-(5-(2,5-difluorophenyl)-4,5-dihydro-1H-pyrazol-1-yl)(1-(6-methoxypyrimidin-4-yl)piperidin-4-yl)methanone;
(S)-ethyl 2-(4-(5-(3,5-difluorophenyl)-4,5-dihydro-1H-pyrazole-1-carbonyl)piperidin-1-yl)oxazole-4-carboxylate;
(S)-ethyl 2-(4-(5-(3,5-difluorophenyl)-4,5-dihydro-1H-pyrazole-1-carbonyl)piperidin-1-yl)oxazole-5-carboxylate;
(S)-6-(4-(5-(5-fluoropyridin-3-yl)-4,5-dihydro-1H-pyrazole-1-carbonyl)piperidin-1-yl)pyrimidine-4-carboxamide;
(S)-2-(4-(5-(5-fluoropyridin-3-yl)-4,5-dihydro-1H-pyrazole-1-carbonyl)piperidin-1-yl)pyrimidine-4-carboxamide;
(S)-(5-(5-fluoropyridin-3-yl)-4,5-dihydro-1H-pyrazol-1-yl)(1-(2-methoxypyrimidin-4-yl)piperidin-4-yl)methanone;
(S)-6-(4-(5-(5-fluoropyridin-3-yl)-4,5-dihydro-1H-pyrazole-1-carbonyl)piperidin-1-yl)pyrimidine-4-carbonitrile;
(S)-(1-(4-amino-5-fluoropyrimidin-2-yl)piperidin-4-yl)(5-(5-fluoropyridin-3-yl)-4,5-dihydro-1H-pyrazol-1-yl)methanone;
(S)-(5-(5-fluoropyridin-3-yl)-4,5-dihydro-1H-pyrazol-1-yl)(1-(6-methoxypyrimidin-4-yl)piperidin-4-yl)methanone;
(S)-(5-(5-fluoropyridin-3-yl)-4,5-dihydro-1H-pyrazol-1-yl)(1-(2-(methylthio)pyrimidin-4-yl)piperidin-4-yl)methanone;
(S)-4-(4-(5-(5-fluoropyridin-3-yl)-4,5-dihydro-1H-pyrazole-1-carbonyl)piperidin-1-yl)pyrimidine-2-carbonitrile;
(S)-(5-(5-fluoropyridin-3-yl)-4,5-dihydro-1H-pyrazol-1-yl)(1-(pyrazolo[1,5-a]pyrimidin-5-yl)piperidin-4-yl)methanone;
(S)-(5-(5-fluoropyridin-3-yl)-4,5-dihydro-1H-pyrazol-1-yl)(1-(imidazo[1,2-b]pyridazin-6-yl)piperidin-4-yl)methanone;
(S)—N-(2-(4-(5-(2,5-difluorophenyl)-4,5-dihydro-1H-pyrazole-1-carbonyl)piperidin-1-yl)pyrimidin-4-yl)acetamide;
(S)—N-(6-(4-(5-phenyl-4,5-dihydro-1H-pyrazole-1-carbonyl)piperidin-1-yl)pyrimidin-4-yl)acetamide;
(S)—N-(6-(4-(5-(3,5-difluorophenyl)-4,5-dihydro-1H-pyrazole-1-carbonyl)piperidin-1-yl)pyrimidin-4-yl)acetamide;
(S)-(1-(5-fluoro-4-(4-methylpiperazin-1-yl)pyrimidin-2-yl)piperidin-4-yl)(5-phenyl-4,5-dihydro-1H-pyrazol-1-yl)methanone;
(S)-(1-(2-(dimethylamino)pyrimidin-4-yl)piperidin-4-yl)(5-phenyl-4,5-dihydro-1H-pyrazol-1-yl)methanone;
(S)-2-(4-(5-(2,5-difluorophenyl)-4,5-dihydro-1H-pyrazole-1-carbonyl)piperidin-1-yl)pyrimidine-5-carboxamide;
(S)-5-chloro-2-(4-(5-(5-fluoropyridin-3-yl)-4,5-dihydro-1H-pyrazole-1-carbonyl)piperidin-1-yl)pyrimidine-4-carboxamide;
(S)—N-cyclopropyl-2-(4-(5-phenyl-4,5-dihydro-1H-pyrazole-1-carbonyl)piperidin-1-yl)pyrimidine-4-carboxamide;
(S)—N-(2-hydroxyethyl)-2-(4-(5-phenyl-4,5-dihydro-1H-pyrazole-1-carbonyl)piperidin-1-yl)pyrimidine-4-carboxamide;
(S)-(1-(5-fluoro-4-(2-morpholinoethoxy)pyrimidin-2-yl)piperidin-4-yl)(5-phenyl-4,5-dihydro-1H-pyrazol-1-yl)methanone;
(S)-(1-(5-hydroxypyrimidin-2-yl)piperidin-4-yl)(5-phenyl-4,5-dihydro-1H-pyrazol-1-yl)methanone;
(S)-(1-(6-(5-methyl-1,3,4-oxadiazol-2-yl)pyrimidin-4-yl)piperidin-4-yl)(5-phenyl-4,5-dihydro-1H-pyrazol-1-yl)methanone;
(S)-(1-(4-(hydroxymethyl)pyrimidin-2-yl)piperidin-4-yl)(5-phenyl-4,5-dihydro-1H-pyrazol-1-yl)methanone;
(S)-2-(4-(5-phenyl-4,5-dihydro-1H-pyrazole-1-carbonyl)piperidin-1-yl)pyrimidine-5-carboxamide;
(S)-2-(4-(5-(3,5-difluorophenyl)-4,5-dihydro-1H-pyrazole-1-carbonyl)piperidin-1-yl)oxazole-5-carboxamide;
(S)-2-(4-(5-(3,5-difluorophenyl)-4,5-dihydro-1H-pyrazole-1-carbonyl)piperidin-1-yl)oxazole-5-carbonitrile;
(S)-2-(4-(5-(3,5-difluorophenyl)-4,5-dihydro-1H-pyrazole-1-carbonyl)piperidin-1-yl)oxazole-4-carboxamide;
(S)-2-(4-(5-(3,5-difluorophenyl)-4,5-dihydro-1H-pyrazole-1-carbonyl)piperidin-1-yl)oxazole-4-carbonitrile;
(S)-2-(4-(5-phenyl-4,5-dihydro-1H-pyrazole-1-carbonyl)piperidin-1-yl)oxazole-4-carboxamide;
(S)-2-(4-(5-phenyl-4,5-dihydro-1H-pyrazole-1-carbonyl)piperidin-1-yl)oxazole-4-carbonitrile;
(S)-2-(4-(5-phenyl-4,5-dihydro-1H-pyrazole-1-carbonyl)piperidin-1-yl)oxazole-5-carbonitrile;
(S)-(1-(7H-purin-2-yl)piperidin-4-yl)(5-(3,5-difluorophenyl)-4,5-dihydro-1H-pyrazol-1-yl)methanone;
(S)-(1-(4-(4,5-dihydro-1H-imidazol-2-yl)pyrimidin-2-yl)piperidin-4-yl)(5-phenyl-4,5-dihydro-1H-pyrazol-1-yl)methanone,
(S)-(4-methylpiperazin-1-yl)(2-(4-(5-phenyl-4,5-dihydro-1H-pyrazole-1-carbonyl)piperidin-1-yl)pyrimidin-4-yl)methanone;
(S)—N,N-diethyl-2-(4-(5-phenyl-4,5-dihydro-1H-pyrazole-1-carbonyl)piperidin-1-yl)pyrimidine-4-carboxamide;
(S)—N,N-dimethyl-2-(4-(5-phenyl-4,5-dihydro-1H-pyrazole-1-carbonyl)piperidin-1-yl)pyrimidine-4-carbohydrazide;
(S)—N-(1-acetylpiperidin-4-yl)-2-(4-(5-phenyl-4,5-dihydro-1H-pyrazole-1-carbonyl)piperidin-1-yl)pyrimidine-4-carboxamide;
(S)-(1-(4-(morpholine-4-carbonyl)pyrimidin-2-yl)piperidin-4-yl)(5-phenyl-4,5-dihydro-1H-pyrazol-1-yl)methanone;
(S)-(5-phenyl-4,5-dihydro-1H-pyrazol-1-yl)(1-(4-(piperazine-1-carbonyl)pyrimidin-2-yl)piperidin-4-yl)methanone;
(S)-2-(4-(5-phenyl-4,5-dihydro-1H-pyrazole-1-carbonyl)piperidin-1-yl)-N-(piperidin-4-yl)pyrimidine-4-carboxamide;
(S)-(1-(4-(2H-tetrazol-5-yl)pyrimidin-2-yl)piperidin-4-yl)(5-phenyl-4,5-dihydro-1H-pyrazol-1-yl)methanone;
(S)-(1-(4-(2H-tetrazol-5-yl)pyrimidin-2-yl)piperidin-4-yl)(5-(5-fluoropyridin-3-yl)-4,5-dihydro-1H-pyrazol-1-yl)methanone;
3-(5-fluoro-2-(4-((S)-5-phenyl-4,5-dihydro-1H-pyrazole-1-carbonyl)piperidin-1-yl)pyrimidin-4-yl)pyrrolidin-2-one;
3-(5-fluoro-2-(4-((S)-5-(5-fluoropyridin-3-yl)-4,5-dihydro-1H-pyrazole-1-carbonyl)piperidin-1-yl)pyrimidin-4-yl)pyrrolidin-2-one;
(S)-2-((2-(4-(5-phenyl-4,5-dihydro-1H-pyrazole-1-carbonyl)piperidin-1-yl)pyrimidin-4-yl)oxy)acetic acid;
(S)-(1-(4-((2H-tetrazol-5-yl)methoxy)-5-fluoropyrimidin-2-yl)piperidin-4-yl)(5-phenyl-4,5-dihydro-1H-pyrazol-1-yl)methanone;
(S)-(1-(5-fluoro-4-(2-hydroxyethoxy)pyrimidin-2-yl)piperidin-4-yl)(5-phenyl-4,5-dihydro-1H-pyrazol-1-yl)methanone;
(S)-(1-(6-ethynylpyrimidin-4-yl)piperidin-4-yl)(5-phenyl-4,5-dihydro-1H-pyrazol-1-yl)methanone;
(S)-(5-(3,5-difluorophenyl)-4,5-dihydro-1H-pyrazol-1-yl)(1-(6-ethynylpyrimidin-4-yl)piperidin-4-yl)methanone;
(S)-3-(1-(1-(6-ethynylpyrimidin-4-yl)piperidine-4-carbonyl)-4,5-dihydro-1H-pyrazol-5-yl)benzonitrile;
(S)-5-fluoro-6-(4-(5-phenyl-4,5-dihydro-1H-pyrazole-1-carbonyl)piperidin-1-yl)pyrimidine-4-carboxylic acid;
(S)-5-fluoro-6-(4-(5-(5-fluoropyridin-3-yl)-4,5-dihydro-1H-pyrazole-1-carbonyl)piperidin-1-yl)pyrimidine-4-carboxamide;
(S)-5-fluoro-2-(4-(5-(5-fluoropyridin-3-yl)-4,5-dihydro-1H-pyrazole-1-carbonyl)piperidin-1-yl)pyrimidine-4-carboxamide;
(S)-5-fluoro-2-(4-(5-phenyl-4,5-dihydro-1H-pyrazole-1-carbonyl)piperidin-1-yl)pyrimidine-4-carboxamide;
(S)—N,N-diethyl-5-fluoro-6-(4-(5-phenyl-4,5-dihydro-1H-pyrazole-1-carbonyl)piperidin-1-yl)pyrimidine-4-carboxamide;
(S)-6-(4-(5-(3,5-difluorophenyl)-4,5-dihydro-1H-pyrazole-1-carbonyl)piperidin-1-yl)-5-fluoropyrimidine-4-carboxylic acid;
(S)-6-(4-(5-(3,5-difluorophenyl)-4,5-dihydro-1H-pyrazole-1-carbonyl)piperidin-1-yl)-5-fluoropyrimidine-4-carboxamide;
(R)-3-(5-fluoro-2-(4-((S)-5-(5-fluoropyridin-3-yl)-4,5-dihydro-1H-pyrazole-1-carbonyl)piperidin-1-yl)pyrimidin-4-yl)pyrrolidin-2-one;
(S)-2-((5-fluoro-2-(4-(5-phenyl-4,5-dihydro-1H-pyrazole-1-carbonyl)piperidin-1-yl)pyrimidin-4-yl)oxy)acetamide;
(1-(4-(morpholin-3-yl)pyrimidin-2-yl)piperidin-4-yl)((S)-5-phenyl-4,5-dihydro-1H-pyrazol-1-yl)methanone;
(S)-2-(5-fluoro-2-(4-(5-phenyl-4,5-dihydro-1H-pyrazole-1-carbonyl)piperidin-1-yl)pyrimidin-4-yl)acetamide;
or a pharmaceutically acceptable salt thereof.

In one embodiment, this invention is directed to (S)-5-fluoro-6-(4-(5-phenyl-4,5-dihydro-1H-pyrazole-1-carbonyl)piperidin-1-yl)pyrimidine-4-carboxylic acid, or a pharmaceutically acceptable salt thereof. In another embodiment, this invention is directed to (S)-6-(4-(5-(3,5-difluorophenyl)-4,5-dihydro-1H-pyrazole-1-carbonyl)piperidin-1-yl)pyrimidinel-4-carbonitrile. In another embodiment, this invention is directed to (S)-(5-(3,5-difluorophenyl)-4,5-dihydro-1H-pyrazol-1-yl)(1-(5-methyl-1,3,4-oxadiazol-2-yl)piperidin-4-yl)methanone. In still another embodiment, this invention is directed to (S)-6-(4-(5-(5-fluoropyridin-3-yl)-4,5-dihydro-1H-pyrazole-1-carbonyl)piperidin-1-yl)pyrimidine-4-carboxamide.

The invention also includes various deuterated forms of the compounds of Formula (I) and Formula (II). Each available hydrogen atom attached to a carbon atom may be independently replaced with a deuterium atom. A person of ordinary skill in the art will know how to synthesize deuterated forms of the compounds of Formula (I) and Formula (II). For example, commercially available deuterated starting materials may be employed in the preparation of deuterated analogs of the compounds of Formula (I) and Formula (II) or they may be synthesized using conventional techniques employing deuterated reagents (e.g. by reduction using lithium aluminum deuteride or sodium borodeuteride or by metal-halogen exchange followed by quenching with D₂O or methanol-d₃).

The skilled artisan will appreciate that solvates (particularly hydrates) of a compound of Formula (I) and Formula (II), including solvates of salts of a compound of Formula (I) and Formula (II), may be formed when solvent molecules are incorporated into the crystalline lattice during crystallization. The present invention includes within its scope all possible stoichiometric and non-stoichiometric salt and/or hydrate forms.

When a disclosed compound or its salt is named or depicted by structure, it is to be understood that the compound or salt, including solvates (particularly hydrates) thereof, may exist in crystalline forms, non-crystalline forms or a mixture thereof. The compound or salt, or solvates (particularly, hydrates) thereof, may also exhibit polymorphism (i.e. the capacity to occur in different crystalline forms). These different crystalline forms are typically known as “polymorphs.” It is to be understood that when named or depicted by structure, the disclosed compound, or solvates (particularly, hydrates) thereof, also include all polymorphs thereof. Polymorphs have the same chemical composition but differ in packing, geometrical arrangement, and other descriptive properties of the crystalline solid state. Polymorphs, therefore, may have different physical properties such as shape, density, hardness, deformability, stability, and dissolution properties. Polymorphs typically exhibit different melting points, IR spectra, and X-ray powder diffraction patterns, which may be used for identification. One of ordinary skill in the art will appreciate that different polymorphs may be produced, for example, by changing or adjusting the conditions used in crystallizing/recrystallizing the compound.

The present invention is directed to crystalline (S)-6-(4-(5-(5-fluoropyridin-3-yl)-4,5-dihydro-1H-pyrazole-1-carbonyl)piperidin-1-yl)pyrimidine-4-carboxamide (hereinafter “Compound A”).

In one embodiment, a crystalline form of (S)-6-(4-(5-(5-fluoropyridin-3-yl)-4,5-dihydro-1H-pyrazole-1-carbonyl)piperidin-1-yl)pyrimidine-4-carboxamide (Compound A—Form 1) is characterized by an X-ray powder diffraction (XRPD) pattern comprising at least five diffraction angles, when measured using Cu K_{α} radiation, selected from a group consisting of about 13.2, 18.5, 21.7, 22.7, 26.3, and 27.7 degrees 2θ. In another embodiment, Compound A—Form 1 is characterized by an X-ray powder diffraction (XRPD) pattern comprising at least four diffraction angles or at least three diffraction angles, when measured using Cu Kα radiation, selected from a group consisting of about 13.2, 18.5, 21.7, 22.7, 26.3, and 27.7 degrees 2θ. In another embodiment, Compound A—Form 1 is characterized by an X-ray powder diffraction (XRPD) pattern comprising at least three diffraction angles, when measured using Cu K_{α} radiation, selected from a group consisting of about 13.2, 18.5, 21.7, 22.7, 26.3, and 27.7 degrees 2θ.

In another embodiment, Compound A—Form 1 is characterized by an X-ray powder diffraction (XRPD) pattern comprising diffraction angles, when measured using Cu K_{α} radiation, of about 13.2, 18.5, 21.7, 22.7, and 27.7 degrees 2θ. In yet another embodiment, Compound A—Form 1 is characterized by an X-ray powder diffraction (XRPD) pattern substantially in accordance with FIG. 13.

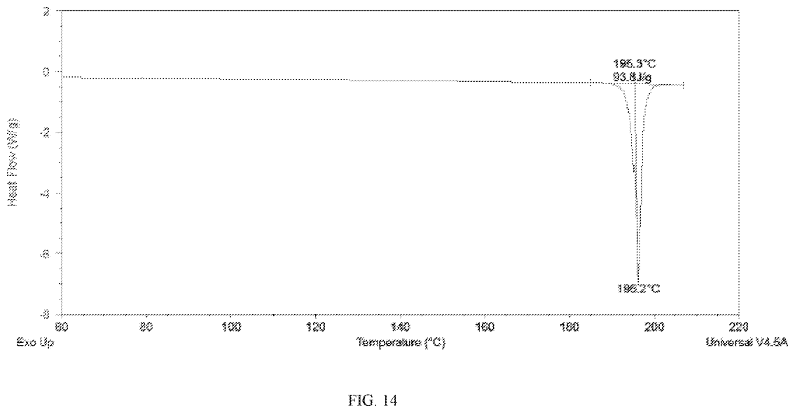

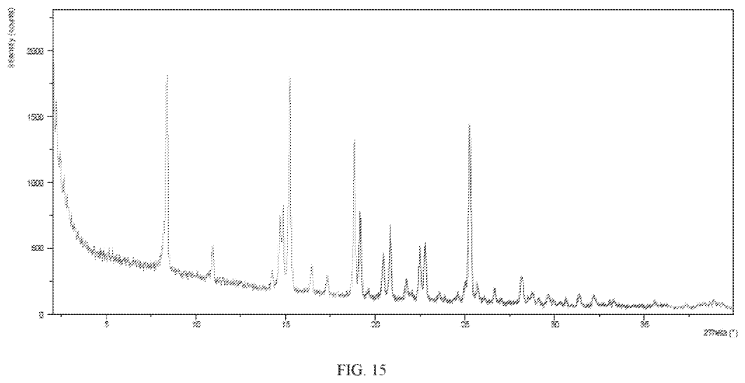

In further embodiments, Compound A—Form 1 is characterized by a differential scanning calorimetry trace substantially in accordance with FIG. 14.

In still further embodiments, as a person having ordinary skill in the art will understand, Compound A—Form 1 is characterized by any combination of the analytical data characterizing the aforementioned embodiments. For example, in one embodiment, Compound A—Form 1 is characterized by an X-ray powder diffraction (XRPD) pattern substantially in accordance with FIG. 13 and a differential scanning calorimetry trace substantially in accordance with FIG. 14. In another embodiment, Compound A—Form 1 is characterized by an X-ray powder diffraction (XRPD) pattern comprising diffraction angles, when measured using Cu K_{α} radiation, of about 13.2, 18.5, 21.7, 22.7, and 27.7 degrees 2θ, and a differential scanning calorimetry trace substantially in accordance with FIG. 14.

An XRPD pattern will be understood to comprise a diffraction angle (expressed in degrees 2θ) of “about” a value specified herein when the XRPD pattern comprises a diffraction angle within ±0.1 degrees 2θ of the specified value. Further, it is well known and understood to those skilled in the art that the apparatus employed, humidity, temperature, orientation of the powder crystals, and other parameters involved in obtaining an X-ray powder diffraction (XRPD) pattern may cause some variability in the appearance, intensities, and positions of the lines in the diffraction pattern. An X-ray powder diffraction pattern that is “substantially in accordance” with that of FIG. 13 provided herein is an XRPD pattern that would be considered by one skilled in the art to represent a compound possessing the same crystal form as the compound that provided the XRPD pattern of FIG. 13. That is, the XRPD pattern may be identical to that of FIG. 13, or more likely it may be somewhat different. Such an XRPD pattern may not necessarily show each of the lines of the diffraction pattern presented herein, and/or may show a slight change in appearance, intensity, or a shift in position of said lines resulting from differences in the conditions involved in obtaining the data. A person skilled in the art is capable of determining if a sample of a crystalline compound has the same form as, or a different form from, a form disclosed herein by comparison of their XRPD patterns. For example, one skilled in the art can overlay an XRPD pattern of a sample of a crystalline form of ((S)-6-(4-(5-(5-fluoropyridin-3-yl)-4,5-dihydro-1H-pyrazole-1-carbonyl)piperidin-1-yl)pyrimidine-4-carboxamide, with FIG. 13 and, using expertise and knowledge in the art, readily determine whether the XRPD pattern of the sample is substantially in accordance with the XRPD pattern of Compound A—Form 1. If the XRPD pattern is substantially in accordance with FIG. 13, the sample form can be readily and accurately identified as having the same form as Compound A—Form 1.

It is to be understood that the references herein to a compound of Formula (I) or Formula (II), or a salt thereof, includes a compound of Formula (I) or Formula (II) as a free base, acid, or as a salt thereof, for example as a pharmaceutically acceptable salt thereof. Thus, in one embodiment, the invention is directed to a compound of Formula (I) or Formula (II). In a further embodiment, the invention is directed to a pharmaceutically acceptable salt of a compound of Formula (I) or Formula (II). In a further embodiment, the invention is directed to a compound of Formula (I) or Formula (II), or a pharmaceutically acceptable salt thereof.

Because of their potential use in medicine, it will be appreciated that a salt of a compound of Formula (I) or Formula (II) is preferably pharmaceutically acceptable. Suitable pharmaceutically acceptable salts can include acid or base addition salts.

As used herein, the term “pharmaceutically acceptable” means a compound which is suitable for pharmaceutical use. Salts and solvates (e.g. hydrates and hydrates of salts) of the compounds of Formulas (I) and (II) which are suitable for use in medicine are those wherein the counterion or associated solvent is pharmaceutically acceptable.

Pharmaceutically acceptable salts include, amongst others, those described in Berge, J. Pharm. Sci., 66, 1-19, (1977) or those listed in P. H. Stahl and C. G. Wermuth, editors, Handbook of Pharmaceutical Salts; Properties, Selection and Use, Second Edition Stahl/Wermuth: Wiley—VCH/VHCA (2011) (see http://www.wiley.com/WileyCDA/WileyTitle/productCd-3906390519.html).

Suitable pharmaceutically acceptable salts can include acid or base addition salts.

Such base addition salts can be formed by reaction of a compound of Formula (I) or Formula (II) (which, for example, contains a carboxylic acid or other acidic functional group) with the appropriate base, optionally in a suitable solvent such as an organic solvent, to give the salt which can be isolated by a variety of methods, including crystallisation and filtration.

Such acid addition salts can be formed by reaction of a compound of Formula (I) or Formula (II) (which, for example contains a basic amine or other basic functional group) with the appropriate acid, optionally in a suitable solvent such as an organic solvent, to give the salt which can be isolated by a variety of methods, including crystallisation and filtration.

Salts may be prepared in situ during the final isolation and purification of a compound of Formula (I) or Formula (II). If a basic compound of Formula (I) or Formula (II) is isolated as a salt, the corresponding free base form of that compound may be prepared by any suitable method known to the art, including treatment of the salt with an inorganic or organic base, suitably an inorganic or organic base having a higher pKₐ than the free base form of the compound. Similarly, if a compound of Formula (I) or Formula (II) containing a carboxylic acid or other acidic functional group is isolated as a salt, the corresponding free acid form of that compound may be prepared by any suitable method known to the art, including treatment of the salt with an inorganic or organic acid. This invention also provides for the conversion of one salt of a compound of this invention, e.g., a hydrochloride salt, into another salt of a compound of this invention, e.g., a sulfate salt.

It will be understood that if a compound of Formula (I) or Formula (II) contains two or more basic moieties, the stoichiometry of salt formation may include 1, 2 or more equivalents of acid. Such salts would contain 1, 2 or more acid counterions, for example, a dihydrochloride salt.

Stoichiometric and non-stoichiometric forms of a pharmaceutically acceptable salt of a compound of Formula (I) or Formula (II) are included within the scope of the invention, including sub-stoichiometric salts, for example where a counterion contains more than one acidic proton.

Representative pharmaceutically acceptable acid addition salts include, but are not limited to, 4-acetamidobenzoate, acetate, adipate, alginate, ascorbate, aspartate, benzenesulfonate (besylate), benzoate, bisulfate, bitartrate, butyrate, calcium edetate, camphorate, camphorsulfonate (camsylate), caprate (decanoate), caproate (hexanoate), caprylate (octanoate), cinnamate, citrate, cyclamate, digluconate, 2,5-dihydroxybenzoate, disuccinate, dodecylsulfate (estolate), edetate (ethylenediaminetetraacetate), estolate (lauryl sulfate), ethane-1,2-disulfonate (edisylate), ethanesulfonate (esylate), formate, fumarate, galactarate (mucate), gentisate (2,5-dihydroxybenzoate), glucoheptonate (gluceptate), gluconate, glucuronate, glutamate, glutarate, glycerophosphorate, glycolate, hexylresorcinate, hippurate, hydrabamine (N,N′-di(dehydroabietyl)-ethylenediamine), hydrobromide, hydrochloride, hydroiodide, hydroxynaphthoate, isobutyrate, lactate, lactobionate, laurate, malate, maleate, malonate, mandelate, methanesulfonate (mesylate), methylsulfate, mucate, naphthalene-1,5-disulfonate (napadisylate), naphthalene-2-sulfonate (napsylate), nicotinate, nitrate, oleate, palmitate, p-aminobenzenesulfonate, p-aminosalicyclate, pamoate (embonate), pantothenate, pectinate, persulfate, phenylacetate, phenylethylbarbiturate, phosphate, polygalacturonate, propionate, p-toluenesulfonate (tosylate), pyroglutamate, pyruvate, salicylate, sebacate, stearate, subacetate, succinate, sulfamate, sulfate, tannate, tartrate, teoclate (8-chlorotheophyllinate), thiocyanate, triethiodide, undecanoate, undecylenate, and valerate.

Representative pharmaceutically acceptable base addition salts include, but are not limited to, aluminium, 2-amino-2-(hydroxymethyl)-1,3-propanediol (TRIS), arginine, benethamine (N-benzylphenethylamine), benzathine (N,N′-dibenzylethylenediamine), bis-(2-hydroxyethyl)amine, bismuth, calcium, chloroprocaine, choline, clemizole (1-p chlorobenzyl-2-pyrrolildine-1′-ylmethylbenzimidazole), cyclohexylamine, dibenzylethylenediamine, diethylamine, diethyltriamine, dimethylamine, dimethylethanolamine, dopamine, ethanolamine, ethylenediamine, L-histidine, iron, isoquinoline, lepidine, lithium, lysine, magnesium, meglumine (N-methylglucamine), piperazine, piperidine, potassium, procaine, quinine, quinoline, sodium, strontium, t-butylamine, tromethamine (tris(hydroxymethyl)aminomethane), and zinc.

It will be understood that if a compound of Formula (I) or Formula (II) contained two or more basic moieties, the stoichiometry of salt formation may include 1, 2 or more equivalents of acid. Such salts would contain 1, 2 or more acid counterions, for example, a diacetate or a dihydrochloride salt.

Because the compounds of Formulas (I) and (II), or a pharmaceutically acceptable salt thereof, are intended for use in pharmaceutical compositions it will readily be understood that they are each preferably provided in substantially pure form, for example at least 60% pure, more suitably at least 75% pure and preferably at least 85%, especially at least 98% pure (% are on a weight for weight basis). Impure preparations of the compounds may be used for preparing the more pure forms used in the pharmaceutical compositions.

The compounds of this invention may be particularly useful for the treatment of RIP1 kinase-mediated diseases or disorders. Such RIP1 kinase-mediated diseases or disorders are diseases or disorders that are mediated by activation of RIP1 kinase, and as such, are diseases or disorders where inhibition of RIP1 kinase would provide benefit.

In this invention, RIP1 kinase-mediated diseases or disorders are diseases or disorders that are mediated by activation of RIP1 kinase, and as such, are diseases or disorders where inhibition of RIP1 kinase would provide benefit. Such RIP1 kinase-mediated diseases or disorders are diseases/disorders which are likely to be regulated at least in part by programmed necrosis, apoptosis or the production of inflammatory cytokines, particularly inflammatory bowel disease (including Crohn's disease and ulcerative colitis), psoriasis, retinal detachment, retinal degeneration, retinitis pigmentosa, macular degeneration, age-related macular degeneration, pancreatitis, atopic dermatitis, arthritis (including rheumatoid arthritis, spondyloarthritis, gout, juvenile idiopathic arthritis (systemic onset juvenile idiopathic arthritis (SoJIA)), psoriatic arthritis), lupus, systemic lupus erythematosus (SLE), Sjogren's syndrome, systemic scleroderma, anti-phospholipid syndrome (APS), vasculitis, osteoarthritis, liver damage/diseases (non-alcohol steatohepatitis (NASH), alcohol steatohepatitis (ASH), autoimmune hepatitis, autoimmune hepatobiliary diseases, primary sclerosing cholangitis (PSC), acetaminophen toxicity, hepatotoxicity), non-alcohol steatohepatitis (NASH), alcohol steatohepatitis (ASH), autoimmune hepatitis, non-alcoholic fatty liver disease (NAFLD), kidney damage/injury (nephritis, renal transplant, surgery, administration of nephrotoxic drugs e.g. cisplatin, acute kidney injury (AKI)) Celiac disease, autoimmune idiopathic thrombocytopenic purpura (autoimmune ITP), transplant rejection (rejection of transplant organs, tissues and cells), ischemia reperfusion injury of solid organs, sepsis, systemic inflammatory response syndrome (SIRS), cerebrovascular accident (CVA, stroke), myocardial infarction (MI), atherosclerosis, Huntington's disease, Alzheimer's disease, Parkinson's disease, amyotrophic lateral sclerosis (ALS), progressive supranuclear palsy (PSP), neonatal brain injury, neonatal hypoxic brain injury, ischemic brain injury, traumatic brain injury allergic diseases (including asthma and atopic dermatitis), peripheral nerve injury, burns, multiple sclerosis, type I diabetes, type II diabetes, obesity, Wegener's granulomatosis, pulmonary sarcoidosis, Behcet's disease, interleukin-1 converting enzyme (ICE, also known as caspase-1) associated fever syndrome, chronic obstructive pulmonary disease (COPD), cigarette smoke-induced damage, cystic fibrosis, tumor necrosis factor receptor-associated periodic syndrome (TRAPS), a neoplastic tumor, peridontitis, NEMO-mutations (mutations of NF-kappa-B essential modulator gene (also known as IKK gamma or IKKG)), particularly, NEMO-deficiency syndrome, HOIL-1 deficiency (also known as RBCK1) heme-oxidized IRP2 ubiquitin ligase-1 deficiency), linear ubiquitin chain assembly complex (LUBAC) deficiency syndrome, hematological and solid organ malignancies, bacterial infections and viral infections (such as influenza, staphylococcus, and mycobacterium (tuberculosis)), and Lysosomal storage diseases (particularly, Gaucher disease, and including GM2 gangliosidosis, alpha-mannosidosis, aspartylglucosaminuria, cholesteryl ester storage disease, chronic hexosaminidase A deficiency, cystinosis, Danon disease, Fabry disease, Farber disease, fucosidosis, galactosialidosis, GM1 gangliosidosis, mucolipidosis, infantile free sialic acid storage disease, juvenile hexosaminidase A deficiency, Krabbe disease, lysosomal acid lipase deficiency, metachromatic leukodystrophy, mucopolysaccharidoses disorders, multiple sulfatase deficiency, Niemann-Pick disease, neuronal ceroid lipofuscinoses, Pompe disease, pycnodysostosis, Sandhoff disease, Schindler disease, sialic acid storage disease, Tay-Sachs, and Wolman disease), Stevens-Johnson syndrome, toxic epidermal necrolysis, glaucoma, spinal cord injury, fibrosis, complement-mediated cytotoxicity, pancreatic ductal adenocarcinoma, hepatocellular carcinoma, mesothelioma, melanoma, metastasis, breast cancer, non-small cell lung carcinoma (NSCLC), radiation induced necrosis, ischemic kidney damage, ophthalmologic ischemia, intracerebral hemorrhage, subarachnoid hemorrhage, acute liver failure and radiation protection/mitigation, auditory disorders such as noise-induced hearing loss and drugs associated with ototoxicity such as cisplatin, or for the treatment of cells ex vivo to preserve vitality and function.

The compounds of the invention, particularly the compounds of Formula (I) and Formula (II), or a pharmaceutically acceptable salt thereof, may be particularly useful for the treatment of the following RIP1 kinase-mediated diseases or disorders: inflammatory bowel disease (including Crohn's disease and ulcerative colitis), psoriasis, retinal detachment, retinal degeneration, retinitis pigmentosa, macular degeneration, age-related macular degeneration, pancreatitis, atopic dermatitis, arthritis (including rheumatoid arthritis, spondyloarthritis, gout, systemic onset juvenile idiopathic arthritis (SoJIA), psoriatic arthritis), lupus, systemic lupus erythematosus (SLE), Sjogren's syndrome, systemic scleroderma, anti-phospholipid syndrome (APS), vasculitis, osteoarthritis, liver damage/diseases (non-alcohol steatohepatitis (NASH), alcohol steatohepatitis (ASH), autoimmune hepatitis, autoimmune hepatobiliary diseases, primary sclerosing cholangitis (PSC), acetaminophen toxicity, hepatotoxicity), non-alcoholic steatohepatitis (NASH), alcoholic steatohepatitis (ASH), autoimmune hepatitis, non-alcoholic fatty liver disease (NAFLD), kidney damage/injury (nephritis, renal transplant, surgery, administration of nephrotoxic drugs e.g. cisplatin, acute kidney injury (AKI)) Celiac disease, autoimmune idiopathic thrombocytopenic purpura (autoimmune ITP), transplant rejection (rejection of transplant organs, tissues and cells), ischemia reperfusion injury of solid organs, sepsis, systemic inflammatory response syndrome (SIRS), cerebrovascular accident (CVA, stroke), myocardial infarction (MI), atherosclerosis, Huntington's disease, Alzheimer's disease, Parkinson's disease, amyotrophic lateral sclerosis (ALS), progressive supranuclear palsy (PSP), neonatal brain injury, neonatal hypoxic brain injury, traumatic brain injury, allergic diseases (including asthma and atopic dermatitis), peripheral nerve injury, burns, multiple sclerosis, type I diabetes, type II diabetes, obesity, Wegener's granulomatosis, pulmonary sarcoidosis, Behcet's disease, interleukin-1 converting enzyme (ICE, also known as caspase-1) associated fever syndrome, chronic obstructive pulmonary disease (COPD), cigarette smoke-induced damage, cystic fibrosis, tumor necrosis factor receptor-associated periodic syndrome (TRAPS), a neoplastic tumor, melanoma, metastasis, breast cancer, non-small cell lung carcinoma (NSCLC), radiation induced necrosis, ischemic kidney damage, ophthalmologic ischemia, intracerebral hemorrhage, subarachnoid hemorrhage, peridontitis, NEMO-mutations (mutations of NF-kappa-B essential modulator gene (also known as IKK gamma or IKKG)), particularly, NEMO-deficiency syndrome, HOIL-1 deficiency ((also known as RBCK1) heme-oxidized IRP2 ubiquitin ligase-1 deficiency), linear ubiquitin chain assembly complex (LUBAC) deficiency syndrome, hematological and solid organ malignancies, bacterial infections and viral infections (such as influenza, staphylococcus, and mycobacterium (tuberculosis)), and Lysosomal storage diseases (particularly, Gaucher disease, and including GM2 gangliosidosis, alpha-mannosidosis, aspartylglucosaminuria, cholesteryl ester storage disease, chronic hexosaminidase A deficiency, cystinosis, Danon disease, Fabry disease, Farber disease, fucosidosis, galactosialidosis, GM1 gangliosidosis, mucolipidosis, infantile free sialic acid storage disease, juvenile hexosaminidase A deficiency, Krabbe disease, lysosomal acid lipase deficiency, metachromatic leukodystrophy, mucopolysaccharidoses disorders, multiple sulfatase deficiency, Niemann-Pick disease, neuronal ceroid lipofuscinoses, Pompe disease, pycnodysostosis, Sandhoff disease, Schindler disease, sialic acid storage disease, Tay-Sachs, and Wolman disease), spinal cord injury, Stevens-Johnson syndrome, fibrosis, complement-mediated cytotoxicity, toxic epidermal necrolysis, and/or for the treatment of cells ex vivo to preserve vitality and function.

The compounds of the invention, particularly the compounds of Formula (I) and Formula (II), or a pharmaceutically acceptable salt thereof, may be particularly useful for the treatment of the following RIP1 kinase-mediated diseases or disorders, that is, diseases/disorders which are likely to be regulated at least in part by RIP1 kinase activity, particularly inflammatory bowel disease (including Crohn's disease and ulcerative colitis), rheumatoid arthritis, chronic obstructive pulmonary disease (COPD), asthma, cigarette smoke-induced damage, cystic fibrosis, psoriasis, retinal detachment, retinal degeneration, retinitis pigmentosa, macular degeneration, atopic dermatitis, burn injury, periodontitis, a bacterial or viral infection (an infection with a pathogen including but not limited to influenza, staphylococcus, and/or mycobacterium (tuberculosis), systemic scleroderma (particularly, topical treatment of hardened and/or tightened skin areas), and/or ischemia reperfusion injury of solid organs/transplant rejection (particularly, topical treatment of donor organ (particularly kidney, liver, and heart and/or lung transplants), infusion of organ recipient), and topical treatment of bowels.

The compounds of the invention, particularly the compounds of Formula (I) and Formula (II), or a pharmaceutically acceptable salt thereof, may be useful for the treatment of glaucoma.

The compounds of the invention, particularly the compounds of Formulas (I) and (II), or a pharmaceutically acceptable salt thereof, may be particularly useful for treatment of pancreatic ductal adenocarcinoma, hepatocellular carcinoma, mesothelioma, or melanoma.

The compounds of the invention, particularly the compounds of Formulas (I) and (II), or a pharmaceutically acceptable salt thereof, may be particularly useful for the treatment of the following RIP1 kinase-mediated disease or disorder: rheumatoid arthritis, inflammatory bowel disease (including Crohn's disease and ulcerative colitis), and psoriasis.

The treatment of the above-noted diseases/disorders may concern, more specifically, the amelioration of organ injury or damage sustained as a result of the noted diseases/disorders. For example, the compounds of this invention may be particularly useful for amelioration of brain tissue injury or damage following ischemic brain injury or traumatic brain injury, or for amelioration of heart tissue injury or damage following myocardial infarction, or for amelioration of brain tissue injury or damage associated with Huntington's disease, Alzheimer's disease or Parkinson's disease, or for amelioration of liver tissue injury or damage associated with non-alcohol steatohepatitis, alcohol steatohepatitis, autoimmune hepatitis autoimmune hepatobiliary diseases, or primary sclerosing cholangitis, or overdose of acetaminophen.

The compounds of this invention may be particularly useful for the amelioration of organ injury or damage sustained as a result of radiation therapy, or amelioration of spinal tissue injury or damage following spinal cord injury or amelioration of liver tissue injury or damage associated acute liver failure. The compounds of this invention may be particularly useful for amelioration of auditory disorders, such as noise-induced hearing loss or auditory disorders following the administration of ototoxic drugs or substances e.g. cisplatin.

The compounds of this invention may be particularly useful for amelioration of solid organ tissue (particularly kidney, liver, and heart and/or lung) injury or damage following transplant or the administration of nephrotoxic drugs or substances e.g. cisplatin. It will be understood that amelioration of such tissue damage may be achieved where possible, by pre-treatment with a compound of Formula (I) or Formula (II), or a pharmaceutically acceptable salt thereof; for example, by pre-treatment of a patient prior to administration of cisplatin or pre-treatment of an organ or the organ recipient prior to transplant surgery. Amelioration of such tissue damage may be achieved by treatment with a compound of Formula (I) or Formula (II), or a pharmaceutically acceptable salt thereof, during transplant surgery. Amelioration of such tissue damage may also be achieved by short-term treatment of a patient with a compound of Formula (I) or Formula (II), or a pharmaceutically acceptable salt thereof, after transplant surgery.

In one embodiment, the compounds of the invention, particularly the compounds of Formulas (I) and (II), or a pharmaceutically acceptable salt thereof, may be useful for the treatment of retinal detachment, macular degeneration, and retinitis pigmentosa.

In another embodiment, the compounds of the invention, particularly the compounds of Formulas (I) and (II), or a pharmaceutically acceptable salt thereof, may be useful for the treatment of multiple sclerosis.

In one embodiment, the compounds of the invention, particularly the compounds of Formulas (I) and (II), or a pharmaceutically acceptable salt thereof, may be useful for the treatment of traumatic brain injury.

In another embodiment, the compounds of the invention, particularly the compounds of Formulas (I) and (II), or a pharmaceutically acceptable salt thereof, may be useful for the treatment of Huntington's Disease, Alzheimer's Disease, amyotrophic lateral sclerosis, and Niemann-Pick disease.

In another embodiment, the compounds of the invention, particularly the compounds of Formula (I) and Formula (II), or a pharmaceutically acceptable salt thereof, may be useful for the treatment of amyotrophic lateral sclerosis (ALS), progressive supranuclear palsy (PSP), and Alzheimer's disease.

In another embodiment, the compounds of the invention, particularly the compounds of Formulas (I) and (II), or a pharmaceutically acceptable salt thereof, may be useful for the treatment of age-related macular degeneration.

The treatment of retinal detachment, macular degeneration, retinitis pigmentosa, multiple sclerosis, traumatic brain injury, Huntington's Disease, Alzheimer's Disease, amyotrophic lateral sclerosis, and Niemann-Pick disease may concern, more specifically, the amelioration of organ injury or damage sustained as a result of these diseases/disorders. For example, the compounds of this invention may be particularly useful for amelioration of brain tissue injury or damage following traumatic brain injury, or for amelioration of brain tissue injury or damage associated of Huntington's Disease, Alzheimer's Disease, amyotrophic lateral sclerosis, and Niemann-Pick disease.

In another embodiment, the compounds of the invention, particularly the compounds of Formulas (I) and (II), or a pharmaceutically acceptable salt thereof, may be useful for the treatment of retinal detachment, macular degeneration, and retinitis pigmentosa, and the amelioration of brain tissue injury or damage as a result of multiple sclerosis, traumatic brain injury, Huntington's Disease, Alzheimer's Disease, amyotrophic lateral sclerosis, and Niemann-Pick disease.

In another embodiment, the compounds of the invention, particularly the compounds of Formulas (I) and (II), or a pharmaceutically acceptable salt thereof, may be useful for the treatment of Crohn's disease, ulcerative colitis, psoriasis, rheumatoid arthritis, spondyloarthritis, systemic onset juvenile idiopathic arthritis (SoJIA), and osteoarthritis.

In yet another embodiment, the compounds of this invention, particularly the compounds of Formula (I) and Formula (II), or a pharmaceutically acceptable salt thereof, may be useful for the treatment of psoriasis, rheumatoid arthritis, and ulcerative and colitis.

In another embodiment, the compounds of this invention, particularly the compounds of Formula (I) and Formula (II), or a pharmaceutically acceptable salt thereof, may be useful for the treatment of lupus, inflammatory bowel disease (IBD), Crohn's disease, and ulcerative colitis.

In another embodiment, the compounds of the invention, particularly the compounds of Formulas (I) and (II), or a pharmaceutically acceptable salt thereof, may be useful for the treatment of cerebrovascular accident (CVA, stroke), Huntington's disease, Alzheimer's disease, amyotrophic lateral sclerosis (ALS), traumatic brain injury, multiple sclerosis, Gaucher disease, Niemann-Pick disease, and spinal cord injury.

In another embodiment, the compounds of the invention, particularly the compounds of Formulas (I) and (II), or a pharmaceutically acceptable salt thereof, may be useful for the treatment of amyotrophic lateral sclerosis (ALS).

In another embodiment, the compounds of the invention, particularly the compounds of Formulas (I) and (II), or a pharmaceutically acceptable salt thereof, may be useful for the treatment of multiple sclerosis.

In another embodiment, the compounds of the invention, particularly the compounds of Formula (I) and Formula (II), or a pharmaceutically acceptable salt thereof, may be useful for the treatment of pancreatic ductal adenocarcinoma (PDAC), metastasis, melanoma, breast cancer, non-small cell lung carcinoma (NSCLC), and radiation induced necrosis.

In another embodiment, the compounds of the invention, particularly the compounds of Formula (I) and Formula (II), or a pharmaceutically acceptable salt thereof, may be useful for the treatment of pancreatic ductal adenocarcinoma (PDAC), metastasis, melanoma, breast cancer, and non-small cell lung carcinoma (NSCLC).

In another embodiment, the compounds of the invention, particularly the compounds of Formula (I) and Formula (II), or a pharmaceutically acceptable salt thereof, may be useful for the treatment of pancreatic ductal adenocarcinoma (PDAC).

In another embodiment, the compounds of the invention, particularly the compounds of Formula (I) and Formula (II), or a pharmaceutically acceptable salt thereof, may be useful for the treatment of intracerebral hemorrhage and subarachnoid hemorrhage.

In another embodiment, the compounds of the invention, particularly the compounds of Formula (I) and Formula (II), or a pharmaceutically acceptable salt thereof, may be useful for the treatment of type II diabetes and obesity.

In another embodiment, the compounds of the invention, particularly the compounds of Formula (I) and Formula (II), or a pharmaceutically acceptable salt thereof, may be useful for the treatment of atherosclerosis.

In another embodiment, the compounds of the invention, particularly the compounds of Formula (I) and Formula (II), or a pharmaceutically acceptable salt thereof, may be useful for the treatment of vasculitis.

In another embodiment, the compounds of the invention, particularly the compounds of Formula (I) and Formula (II), or a pharmaceutically acceptable salt thereof, may be useful for the treatment of burns.

In another embodiment, the compounds of the invention, particularly the compounds of Formula (I) and Formula (II), or pharmaceutically acceptable salt thereof, may be useful for the treatment of ischemic kidney damage, ophthalmologic ischemia, intracerebral hemorrhage, and subarachnoid hemorrhage.

In another embodiment, the compounds of the invention, particularly the compounds of Formula (I) and Formula (II), or pharmaceutically acceptable salt thereof, may be useful f or the treatment of non-alcoholic steatohepatitis (NASH), alcoholic steatohepatitis (ASH), autoimmune hepatitis, and non-alcoholic fatty liver disease (NAFLD).

The compounds of the invention, particularly the compounds of Formulas (I) and (II), or a pharmaceutically acceptable salt thereof, may be particularly useful for the treatment of the following RIP1 kinase-mediated diseases or disorders. In one aspect the human has a solid tumor. In one aspect the tumor is selected from head and neck cancer, gastric cancer, melanoma, renal cell carcinoma (RCC), esophageal cancer, non-small cell lung carcinoma (NSCLC), prostate cancer, colorectal cancer, ovarian cancer, pancreatic cancer, and pancreatic ductal adenocarcinoma. In one aspect the human has one or more of the following: colorectal cancer (CRC), esophageal cancer, cervical, bladder, breast cancer, head and neck cancer, ovarian cancer, melanoma, renal cell carcinoma (RCC), EC squamous cell carcinoma, non-small cell lung carcinoma, mesothelioma, prostate cancer, and pancreatic ductal adenocarcinoma. In another aspect, the human has a liquid tumor such as diffuse large B cell lymphoma (DLBCL), multiple myeloma, chronic lyphomblastic leukemia (CLL), follicular lymphoma, acute myeloid leukemia and chronic myelogenous leukemia.

The present disclosure also relates to a method for treating or lessening the severity of a cancer selected from: brain (gliomas), glioblastomas, astrocytomas, Bannayan-Zonana syndrome, Cowden disease, Lhermitte-Duclos disease, breast cancer, triple negative breast cancer, inflammatory breast cancer, Wilm's tumor, Ewing's sarcoma, Rhabdomyosarcoma, ependymoma, medulloblastoma, colon cancer, head and neck cancer (including squamous cell carcinoma of head and neck), kidney cancer, lung cancer (including lung squamous cell carcinoma, lung adenocarcinoma, lung small cell carcinoma, and non-small cell lung carcinoma), liver cancer (including hepatocellular carcinoma), melanoma, ovarian cancer, pancreatic cancer (including squamous pancreatic cancer), prostate cancer, sarcoma, osteosarcoma, giant cell tumor of bone, thyroid cancer, lymphoblastic T-cell leukemia, chronic myelogenous leukemia, chronic lymphocytic leukemia, hairy-cell leukemia, acute lymphoblastic leukemia, acute myelogenous leukemia, chronic neutrophilic leukemia, acute lymphoblastic T-cell leukemia, plasmacytoma, immunoblastic large cell leukemia, mantle cell leukemia, multiple myeloma megakaryoblastic leukemia, multiple myeloma, acute megakaryocytic leukemia, promyelocytic leukemia, erythroleukemia, malignant lymphoma, Hodgkin's lymphoma, Non-Hodgkin's lymphoma, lymphoblastic T cell lymphoma, Burkitt's lymphoma, follicular lymphoma, neuroblastoma, bladder cancer, urothelial cancer, lung cancer, vulval cancer, cervical cancer, endometrial cancer, cancer of the uterus, renal cancer (including kidney clear cell cancer, kidney papillary cancer, renal cell carcinoma), mesothelioma, esophageal cancer, salivary gland cancer, hepatocellular cancer, gastric cancer, nasopharangeal cancer, buccal cancer, cancer of the mouth, GIST (gastrointestinal stromal tumor) and testicular cancer.

Specific examples of clinical conditions based on hematologic tumors include leukemias such as chronic myelocytic leukemia, acute myelocytic leukemia, chronic lymphocytic leukemia and acute lymphocytic leukemia; plasma cell malignancies such as multiple myeloma, MGUS and Waldenstrom's macroglobulinemia; lymphomas such as non-Hodgkin's lymphoma, Hodgkin's lymphoma; and the like.

The cancer may be any cancer in which an abnormal number of blast cells or unwanted cell proliferation is present or that is diagnosed as a hematological cancer, including both lymphoid and myeloid malignancies. Myeloid malignancies include, but are not limited to, acute myeloid (or myelocytic or myelogenous or myeloblastic) leukemia (undifferentiated or differentiated), acute promyeloid (or promyelocytic or promyelogenous or promyeloblastic) leukemia, acute myelomonocytic (or myelomonoblastic) leukemia, acute monocytic (or monoblastic) leukemia, erythroleukemia and megakaryocytic (or megakaryoblastic) leukemia. These leukemias may be referred together as acute myeloid (or myelocytic or myelogenous) leukemia (AML). Myeloid malignancies also include myeloproliferative disorders (MPD) which include, but are not limited to, chronic myelogenous (or myeloid) leukemia (CML), chronic myelomonocytic leukemia (CMML), essential thrombocythemia (or thrombocytosis), and polcythemia vera (PCV). Myeloid malignancies also include myelodysplasia (or myelodysplastic syndrome or MDS), which may be referred to as refractory anemia (RA), refractory anemia with excess blasts (RAEB), and refractory anemia with excess blasts in transformation (RAEBT); as well as myelofibrosis (MFS) with or without agnogenic myeloid metaplasia.

Specific examples of clinical conditions based on hematologic tumors include leukemias such as chronic myelocytic leukemia, acute myelocytic leukemia, chronic lymphocytic leukemia and acute lymphocytic leukemia; plasma cell malignancies such as multiple myeloma, MGUS and Waldenstrom's macroglobulinemia; lymphomas such as non-Hodgkin's lymphoma, Hodgkin's lymphoma; and the like.

Hematopoietic cancers also include lymphoid malignancies, which may affect the lymph nodes, spleens, bone marrow, peripheral blood, and/or extranodal sites. Lymphoid cancers include B-cell malignancies, which include, but are not limited to, B-cell non-Hodgkin's lymphomas (B-NHLs). B-NHLs may be indolent (or low-grade), intermediate-grade (or aggressive) or high-grade (very aggressive). Indolent B cell lymphomas include follicular lymphoma (FL); small lymphocytic lymphoma (SLL); marginal zone lymphoma (MZL) including nodal MZL, extranodal MZL, splenic MZL and splenic MZL with villous lymphocytes; lymphoplasmacytic lymphoma (LPL); and mucosa-associated-lymphoid tissue (MALT or extranodal marginal zone) lymphoma. Intermediate-grade B-NHLs include mantle cell lymphoma (MCL) with or without leukemic involvement, diffuse large cell lymphoma (DLBCL), follicular large cell (or grade 3 or grade 3B) lymphoma, and primary mediastinal lymphoma (PML). High-grade B-NHLs include Burkitt's lymphoma (BL), Burkitt-like lymphoma, small non-cleaved cell lymphoma (SNCCL) and lymphoblastic lymphoma. Other B-NHLs include immunoblastic lymphoma (or immunocytoma), primary effusion lymphoma, HIV associated (or AIDS related) lymphomas, and post-transplant lymphoproliferative disorder (PTLD) or lymphoma. B-cell malignancies also include, but are not limited to, chronic lymphocytic leukemia (CLL), prolymphocytic leukemia (PLL), Waldenstrom's macroglobulinemia (WM), hairy cell leukemia (HCL), large granular lymphocyte (LGL) leukemia, acute lymphoid (or lymphocytic or lymphoblastic) leukemia, and Castleman's disease. NHL may also include T-cell non-Hodgkin's lymphoma s(T-NHLs), which include, but are not limited to T-cell non-Hodgkin's lymphoma not otherwise specified (NOS), peripheral T-cell lymphoma (PTCL), anaplastic large cell lymphoma (ALCL), angioimmunoblastic lymphoid disorder (AILD), nasal natural killer (NK) cell/T-cell lymphoma, gamma/delta lymphoma, cutaneous T cell lymphoma, mycosis fungoides, and Sezary syndrome.

Hematopoietic cancers also include Hodgkin's lymphoma (or disease) including classical Hodgkin's lymphoma, nodular sclerosing Hodgkin's lymphoma, mixed cellularity Hodgkin's lymphoma, lymphocyte predominant (LP) Hodgkin's lymphoma, nodular LP Hodgkin's lymphoma, and lymphocyte depleted Hodgkin's lymphoma. Hematopoietic cancers also include plasma cell diseases or cancers such as multiple myeloma (MM) including smoldering MM, monoclonal gammopathy of undetermined (or unknown or unclear) significance (MGUS), plasmacytoma (bone, extramedullary), lymphoplasmacytic lymphoma (LPL), Waldenstrom's Macroglobulinemia, plasma cell leukemia, and primary amyloidosis (AL). Hematopoietic cancers may also include other cancers of additional hematopoietic cells, including polymorphonuclear leukocytes (or neutrophils), basophils, eosinophils, dendritic cells, platelets, erythrocytes and natural killer cells. Tissues which include hematopoietic cells referred herein to as “hematopoietic cell tissues” include bone marrow; peripheral blood; thymus; and peripheral lymphoid tissues, such as spleen, lymph nodes, lymphoid tissues associated with mucosa (such as the gut-associated lymphoid tissues), tonsils, Peyer's patches and appendix, and lymphoid tissues associated with other mucosa, for example, the bronchial linings.

Treatment of RIP1-mediated disease conditions may be achieved using a compound of the invention, particularly a compound of Formula (I) or Formula (II), or a pharmaceutically acceptable salt thereof, of as a monotherapy, or in dual or multiple combination therapy, particularly for the treatment of refractory cases, such as in combination with other anti-inflammatory and/or anti-TNF agents, which may be administered in therapeutically effective amounts as is known in the art.

The compounds of the invention, particularly the compounds of Formula (I) and Formula (II), or a pharmaceutically acceptable salt thereof, may be employed alone or in combination with one or more other therapeutic agents, e.g., pharmaceutically active compounds or biologic products (e.g., monoclonal antibodies). Combination therapies according to the present invention thus comprise the administration of at least one compound of the invention, particularly a compound of Formula (I) or Formula (II), or a pharmaceutically acceptable salt thereof, and at least one other therapeutically active agent. Combination therapies according to the present invention comprise the administration of at least one compound of the invention, particularly a compound of Formula (I) or Formula (II), or a pharmaceutically acceptable salt thereof, and at least one other therapeutic ally active agent, specifically one or two other therapeutically active agents, more specifically one other therapeutically active agent.

For example, amelioration of tissue damage may be achieved by treatment with a compound of Formula (I) or Formula (II), or a pharmaceutically acceptable salt thereof, and at least one other therapeutically active agent during transplant surgery. Amelioration of tissue damage may also be achieved by short-term treatment of a patient with a compound of Formula (I) or (II), or a pharmaceutically acceptable salt thereof, and at least one other therapeutic ally active agent after transplant surgery. Amelioration of tissue damage ex vivo, that is ex vivo preservation of tissues, organs and cells may also be achieved by short-term treatment of tissues, organs and cells with a compound of Formula (I) or Formula (II), or a pharmaceutically acceptable salt thereof, and at least one other therapeutic ally active agent, prior to or during transplant surgery.

The compound(s) of the invention, particularly the compounds of Formula (I) and Formula (II), or pharmaceutically acceptable salts thereof, and the other therapeutic agent(s) may be administered together in a single pharmaceutical composition or separately and, when administered separately this may occur simultaneously or sequentially in any order. The amounts of the compound(s) of the invention, particularly a compound of Formula (I) or Formula (II), or pharmaceutically acceptable salts thereof, and the other therapeutic agent(s) and the relative timings of administration will be selected in order to achieve the desired combined therapeutic effect. Thus, in a further aspect, there is provided a combination comprising a compound of the invention, particularly a compound of Formula (I) or Formula (II), or a pharmaceutically acceptable salt thereof, together with one or more other therapeutic agents, specifically one or two other therapeutically active agents, more specifically one other therapeutically active agent. In one aspect, there is provided a combination comprising (S)-5-fluoro-6-(4-(5-phenyl-4,5-dihydro-1H-pyrazole-1-carbonyl)piperidin-1-yl)pyrimidine-4-carboxylic acid, or a pharmaceutically acceptable salt thereof, together with one or more other therapeutic agents, specifically one or two other therapeutically active agents, more specifically one other therapeutically active agent. In one aspect, there is provided a combination comprising (S)-6-(4-(5-(3,5-difluorophenyl)-4,5-dihydro-1H-pyrazole-1-carbonyl)piperidin-1-yl)pyrimidine-4-carbonitrile, or a pharmaceutically acceptable salt thereof, together with one or more other therapeutic agents, specifically one or two other therapeutically active agents, more specifically one other therapeutically active agent. In another aspect, there is provided a combination comprising (S)-(5-(3,5-difluorophenyl)-4,5-dihydro-1H-pyrazol-1-yl)(1-(5-methyl-1,3,4-oxadiazol-2-yl)piperidin-4-yl)methanone, or a pharmaceutically acceptable salt thereof, together with one or more other therapeutic agents, specifically one or two other therapeutically active agents, more specifically one other therapeutically active agent. In one aspect, there is provided a combination comprising (S)-6-(4-(5-(5-fluoropyridin-3-yl)-4,5-dihydro-1H-pyrazole-1-carbonyl)piperidin-1-yl)pyrimidine-4-carboxamide, or a pharmaceutically acceptable salt thereof, together with one or more other therapeutic agents, specifically one or two other therapeutically active agents, more specifically one other therapeutically active agent.

Thus, in one aspect of this invention, a compound of the invention, particularly a compound of Formula (I) or Formula (II), or a pharmaceutically acceptable salt thereof, or a pharmaceutical composition comprising a compound of the invention, particularly a compound of Formula (I) or Formula (II), or a pharmaceutically acceptable salt thereof, may be used in combination with or include one or more other therapeutic agents, for example an anti-inflammatory agent and/or an anti-TNF agent.

The pharmaceutical compositions of the invention typically contain one compound of the invention. However, in certain embodiments, the pharmaceutical compositions of the invention contain more than one compound of the invention. In other embodiments, the pharmaceutical compositions of the invention may comprise one or more additional therapeutic agents, specifically one or two other therapeutically active agents, more specifically one other therapeutically active agent.

A compound that inhibits RIP1 kinase, particularly a compound of Formula (I) or Formula (II), or a pharmaceutically acceptable salt thereof, may be administered in combination with other anti-inflammatory agents for any of the indications above, including oral or topical corticosteroids, anti-TNF agents, 5-aminosalicyclic acid and mesalamine preparations, hydroxycloroquine, thiopurines, methotrexate, cyclophosphamide, cyclosporine, calcineurin inhibitors, mycophenolic acid, mTOR inhibitors, JAK inhibitors, Syk inhibitors, anti-inflammatory biologic agents, including anti-IL6 biologics, anti-IL1 agents, anti-IL17 biologics, anti-CD22, anti-integrin agents, anti-IFNa, anti-CD20 or CD4 biologics and other cytokine inhibitors or biologics to T-cell or B-cell receptors or interleukins.

In the treatment of CVA, a compound that inhibits RIP1 kinase, particularly a compound of Formula (I) or Formula (II), or a pharmaceutically acceptable salt thereof, may be administered in combination with antiplatelets (e.g., aspirin, clopidogrel (Plavix®), dipyridamole (Persantine®), ticolpidine (Ticlid®); aspirin and omeprazole (Ysprala®)), anticoagulants (e.g., warfarin (Coumadin®), Heparin®, dabigitran (Pradaxa®), apixaban (Eliquis®), Rivaroxaban®), antihypertensives—diruetics (e.g., Hygroton®, Lasix®, Esidrix®, Hydrodiuril®, Microzide®, Lozol®, Mykrox®, Zaroxolyn®, Midarmar®, Aldactone®, Dyrenium®, Bumex®, Moduretic®, Aldatazide®, Dyazide®, Maxzide®), other antihypertensives—beta blockers, ace inhibitors, angiotensin II receptor blockers, calcium channel blockers, alpha blockers, alpha2 receptor agonist, combined alpha and beta-blockers, central agonists, peripheral adrenergic inhibitors, blood vessel dilators, or tissue plasminogen activator (Alteplase®).

In the treatment of SIRS, a compound that inhibits RIP1 kinase, particularly a compound of Formula (I) or Formula (II), or a pharmaceutically acceptable salt thereof, may be administered in combination with a broad-spectrum antibiotic (such as vacomycin) or other anti-MRSA therapy (cefeprime (Maxipime®), piperacillin/tazobactam (Zosyn®), carbapenem (imipenem, meropenem, doripenem), quinolones (ciprofloxacin, levofloxacin, ofloxacin, moxifloxacin, etc.), or low dose steroids such as hydrocortisones.

In the treatment of inflammatory bowel disease (particularly, Crohn's disease and/or ulcerative colitis), a compound that inhibits RIP1 kinase, particularly a compound of Formula (I) or Formula (II), or a pharmaceutically acceptable salt thereof, may be administered in combination with vedolizumab (Entyvio®), alicaforsen, remestemcel-L (Prochymal®), etrolizumab, eldelumab, or bertilimumab.

In the treatment of psoriasis, a compound that inhibits RIP1 kinase, particularly a compound of Formula (I) or Formula (II), or a pharmaceutically acceptable salt thereof, may be administered in combination with ixekizumab, tildrakizumab (MK-3222), secukinumab (AIN457), Alefacept (Amevive®), calcipotriene and betamethasone dipropionate (Enstilar®), prednisone (Rayos®), tazorac topical gel, Methotrexate (Trexall®, Rheumatrex®, Folex PFS®, Otrexup®, Rasuvo®, Methotrexate LPF Sodium®), Cyclosporine®, fumaric acid, Acitretin®, Tretinate®, UVA, UVB, Psoralen, coal tar, TNF inhibitors (Etanercept (Enbrel®), Infliximab (Remicade®), adalimumab (Humira®); certolizumab pegol (Cimzia®)), PDE-4 inhibitors (apremilast (Otezla®)), JAK inhibitors (Tofacitinib (Xeljanz® CP-690550), IL 12/23 (ustekinumab (Stelara®)), IL17 (secukinumab (Coxentyx®), ixekizumab (Taltz®), brodalumab with AMG-827), IL23 (tildrakizumab with MK-3222, guselkumab CNTO-1959, BI 655066, itolizumab (Alzumab®), biosimilars infliximab (Remsima (Inflectra®), Sandoz GP 11111), biosimilars rituximab (CT-P10 (Mabthera®), PF-05280586 (MabThera®)), biosimilars etanercept (CHS-2014), biosimilars adalimumab (GP-2017), M-518101 topical vitamin D; Maruho GK-664, or CT-327 (topical Tropomyosin-receptor kinase A), CF-101, or dimethyl fumarate LAS-41008.

In the treatment of periodonitis, a compound that inhibits RIP1 kinase, particularly a compound of Formula (I) or Formula (II), or a pharmaceutically acceptable salt thereof, may be administered in combination with an antimicrobial agent, (such as chlorhexidine (Peridex®, PerioChip®, PerioGard®, etc.)) or an antibiotic (such as doxycycline (Vibrox®, Periostat®, Monodox®, Oracea®, Doryx®, etc.), or minocycline (Dynacin®, Minocin®, Arestin®, Dynacin®, etc.).

In the treatment of asthma, a compound that inhibits RIP1 kinase, particularly a compound of Formula (I) or Formula (II), or a pharmaceutically acceptable salt thereof, may be administered in combination with an inhaled corticosteroid (ICS) such as fluticasone proprionate (Flovent®), fluticasone furoate (Veramyst®/Avamys®), beclomethasone dipropionate (QVAR®), budesonide (Pulmicort), trimcinolone acetonide (Azmacort®), flunisolide (Aerobid®), mometasone fuorate (Asmanex® Twisthaler®), or Ciclesonide (Alvesco®), a long acting beta agonist (LABA) such as formoterol fumarate (Foradil®), salmeterol xinafoate (Serevent®), indacaterol (Arcapta®Neohaler®); a combination of an ICS and LABA (such as fluticasone furoate and vilanterol (Breo Ellipta®/Relvar Ellipta®), formoterol/budesonide inhalation (Symbicort®), mometasone furoate/formoterol fumarate dihydrate (Dulera®), beclomethasone dipropionate/formoterol (Inuvair®), fluticasone propionate/eformoterol fumarate dehydrate (Flutiform®), and fluticasone propionate/salmeterol (Advair®), a short acting beta agonist ((SABA) such as salbutamol dry-powder inhalation, albuterol sulfate (ProAir®, Proventil HFA®, Ventolin HFA®, AccuNeb® Inhalation Solution), levalbuterol tartrate (Xopenex® HFA), an antimuscarinic agent such as ipratropium bromide (Atrovent® HFA); an antimuscarinic in combination with a beta-agonist such as ipratropium bromide/albuterol (Combivent® Respimat®); a long-acting muscarinic antagonist ((LAMA) such as umeclidinium bromide (Incruse®) or tiotropium bromide (Spiriva®HandiHaler; a combination of a LAMA and a LABA, such as umeclidinium bromide and vilanterol (Anoro®) a leukotriene modifier (such as montelukast sodium (Singulair®), zafirlukast (Accolate®), or zileuton (Zyflo®), and anti-IgE (such as omalizumab (Xolair®)), a methylxanthine bronchodilator (such as theophylline (Accurbron®, Aerolate®, Aquaphyllin®, Asbron®, Bronkodyl®, Duraphyl®, Elixicon®, Elixomin®, Labid®, Lanophyllin®, Quibron-T®, Slo-Bid®, Slo-Phyllin®, Somophyllin®, Sustaire®, Synophylate®, T-Phyll®, Theo-24®, Theo-Dur®, Theobid®, Theochron®, Theoclear®, Theolair®, Theolixir®, Theophyl®, Theovent®, Uni-dur®, Uniphyl®), a mast cell inhibitor (such as cromulyn sodium (Nasalcrom®) and nedocromil sodium (Tilade®)).

Other agents that may be suitable for use in combination therapy in the treatment of asthma include a protein tyrosine kinase inhibitor (masitinib), CRTH2/D-prostanoid receptor antangonist (AMG 853), an epinephrine inhalation aerosol (E004), reslizumab, Vectura's VR506, lebrikizumab (RG3637), a combination phosphodiesterase (PDE)-3 and (PDE)-4 inhibitor (RPL554).

In the treatment of COPD, a compound that inhibits RIP1 kinase, particularly a compound of Formula (I) or Formula (II), or a pharmaceutically acceptable salt thereof, may be administered in combination with a LABA (such as salmeterol xinafoate (Serevent), aformoterol tartrate (Brovana®), formoterol fumarate inhalation powder (Foradil®), indacterol maleate (Arcapta® Neohaler®), a long-acting inhaled anticholinergic (or muscarinic antagonist, such as umeclidinium (Incruse Ellipta®), tiotropium bromide (Spiriva®), and aclidinium bromide (Tudorza® Pressair®), a phosphodiesterase (PDE-r) inhibitor (such as roflumilast, Daliresp®), a combination ICS/LABA (such as fluticasone furoate and vilanterol (Breo Ellipta®/Relvar Ellipta®), fluticasone propionate/salmeterol (Advair®), budesonide/formoterol (Symbicort®), mometasone/formoterol (Dulera®), or fluticasone propionate/eformoterol fumarate dehydrate (Flutiform®); an antimuscarinic such as such as ipratropium bromide (Atrovent®); an antimuscarinic in combination with a beta-agonist such as ipratropium bromide/albuterol (Combivent® Respimat®); a long-acting antimuscarinic such as umeclidinium bromide (Incruse®) or tiotropium bromide (Spiriva®); umeclidinium/vilanterol (Anoro Ellipta®); a combination of a LAMA and a LABA, such as umeclidinium bromide and vilanterol (Anoro®).

Other agents that may be suitable for use in combination therapy in the treatment of COPD include SCH527123 (a CXCR2 antagonist), glycoprronium bromide ((NVA237) Seebri® Breezhaler®), glycopyrronium bromide and indacaterol maleate ((QVA149) Ultibro® Breezhaler®), glycopyrrolate and formoterol fumarate (PT003), indacaterol maleate (QVA149), olodaterol (Striverdi® Respimat®), tiotropium (Spiriva®)/olodaterol (Striverdi® Respimat®), and aclidinium/formoterol inhalation.

In the treatment of a mycobacterium infection (tuberculosis), a compound that inhibits RIP1 kinase, particularly a compound of Formula (I) or Formula (II), or a pharmaceutically acceptable salt thereof, may be administered in combination with an antimycobacterial agent (such as isoniazid (INH), ehambutol (Myambutol®), rifampin (Rifadin®), and pyrazinamide (PZA)) a bactericidal antibiotic (such as rifabutin (Mycobutin®) or rifapentine (Priftin®)), an aminoglycoside (Capreomycin®), a fluorquinolone (levofloxacin, moxifloxicin, ofloxacin), thioamide (ehionamide), cyclosporine (Sandimmune®), para-aminosalicyclic acid (Paser®), cycloserine (Seromycin®), kanamycin (Kantrex®), streptomycin, viomycin, capreomycin (Capastat®)), bedaquiline fumarate (Sirturo®), oxazolidinone (Sutezolid®), or delamanid (OPC-67683).

In the treatment of systemic scleroderma, a compound that inhibits RIP1 kinase, particularly a compound of Formula (I) or Formula (II), or a pharmaceutically acceptable salt thereof, may be administered in combination with an oral corticosteroid (such as prednisolone (Delatsone®, Orapred, Millipred, Omnipred, Econopred, Flo-Pred), an immunosuppressive agent (such as methotrexate (Rhuematrex®, Trexall®), cyclosporine (Sandimmune®), anti-thymocyte globulin (Atgam®), mycophenolate mofetil (CellCept®), cyclophosphamide (Cytoxan®), FK506 (tacrolimus), thalidomide (Thalomid®), chlorambucil (Leukeran®), azathioprine (Imuran®, Azasan®)), a calcium channel blocker (such as nifedipine (Procardia®, Adalat®) or nicardipine (Cardene®), a topical emollient (nitroglycerin ointment), an ACE inhibitor (such as lisinopril (Zestril®, diltaizem (Cardizem®, Cardizem SR®, Cardizem CD®, Cardia®, Dilacor®, Tiazac®)), a serotonin reuptake inhibitor (such as fluoxetine (Prozac®)), an endothelin-1 receptor inhibitor (such as bosentan (Tracleer®) or epoprostenol (Flolan®, Veletri®, Prostacyclin®)) an anti-fibrotic agent (such as colchicines (Colcrys®), para-aminobenzoic acid (PABA), dimethyl sulfoxide (KMSO), and D-penicillamine (Cuprimine®, Depen®), interferon alpha and interferon gamma (INF-g)), a proton-pump Inhibitor (such as omeprazole (Prilosec®), metoclopramide (Reglan®), lansoprazole (Prevacid®), esomeprazole (Nexium®), pantoprazole (Protonix®), rabeprazole (Aciphex®)) or imatinib (Gleevec®) ARG201 (arGentis Pharmaceutical), belimumab (Benlysta®), tocilizumab (Actema®).

In the treatment of cystic fibrosis, a compound that inhibits RIP1 kinase, particularly a compound of Formula (I) or Formula (II), or a pharmaceutically acceptable salt thereof, may be administered in combination with a cystic fibrosis transmembrane conductance regulator (CFTR) potentiator (ivacftor (Kalydeco®)) a mucolytic agent (such as dornase alpha (Pulmozyme®)), pancreatic enzymes (such as Pancrelipase (Creon®, Pancreaze®, Ultresa®, Zenpep®)), a bronchodilator (such as albuterol (AccuNeb®, ProAir®, Proventil HFA®, VoSpire ER®, Ventolin HFA®)), an antibiotic (including inhaled, oral or parenteral, such as tobramycin solution for inhalation (TOBI®, Bethkis®, TOBI Podhaler®), aztreonam inhalation (Azactam®, Cayston®), colistimethate sodium (Coly-Mycin®), cephalosporins (cefadroxil monohydrate (Duricef®), cefazolin (Kefzol®), cephalexin (Keflex®), cefazolin (Ancef®, etc.), fluoroquinolones (moxifloxacin, levofloxacin, gemifloxacin, etc.), azithromycin (Zithromax®), gentamicin (Garamycin®), piperacillin/tazobacam (Zosyn®), cephalexin (Keflex), ceftazidime (Fortaz, Tazicef), ciprofloxin (Cipro XR, Proquin XR), trimethoprim/sulfamethoxazolyl (Bactrim DS, Septra DS), chloramphenicol)), or ivacftor (Kalydeco®)/lumacaftor (VX-809), ataluren (Translarna®), or with tiopropium bromide (Spiriva® Handihaler®) as add on to standard therapy.

In the treatment of retinitis pigmentosa, a compound that inhibits RIP1 kinase, particularly a compound of Formula (I) or Formula (II), or a pharmaceutically acceptable salt thereof, may be administered in combination with a ciliary neurtotrophic growth factor (NT-501-CNTF) or gene transfer agent, UshStat®.

In the treatment of macular degeneration, a compound that inhibits RIP1 kinase, particularly a compound of Formula (I) or Formula (II), or a pharmaceutically acceptable salt thereof, may be administered in combination with opthalmalic intravitreal injections (afibercept (Eylea®)) or with an anti-vascular endothelial growth factor (VEGF) inhibitor (such as ranibizumab (Lucentis®) or pegaptanib sodium (Macugen®)), a ciliary neurotrophic growth factor agent (NT501), iSONEP®, or bevacizumab (Avastin®).

In the treatment of influenza, a compound that inhibits RIP1 kinase, particularly a compound of Formula (I) or Formula (II), or a pharmaceutically acceptable salt thereof, may be administered in combination with a trivalent (IIV3) inactivated influenza vaccine (such as Afluria®, Fluarix®, Flucelvax®, FluLaval®, Fluvirin®, Fluzone®), a quadrivalent (IIV4) inactivated influenza vaccine (such as Fluarix® Quadrivalent, Flulaval® Quadrivalent, Fluzone® Quadrivalent), a trivalent recombinant influenza vaccine (such as FluBlok®), a quadrivalent live attenuated influenza vaccine (such as FluMist® Quadrivalent), an antiviral agent (such as oseltamivir (Tamiflu®), zanamivir (Relenza®), rimantadine (Flumadine®), or amantadine (Symmetrel®)), or Fluad®, Fludase, FluNhance®, Preflucel, or VaxiGrip®.

In the treatment of a staphylococcus infection, a compound that inhibits RIP1 kinase, particularly a compound of Formula (I) or Formula (II), or a pharmaceutically acceptable salt thereof, may be administered in combination with an antibiotic (such as a β-Lactam cephalosporin (Duricef®, Kefzol®, Ancef®, Biocef®, etc.), nafcillin (Unipen®), a sulfonamide (sulfamethoxazolyl and trimethoprim (Bacrim®, Septra®,) sulfasalazine (Azulfidine®), acetyl sulfisoxazolyl (Gantrisin®, etc.), or vancomycin (Vancocin®)).

In the treatment of transplant rejection, a compound that inhibits RIP1 kinase, particularly a compound of Formula (I) or Formula (II), or a pharmaceutically acceptable salt thereof, may be administered in combination with a high-dose corticosteroid (such as prednisone (Deltasone®), methylprednisolone (SoluMedrol®) etc.) a calcineurin inhibitor (such as cyclosporine (Sandimmune®, Neoral®, Gengraf®), tacrolimus (Prograf®, Astragraf XL®)), an mTor inhibitor (such as sirolimus (Rapamune®) or everolimus (Afinitor®)), an anti-proliferative agent (such as azathioprine (Imuran®, Azasan®), mycophenolate mofetil (CellCept®), or mycophenolate sodium (Myfortic®)), a monoclonal antibody (such as muromonab-CD3 (Orthoclone OKT3®)), an interleukine-2 receptor antagonist ((Basiliximab®, Simulect®), daclizumab (Zenapax®), or rituximab (Rituxan®)), a polyclonal anti-T-cell antibody (such as anti-thymocyte gamma globulin-equine (Atgam®), or antithymocyte globulin-rabbit (Thymoglobulin®)) an anti-CD40 antagonist (ASKP-1240), a JAK inhibitor (ASP015K), or an anti-TCR murine mAb (TOL101).

In the treatment of atopic dermatitis, a compound that inhibits RIP1 kinase, particularly a compound of Formula (I) or Formula (II), or a pharmaceutically acceptable salt thereof, may be administered in combination with a topical immunomodulator or calcineurin inhibitor (such as pimecrolimus (Elidel®) or tacrolimus ointment (Protopic®)), a topical corticosteroid (such as hydrocortizone (Synacort®, Westcort®), betamethasone (Diprolene®), flurandrenolide (Cordan®), fluticasone (Cutivate®), triamcinolone (Kenalog®), fluocinonide (Lidex®), and clobetasol (Temovate®)), an oral corticosteroid (such as hydrocortisone (Cortef®), methylprednisolone (Medrol®), or prednisolone (Pediapred®, Prelone®), an immunosuppressant (such as cyclosporine (Neoral®) or interferon gamma (Alferon N®, Infergen®, Intron A, Roferon-A®)), an antihistamine (for itching such as Atarax®, Vistaril®, Benadryl®), an antibiotic (such as penicillin derivatives flucloxacillin (Floxapen®) or dicloxacillin (Dynapen®), erythromycin (Eryc®, T-Stat®, Erythra-Derm®, etc.)), anon-steroidal immunosuppressive agent (such as azathioprine (Imuran®, Azasan®), methotrexate (Rhuematrex®, Trexall®), cyclosporin (Sandimmune®), or mycophenolate mofetil (CellCept®)).

In the treatment of spondyloarthritis, a compound that inhibits RIP 1 kinase, particularly a compound of Formula (I) or Formula (II), or a pharmaceutically acceptable salt thereof, may be administered in combination with NSAIDs, DMARDs such as Sulfasalazine®, Methotrexate®, and corticosteroids; prednisolone delayed-release tablets (Rayos®), TNF inhibitors (Enbrel®, Remicade®, Humira® and Simponi®), or IL-17A (Cosentyx®).

In the treatment of systemic onset juvenile idiopathic arthritis (sJIA), a compound that inhibits RIP 1 kinase, particularly a compound of Formula (I) or Formula (II), or a pharmaceutically acceptable salt thereof, may be administered in combination with NSAIDs such as Celebrex®, diclofenac (Voltaran®), ibuprofen (Advil®, Motrin®), naproxen (Aleve, Naprosyn®), corticosteroids (prednisone, glucocorticoids), Methotrexate®, or biologics (ankinra (Kineret®), tocilizumab (Actemra®), canakinumab (ILARIS®)).

In the treatment of osteoarthritis, a compound that inhibits RIP 1 kinase, particularly a compound of Formula (I) or Formula (II), or a pharmaceutically acceptable salt thereof, may be administered in combination with analgesics and NSAIDs (acetaminophen, opioid narcotics (e.g., Tramadol®, Vicodin®, Darvon®, Percocet®); ibuprofen and famotidine (Duexis®); Etadolac®; naproxen sodium (Naprelan®), diclofenac sodium topical solution (Pennsaid®); sodium hyaluronate (Supartz®); meloxicam (Vivlodex®, Mobic®); acetaminophen, ibuprofen, aspirin, Celecoxib®, COX-2 (Celebrex®), valdecoxib (Bextra®)), corticosteroid injections, hyaluronic acid injection (Gelsyn-3®); hylan GF 20 (Synvisc,® Synvisc-One®), or duloxetine hydrochloride (Cymbalta®).

In the treatment of or medication management of Huntington's disease, a compound that inhibits RIP 1 kinase, particularly a compound of Formula (I) or Formula (II), or a pharmaceutically acceptable salt thereof, may be administered in combination with tetrabenazine (Xenazine®), antipsychotic drugs (haloperidol (Haldol®), chlorpromazine HCL (Thorazine®), risperidone (Risperdal®) and quetiapine (Seroquel®)), drugs to suppress chorea (amantadine, devetiracetam (Keppra®), clonazepam (Klonopin®)), antidepressants (citalopram (Celexa®, Lexapro®), fluoxetine (Prozac®, Sarafem®), sertraline (Zoloft®)), antipsychotics (quetiapine (Seroquel®), risperidone (Risperdal®), olanzapine (Zyprexa®)), or mood-stabilizing drugs (vaproate (Depacon®), carbamazepine (Carbatrol®, Epitol®, Equetro®), lamotrigine (Lamictal®)).

In the treatment of Alzheimer's, a compound that inhibits RIP 1 kinase, particularly a compound of Formula (I) or Formula (II), or a pharmaceutically acceptable salt thereof, may be administered in combination with Donepzil hydrocholoride (Aricept®), Rivastigmine tartrate (Exelon®), caprylidene (Axona®), butoconazole nitrate 2% (Femstat 3®), Galantamine hydrobromide (Razadyne®, Reminyl®), Memantine HCL (Namenda®), memantine hydrocholoride extended release+donepezil hydrochloride (Namzaric®), Solanezumab, beta-secretase with Merck (MK-8931), beta-secretase with Cerespir (CSP-1103), or drugs that targets tau protein (AADvac1).

In the treatment of Amyotrophic lateral sclerosis (ALS), a compound that inhibits RIP 1 kinase, particularly a compound of Formula (I) or Formula (II), or a pharmaceutically acceptable salt thereof, may be administered in combination with a glutamate blocker (Riluzole (Rilutek®)),

In the treatment of symptoms with ALS, a compound that inhibits RIP 1 kinase, particularly a compound of Formula (I) or Formula (II), or a pharmaceutically acceptable salt thereof, may be administered in combination with quinidine (Nuedexta®), anticholinergics (Amitriptyline®, Artane®, scopolamine patch (Transderm Scop®)), sympathomimetics (pseudoephedrine), mucolytics (guaifenesin), or analgesics (tramadol (Ultram®); ketorolac (Toradol®); morphine; fentanyl patch (Duragesic®)).

In the treatment of multiple sclerosis, a compound that inhibits RIP 1 kinase, particularly a compound of Formula (I) or Formula (II), or a pharmaceutically acceptable salt thereof, may be administered in combination with corticosteroids (prednisone, methylprednisolone), Interferon Beta 1-A (Avonex®, Extavia®, Rebif®, Betaseron®), peginterferon beta-1A (Plegridy®), Glatiramer acetate (Copaxone®); glatiramer acetate (Glatopa®—generic equivalent of Copaxone); Dimethyl fumarate (Tecfidera®); Fingolimod (Gilenya®); teriflunomide (Aubagio®); dalfampridine (Ampyra®); daclizumab (Zinbryta); alemtuzumab (Lemtrada®); natalizumab (Tysabri®); or mitoxantrone hydrochloride (Novantrone®).

In the treatment of gaucher disease, a compound that inhibits RIP 1 kinase, particularly a compound of Formula (I) or Formula (II), or a pharmaceutically acceptable salt thereof, may be administered in combination with enzyme replacement therapy (imiglucerase (Cerezyme®), velaglucerase alfa (VPRIV®), taliglucerase alfa (Elelyso®)) or substrate reduction therapy (miglustat (Zavesca®), eliglustat (Cerdelga®)).

In the treatment of Niemann-Pick Disease, a compound that inhibits RIP 1 kinase, particularly a compound of Formula (I) or Formula (II), or a pharmaceutically acceptable salt thereof, may be administered in combination with bone marrow transplant, enzyme replacement therapy, gene therapy, miglustat (Zavesca®), Arimoclomol (BRX-345), NCT02612129, Hydroxypropyl-beta-cyclodexin (HPbCD), NCT01747135, or Hydroxypropyl-β-cyclodextrin (VTS-2702) (NCT02534844).

In the treatment of rheumatoid arthritis, a compound that inhibits RIP 1 kinase, particularly a compound of Formula (I) or Formula (II), or a pharmaceutically acceptable salt thereof, may be administered in combination with Tocilizumab (Actemra®), Arava, sulfasalazine delayed release tablets (Azulfidine EN-tabs®, Bextra, certolizumab pegol (Cimzia®), ibuprofen and famotidine (Duexis®), naproxen sodium (Etodolac®), adalimumab (Humira®), Kineret; etodolac (Lodine®), naproxen sodium (Naprelan), abatacept (Orencia), prednisone (Rayos®), inflimimab (Remicade®), golimuma (Simponi®), rofecoxib (Vioxx®), tofacitinib (Xeljanz®), methotrexate (Trexall®, Rheumatrex®, Folex PFS®, Otrexup®, Rasuvo®, Methotrexate LPF Sodium®, selective JAK1 & JAK2 inhbitor (baracitinib), antisense oligonucleotide (alicafosen), biosimilars for infliximab (Remsima (Inflectra®)), GS-071 infliximab (Aprogen), SB2 infliximab, PF-06438179 infliximab, GP 11111, biosimilars for rituximab (CT-P10 rituximab Celltrion), BI-695500, GP-2013, PF-05280586, biosimilars for etanercept (etanercept SB4 (Brenzys™), Benepali®; CHS-0214 etanercept, GP-2015, biosimilars for adalimumab (ABP-501 adalimumab, BI-695501, Samsung SB5, GP-2017. PF-06410293, Momenta M923, or biosimilar for abatacept (M834).

In the treatment of ulcerative colitis, a compound that inhibits RIP 1 kinase, particularly a compound of Formula (I) or Formula (II), or a pharmaceutically acceptable salt thereof, may be administered in combination with alicafosen, Mesalamine (Asacol®), balsalazide disodium (Colazal®), vedolizumab (Entyvio®), golimumab (Simponi®), budesonide (Uceris®), adalimumab (Humira®), RG-7413 (alpha4beta7 integrin), CNTO-1275 (ustekinumab), biosimiar infliximab (Remsima (Inflectra®)), BMS eldelumab (CXCL 10), or Immune Pharma bertilimumab (CCR3).

In the treatment of Crohn's disease, a compound that inhibits RIP 1 kinase, particularly a compound of Formula (I) or Formula (II), or a pharmaceutically acceptable salt thereof, may be administered in combination with Remestemcel-L (Prochymal®), vedolizumab (Entyvio®), ustekinumab (Stelara®), certolizumab pegol (Cimzia®), natalizumab (Tysabri®), budesonide (Entocort EC®), anti-inflammatories (mesalamine (Lialda®, Apriso®, Canasa®, Asacol®, Rowasa®), sulfasalazine (Azulfidine®)), steroids (hydrocortisone, prednisone), immunosuppressants (methotrexate (Trexall®, Rasuvo®, Rheumatrex®), infliximab (Remicade®), azathioprine (Azasan®, Imuran®), adalimumab (Humira®), mercaptopurine (Purinethol®, Purixan®); cyclosporine (Gengraf®, Neoral®, Sandimuune®); tacrolimus (Astagraf XL®, Hecoria®)), or antibiotics (metronidazole (Flagyl®, Metrogel®, Noritate®, MetroCream®, Rosadan®, MetroLotion®); ciprofloxacin (Cipro®)).

In one embodiment of this invention, the at least one other therapeutically active agent is selected from a thrombolytic agent, a tissue plasminogen activator, an anticoagulant, and a platelet aggregation inhibitor. In another embodiment, the at least one other therapeutically active agent is selected from heparin, coumadin, clopidrogel, dipyridamole, ticlopidine HCL, eptifibatide, and aspirin. In one embodiment, the RIP1 kinase-mediated disease or disorder treated with these agents is a cerebrovascular accident.

In one embodiment of this invention, the at least one other therapeutically active agent is selected from broad-spectrum antibiotic, anti-MRSA therapy and a low dose steroid. In another embodiment, the at least one other therapeutically active agent is selected from vacomycin, cefeprime, a combination of piperacillin and tazobactam, imipenem, meropenem, doripenem, ciprofloxacin, levofloxacin, ofloxacin, moxifloxacin, and hydrocortisone. In one embodiment, the RIP1 kinase-mediated disease or disorder treated with these agents is systemic inflammatory response syndrome.

In one embodiment of this invention, the at least one other therapeutically active agent is alicaforse or remestemcel-L. In one embodiment, the RIP1 kinase-mediated disease or disorder treated with these agents is Crohn's disease or ulcerative colitis.

In one embodiment of this invention, the at least one other therapeutically active agent is ixekizumab, or tildrakizumab. In one embodiment, the RIP1 kinase-mediated disease or disorder treated with these agents is psoriasis.

In one embodiment of this invention, the at least one other therapeutically active agent is an antimicrobial agent or an antibiotic. In another embodiment, the at least one other therapeutically active agent is selected from chlorhexidine, doxycycline and minocycline. In one embodiment, the RIP1 kinase-mediated disease or disorder treated with these agents is periodonitis.

In one embodiment of this invention, the at least one other therapeutically active agent is selected from an inhaled corticosteroid, a long acting beta agonist, a combination of an inhaled corticosteroid and a long acting beta agonist, a short acting beta agonist, a leukotriene modifier, an anti-IgE, a methylxanthine bronchodilator, a mast cell inhibitor, and a long-acting muscarinic antagonist. In another embodiment, the at least one other therapeutically active agent is selected from fluticasone proprionate, beclomethasone dipropionate, budesonide, trimcinolone acetonide, flunisolide, mometasone fuorate, or ciclesonide, formoterol fumarate, salmeterol xinafoate, a combination of fluticasone furoate and vilanterol, a combination of formoterol and budesonide inhalation, a combination of beclomethasone dipropionate and formoterol, a combination of fluticasone propionate and salmeterol, albuterol sulfate, levalbuterol tartrate, a combination of ipratropium bromide and albuterol, ipratropium bromide, montelukast sodium, zafirlukast, zileuton, omalizumab theophylline, cromulyn sodium, nedocromil sodium, and a combination of mometasone furoate and formoterol fumarate dihydrate. In another embodiment, the at least one other therapeutically active agent is selected from protein tyrosine kinase inhibitor, a CRTH2/D-prostanoid receptor antangonist, an epinephrine inhalation aerosol, and a combination of a phosphodiesterase-3 inhibitor and a phosphodiesterase-4 inhibitor. In another embodiment, the at least one other therapeutically active agent is selected from masitinib, AMG 853, indacaterol, E004, a combination of fluticasone furoate and fluticasone proprionate, a combination of vinanterol fluticasone furoate, a combination of fluticasone propionate and eformoterol fumarate dehydrate, reslizumab, salbutamol, tiotropium bromide, a combination of formoterol and budesonide, fluticasone furoate, VR506, lebrikizumab, and RPL554. In one embodiment, the RIP1 kinase-mediated disease or disorder treated with these agents is asthma.

In one embodiment of this invention, the at least one other therapeutically active agent is selected from a long acting beta agonist, a long-acting inhaled anticholinergic or muscarinic antagonist, a phosphodiesterase inhibitor, a combination an inhaled corticosteroid long acting beta agonist, a short acting beta agonist, and an inhaled corticosteroid. In another embodiment, the at least one other therapeutically active agent is selected from salmeterol xinafoate, a combination of umeclidinium and vilanterol, umeclidinium, aformoterol tartrate, formoterol fumarate, indacterol maleate, a combination of fluticasone propionate and eformoterol fumarate dehydrate, tiotropium bromide, aclidinium bromide, roflumilast, a combination of fluticasone furoate and vilanterol, a combination of fluticasone propionate and salmeterol, a combination of budesonide and formoterol, a combination of mometasone and formoterol, a combination of ipratropium bromide and albuterol sulfate, a combination of albuterol and ipratropium, ipratropium bromide, albuterol sulfate, budesonide, fluticasone propionate, and beclometasone dipropionate. In another embodiment, the at least one other therapeutically active agent is selected from SCH527123, glycoprronium bromide, a combination of glycopyrronium bromide and indacaterol maleate, a combination of glycopyrrolate and formoterol fumarate, indacaterol maleate, olodaterol, tiotropium, olodaterol, and a combination of aclidinium and formoterol. In one embodiment, the RIP1 kinase-mediated disease or disorder treated with these agents is COPD.

In one embodiment of this invention, the at least one other therapeutically active agent is an antimycobacterial agent or a bactericidal antibiotic. In another embodiment, the at least one other therapeutically active agent is selected from isoniazid, ehambutol, rifampin, pyrazinamide, rifabutin, rifapentine, capreomycin, levofloxacin, moxifloxicin, ofloxacin, ehionamide, cycloserine, kanamycin, streptomycin, viomycin, bedaquiline fumarate, PNU-100480, and delamanid. In one embodiment, the RIP1 kinase-mediated disease or disorder treated with these agents is a mycobacterium infection.

In one embodiment of this invention, the at least one other therapeutically active agent is selected from an oral corticosteroid, anti-thymocyte globulin, thalidomide, chlorambucil, a calcium channel blocker, a topical emollient, an ACE inhibitor, a serotonin reuptake inhibitor, an endothelin-1 receptor inhibitor, an anti-fibrotic agent, a proton-pump inhibitor or imatinib, ARG201, and tocilizumab. In another embodiment, the at least one other therapeutically active agent is selected from prednisolone, anti-thymocyte globulin, FK506 (tacrolimus), thalidomide, chlorambucil, nifedipine, nicardipine, nitroglycerin ointment, lisinopril, diltaizem, fluoxetine, bosentan, epoprostenol, colchicines, para-aminobenzoic acid, dimethyl sulfoxide, D-penicillamine, interferon alpha, interferon gamma (INF-g)), omeprazole, metoclopramide, lansoprazole, esomeprazole, pantoprazole, rabeprazole, imatinib, ARG201, and tocilizumab. In one embodiment, the RIP1 kinase-mediated disease or disorder treated with these agents is systemic scleroderma.

In one embodiment of this invention, the at least one other therapeutically active agent is selected from a cystic fibrosis transmembrane conductance regulator potentiator, a mucolytic agent, pancreatic enzymes, a bronchodilator, an antibiotic, or ivacftor/lumacaftor, ataluren, and tiopropium bromide. In another embodiment, the at least one other therapeutically active agent is selected from ivacftor, dornase alpha, pancrelipase, albuterol, tobramycin, aztreonam, colistimethate sodium, cefadroxil monohydrate, cefazolin, cephalexin, cefazolin, moxifloxacin, levofloxacin, gemifloxacin, azithromycin, gentamicin, piperacillin/tazobacam, ceftazidime, ciprofloxin, trimethoprim/sulfamethoxazolyl, chloramphenicol, or ivacftor/lumacaftor, ataluren, and tiopropium bromide. In one embodiment, the RIP1 kinase-mediated disease or disorder treated with these agents is cystic fibrosis.

In one embodiment of this invention, the at least one other therapeutically active agent is a ciliary neurtotrophic growth factor or a gene transfer agent. In another embodiment, the at least one other therapeutically active agent is NT-501-CNTF or a gene transfer agent encoding myosin VIIA (MY07A). In one embodiment, the RIP1 kinase-mediated disease or disorder treated with these agents is retinitis pigmentosa.

In one embodiment of this invention, the at least one other therapeutically active agent is selected from opthalmalic intravitreal injections, an anti-vascular endothelial growth factor inhibitor, and a ciliary neurotrophic growth factor agent. In another embodiment, the at least one other therapeutically active agent is selected from afibercept, ranibizumab, pegaptanib sodium, NT501, humanized sphingomab, and bevacizumab. In one embodiment, the RIP1 kinase-mediated disease or disorder treated with these agents is macular degeneration.

In one embodiment of this invention, the at least one other therapeutically active agent is selected from a trivalent (IIV3) inactivated influenza vaccine, a quadrivalent (IIV4) inactivated influenza vaccine, a trivalent recombinant influenza vaccine, a quadrivalent live attenuated influenza vaccine, an antiviral agent, or inactivated influenza vaccine. In another embodiment, the at least one other therapeutically active agent is selected from oseltamivir, zanamivir, rimantadine, or amantadine. In one embodiment, the RIP1 kinase-mediated disease or disorder treated with these agents is influenza.

In one embodiment of this invention, the at least one other therapeutically active agent is selected from a β-Lactam, nafcillin, sulfamethoxazolylm, trimethoprim, sulfasalazine, acetyl sulfisoxazolyl, and vancomycin. In one embodiment, the RIP1 kinase-mediated disease or disorder treated with these agents is a staphylococcus infection.

In one embodiment of this invention, the at least one other therapeutically active agent is selected from a monoclonal antibody, a polyclonal anti-T-cell antibody, an anti-thymocyte gamma globulin-equine antibody, an antithymocyte globulin-rabbit antibody, an anti-CD40 antagonist, a JAK inhibitor, and an anti-TCR murine mAb. In another embodiment, the at least one other therapeutically active agent is selected from muromonab-CD3, ASKP-1240, ASP015K, and TOL101. In one embodiment, the RIP1 kinase-mediated disease or disorder treated with these agents is transplant rejection.

In one embodiment of this invention, the at least one other therapeutically active agent is selected from a topical immunomodulator or calcineurin inhibitor, a topical corticosteroid, an oral corticosteroid, an interferon gamma, an antihistamine, or an antibiotic. In another embodiment, the at least one other therapeutically active agent is selected from pimecrolimus, tacrolimus, hydrocortizone, betamethasone, flurandrenolide, fluticasone, triamcinolone, fluocinonide, clobetasol, hydrocortisone, methylprednisolone, prednisolone, an interferon alpha protein, a recombinant synthetic type I interferon, interferon alpha-2a, interferon alpha-2b, hydroxyzine, diphenhydramine, flucloxacillin, dicloxacillin, and erythromycin. In one embodiment, the RIP1 kinase-mediated disease or disorder treated with these agents is atopic dermatitis.

In another embodiment, a compound that inhibits RIP 1 kinase, particularly a compound of Formula (I) or Formula (II), or a pharmaceutically acceptable salt thereof, may be administered to a patient in need thereof, in combination with at least one other therapy and/or with at least one other active therapeutic agent that is considered standard of care (U.S. Department of Health and Human Services, Agency for Healthcare Research and Quality, National Guideline Clearinghouse, https://www.guideline.gov/ and World Health Organization, http://www.who.int/management/quality/standards/en/) for any of the diseases and/or disorders recited herein.

A compound that inhibits RIP 1 kinase, particularly a compound of Formula (I) or Formula (II), or a pharmaceutically acceptable salt thereof, may be administered to a patient in need thereof, in combination with at least one other active therapeutic agent, wherein the at least one other active therapeutic agent is: a corticosteroid [administered orally, topically, by injection, or as a suppository; prednisone, methylprednisolone, prednisolone, budesonide, betamethasone, dexamethasone, hydrocortisone, triamcinolone, fluticasone (fluticasone furoate, fluticasone propionate), fludroxycortide (flurandrenolide, flurandrenolone), fluocinonide, clobetasol (clobetasol propionate)], an anti-TNF biologic agent (etanecerpt, adalimumab, infliximab, certolizumab, golimumab), an other biologic agent (vedolizumab, etrolizumab, eldelumab, or bertilimumab), biosimilars to any of the above biologic agents, a PDE-4 inhibitor (apremilast), 5-aminosalicyclic acid (mesalazine/mesalamine; sulfasalazine, balsalazide), a DMARD (a disease-modifying anti-rheumatic drug: methotrexate, hydroxychloroquine, sulfasalazine, leflunomide), a thiopurine (azathioprine, mercaptopurine), a JAK inhibitor (tofacitinib, Baracitinib), an NSAID (aspirin, acetaminophen, ibuprofen, naproxen (naproxen sodium), etodolac, celecoxib, diclofenac, meloxicam), an anti-IL6 biologic agent (tocilizumab), an anti-IL1 biologic agent (anakinra, canakinumab, rilonacept), an anti-IL12 or IL23 biologic agent (ustekinumab, risankizumab, guselkumab, tildrakizumab), an anti-CD6 biologic agent (itolizumab), an anti-integrin agent (natalizumab (Tysabri®), etrolizumab), an anti-IL17 biologic agent (secukinumab, ixekizumab, brodalumab), an anti-CD22 biologic agent (epratuzumab), an anti-CD20 biologic agent (rituximab, ofatumumab), an anti-CD20 or CD4 biologic agent and other cytokine inhibitor or biologic to T-cell or B-cell receptors or interleukins, T cell inhibitors (abatacept) a calcineurin inhibitor (cyclosporine, pimecrolimus, tacrolimus), acitretin, fumaric acid, dimethyl fumarate, cyclophosphamide, cyclosporine (or ciclosporin), methotrexate, mycophenolic acid (or mycophenolate mofetil), topical vitamin D (calcipotriol or calcipotriene), an mTOR inhibitor (temsirolimus, everolimus), a Syk inhibitor (fostamatinib), an anti-IFNa biologic agent (sifalimumab), a retinoid (tazarotene), coal tar preparations, aryl hydrocarbon receptor agonist or modulating agent (tapinarof), hydroxyurea, 6-tioguanineor light therapy with or without psoralen.

Examples of other active therapeutic agents that may be used in combination with a compound of this invention for the treatment of ulcerative colitis and/or Crohn's disease include vedolizumab, etrolizumab, eldelumab, or bertilimumab.

Examples of other suitable biologic agents include abatacept, belimumab, and alicafosen. Examples of other active therapeutic agent that may be used in combination with a compound of this invention include baracitinib and Remestemcel-L.

A compound that inhibits RIP 1 kinase, particularly a compound of Formula (I) or Formula (II), or a pharmaceutically acceptable salt thereof, may be administered to a pediatric or an adult patient in need thereof, in combination with at least one other therapy, for example, in combination with UVA and/or UVB phototherapy as indicated for the treatment of psoriasis.

In the treatment of pediatric and/or adult psoriasis, a compound that inhibits RIP 1 kinase, particularly a compound of Formula (I) or Formula (II), or a pharmaceutically acceptable salt thereof, may be administered to reduce the signs and symptoms including body surface area, pruritis, nail disease, and scalp involvement, and to improve quality of life, in pediatric and/or adult patients with moderate to severe psoriasis.

In the treatment of pediatric and/or adult psoriasis, a compound that inhibits RIP 1 kinase, particularly a compound of Formula (I) or Formula (II), or a pharmaceutically acceptable salt thereof, may be administered as initial treatment or after treatment with another agent in pediatric and/or adult patients with moderate to severe psoriasis.

In the treatment of pediatric and/or adult psoriasis, a compound that inhibits RIP 1 kinase, particularly a compound of Formula (I) or Formula (II), or a pharmaceutically acceptable salt thereof, may be administered to maintain reductions in signs and symptoms and improvements in quality of life after treatment with another agent in pediatric and/or adult patients with moderate to severe psoriasis.

A compound that inhibits RIP 1 kinase, particularly a compound of Formula (I) or Formula (II), or a pharmaceutically acceptable salt thereof, may be administered for the treatment of moderately to severely active rheumatoid arthritis.

In the treatment of rheumatoid arthritis, a compound that inhibits RIP 1 kinase, particularly a compound of Formula (I) or Formula (II), or a pharmaceutically acceptable salt thereof, may be administered to reduce the signs and symptoms, to induce a major clinical response, to inhibit the progression of structural damage, or to improve physical function in a patient, particularly an adult patient with moderately to severely active rheumatoid arthritis.

In the treatment of rheumatoid arthritis, a compound that inhibits RIP 1 kinase, particularly a compound of Formula (I) or Formula (II), or a pharmaceutically acceptable salt thereof, may be administered alone or in combination with methotrexate or other non-biologic disease-modifying anti-rheumatic drugs (DMARDs). In a specific embodiment, a compound that inhibits RIP 1 kinase, particularly a compound of Formula (I) or Formula (II), or a pharmaceutically acceptable salt thereof, may be administered alone or in combination with methotrexate, or corticosteroids in the treatment of rheumatoid arthritis.

In the treatment of juvenile idiopathic arthritis (JIA), a compound that inhibits RIP 1 kinase, particularly a compound of Formula (I) or Formula (II), or a pharmaceutically acceptable salt thereof, may be administered to reduce the signs and symptoms of moderately to severely active polyarticular juvenile idiopathic arthritis in patients 2 years of age and older.

In the treatment of juvenile idiopathic arthritis, particularly polyarticular juvenile idiopathic arthritis, a compound that inhibits RIP 1 kinase, particularly a compound of Formula (I) or Formula (II), or a pharmaceutically acceptable salt thereof, may be administered alone or in combination with methotrexate.

In the treatment of psoriatic arthritis, a compound that inhibits RIP 1 kinase, particularly a compound of Formula (I) or Formula (II), or a pharmaceutically acceptable salt thereof, may be administered to reduce the signs and symptoms, inhibiting the progression of structural damage, of active arthritis, and/or to improve physical function in adult patients with psoriatic arthritis.

In the treatment of psoriatic arthritis, a compound that inhibits RIP 1 kinase, particularly a compound of Formula (I) or Formula (II), or a pharmaceutically acceptable salt thereof, may be administered alone or in combination with methotrexate, corticosteroids, or other non-biologic disease-modifying anti-rheumatic drugs (DMARDs).

In a specific embodiment, a compound that inhibits RIP 1 kinase, particularly a compound of Formula (I) or Formula (II), or a pharmaceutically acceptable salt thereof, may be administered alone or in combination with methotrexate for the treatment of psoriatic arthritis.

In the treatment of psoriatic arthritis, a compound that inhibits RIP 1 kinase, particularly a compound of Formula (I) or Formula (II), or a pharmaceutically acceptable salt thereof, may be administered to a patient, particularly an adult patient with moderate to severe chronic plaque psoriasis, who is a candidate for systemic therapy or phototherapy.

In the treatment of axial Spondyloarthritis and ankylosing spondylitis, a compound that inhibits RIP 1 kinase, particularly a compound of Formula (I) or Formula (II), or a pharmaceutically acceptable salt thereof, may be administered to reduce the signs and symptoms of active ankylosing spondylitis in a patient, either an adult or a pediatric patient, in need thereof.

In the treatment of axial Spondyloarthritis and ankylosing spondylitis, a compound that inhibits RIP 1 kinase, particularly a compound of Formula (I) or Formula (II), or a pharmaceutically acceptable salt thereof, may be administered alone or in combination with methotrexate, corticosteroids, or other non-biologic disease-modifying anti-rheumatic drugs (DMARDs).

In the treatment of Crohn's Disease, a compound that inhibits RIP 1 kinase, particularly a compound of Formula (I) or Formula (II), or a pharmaceutically acceptable salt thereof, may be administered to reduce the signs and symptoms of Crohn's disease. A compound that inhibits RIP 1 kinase, particularly a compound of Formula (I) or Formula (II), or a pharmaceutically acceptable salt thereof, may be administered to induce or maintain a clinical response (clinical remission) in a patient, particularly an adult patient with moderately to severely active Crohn's disease.

In the treatment of Crohn's Disease, a compound that inhibits RIP1 kinase, particularly a compound of Formula (I) or Formula (II), or a pharmaceutically acceptable salt thereof, may be administered to reduce the signs and symptoms of Crohn's disease. A compound that inhibits RIP 1 kinase, particularly a compound of Formula (I) or Formula (II), or a pharmaceutically acceptable salt thereof, may be administered to induce or maintain a clinical response (clinical remission) in a patient, particularly a pediatric patient 6 years of age and older with moderately to severely active Crohn's disease.

A compound that inhibits RIP 1 kinase, particularly a compound of Formula (I) or Formula (II), or a pharmaceutically acceptable salt thereof, may be administered to reduce the signs and symptoms of Crohn's disease, particularly, moderately to severely active Crohn's disease, in a patient who has had an inadequate response to corticosteroids or immunomodulators such as azathioprine, 6-mercaptopurine, or methotrexate.

A compound that inhibits RIP 1 kinase, particularly a compound of Formula (I) or Formula (II), or a pharmaceutically acceptable salt thereof, may be administered to treat a patient, particularly an adult patient or a pediatric patient 6 years and older, with moderately to severely active ulcerative colitis.

In the treatment of ulcerative colitis, a compound that inhibits RIP 1 kinase, particularly a compound of Formula (I) or Formula (II), or a pharmaceutically acceptable salt thereof, may be administered to induce and/or sustain clinical remission in a patient, particularly an adult patient or a pediatric patient 6 years and older, with moderately to severely active ulcerative colitis.

A compound that inhibits RIP 1 kinase, particularly a compound of Formula (I) or Formula (II), or a pharmaceutically acceptable salt thereof, may be administered to induce and/or sustain a clinical response (clinical remission) in a patient, particularly a patient with moderately to severely active ulcerative colitis, who has had an inadequate response to immunosuppressants such as aminosalicylates, corticosteroids, azathioprine or 6-mercaptopurine (6-MP).

A compound that inhibits RIP 1 kinase, particularly a compound of Formula (I) or Formula (II), or a pharmaceutically acceptable salt thereof, may be administered for the treatment of moderate to severe hidradenitis suppurativa.

A compound that inhibits RIP 1 kinase, particularly a compound of Formula (I) or Formula (II), or a pharmaceutically acceptable salt thereof, may be administered for the treatment of uveitis, particularly non-infectious intermediate, posterior and panuveitis, in a patient, particularly an adult patient, in need thereof.

Accordingly, one embodiment of this invention is directed to a method of inhibiting RIP1 kinase comprising contacting a cell with a compound of the invention. Another embodiment of this invention is a method of inhibiting RIP1 kinase comprising contacting a cell with a compound of Formula (I) or Formula (II) or a pharmaceutically acceptable salt thereof. A particular embodiment of this invention is to a method of inhibiting RIP1 kinase comprising contacting a cell with a compound of Formula (II) or Formula (II) or a pharmaceutically acceptable salt thereof.

In another embodiment, the invention is directed to a method of treating a RIP1 kinase-mediated disease or disorder (for example, a disease or disorder recited herein) comprising administering a therapeutically effective amount of a compound of Formula (I) or Formula (II), or a pharmaceutically acceptable salt thereof, to a human in need thereof. In a particular embodiment, the invention is directed to a method of treating a RIP1 kinase-mediated disease or disorder (for example, a disease or disorder recited herein) comprising administering a therapeutically effective amount of a compound disclosed herein, or a pharmaceutically acceptable salt thereof, to a human in need thereof. In one specific embodiment, the invention is directed to a method of treating a RIP1 kinase-mediated disease or disorder (specifically, a disease or disorder recited herein) comprising administering a therapeutically effective amount of (S)-5-fluoro-6-(4-(5-phenyl-4,5-dihydro-1H-pyrazole-1-carbonyl)piperidin-1-yl)pyrimidine-4-carboxylic acid, or a pharmaceutically acceptable salt thereof, to a human in need thereof. In another specific embodiment, the invention is directed to a method of treating a RIP1 kinase-mediated disease or disorder (specifically, a disease or disorder recited herein) comprising administering a therapeutically effective amount of (S)-6-(4-(5-(3,5-difluorophenyl)-4,5-dihydro-1H-pyrazole-1-carbonyl)piperidin-1-yl)pyrimidine-4-carbonitrile, or a pharmaceutically acceptable salt thereof, to a human in need thereof. In another specific embodiment, the invention is directed to a method of treating a RIP1 kinase-mediated disease or disorder (specifically, a disease or disorder recited herein) comprising administering a therapeutically effective amount of (S)-(5-(3,5-difluorophenyl)-4,5-dihydro-1H-pyrazol-1-yl)(1-(5-methyl-1,3,4-oxadiazol-2-yl)piperidin-4-yl)methanone, or a pharmaceutically acceptable salt thereof, to a human in need thereof. In another specific embodiment, the invention is directed to a method of treating a RIP1 kinase-mediated disease or disorder (specifically, a disease or disorder recited herein) comprising administering a therapeutically effective amount of (S)-6-(4-(5-(5-fluoropyridin-3-yl)-4,5-dihydro-1H-pyrazole-1-carbonyl)piperidin-1-yl)pyrimidine-4-carboxamide, or a pharmaceutically acceptable salt thereof, to a human in need thereof. In another specific embodiment, the invention is directed to a method of treating a RIP1 kinase-mediated disease or disorder (specifically, a disease or disorder recited herein) comprising administering a therapeutically effective amount of (S)-5-fluoro-6-(4-(5-phenyl-4,5-dihydro-1H-pyrazole-1-carbonyl)piperidin-1-yl)pyrimidine-4-carboxylic acid, to a human in need thereof. In another specific embodiment, the invention is directed to a method of treating a RIP1 kinase-mediated disease or disorder (specifically, a disease or disorder recited herein) comprising administering a therapeutically effective amount of (S)-6-(4-(5-(3,5-difluorophenyl)-4,5-dihydro-1H-pyrazole-1-carbonyl)piperidin-1-yl)pyrimidine-4-carbonitrile, to a human in need thereof. In another specific embodiment, the invention is directed to a method of treating a RIP1 kinase-mediated disease or disorder (specifically, a disease or disorder recited herein) comprising administering a therapeutically effective amount of (S)-(5-(3,5-difluorophenyl)-4,5-dihydro-1H-pyrazol-1-yl)(1-(5-methyl-1,3,4-oxadiazol-2-yl)piperidin-4-yl)methanone, to a human in need thereof. In another specific embodiment, the invention is directed to a method of treating a RIP1 kinase-mediated disease or disorder (specifically, a disease or disorder recited herein) comprising administering a therapeutically effective amount of (S)-6-(4-(5-(5-fluoropyridin-3-yl)-4,5-dihydro-1H-pyrazole-1-carbonyl)piperidin-1-yl)pyrimidine-4-carboxamide, to a human in need thereof.

This invention also provides a compound of Formula (I) or Formula (II), or a pharmaceutically acceptable salt thereof, for use in therapy. This invention provides a compound of Formula (I) or Formula (II), or a pharmaceutically acceptable salt thereof, for use in the treatment of a RIP1 kinase-mediated disease or disorder (for example, a disease or disorder recited herein). Specifically, this invention provides a compound described herein, or a pharmaceutically acceptable salt thereof, for use in therapy. More specifically, this invention provides (S)-5-fluoro-6-(4-(5-phenyl-4,5-dihydro-1H-pyrazole-1-carbonyl)piperidin-1-yl)pyrimidine-4-carboxylic acid, or a pharmaceutically acceptable salt thereof, for use in therapy. More specifically, this invention provides (S)-6-(4-(5-(3,5-difluorophenyl)-4,5-dihydro-1H-pyrazole-1-carbonyl)piperidin-1-yl)pyrimidine-4-carbonitrile, or a pharmaceutically acceptable salt thereof, for use in therapy. More specifically, this invention provides (S)-(5-(3,5-difluorophenyl)-4,5-dihydro-1H-pyrazol-1-yl)(1-(5-methyl-1,3,4-oxadiazol-2-yl)piperidin-4-yl)methanone, or a pharmaceutically acceptable salt thereof, for use in therapy. More specifically, this invention provides (S)-6-(4-(5-(5-fluoropyridin-3-yl)-4,5-dihydro-1H-pyrazole-1-carbonyl)piperidin-1-yl)pyrimidine-4-carboxamide, or a pharmaceutically acceptable salt thereof, for use in therapy. More specifically, this invention provides (S)-5-fluoro-6-(4-(5-phenyl-4,5-dihydro-1H-pyrazole-1-carbonyl)piperidin-1-yl)pyrimidine-4-carboxylic acid for use in therapy. More specifically, this invention provides (S)-6-(4-(5-(3,5-difluorophenyl)-4,5-dihydro-1H-pyrazole-1-carbonyl)piperidin-1-yl)pyrimidine-4-carbonitrile for use in therapy. More specifically, this invention provides (S)-(5-(3,5-difluorophenyl)-4,5-dihydro-1H-pyrazol-1-yl)(1-(5-methyl-1,3,4-oxadiazol-2-yl)piperidin-4-yl)methanone for use in therapy. More specifically, this invention provides (S)-6-(4-(5-(5-fluoropyridin-3-yl)-4,5-dihydro-1H-pyrazole-1-carbonyl)piperidin-1-yl)pyrimidine-4-carboxamide for use in therapy.

In another embodiment, this invention provides a compound of the invention for use in the treatment of a RIP1 kinase-mediated disease or disorder, specifically, a disease or disorder recited herein. This invention provides a compound described herein, or a pharmaceutically acceptable salt thereof, for use in the treatment of a RIP1 kinase-mediated disease or disorder, specifically, a disease or disorder recited herein. In another specific embodiment, this invention provides (S)-5-fluoro-6-(4-(5-phenyl-4,5-dihydro-1H-pyrazole-1-carbonyl)piperidin-1-yl)pyrimidine-4-carboxylic acid, or a pharmaceutically acceptable salt thereof, for use in the treatment of a RIP1 kinase-mediated disease or disorder, specifically a disease or disorder recited herein. In another specific embodiment, this invention provides (S)-6-(4-(5-(3,5-difluorophenyl)-4,5-dihydro-1H-pyrazole-1-carbonyl)piperidin-1-yl)pyrimidine-4-carbonitrile, or a pharmaceutically acceptable salt thereof, for use in the treatment of a RIP1 kinase-mediated disease or disorder, specifically a disease or disorder recited herein. In another specific embodiment, this invention provides (S)-(5-(3,5-difluorophenyl)-4,5-dihydro-1H-pyrazol-1-yl)(1-(5-methyl-1,3,4-oxadiazol-2-yl)piperidin-4-yl)methanone, or a pharmaceutically acceptable salt thereof, for use in the treatment of a RIP1 kinase-mediated disease or disorder, specifically a disease or disorder recited herein. In another specific embodiment, this invention provides (S)-6-(4-(5-(5-fluoropyridin-3-yl)-4,5-dihydro-1H-pyrazole-1-carbonyl)piperidin-1-yl)pyrimidine-4-carboxamide, or a pharmaceutically acceptable salt thereof, for use in the treatment of a RIP1 kinase-mediated disease or disorder, specifically a disease or disorder recited herein. In another specific embodiment, this invention provides (S)-5-fluoro-6-(4-(5-phenyl-4,5-dihydro-1H-pyrazole-1-carbonyl)piperidin-1-yl)pyrimidine-4-carboxylic acid for use in the treatment of a RIP1 kinase-mediated disease or disorder, specifically a disease or disorder recited herein. In another specific embodiment, this invention provides (S)-6-(4-(5-(3,5-difluorophenyl)-4,5-dihydro-1H-pyrazole-1-carbonyl)piperidin-1-yl)pyrimidine-4-carbonitrile for use in the treatment of a RIP1 kinase-mediated disease or disorder, specifically a disease or disorder recited herein. In another specific embodiment, this invention provides (S)-(5-(3,5-difluorophenyl)-4,5-dihydro-1H-pyrazol-1-yl)(1-(5-methyl-1,3,4-oxadiazol-2-yl)piperidin-4-yl)methanone for use in the treatment of a RIP1 kinase-mediated disease or disorder, specifically a disease or disorder recited herein. In another specific embodiment, this invention provides (S)-6-(4-(5-(5-fluoropyridin-3-yl)-4,5-dihydro-1H-pyrazole-1-carbonyl)piperidin-1-yl)pyrimidine-4-carboxamide for use in the treatment of a RIP1 kinase-mediated disease or disorder, specifically a disease or disorder recited herein.

This invention specifically provides for the use of a compound of Formula (I) or Formula (II), or a pharmaceutically acceptable salt thereof, as an active therapeutic substance. More specifically, this invention provides for the use of the compounds described herein for the treatment of a RIP1 kinase-mediated disease or disorder, specifically, a disease or disorder recited herein. Accordingly, the invention provides for the use of a compound of Formula (I) or Formula (II) as an active therapeutic substance in the treatment of a human in need thereof with a RIP1 kinase-mediated disease or disorder, specifically, a disease or disorder recited herein. In one embodiment, this invention provides for the use of (S)-5-fluoro-6-(4-(5-phenyl-4,5-dihydro-1H-pyrazole-1-carbonyl)piperidin-1-yl)pyrimidine-4-carboxylic acid, or a pharmaceutically acceptable salt thereof, as an active therapeutic substance for the treatment of a RIP1 kinase-mediated disease or disorder, specifically a disease or disorder recited herein. In one embodiment, this invention provides for the use of (S)-6-(4-(5-(3,5-difluorophenyl)-4,5-dihydro-1H-pyrazole-1-carbonyl)piperidin-1-yl)pyrimidine-4-carbonitrile, or a pharmaceutically acceptable salt thereof, as an active therapeutic substance for the treatment of a RIP1 kinase-mediated disease or disorder, specifically a disease or disorder recited herein. In one embodiment, this invention provides for the use of (S)-(5-(3,5-difluorophenyl)-4,5-dihydro-1H-pyrazol-1-yl)(1-(5-methyl-1,3,4-oxadiazol-2-yl)piperidin-4-yl)methanone, or a pharmaceutically acceptable salt thereof, as an active therapeutic substance for the treatment of a RIP1 kinase-mediated disease or disorder, specifically a disease or disorder recited herein. In one embodiment, this invention provides for the use of (S)-6-(4-(5-(5-fluoropyridin-3-yl)-4,5-dihydro-1H-pyrazole-1-carbonyl)piperidin-1-yl)pyrimidine-4-carboxamide, or a pharmaceutically acceptable salt thereof, as an active therapeutic substance for the treatment of a RIP1 kinase-mediated disease or disorder, specifically a disease or disorder recited herein. In a more specific embodiment, this invention provides for the use of (S)-5-fluoro-6-(4-(5-phenyl-4,5-dihydro-1H-pyrazole-1-carbonyl)piperidin-1-yl)pyrimidine-4-carboxylic acid as an active therapeutic substance for the treatment of a RIP1 kinase-mediated disease or disorder, specifically a disease or disorder recited herein. In a more specific embodiment, this invention provides for the use of (S)-6-(4-(5-(3,5-difluorophenyl)-4,5-dihydro-1H-pyrazole-1-carbonyl)piperidin-1-yl)pyrimidine-4-carbonitrile as an active therapeutic substance for the treatment of a RIP1 kinase-mediated disease or disorder, specifically a disease or disorder recited herein. In a more specific embodiment, this invention provides for the use of (S)-(5-(3,5-difluorophenyl)-4,5-dihydro-1H-pyrazol-1-yl)(1-(5-methyl-1,3,4-oxadiazol-2-yl)piperidin-4-yl)methanone as an active therapeutic substance for the treatment of a RIP1 kinase-mediated disease or disorder, specifically a disease or disorder recited herein. In a more specific embodiment, this invention provides for the use of (S)-6-(4-(5-(5-fluoropyridin-3-yl)-4,5-dihydro-1H-pyrazole-1-carbonyl)piperidin-1-yl)pyrimidine-4-carboxamide as an active therapeutic substance for the treatment of a RIP1 kinase-mediated disease or disorder, specifically a disease or disorder recited herein.

The invention further provides for the use of a compound of Formula (I) or Formula (II), or a pharmaceutically acceptable salt thereof, in the manufacture of a medicament for use in the treatment of a RIP1 kinase-mediated disease or disorder, for example the diseases and disorders recited herein. Specifically, the invention also provides for the use of a compound described herein, or a pharmaceutically acceptable salt thereof, in the manufacture of a medicament for use in the treatment of a RIP1 kinase-mediated disease or disorder, for example the diseases and disorders recited herein. In one embodiment, the invention provides for the use of (S)-5-fluoro-6-(4-(5-phenyl-4,5-dihydro-1H-pyrazole-1-carbonyl)piperidin-1-yl)pyrimidine-4-carboxylic acid, or a pharmaceutically acceptable salt thereof, in the manufacture of a medicament for use in the treatment of a RIP1 kinase-mediated disease or disorder, for example the diseases and disorders recited herein. In one embodiment, the invention provides for the use of (S)-6-(4-(5-(3,5-difluorophenyl)-4,5-dihydro-1H-pyrazole-1-carbonyl)piperidin-1-yl)pyrimidine-4-carbonitrile, or a pharmaceutically acceptable salt thereof, in the manufacture of a medicament for use in the treatment of a RIP1 kinase-mediated disease or disorder, for example the diseases and disorders recited herein. In one embodiment, the invention provides for the use of (S)-(5-(3,5-difluorophenyl)-4,5-dihydro-1H-pyrazol-1-yl)(1-(5-methyl-1,3,4-oxadiazol-2-yl)piperidin-4-yl)methanone, or a pharmaceutically acceptable salt thereof, in the manufacture of a medicament for use in the treatment of a RIP1 kinase-mediated disease or disorder, for example the diseases and disorders recited herein. In one embodiment, the invention provides for the use of (S)-6-(4-(5-(5-fluoropyridin-3-yl)-4,5-dihydro-1H-pyrazole-1-carbonyl)piperidin-1-yl)pyrimidine-4-carboxamide, or a pharmaceutically acceptable salt thereof, in the manufacture of a medicament for use in the treatment of a RIP1 kinase-mediated disease or disorder, for example the diseases and disorders recited herein. In another embodiment, the invention provides for the use of (S)-5-fluoro-6-(4-(5-phenyl-4,5-dihydro-1H-pyrazole-1-carbonyl)piperidin-1-yl)pyrimidine-4-carboxylic acid in the manufacture of a medicament for use in the treatment of a RIP1 kinase-mediated disease or disorder, for example the diseases and disorders recited herein. In another embodiment, the invention provides for the use of (S)-6-(4-(5-(3,5-difluorophenyl)-4,5-dihydro-1H-pyrazole-1-carbonyl)piperidin-1-yl)pyrimidine-4-carbonitrile in the manufacture of a medicament for use in the treatment of a RIP1 kinase-mediated disease or disorder, for example the diseases and disorders recited herein. In another embodiment, the invention provides for the use of (S)-(5-(3,5-difluorophenyl)-4,5-dihydro-1H-pyrazol-1-yl)(1-(5-methyl-1,3,4-oxadiazol-2-yl)piperidin-4-yl)methanone in the manufacture of a medicament for use in the treatment of a RIP1 kinase-mediated disease or disorder, for example the diseases and disorders recited herein. In another embodiment, the invention provides for the use of (S)-6-(4-(5-(5-fluoropyridin-3-yl)-4,5-dihydro-1H-pyrazole-1-carbonyl)piperidin-1-yl)pyrimidine-4-carboxamide in the manufacture of a medicament for use in the treatment of a RIP1 kinase-mediated disease or disorder, for example the diseases and disorders recited herein.

A therapeutically “effective amount” is intended to mean that amount of a compound that, when administered to a patient in need of such treatment, is sufficient to effect treatment, as defined herein. Thus, e.g., a therapeutically effective amount of a compound of Formula (I) or Formula (II), or a pharmaceutically acceptable salt thereof, is a quantity of an inventive agent that, when administered to a human in need thereof, is sufficient to modulate and/or inhibit the activity of RIP1 kinase such that a disease condition which is mediated by that activity is reduced, alleviated or prevented. The amount of a given compound that will correspond to such an amount will vary depending upon factors such as the particular compound (e.g., the potency (pIC₅₀), efficacy (EC₅₀), and the biological half-life of the particular compound), disease condition and its severity, the identity (e.g., age, size and weight) of the patient in need of treatment, but can nevertheless be routinely determined by one skilled in the art. Likewise, the duration of treatment and the time period of administration (time period between dosages and the timing of the dosages, e.g., before/with/after meals) of the compound will vary according to the identity of the mammal in need of treatment (e.g., weight), the particular compound and its properties (e.g., pharmacokinetic properties), disease or disorder and its severity and the specific composition and method being used, but can nevertheless be determined by one of skill in the art.

“Treating” or “treatment” is intended to mean at least the mitigation of a disease or disorder in a patient. The methods of treatment for mitigation of a disease or disorder include the use of the compounds in this invention in any conventionally acceptable manner, for example for prevention, retardation, prophylaxis, therapy or cure of a RIP1 kinase mediated disease or disorder, as described hereinabove.

The compounds of the invention may be administered by any suitable route of administration, including both systemic administration and topical administration. Systemic administration includes oral administration, parenteral administration, transdermal administration, rectal administration, and administration by inhalation. Parenteral administration refers to routes of administration other than enteral, transdermal, or by inhalation, and is typically by injection or infusion. Parenteral administration includes intravenous, intramuscular, and subcutaneous injection or infusion. Inhalation refers to administration into the patient's lungs whether inhaled through the mouth or through the nasal passages. Topical administration includes application to the skin.

The compounds of the invention may be administered once or according to a dosing regimen wherein a number of doses are administered at varying intervals of time for a given period of time. For example, doses may be administered one, two, three, or four times per day. Doses may be administered until the desired therapeutic effect is achieved or indefinitely to maintain the desired therapeutic effect. Suitable dosing regimens for a compound of the invention depend on the pharmacokinetic properties of that compound, such as absorption, distribution, and half-life, which can be determined by the skilled artisan. In addition, suitable dosing regimens, including the duration such regimens are administered, for a compound of the invention depend on the disease or disorder being treated, the severity of the disease or disorder being treated, the age and physical condition of the patient being treated, the medical history of the patient to be treated, the nature of concurrent therapy, the desired therapeutic effect, and like factors within the knowledge and expertise of the skilled artisan. It will be further understood by such skilled artisans that suitable dosing regimens may require adjustment given an individual patient's response to the dosing regimen or over time as individual patient needs change. Total daily dosages range from 1 mg to 2000 mg.

For use in therapy, the compounds of the invention will be normally, but not necessarily, formulated into a pharmaceutical composition prior to administration to a patient. Accordingly, the invention also is directed to pharmaceutical compositions comprising a compound of the invention and one or more pharmaceutically acceptable excipients. The invention is directed to a pharmaceutical composition comprising a compound of Formula (I) or Formula (II), or a pharmaceutically acceptable salt thereof, and one or more pharmaceutically acceptable excipients. In one embodiment, there is provided a pharmaceutical composition comprising (S)-5-fluoro-6-(4-(5-phenyl-4,5-dihydro-1H-pyrazole-1-carbonyl)piperidin-1-yl)pyrimidine-4-carboxylic acid, or a pharmaceutically acceptable salt thereof, and one or more pharmaceutically acceptable excipients. In one embodiment, there is provided a pharmaceutical composition comprising (S)-6-(4-(5-(3,5-difluorophenyl)-4,5-dihydro-1H-pyrazole-1-carbonyl)piperidin-1-yl)pyrimidine-4-carbonitrile, or a pharmaceutically acceptable salt thereof, and one or more pharmaceutically acceptable excipients. In one embodiment, there is provided a pharmaceutical composition comprising (S)-(5-(3,5-difluorophenyl)-4,5-dihydro-1H-pyrazol-1-yl)(1-(5-methyl-1,3,4-oxadiazol-2-yl)piperidin-4-yl)methanone, or a pharmaceutically acceptable salt thereof, and one or more pharmaceutically acceptable excipients. In one embodiment, there is provided a pharmaceutical composition comprising (S)-6-(4-(5-(5-fluoropyridin-3-yl)-4,5-dihydro-1H-pyrazole-1-carbonyl)piperidin-1-yl)pyrimidine-4-carboxamide, or a pharmaceutically acceptable salt thereof, and one or more pharmaceutically acceptable excipients. In another embodiment, there is provided a pharmaceutical composition comprising (S)-5-fluoro-6-(4-(5-phenyl-4,5-dihydro-1H-pyrazole-1-carbonyl)piperidin-1-yl)pyrimidine-4-carboxylic acid, and one or more pharmaceutically acceptable excipients. In another embodiment, there is provided a pharmaceutical composition comprising (S)-6-(4-(5-(3,5-difluorophenyl)-4,5-dihydro-1H-pyrazole-1-carbonyl)piperidin-1-yl)pyrimidine-4-carbonitrile, and one or more pharmaceutically acceptable excipients. In another embodiment, there is provided a pharmaceutical composition comprising (S)-(5-(3,5-difluorophenyl)-4,5-dihydro-1H-pyrazol-1-yl)(1-(5-methyl-1,3,4-oxadiazol-2-yl)piperidin-4-yl)methanone, and one or more pharmaceutically acceptable excipients. In another embodiment, there is provided a pharmaceutical composition comprising (S)-6-(4-(5-(5-fluoropyridin-3-yl)-4,5-dihydro-1H-pyrazole-1-carbonyl)piperidin-1-yl)pyrimidine-4-carboxamide, and one or more pharmaceutically acceptable excipients.

The invention is further directed to a pharmaceutical composition comprising a compound of Formula (I) or Formula (II), or a pharmaceutically acceptable salt thereof, and one or more pharmaceutically acceptable excipients and at least one other therapeutically active agent, specifically one or two other therapeutically active agents, more specifically one other therapeutically active agent. In one embodiment, there is provided a pharmaceutical composition comprising (S)-5-fluoro-6-(4-(5-phenyl-4,5-dihydro-1H-pyrazole-1-carbonyl)piperidin-1-yl)pyrimidine-4-carboxylic acid, or a pharmaceutically salt thereof, one or more pharmaceutically acceptable excipients, and at least one other therapeutically active agent, specifically one or two other therapeutically active agents, more specifically one other therapeutically active agent. In one embodiment, there is provided a pharmaceutical composition comprising (S)-6-(4-(5-(3,5-difluorophenyl)-4,5-dihydro-1H-pyrazole-1-carbonyl)piperidin-1-yl)pyrimidine-4-carbonitrile, or a pharmaceutically salt thereof, one or more pharmaceutically acceptable excipients, and at least one other therapeutically active agent, specifically one or two other therapeutically active agents, more specifically one other therapeutically active agent. In one embodiment, there is provided a pharmaceutical composition comprising (S)-(5-(3,5-difluorophenyl)-4,5-dihydro-1H-pyrazol-1-yl)(1-(5-methyl-1,3,4-oxadiazol-2-yl)piperidin-4-yl)methanone, or a pharmaceutically salt thereof, one or more pharmaceutically acceptable excipients, and at least one other therapeutically active agent, specifically one or two other therapeutically active agents, more specifically one other therapeutically active agent. In one embodiment, there is provided a pharmaceutical composition comprising (S)-6-(4-(5-(5-fluoropyridin-3-yl)-4,5-dihydro-1H-pyrazole-1-carbonyl)piperidin-1-yl)pyrimidine-4-carboxamide, or a pharmaceutically salt thereof, one or more pharmaceutically acceptable excipients, and at least one other therapeutically active agent, specifically one or two other therapeutically active agents, more specifically one other therapeutically active agent. In another embodiment, there is provided a pharmaceutical composition comprising (S)-5-fluoro-6-(4-(5-phenyl-4,5-dihydro-1H-pyrazole-1-carbonyl)piperidin-1-yl)pyrimidine-4-carboxylic acid, one or more pharmaceutically acceptable excipients, and at least one other therapeutically active agent, specifically one or two other therapeutically active agents, more specifically one other therapeutically active agent. In another embodiment, there is provided a pharmaceutical composition comprising (S)-6-(4-(5-(3,5-difluorophenyl)-4,5-dihydro-1H-pyrazole-1-carbonyl)piperidin-1-yl)pyrimidine-4-carbonitrile, one or more pharmaceutically acceptable excipients, and at least one other therapeutically active agent, specifically one or two other therapeutically active agents, more specifically one other therapeutically active agent. In another embodiment, there is provided a pharmaceutical composition comprising (S)-(5-(3,5-difluorophenyl)-4,5-dihydro-1H-pyrazol-1-yl)(1-(5-methyl-1,3,4-oxadiazol-2-yl)piperidin-4-yl)methanone, one or more pharmaceutically acceptable excipients, and at least one other therapeutically active agent, specifically one or two other therapeutically active agents, more specifically one other therapeutically active agent. In another embodiment, there is provided a pharmaceutical composition comprising (S)-6-(4-(5-(5-fluoropyridin-3-yl)-4,5-dihydro-1H-pyrazole-1-carbonyl)piperidin-1-yl)pyrimidine-4-carboxamide, one or more pharmaceutically acceptable excipients, and at least one other therapeutically active agent, specifically one or two other therapeutically active agents, more specifically one other therapeutically active agent.

The pharmaceutical compositions of the invention may be prepared and packaged in bulk form wherein an effective amount of a compound of the invention can be extracted and then given to the patient such as with powders, syrups, and solutions for injection. Alternatively, the pharmaceutical compositions of the invention may be prepared and packaged in unit dosage form. A dose of the pharmaceutical composition contains at least a therapeutically effective amount of a compound of this invention (i.e., a compound of Formula (I) or Formula (II), or a pharmaceutically acceptable salt, thereof). When prepared in unit dosage form, the pharmaceutical compositions may contain from 1 mg to 1000 mg of a compound of this invention.

As provided herein, unit dosage forms (pharmaceutical compositions) containing from 1 mg to 1000 mg of a compound of the invention may be administered one, two, three, or four times per day, preferably one, two, or three times per day, and more preferably, one or two times per day, to effect treatment of a RIP1 kinase-mediated disease or disorder.

As used herein, “pharmaceutically acceptable excipient” means a material, composition or vehicle involved in giving form or consistency to the composition. Each excipient must be compatible with the other ingredients of the pharmaceutical composition when commingled such that interactions which would substantially reduce the efficacy of the compound of the invention when administered to a patient and interactions which would result in pharmaceutical compositions that are not pharmaceutically acceptable are avoided. In addition, each excipient must of course be of sufficiently high purity to render it pharmaceutically acceptable.

The compounds of the invention and the pharmaceutically acceptable excipient or excipients will typically be formulated into a dosage form adapted for administration to the patient by the desired route of administration. Conventional dosage forms suitable for use with the compounds of this invention include those adapted for (1) oral administration such as tablets, capsules, caplets, pills, troches, powders, syrups, elixirs, suspensions, solutions, emulsions, sachets, and cachets; (2) parenteral administration such as sterile solutions, suspensions, and powders for reconstitution; (3) transdermal administration such as transdermal patches; (4) rectal administration such as suppositories; (5) inhalation such as aerosols and solutions; and (6) topical administration such as creams, ointments, lotions, solutions, pastes, sprays, foams, and gels.

Suitable pharmaceutically acceptable excipients will vary depending upon the particular dosage form chosen. In addition, suitable pharmaceutically acceptable excipients may be chosen for a particular function that they may serve in the composition. For example, certain pharmaceutically acceptable excipients may be chosen for their ability to facilitate the production of uniform dosage forms. Certain pharmaceutically acceptable excipients may be chosen for their ability to facilitate the production of stable dosage forms. Certain pharmaceutically acceptable excipients may be chosen for their ability to facilitate the carrying or transporting the compound or compounds of the invention once administered to the patient from one organ, or portion of the body, to another organ, or portion of the body. Certain pharmaceutically acceptable excipients may be chosen for their ability to enhance patient compliance.

Suitable pharmaceutically acceptable excipients include the following types of excipients: diluents, fillers, binders, disintegrants, lubricants, glidants, granulating agents, coating agents, wetting agents, solvents, co-solvents, suspending agents, emulsifiers, sweeteners, flavoring agents, flavor masking agents, coloring agents, anti-caking agents, humectants, chelating agents, plasticizers, viscosity increasing agents, antioxidants, preservatives, stabilizers, surfactants, and buffering agents. The skilled artisan will appreciate that certain pharmaceutically acceptable excipients may serve more than one function and may serve alternative functions depending on how much of the excipient is present in the formulation and what other ingredients are present in the formulation.

Skilled artisans possess the knowledge and skill in the art to enable them to select suitable pharmaceutically acceptable excipients in appropriate amounts for use in the invention. In addition, there are a number of resources that are available to the skilled artisan which describe pharmaceutically acceptable excipients and may be useful in selecting suitable pharmaceutically acceptable excipients. Examples include Remington's Pharmaceutical Sciences (Mack Publishing Company), The Handbook of Pharmaceutical Additives (Gower Publishing Limited), and The Handbook of Pharmaceutical Excipients (the American Pharmaceutical Association and the Pharmaceutical Press).

The pharmaceutical compositions of the invention are prepared using techniques and methods known to those skilled in the art. Some of the methods commonly used in the art are described in Remington's Pharmaceutical Sciences (Mack Publishing Company). Accordingly, another embodiment of this invention is a method of preparing a pharmaceutical composition comprising the step of admixing a compound of Formula (I) or Formula (II), or a pharmaceutically acceptable salt, thereof, with one or more pharmaceutically acceptable excipients.

In one aspect, the invention is directed to a topical dosage form such as a cream, ointment, lotion, paste, or gel comprising an effective amount of a compound of the invention and one or more pharmaceutically acceptable excipients. Lipophilic formulations, such as anhydrous creams and ointments, generally will have a base derived from fatty alcohols, and polyethylene glycols. Additional additives include alcohols, non-ionic surfactants, and antioxidants. For ointments, the base normally will be an oil or mixture of oil and wax, e.g., petrolatum. Also, an antioxidant normally will be included in minor amounts. Because the compositions are applied topically and the effective dosage can be controlled by the total composition applied, the percentage of active ingredient in the composition can vary widely. Convenient concentrations range from 0.5% to 20%.

Topically applied gels can also be a foamable suspension gel comprising a compound of the invention, as an active agent, one or more thickening agents, and optionally, a dispersing/wetting agent, a pH-adjusting agent, a surfactant, a propellent, an antioxidant, an additional foaming agent, a chelating/sequestering agent, a solvent, a fragrance, a coloring agent, a preservative, wherein the gel is aqueous and forms a homogenous foam.

In one aspect, the invention is directed to a topical dosage form that can be administered by inhalation, that is, by intranasal and oral inhalation administration. Appropriate dosage forms for such administration, such as an aerosol formulation or a metered dose inhaler, may be prepared by conventional techniques. Intranasal sprays may be formulated with aqueous or non-aqueous vehicles with the addition of agents such as thickening agents, buffer salts or acid or alkali to adjust the pH, isotonicity adjusting agents or anti-oxidants. Solutions for inhalation by nebulization may be formulated with an aqueous vehicle with the addition of agents such as acid or alkali, buffer salts, isotonicity adjusting agents or antimicrobials.

Formulations for administration by inhalation or foamable gel often require the use of a suitable propellant. Capsules and cartridges of e.g. gelatin for use in an inhaler or insufflator may be formulated using a suitable powder base such as lactose or starch.

In another aspect, the invention is directed to a solid oral dosage form such as a tablet or capsule comprising an effective amount of a compound of the invention and a diluent or filler. Suitable diluents and fillers include lactose, sucrose, dextrose, mannitol, sorbitol, starch (e.g. corn starch, potato starch, and pre-gelatinized starch), cellulose and its derivatives (e.g. microcrystalline cellulose), calcium sulfate, and dibasic calcium phosphate. The oral solid dosage form may further comprise a binder. Suitable binders include starch (e.g. corn starch, potato starch, and pre-gelatinized starch), gelatin, acacia, sodium alginate, alginic acid, tragacanth, guar gum, povidone, and cellulose and its derivatives (e.g. microcrystalline cellulose). The oral solid dosage form may further comprise a disintegrant. Suitable disintegrants include crospovidone, sodium starch glycolate, croscarmelose, alginic acid, and sodium carboxymethyl cellulose. The oral solid dosage form may further comprise a lubricant. Suitable lubricants include stearic acid, magnesium stearate, calcium stearate, and talc.

### Definitions

As used herein, the term “alkyl” represents a saturated, straight or branched hydrocarbon group having the specified number of carbon atoms. The term “(C₂-C₆)alkyl” refers to an alkyl moiety containing from 2 to 6 carbon atoms. Exemplary alkyls include, but are not limited to methyl, ethyl, n-propyl, isopropyl, n-butyl, isobutyl, s-butyl, and t-butyl.

When a substituent term such as “alkyl” is used in combination with another substituent term, for example as in “(C₄-C₆)cycloalkyl-alkyl-”, the linking substituent term (e.g., alkyl) is intended to encompass a multi-valent moiety, wherein the point of attachment is through that linking substituent. Generally, the linking substituent is di-valent. An example of a “(C₃-C₇)cycloalkyl-alkyl-” group includes, but is not limited to, cyclopentyl-methyl-.

The term “halo(C₁-C₄)alkyl” represents a group having one or more halogen atoms, which may be the same or different, at one or more carbon atoms of an alkyl moiety containing from 1 to 4 carbon atoms. Examples of “halo(C₁-C₄)alkyl” groups include, but are not limited to, —CF₃ (trifluoromethyl), —CCl₃ (trichloromethyl), 1,1-difluoroethyl, 2,2,2-trifluoroethyl, and hexafluoroisopropyl.

“Alkenyl” refers to straight or branched hydrocarbon group having at least 1 and up to 3 carbon-carbon double bonds. Examples include ethenyl and propenyl.

“Alkoxy” refers to an “alkyl-oxy-” group, containing an alkyl moiety attached through an oxygen linking atom. For example, the term “(C₁-C₄)alkoxy” represents a saturated, straight or branched hydrocarbon moiety having at least 1 and up to 4 carbon atoms attached through an oxygen linking atom. Exemplary “(C₁-C₄)alkoxy” groups include, but are not limited to, methoxy, ethoxy, n-propoxy, isopropoxy, n-butoxy, s-butoxy, and t-butoxy.

The term “halo(C₁-C₄)alkoxy” refers to a “haloalkyl-oxy-” group, containing a “halo(C₁-C₄)alkyl” moiety attached through an oxygen linking atom, which halo(C₁-C₄)alkyl” refers to a moiety having one or more halogen atoms, which may be the same or different, at one or more carbon atoms of an alkyl moiety containing from 1 to 4 carbon atoms. Exemplary “halo(C₁-C₄)alkoxy” groups include, but are not limited to, —OCHF₂ (difluoromethoxy), —OCF₃ (trifluoromethoxy), —OCH₂CF₃ (trifluoroethoxy), and —OCH(CF₃)₂ (hexafluoroisopropoxy).

A carbocyclic group is a cyclic group in which all of the ring members are carbon atoms, which may be saturated, partially unsaturated (non-aromatic) or fully unsaturated (aromatic). The term “carbocyclic” includes cycloalkyl and aryl groups.

“Cycloalkyl” refers to a non-aromatic, saturated, cyclic hydrocarbon group containing the specified number of carbon atoms. For example, the term “(C₃-C₆)cycloalkyl” refers to a non-aromatic cyclic hydrocarbon ring having from three to six ring carbon atoms. Exemplary “(C₃-C₆)cycloalkyl” groups include cyclopropyl, cyclobutyl, cyclopentyl, and cyclohexyl.

The terms “cycloalkyloxy” or “cycloalkoxy” refer to a group containing a cycloalkyl moiety, defined hereinabove, attached through an oxygen linking atom. Exemplary “(C₃-C₆)cycloalkyloxy” groups include cyclopropyloxy, cyclobutyloxy, cyclopentyloxy, and cyclohexyloxy.

“Aryl” refers to a group or moiety comprising an aromatic, monocyclic or bicyclic hydrocarbon radical containing from 6 to 10 carbon ring atoms and having at least one aromatic ring. Examples of “aryl” groups are phenyl, naphthyl, indenyl, and dihydroindenyl (indanyl). Generally, aryl is phenyl.

The term “9-10 membered carbocyclic-aryl” refers to a bicyclic group or moiety specifically comprising a phenyl moiety fused to a 5-6 membered saturated or partially saturated carbocyclic moiety. Examples of “9-10 membered carbocyclic-aryl” groups include dihydroindenyl (indanyl) and tetrahydronaphthyl.

A heterocyclic group is a cyclic group having, as ring members, atoms of at least two different elements, which cyclic group may be saturated, partially unsaturated (non-aromatic) or fully unsaturated (aromatic).

“Heterocycloalkyl” refers to a non-aromatic, monocyclic or bicyclic group containing 3-10 ring atoms, being saturated and containing one or more (generally one or two) ring heteroatoms independently selected from oxygen, sulfur, and nitrogen. Examples of “heterocycloalkyl” groups include, but are not limited to, aziridinyl, thiiranyl, oxiranyl, azetidinyl, oxetanyl, thietanyl, pyrrolidinyl, tetrahydrofuranyl, tetrahydrothienyl, piperidinyl, piperazinyl, tetrahydropyranyl, tetrahydrothiopyranyl, 1,4-dioxanyl, 1,4-oxathiolanyl, 1,4-oxathianyl, 1,4-dithianyl, morpholinyl, and thiomorpholinyl, and dihydroimidazole.

Examples of “4-membered heterocycloalkyl” groups include oxetanyl, thietanyl and azetidinyl.

The term “5-6-membered heterocycloalkyl” represents a nonaromatic, monocyclic group, which is fully saturated, containing 5 or 6 ring atoms, which includes one or two heteroatoms selected independently from oxygen, sulfur, and nitrogen. Illustrative examples of 5 to 6-membered heterocycloalkyl groups include, but are not limited to pyrrolidinyl, piperidinyl, piperazinyl, tetrahydrofuranyl, tetrahydrothienyl, tetrahydropyranyl, tetrahydrothiopyranyl, morpholinyl, thiomorpholinyl, and dihydroimidazole.

“Heteroaryl” represents a group or moiety comprising an aromatic monocyclic or bicyclic radical, containing 5 to 10 ring atoms, including 1 to 4 heteroatoms independently selected from nitrogen, oxygen and sulfur. This term also encompasses bicyclic heterocyclic-aryl groups containing either an aryl ring moiety fused to a heterocycloalkyl ring moiety or a heteroaryl ring moiety fused to a cycloalkyl ring moiety.

Illustrative examples of heteroaryls include, but are not limited to, furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl (pyridyl), oxo-pyridyl (pyridyl-N-oxide), pyridazinyl, pyrazinyl, pyrimidinyl, triazinyl, benzofuranyl, isobenzofuryl, 2,3-dihydrobenzofuryl, 1,3-benzodioxolyl, dihydrobenzodioxinyl, benzothienyl, indolizinyl, indolyl, isoindolyl, dihydroindolyl, benzimidazolyl, dihydrobenzimidazolyl, benzoxazolyl, dihydrobenzoxazolyl, benzothiazolyl, benzoisothiazolyl, dihydrobenzoisothiazolyl, indazolyl, imidazopyridinyl, pyrazolopyridinyl, benzotriazolyl, triazolopyridinyl, purinyl, quinolinyl, tetrahydroquinolinyl, isoquinolinyl, tetrahydroisoquinolinyl, quinoxalinyl, cinnolinyl, phthalazinyl, quinazolinyl, 1,5-naphthyridinyl, 1,6-naphthyridinyl, 1,7-naphthyridinyl, 1,8-naphthyridinyl, and pteridinyl.

The term “9-10 membered heterocyclic-aryl” refers to a bicyclic group or moiety specifically comprising a phenyl moiety fused to a 5-6 membered saturated or partially saturated heterocyclic moiety. Examples of “9-10 membered heterocyclic-aryl” groups include 2,3-dihydrobenzofuryl (dihydrobenzofuranyl), 2,3-dihydrobenzothienyl, 1,3-benzodioxolyl, dihydrobenzodioxinyl (dihydro-1,4-benzodioxinyl), dihydroindolyl, tetrahydroquinolinyl, and tetrahydroisoquinolinyl.

As used herein, “5-6-membered heteroaryl” represents an aromatic monocyclic group containing 5 or 6 ring atoms, including at least one carbon atom and 1 to 4 heteroatoms independently selected from nitrogen, oxygen and sulfur. Selected 5-membered heteroaryl groups contain one nitrogen, oxygen, or sulfur ring heteroatom, and optionally contain 1, 2, or 3 additional nitrogen ring atoms. Selected 6-membered heteroaryl groups contain 1, 2, or 3 nitrogen ring heteroatoms. Examples of 5-membered heteroaryl groups include furyl (furanyl), thienyl, pyrrolyl, imidazolyl, pyrazolyl, triazolyl, tetrazolyl, thiazolyl, isothiazolyl, thiadiazolyl, oxazolyl, isoxazolyl, oxadiazolyl and oxo-oxadiazolyl. Selected 6-membered heteroaryl groups include pyridinyl, oxo-pyridyl, pyrazinyl, pyrimidinyl, pyridazinyl and triazinyl.

As used herein, “9-10-membered heteroaryl” represents an aromatic cyclic group containing 9 or 10 ring atoms, including at least one carbon atom and 1 to 4 heteroatoms independently selected from nitrogen, oxygen and sulfur. Selected 9-membered heteroaryl groups contain one nitrogen, oxygen, or sulfur ring heteroatom, and optionally contain 1, 2, or 3 additional nitrogen ring atoms. Selected 10-membered heteroaryl groups contain 1, 2, or 3 nitrogen ring heteroatoms. Examples of 9-membered heteroaryl groups include 7H-purinyl, 9H-purinyl, pyrazolo[1,5-a]pyrimidinyl, imidazo[1,2-b]pyridazinyl, 1H-pyrazolo[3,4-d]pyrimidinyl, and imidazo[1,2-b]pyridazinyl, benzofuranyl, benzothienyl, benzimidazolyl, benzthiazolyl, indolizinyl, indolyl, isoindolyl, and indazolyl. Selected 10-membered heteroaryl groups include quinolinyl, isoquinolinyl, quinoxalinyl, cinnolinyl, phthalazinyl, quinazolinyl, 1,5-naphthyridinyl, 1,6-naphthyridinyl, 1,7-naphthyridinyl, 1,8-naphthyridinyl, and pteridinyl.

Bicyclic heteroaryl groups include 6,5-fused heteroaryl (9-membered heteroaryl) and 6,6-fused heteroaryl (10-membered heteroaryl) groups. Examples of 6,5-fused heteroaryl (9-membered heteroaryl) groups include benzothienyl, benzofuranyl, indolyl, indolinyl, isoindolyl, isoindolinyl, indazolyl, indolizinyl, isobenzofuryl, 2,3-dihydrobenzofuryl, benzo-1,3-dioxyl, benzoxazolyl, benzothiazolyl, benzimidazolyl, benzoxadiazolyl, benzothiadiazolyl, benzotriazolyl, purinyl and imidazopyridinyl.

Unless otherwise specified, all bicyclic ring systems may be attached at any suitable position on either ring.

The terms “halogen” and “halo” represent chloro, fluoro, bromo, or iodo substituents. “Oxo” represents a double-bonded oxygen moiety; for example, if attached directly to a carbon atom forms a carbonyl moiety (C═O). “Hydroxy” or “hydroxyl” is intended to mean the radical —OH. As used herein, the term “cyano” refers to the group —CN.

As used herein, the term “optionally substituted” indicates that a group (such as an alkyl, cycloalkyl, alkoxy, heterocycloalkyl, aryl, or heteroaryl group) or ring or moiety (such as a carbocyclic or heterocyclic ring or moiety) may be unsubstituted, or the group, ring or moiety may be substituted with one or more substituent(s) as defined. In the case where groups may be selected from a number of alternative groups, the selected groups may be the same or different.

The term “independently” means that where more than one substituent is selected from a number of possible substituents, those substituents may be the same or different.

The term “pharmaceutically acceptable” refers to those compounds (including salts), materials, compositions, and dosage forms which are, within the scope of sound medical judgment, suitable for use in contact with the tissues of human beings and animals without excessive toxicity, irritation, or other problem or complication, commensurate with a reasonable benefit/risk ratio.

The compounds of this invention contain one or more asymmetric centers (also referred to as a chiral center), such as a chiral carbon, or a chiral —SO— moiety. The stereochemistry of the chiral carbon center present in compounds of this invention is generally represented in the compound names and/or in the chemical structures illustrated herein. Compounds of this invention containing one or more chiral centers may be present as racemic mixtures, diastereomeric mixtures, enantiomerically enriched mixtures, diastereomerically enriched mixtures, or as enantiomerically or diastereomerically pure individual stereoisomers.

In those instances where the stereochemistry of the chiral carbon center present in compounds of this invention is not represented in the compound name or in the accompanying chemical structure, it will be understood that the compound is present as a mixture of enantiomers or diastereomers. It is understood that one skilled in the art can obtain either the (R) or (S) isomer of any stereoisomeric compound mixture described herein using the resolution techniques described herein or using other conventional resolution techniques.

Individual stereoisomers of a compound described herein may be resolved (or mixtures of stereoisomers may be enriched) using methods known to those skilled in the art. For example, such resolution may be carried out (1) by formation of diastereoisomeric salts, complexes or other derivatives; (2) by selective reaction with a stereoisomer-specific reagent, for example by enzymatic oxidation or reduction; or (3) by gas-liquid or liquid chromatography in a chiral environment, for example, on a chiral support such as silica with a bound chiral ligand or in the presence of a chiral solvent. The skilled artisan will appreciate that where the desired stereoisomer is converted into another chemical entity by one of the separation procedures described above, a further step is required to liberate the desired form. Alternatively, specific stereoisomers may be synthesized by asymmetric synthesis using optically active reagents, substrates, catalysts or solvents, or by converting one enantiomer to the other by asymmetric transformation.

The alternative definitions for the various groups and substituent groups of Formula(s) (I) and/or (II) provided throughout the specification are intended to particularly describe each compound species disclosed herein, individually, as well as groups of one or more compound species. The scope of this invention includes any combination of these group and substituent group definitions. The compounds of the invention are only those which are contemplated to be “chemically stable” as will be appreciated by those skilled in the art.

As used herein, the terms “compound(s) of the invention” or “compound(s) of this invention” mean a compound of Formula(s) (I) and/or (II) as defined herein, in any form, i.e., any salt or non-salt form (e.g., as a free acid or base form, or as a salt, particularly a pharmaceutically acceptable salt thereof) and any physical form thereof (e.g., including non-solid forms (e.g., liquid or semi-solid forms), and solid forms (e.g., amorphous or crystalline forms, specific polymorphic forms, solvate forms, including hydrate forms (e.g., mono-, di- and hemi-hydrates)), and mixtures of various forms.

Accordingly, included within the present invention are the compounds of Formulas (I) and (II), as defined herein, in any salt or non-salt form and any physical form thereof, and mixtures of various forms. While such are included within the present invention, it will be understood that the compounds of Formulas (I) and (II), as defined herein, in any salt or non-salt form, and in any physical form thereof, may have varying levels of activity, different bioavailabilities and different handling properties for formulation purposes.

“Treating” or “treatment” is intended to mean at least the mitigation of a disease or disorder in a patient. The methods of treatment for mitigation of a disease or disorder include the use of the compounds in this invention in any conventionally acceptable manner, for example for prevention, retardation, prophylaxis, therapy or cure of a RIP1 kinase mediated disease or disorder, as described hereinabove.

As used herein, the term “cancer,” refers to cells that have undergone a malignant transformation that makes them pathological to the host organism. Primary cancer cells can be readily distinguished from non-cancerous cells by well-established techniques, particularly histological examination. The definition of a cancer cell, as used herein, includes not only a primary cancer cell, but any cell derived from a cancer cell ancestor. This includes metastasized cancer cells, and in vitro cultures and cell lines derived from cancer cells. When referring to a type of cancer that normally manifests as a solid tumor, a “clinically detectable” tumor is one that is detectable on the basis of tumor mass; e.g., by procedures such as computed tomography (CT) scan, magnetic resonance imaging (MRI), X-ray, ultrasound or palpation on physical examination, and/or which is detectable because of the expression of one or more cancer-specific antigens in a sample obtainable from a patient. Tumors may be a hematopoietic (or hematologic or hematological or blood-related) cancer, for example, cancers derived from blood cells or immune cells, which may be referred to as “liquid tumors.”

A therapeutically “effective amount” is intended to mean that amount of a compound that, when administered to a patient in need of such treatment, is sufficient to effect treatment, as defined herein. Thus, e.g., a therapeutically effective amount of a compound of Formula (I) or Formula (II), or a pharmaceutically acceptable salt thereof, is a quantity of an inventive agent that, when administered to a human in need thereof, is sufficient to modulate and/or inhibit the activity of RIP1 kinase such that a disease condition which is mediated by that activity is reduced, alleviated or prevented. The amount of a given compound that will correspond to such an amount will vary depending upon factors such as the particular compound (e.g., the potency (pIC₅₀), efficacy (EC₅₀), and the biological half-life of the particular compound), disease condition and its severity, the identity (e.g., age, size and weight) of the patient in need of treatment, but can nevertheless be routinely determined by one skilled in the art. Likewise, the duration of treatment and the time period of administration (time period between dosages and the timing of the dosages, e.g., before/with/after meals) of the compound will vary according to the identity of the mammal in need of treatment (e.g., weight), the particular compound and its properties (e.g., pharmacokinetic properties), disease or disorder and its severity and the specific composition and method being used, but can nevertheless be determined by one of skill in the art.

As used herein, “pharmaceutically acceptable excipient” means a material, composition or vehicle involved in giving form or consistency to the composition. Each excipient must be compatible with the other ingredients of the pharmaceutical composition when commingled such that interactions which would substantially reduce the efficacy of the compound of the invention when administered to a patient and interactions which would result in pharmaceutical compositions that are not pharmaceutically acceptable are avoided. In addition, each excipient must of course be of sufficiently high purity to render it pharmaceutically acceptable.

### Compound Preparation

### Generic Synthesis Schemes

The compounds of this invention may be made by a variety of methods, including well-known standard synthetic methods. Illustrative general synthetic methods are set out below and then specific compounds of the invention are prepared in the working examples. The skilled artisan will appreciate that if a substituent described herein is not compatible with the synthetic methods described herein, the substituent may be protected with a suitable protecting group that is stable to the reaction conditions. The protecting group may be removed at a suitable point in the reaction sequence to provide a desired intermediate or target compound. In all of the schemes described below, protecting groups for sensitive or reactive groups are employed where necessary in accordance with general principles of synthetic chemistry. Protecting groups are manipulated according to standard methods of organic synthesis (T.W. Green and P.G.M. Wuts, Protecting Groups in Organic Synthesis, John Wiley & Sons (1991) incorporated by reference with regard to protecting groups). These groups are removed at a convenient stage of the compound synthesis using methods that are readily apparent to those skilled in the art. The selection of processes as well as the reaction conditions and order of their execution shall be consistent with the preparation of compounds of the present invention. Starting materials are commercially available or are made from commercially available starting materials using methods known to those skilled in the art.

As will be understood by the skilled chemist, references to preparations carried out in a similar manner to, or by the general method of, other preparations, may encompass variations in routine parameters such as time, temperature, workup conditions, minor changes in reagent amounts, etc. The syntheses of intermediates provided in the Examples herein are applicable for producing intermediates of the invention having a variety of R groups employing appropriate precursors, which are protected if needed, to achieve compatibility with the reactions described.

Compounds of Formula (I) and Formula (II) can be prepared according to Scheme 1, Scheme 2, Scheme 3, Scheme 4, or analogous methods. Compounds of Formula (I) and Formula (II) can be prepared according to Scheme 1 or analogous methods. Wittig reaction of an aryl aldehyde of Formula A with (triphenylphosphoranylidene)-acetaldehyde affords an unsaturated aldehyde of Formula B. Reaction of an aldehyde of Formula B with hydrazine provides a dihydropyrazole of Formula C. The coupling of 1-(tert-butoxycarbonyl)piperidine-4-carboxylic acid with the dihydropyrazole of Formula C under amide bond forming conditions affords a compound of Formula D. Removal of the t-butoxycarbonyl group of a compound of Formula D affords a racemic piperdine of Formula E. Treatment of the racemic piperidine of Formula E with a chiral acid (e.g. (1R)-(−)-10-camphorsulfonic acid) provides a chiral amine salt of Formula F. Reaction of a compound of Formula F with and aryl halide or aryl sulfone under nucleophilic aromatic substitution conditions provides a compound of Formula (II).

Alternatively, compounds of Formula (I) and Formula (II) can be prepared through further transformation of a preexisting functional group of another compound of Formula (I) or Formula (II). For example, as in Scheme 2, a compound of Formula (I) or Formula (II) possessing a carboxylate ester (Formula G) may be hydrolyzed to provide a new compound of Formula (I) or Formula (II) possessing a carboxylic acid (Formula H). Additionally, a compound of Formula H may be further transformed through an amide bond forming reaction to afford an alternate compound of Formula (I) or Formula (II) possessing an amide (Formula J).

Alternatively, a compound of Formula (I) or Formula (II) can be prepared from a compound of Formula J according to Scheme 3. Reaction of the primary amide of a compound of Formula J with phosphorous oxychloride provides a compound of Formula (I) or Formula (II) possessing a nitrile (Formula K).

Alternatively, a compound of Formula (I) or Formula (II) may be prepared from another compound of Formula (I) or Formula (II) possessing a preexisting halogen (Formula L) according to Scheme 4. Reaction of a compound of Formula L with a primary or secondary amine under nucleophilic aromatic substitution conditions provides a compound of Formula M.

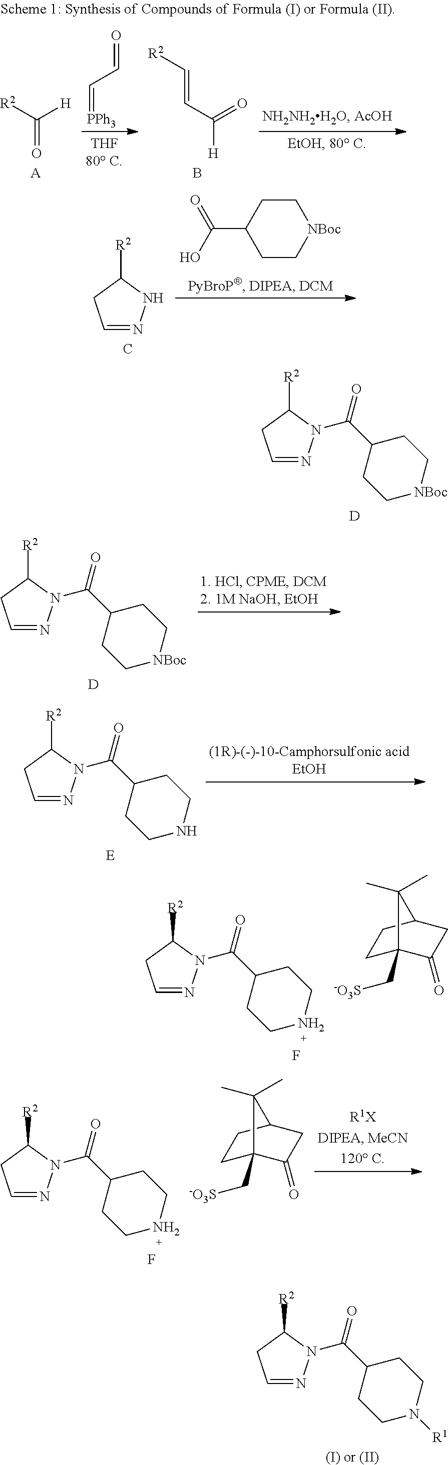

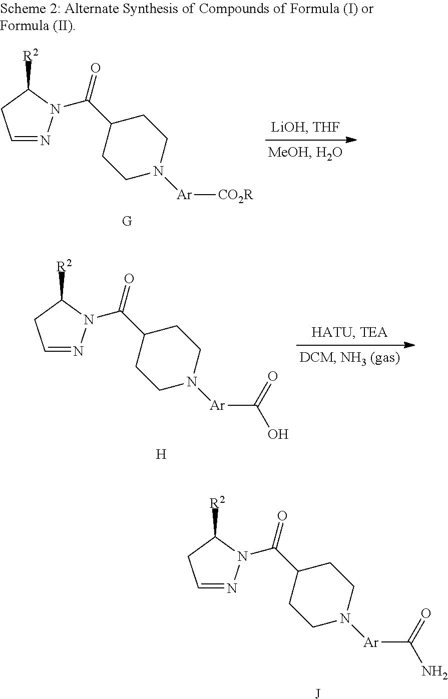

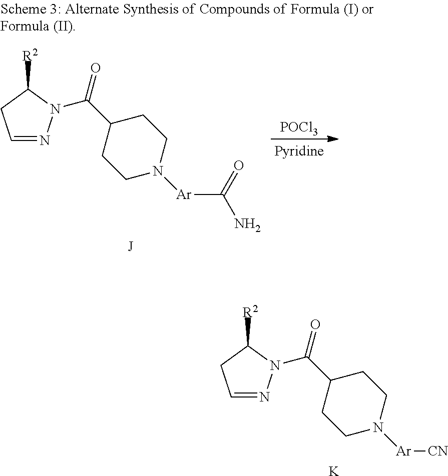

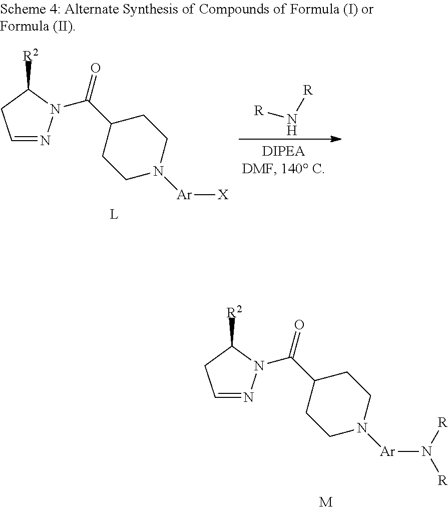

### EXAMPLES

The following examples illustrate the invention. These examples are not intended to limit the scope of the present invention, but rather to provide guidance to the skilled artisan to prepare and use the compounds, compositions, and methods of the present invention. While particular embodiments of the present invention are described, the skilled artisan will appreciate that various changes and modifications can be made without departing from the spirit and scope of the invention.

The reactions described herein are applicable for producing compounds of Formulas (I) and (II) having a variety of different substituent groups (e.g., R¹, R², etc.), as defined herein. The skilled artisan will appreciate that if a particular substituent is not compatible with the synthetic methods described herein, the substituent may be protected with a suitable protecting group that is stable to the reaction conditions. The protecting group may be removed at a suitable point in the reaction sequence to provide a desired intermediate or target compound. Suitable protecting groups and the methods for protecting and de-protecting different substituents using such suitable protecting groups are well known to those skilled in the art; examples of which may be found in T. Greene and P. Wuts, Protecting Groups in Chemical Synthesis (3rd ed.), John Wiley & Sons, NY, 1999.

Names for the intermediate and final compounds described herein were generated using the naming program in ChemDraw, Struct=Name Pro 12.0, as part of ChemBioDraw Ultra, available from CambridgeSoft. 100 CambridgePark Drive, Cambridge, Mass. 02140 USA (www.cambridgesoft.com).

It will be appreciated by those skilled in the art that in certain instances these programs may name a structurally depicted compound as a tautomer of that compound. It is to be understood that any reference to a named compound or a structurally depicted compound is intended to encompass all tautomers of such compounds and any mixtures of tautomers thereof.

¹H NMR spectra were recorded in either CDCl₃ or DMSO-d6 on either a Bruker DPX 400, Bruker Advance DRX, Varian Unity 400 spectrometer or JEOL Delta all working at 400 MHz. The internal standard used was either tetramethylsilane or the residual protonated solvent at 7.25 ppm for CDCl₃ or 2.50 ppm for DMSO-d6. Chemical shifts are reported in parts per million (ppm). Abbreviations for NMR data are as follows: s=singlet, d=doublet, t=triplet, m=multiplet, br s=broad singlet, dd=doublet of doublets, dt=doublet of triplets, tt=triplet of triplets, ddd=doublet of doublet of doublets, sextuplet of d=sextuplet of doublets. J indicates the ¹H NMR coupling constant measured in Hertz.

Mass spectrum was recorded on a Waters ZQ mass spectrometer using alternative-scan positive and negative mode electrospray ionisation. Cone voltage: 20 or 5V.

Chiral HPLC Method 1: on CHIRALPAK®AD-H was using 4.6×150 mm column, Heptane/EtOH 50/50 with 0.1% isopropylamine at 254 nm, at a flow rate of 1 mL/min.

Chiral HPLC Method 2: on CHIRALPAK® IE was using 250×4.6 5 μm, Heptane/EtOH 70/30+0.1% TFA+0.3% TEA, at a flow rate of 1.5 mL/min, at 40° C.

LC/MS Method 1: HPLC was conducted on a X-Select CSH C18 XP column (2.5 μm 30×4.6 mm id) eluting with 0.1% formic acid in water (solvent A) and 0.1% formic acid in acetonitrile (solvent B), using the following elution gradient 0-3 min.: 5% to 100% B, 3-4 min. 100% B, at a flow rate of 1.8 mL/min. at 40° C.

LC/MS Method 2: Analytical HPLC was conducted on a X-Select CSH C18 XP column (2.5 μm 30×4.6 mm id) eluting with 0.1% formic acid in water (solvent A) and 0.1% formic acid in acetonitrile (solvent B), using the following elution gradient 0-4 minutes: 0% to 50% B at a flow rate of 1.8 mL/minute at 40° C.

In the following experimental descriptions, the following abbreviations may be used:

Abbreviation

| | |
|---|---|
| 2-MeTHF | 2-methyltetrahydrofuran |
| Ac₂O | acetic anhydride |
| AcOH | acetic acid |
| aq. | aqueous |
| BOC, tBOC, | tert-butoxycarbonyl |
| Boc | |
| brine | saturated aqueous solution of sodium chloride |
| CDI | 1,1′-carbonyldiimidazole |
| CH₂Cl₂ or | methylene chloride or 1,2-dichloromethane |
| DCM | |
| CH₃CN or | acetonitrile |
| MeCN | |
| CPME | cyclopentyl methyl ether |
| cPrNH₂ | cyclopropylamine |
| Cs₂CO₃ | cesium carbonate |
| Cu(OTf)₂ | copper(II) trifluoromethansulfonate |
| Cy or CyH | cyclohexane |
| DCE | 1,2-dichloroethane |
| DCM | dichloromethane |
| DIBAL or | diisobutylaluminium hydride |
| DIBAL-H | |
| DIEA or | diisopropyl ethylamine |
| DIPEA | |
| DMAP | 4-dimethylaminopyidine |
| DMF | N,N-dimethylformamide |
| DMSO | dimethylsulfoxide |
| Et | ethyl |
| Et₃N or TEA | triethylamine |
| Et₂O | diethyl ether |
| EtOH | ethanol |
| EtOAc | ethyl acetate |
| h, hr | hour(s) |
| HATU | O-(7-Azabenzotriazol-1yl)-N,N,N′,N′-tetramethylyronium |
| | hexafluorophosphate |
| HCl | hydrochloric acid |
| HFIP | 1,1,1,3,3,3-hexafluoro-2-propanol |
| i-Pr₂Net | N′,N′-diisopropylethylamine |
| iPr₂O | diisopropyl ether |
| KOH | potassium hydroxide |
| Kot-Bu | potassium tert-butoxide |
| LCMS | liquid chromatography-mass spectroscopy |
| LiHDMS | lithium hexamethyldisilazide |
| LiOH | lithium hydroxide |
| Me | methyl |
| MeCN | acetonitrile |
| MeOH or | methanol |
| CH₃OH | |
| min | minute(s) |
| MnO₂ | manganese dioxide |
| MTBE | methyl t-butyl ether |
| MS | mass spectrum |
| NaBH₄ | sodium borohydride |
| Na₂CO₃ | sodium carbonate |
| NaH | sodium hydride |
| NaHCO₃ | sodium bicarbonate |
| NaOH | sodium hydroxide |
| Na₂SO₄ | sodium sulfate |
| NH₄Cl | ammonium chloride |
| NH₂NH₂•H₂O | hydrazine monohydrate |
| NH₄OH | ammonium hydroxide |
| NMP | N-methyl-2-pyrrolidone |
| Oxone ® | potassium peroxymonosulfate |
| PdOAc₂ | lead acetate |
| Ph | phenyl |
| PPh₃ | triphenyl phospine |
| POCl₃ | phosphoryl chloride |
| Pr | propyl |
| PyBroP ® | bromotripyrrolidinophosphonium hexafluorophosphate |
| rt | room temperature |
| SOCl₂ | thionyl chloride |
| t-BuOH | tert-butanol |
| TEA | triethylamine |
| TBAF | tetrabutylammonium fluoride |
| TBS | tert-butyl(methoxy)dimethylsilyl |
| TFA | trifluoroacetic acid |
| THF | tetrahydrofuran |
| TMS | trimethylsilyl |
| TsCl | p-toluenesulfonyl chloride, tosyl chloride |
| UPLC | ultra performance liquid chromatography |
| UV | ultraviolet |

### Example 1

### (S)-(1-(4-(benzylthio)pyrimidin-2-yl)piperidin-4-yl)(5-phenyl-4,5-dihydro-1H-pyrazol-1-yl)methanone

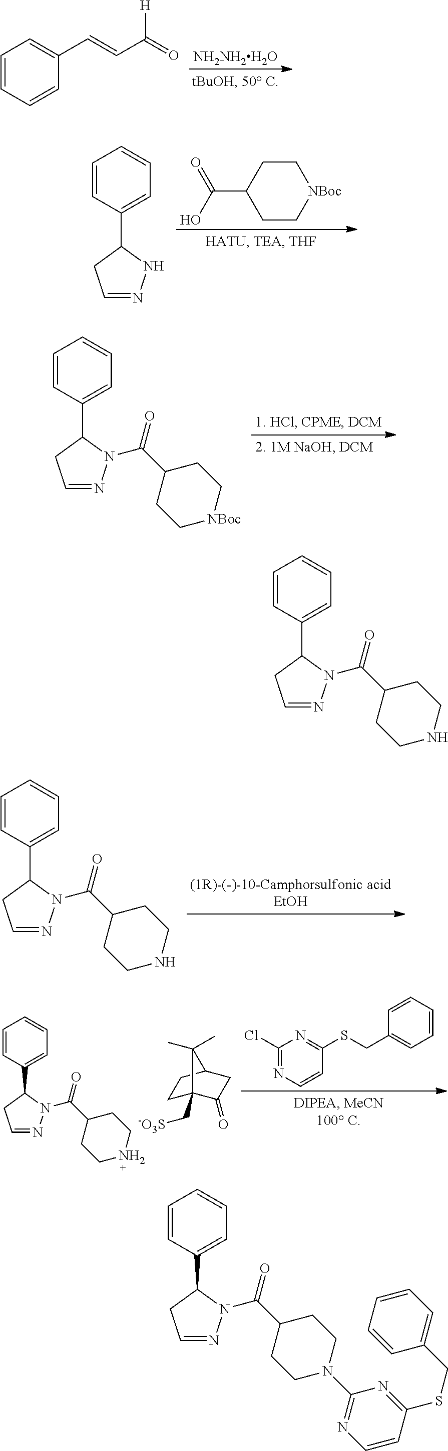

### Step 1

To neat hydrazine monohydrate (150 mL, 3.1 mol) stirred under nitrogen at 50° C. was added a solution of 3-phenylacrylaldehyde (100 mL, 794 mmol) in tBuOH (150 mL). The reaction mixture was stirred at 50° C. for 15 h and evaporated in vacuo to give 5-phenyl-4,5-dihydro-1H-pyrazole (115.7 g, 791 mmol, purity: 57%, recovery: 100%) as a yellow oil. This product was used in next reaction without further purification. LCMS (m/z) 147 (M+H⁺), retention time: 1.38 min, LC/MS Method 1.

### Step 2

To a solution of 5-phenyl-4,5-dihydro-1H-pyrazole (105 g, 718 mmol), 1-(tert-butoxycarbonyl)piperidine-4-carboxylic acid (186.16 g, 796 mmol) and TEA (0.2 L, 1.4 mol) in THF (1 L) stirred under argon at 0° C. was added solid HATU (328 g, 862 mmol) portion wise. The reaction mixture was stirred at rt for 72 h. The reaction mixture was evaporated in vacuo to give a dark red oil. EtOAc (1 L) was added and the resulting organic solution washed successively with ammonium chloride (600 mL), water (2×600 mL) and brine (600 mL). After separation, the organic layer was dried over sodium sulfate and evaporated in vacuo to give a brown oily solid. This residue was crystallized with hot ethanol (solubilization at 60° C.) to afford, after filtration, tert-butyl 4-(5-phenyl-4,5-dihydro-1H-pyrazole-1-carbonyl)piperidine-1-carboxylate (127.4 g, 356 mmol, purity: 100%, recovery: 50%) as a white solid. LCMS (m/z) 358 (M+H⁺), retention time: 2.67 min, LC/MS Method 1.

### Step 3

To a solution of tert-butyl 4-(5-phenyl-4,5-dihydro-1H-pyrazole-1-carbonyl)piperidine-1-carboxylate (127.4 g, 356 mmol) in DCM (1 L) stirred at 0° C. was added a 3 M solution of HCl in CPME (200 mL, 600 mmol) dropwise. The reaction mixture was stirred at rt for 72 h. A 2 M solution of HCl in Et₂O (356 mL, 713 mmol) was added dropwise at 5° C. The reaction mixture was stirred at rt for 15 h. The precipitate was filtered, washed with iPr₂O and dried at 45° C. on a high vacuum to afford (5-phenyl-4,5-dihydro-1H-pyrazol-1-yl)(piperidin-4-yl)methanone, hydrochloride (104.5 g, 356 mmol, purity: 84%, recovery: 100%) as a white solid. LCMS (m/z) 258 (M+H)⁺, retention time: 0.92 min, LC/MS Method 1.

### Step 4

To a suspension of (5-phenyl-4,5-dihydro-1H-pyrazol-1-yl)(piperidin-4-yl)methanone, hydrochloride (104 g, 354 mmol) in DCM (400 mL) stirred at rt was added a 1 M solution of sodium hydroxide in water (460 mL, 460 mmol). The reaction mixture was stirred until complete dissolution. After separation, the aqueous layer was extracted with DCM (1×400 mL). The combined organic layers were washed with brine, dried over sodium sulfate, and evaporated in vacuo to give the free base as a light yellow oil. This residue was partially dissolved in EtOH (500 mL), and solid (1R)-(−)-10-camphorsulfonic acid (82 g, 354 mmol) was added portion wise. The mixture was heated at reflux, EtOH was added by portion of 50-100 mL until complete dissolution, and the mixture was allowed to cool to rt slowly. After 72 h, the solid was filtrated, washed with EtOH and dried at 50° C. on high vacuum to afford (5)-(5-phenyl-4,5-dihydro-1H-pyrazol-1-yl)(piperidin-4-yl)methanone, 1R-(−)-camphor-10-sulphonic acid salt (64.1 g, 131 mmol, purity: 100%, recovery: 37%) as cream-colored crystals. LCMS (m/z) 258 (M+H⁺), retention time: 0.66 min, LC/MS Method 1. Chiral HPLC Method 1: 7.76 min, % ee=100%.

### Step 5

To a suspension of (S)-(5-phenyl-4,5-dihydro-1H-pyrazol-1-yl)(piperidin-4-yl)methanone, (1R)-10-camphorsulphonic acid salt (200 mg, 0.41 mmol) and 4-(benzylthio)-2-chloropyrimidine (102 mg, 0.43 mmol) in MeCN (5 mL) at rt was added neat DIPEA (0.18 mL, 1.02 mmol) in one charge. The reaction vessel was sealed and the mixture was stirred at 100° C. for 1.75 h and evaporated in vacuo. The residue was purified by normal phase column chromatography [CyH/(EtOAC/EtOH 3:1) 100/0 to 90/10] to afford (S)-(1-(4-(benzylthio)pyrimidin-2-yl)piperidin-4-yl) (5-phenyl-4,5-dihydro-1H-pyrazol-1-yl)methanone (45 mg, 0.09 mmol, purity: 100%, recovery: 23%) as an off-white powder. LCMS (m/z) 458 (M+H)⁺, retention time: 2.92 min, LC/MS Method 1. ¹H NMR (400 MHz, DMSO-d6) δ ppm 8.02 (d, J=5.3 Hz, 1H), 7.40 (d, J=7.4 Hz, 2H), 7.28 (m, 7H), 7.11 (d, J=7.2 Hz, 2H), 6.52 (d, J=5.3 Hz, 1H), 5.32 (dd, J=11.8, 4.6 Hz, 1H), 4.67 (d, J=12.9 Hz, 2H), 4.39 (s, 2H), 3.49 (dd, J=18.5, 12.4 Hz, 1H), 3.38 (m, 1H), 3.00 (m, 2H), 2.68 (dd, J=18.9, 3.1 Hz, 1H), 1.88 (d, J=11.6 Hz, 1H), 1.74 (d, J=12.3 Hz, 1H), 1.44 (m, 2H).

Examples 2-76 were synthesized in an analogous manner. For step 5, DIPEA may be substituted for TEA, cesium carbonate, or potassium carbonate; the temperature may vary from 80 to 150° C.; MeCN may be substituted for DMF. In Examples 2 to 7, where the product is racemic, the diastereoselective crystallization of the camphor sulfonice acid salt (step 4) was omitted. For Example 62 and 68 to 76, 3-(3-oxoprop-1-en-1-yl)benzonitrile was synthesized by the procedure described in F. Hirayama et al., Bioorganic & Medicinal Chemistry, 10, 1509-1523 (2002) with THF instead of benzene. For step 2, HATU may be substituted for PyBroP®.

| | | | | LC: retention | |
|---|---|---|---|---|---|
| | | | | time (min); | |
| | | | | LC/MS | MS |
| | | | | Method of | (M + |
| Ex. | Name | Structure | ¹H NMR | analysis | H)⁺ |
| 2 | 2-(4-(5-phenyl-4,5- dihydro-1H- pyrazole-1- carbonyl)piperidin- 1-yl)pyrimidine-5- carbonitrile | 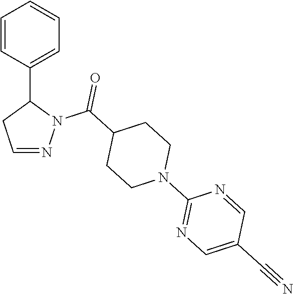 [SMILES: N#Cc1cnc(N2CCC(C(=O)N3N=CCC3c3ccccc3)CC2)nc1] | ¹H NMR (400 MHz, DMSO-d6) δ ppm 8.73 (s, 2H), 7.32 (dd, J = 7.8, 7.2 Hz, 2H), 7.25 (m, 2H), 7.11 (d, J = 7.2 Hz, 2H), 5.31 (dd, J = 11.8, 4.6 Hz, 1H), 4.69 (d, J = 13.5 Hz, 2H), 3.50 (ddd, J = 18.8, 12.0, 4.5 Hz, 1H), 3.43 (tt, J = 11.2, 3.80 Hz, 1H), 3.16 (m, 2H), 2.68 (18.8, 4.7, 1.7 Hz, 1H), 1.94 (d, J = 11.0 Hz, 1H), 1.82 (d, J = 11.0 Hz, 1H), 1.49 (m, 2H) | 2.53 Method 1 | 361 |
| 3 | (1-(4- methoxypyrimidin- 2-yl)piperidin-4- yl)(5-phenyl-4,5- dihydro-1H- pyrazol-1- yl)methanone | 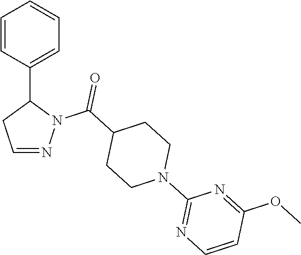 [SMILES: COc1ccnc(N2CCC(C(=O)N3N=CCC3c3ccccc3)CC2)n1] | ¹H NMR (400 MHz, DMSO-d6) δ ppm 8.07 (d, J = 5.6Hz, 1H), 7.32 (dd, 7.6, 7.2 Hz, 2H), 7.24 (m, 2H), 7.12 (d, J = 7.0 Hz, 2H), 6.04 (d, J = 5.6 Hz, 1H), 5.31 (dd, J = 11.8, 4.6 Hz, 1H), 4.65 (d, J = 13.1 Hz, 2H), 3.82 (s, 3H), 3.50 (ddd, J = 18.8, 11.8, 1.5 Hz, 1H), 3.38 (m, 1H), 2.97 (m, 2H), 2.68 (ddd, J = 20.5, 4.6, 1.7 Hz, 1H), 1.88 (d, J = 11.2 Hz, 1H), 1.76 (m, 1H), 1.47 (m, 2H) | 1.64 Method 1 | 366 |
| 4 | (5-phenyl-4,5- dihydro-1H- pyrazol-1-yl)(1-(4- phenylpyrimidin-2- yl)piperidin-4- yl)methanone | 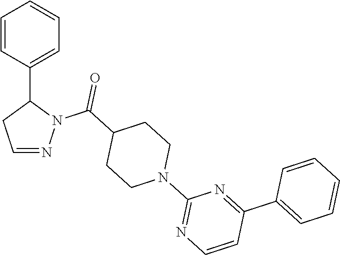 [SMILES: O=C(C1CCN(c2nccc(-c3ccccc3)n2)CC1)N1N=CCC1c1ccccc1] | ¹H NMR (400 MHz, DMSO-d6) δ ppm 8.43 (d, J = 5.1 Hz, 1H), 8.12 (dd, J = 6.6, 3.0 Hz, 2H), 7.51 (m, 3H), 7.32 (dd, J = 7.6, 7.2 Hz, 2H), 7.25 (s, 1H), 7.23 (m, 1H), 7.18 (d, J = 5.1 Hz, 1H), 7.11 (d, J = 7.2 Hz, 2H), 5.32 (dd, J = 11.8, 4.6 Hz, 1H), 4.81 (d, J = 13.1 Hz, 2H), 3.50 (ddd, J = 18.9, 12.0, 1.3 Hz, 1H), 3.41 (tt, 11.6, 3.7 Hz, 1H), 3.05 (m, 2H), 2.68 (ddd, J = 18.9, 4.6, 1.7 Hz, 1H), 1.92 (d, J = 11.2Hz, 1H), 1.80 (d, J = 11.4 Hz, 1H), 1.51 (m, 2H) | 2.89 Method 1 | 412 |
| 5 | 2-(4-(5-phenyl-4,5- dihydro-1H- pyrazole-1- carbonyl)piperidin- 1-yl)pyrimidine-4- carbonitrile | 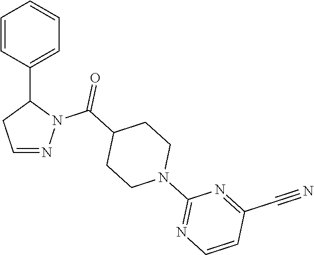 [SMILES: N#Cc1ccnc(N2CCC(C(=O)N3N=CCC3c3ccccc3)CC2)n1] | ¹H NMR (400 MHz, DMSO-d6) δ ppm 8.62 (d, J = 4.7 Hz, 1H), 7.32 (dd, J = 7.6, 7.2 Hz, 2H), 7.23 (m, 2H), 7.11 (m, 3H), 5.31 (dd, J = 11.9, 4.6Hz, 1H), 4.58 (d, J = 12.9 Hz, 2H), 3.50 (ddd, J = 18.8, 11.8, 1.3 Hz, 1H), 3.41 (tt, J = 11.5, 3.7 Hz, 1H), 3.08 (m, 2H), 2.68 (ddd, J = 18.8, 4.6, 1.7 Hz, 1H), 1.92 (d, J = 11.6 Hz, 1H), 1.79 (d, J = 11.8 Hz, 1H), 1.48 (m, 2H) | 2.68 Method 1 | 361 |
| 6 | (1-(4- aminopyrimidin-2- yl)piperidin-4-yl)(5- phenyl-4,5-dihydro- 1H-pyrazol-1- yl)methanone | 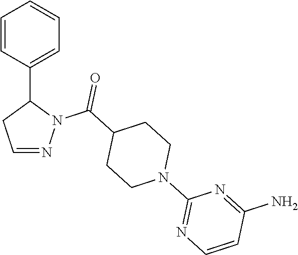 [SMILES: Nc1ccnc(N2CCC(C(=O)N3N=CCC3c3ccccc3)CC2)n1] | ¹H NMR (400 MHz, DMSO-d6) δ ppm 7.72 (d, J = 5.7 Hz, 1H), 7.32 (dd, J = 7.6, 7.2 Hz, 2H), 7.23 (m, 2H), 7.11 (d, J)7.2 Hz, 2H), 3.65 (s, 2H), 5.7 (d, J = 5.7 Hz, 1H), 5.31 (dd, J = 11.9, 4.6 Hz, 1H), 4.62 (d, J = 13.1Hz, 2H), 3.49 (ddd, J = 18.9, 12.0, 1.5 Hz, 1H), 3.32 (m, 1H) 2.82 (m, 2H), 2.67 (ddd, J = 18.9, 4.7, 1.7 Hz, 1H), 1.8 (d, J = 10.8 Hz, 1H), 1.68 (d, J = 11.2Hz, 1H), 1.41 (m, 2H) | 1.41 Method | 351 |
| 7 | (1-(5- methoxypyrimidin- 2-yl)piperidin-4- yl)(5-phenyl-4,5- dihydro-1H- pyrazol-1- yl)methanone | 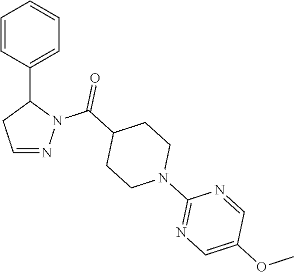 [SMILES: COc1cnc(N2CCC(C(=O)N3N=CCC3c3ccccc3)CC2)nc1] | ¹H NMR (400 MHz, DMSO-d6) δ ppm 8.19 (s, 2H), 7.32 (dd, J = 7.6, 7.2 Hz, 2H), 7.24 (m, 2H), 7.10 (d, J = 7.0 Hz, 2H), 5.31 (dd, J = 11.9, 4.6 Hz, 1H), 4.53 (d, J = 13.1 Hz, 2H), 3.76 (s, 3H), 3.49 (ddd, J = 18.8, 11.8, 1.3Hz, 1H), 3.34 (m, 1H), 2.93 (m, 2H), 2.67 (ddd, J = 18.9, 4.7, 1.7 Hz, 1H), 1.85 (d, J = 11.8 Hz, 1H), 1.72 (d, J = 11.8 Hz, 1H), 1.46 (m, 2H) | 2.43 Method 1 | 366 |
| 8 | (S)-(1-(5- (methylsulfonyl)pyrimidin- 2-yl)piperidin-4-yl)(5- phenyl-4,5-dihydro- 1H-pyrazol-1- yl)methanone | 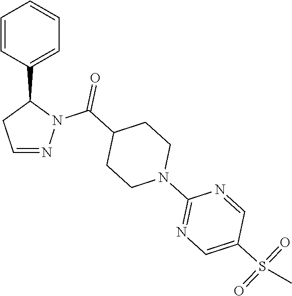 [SMILES: CS(=O)(=O)c1cnc(N2CCC(C(=O)N3N=CC[C@H]3c3ccccc3)CC2)nc1] | ¹H NMR (400 MHz, DMSO-d6) δ ppm 8.70 (s, 2H), 7.32 (dd, J = 7.6, 7.2 Hz, 2H), 7.24 (m, 2H), 7.11 (d, J = 7.2 Hz, 2H), 5.32 (dd, J = 12.0, 4.6 Hz, 1H), 4.74 (d, J = 10.8 Hz, 2H), 3.47 (m, 2H), 3.22 (s, 3H), 3.20 (m, 2H), 2.69 (ddd, J = 18.9, 4.6, 1.5 Hz, 1H), 1.95 (d, J = 11.8Hz, 1H), 1.83 (d, J = 11.6Hz, 1H), 1.50 (m, 2H) | 2.30 Method 1 | 414 |
| 9 | (S)-(1-(7H-purin-2- yl)piperidin-4-yl)(5- phenyl-4,5-dihydro- 1H-pyrazol-1- yl)methanone | 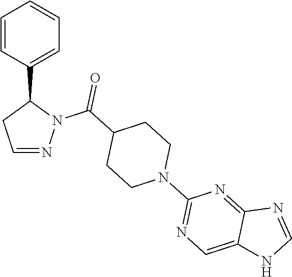 [SMILES: O=C(C1CCN(c2ncc3[nH]cnc3n2)CC1)N1N=CC[C@H]1c1ccccc1] | ¹H NMR (400 MHz, DMSO-d6) δ ppm 12.69 (s, 1H), 8.69 (s, 1H), 8.08 (s, 1H), 7.32 (dd, J = 7.6, 7.2 Hz, 2H), 7.23 (m, 2H), 7.11 (d, J = 7.2 Hz, 2H), 5.31 (dd, J = 11.8, 4.4 Hz, 1H), 4.68 (d, J = 12.3 Hz, 2H), 3.50 (dd, J = 18.6, 12.3 Hz, 1H), 3.38 (m, 1H), 3.01 (m, 2H), 2.68 (dd, J = 18.8, 3.2 Hz, 1H), 1.88 (d, J = 11.6 Hz, 1H), 1.76 (d, J = 11.6Hz, 1H), 1.50 (m, 2H) | 1.77 Method 1 | 376 |
| 10 | (S)-methyl 2-(4-(5- phenyl-4,5-dihydro- 1H-pyrazole-1- carbonyl)piperidin- 1-yl)pyrimidine-5- carboxylate | 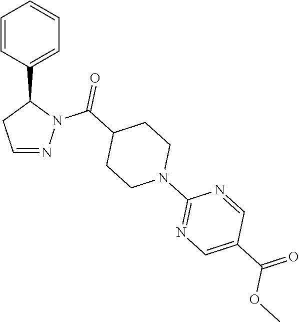 [SMILES: COC(=O)c1cnc(N2CCC(C(=O)N3N=CC[C@H]3c3ccccc3)CC2)nc1] | ¹H NMR (400 MHz, DMSO-d6) δ ppm 8.78 (s, 2H), 7.32 ( dd, J = 7.6, 7.0 Hz, 2H), 7.23 (m, 2H), 7.11 (d, J = 7.2 Hz, 2H), 5.32 (dd, J = 11.9, 4.6 Hz, 1H), 4.74 (d, J = 13.3 Hz, 2H), 3.80 (s, 3H), 3.50 (ddd, J = 18.8, 12.0, 1.5 Hz, 1H), 3.43 (tt, J = 11.2, 3.8 Hz, 1H), 3.14 (m, 2H), 2.69 (ddd, J = 18.8, 4.7, 1.7 Hz, 1H), 1.94 (d, J = 11.2 Hz, 1H), 1.82 (d, J = 11.2 Hz, 1H), 1.49 (m, 2H) | 2.57 Method 1 | 394 |
| 11 | (S)-(1-(2- aminopyrimidin-4- yl)piperidin-4-yl)(5- phenyl-4,5-dihydro- 1H-pyrazol-1- yl)methanone | 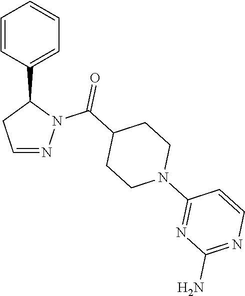 [SMILES: Nc1nccc(N2CCC(C(=O)N3N=CC[C@H]3c3ccccc3)CC2)n1] | ¹H NMR (400 MHz, DMSO-d6) δ ppm 7.76 (s, 1H), 7.32 (t, J = 7.4 Hz, 2H), 7.24 (m, 2H), 7.11 (d, J = 7.2 Hz, 2H), 6.54 (s, 2H), 6.2 (d, J = 6.5 Hz, 1H), 5.31 (dd, J = 11.9, 4.5 Hz, 1H), 4.36 (d, J = 12.0 Hz, 2H), 3.50 (dd, J = 18.9, 12.1 Hz, 1H), 3.40 (m, 1H), 3.03 (m, 2H), 2.68 (m, 1H), 1.82 (m, 2H), 1.46 (m, 2H) | 1.43 Method 1 | 351 |
| 12 | (S)-(1-(6- methoxypyrimidin- 4-yl)piperidin-4- yl)(5-phenyl-4,5- dihydro-1H- pyrazol-1- yl)methanone | 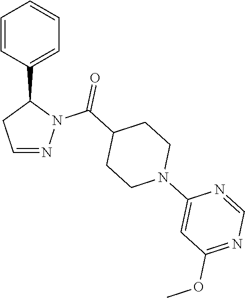 [SMILES: COc1cc(N2CCC(C(=O)N3N=CC[C@H]3c3ccccc3)CC2)ncn1] | ¹H NMR (400 MHz, DMSO-d6) δ ppm 8.23 (s, 1H), 7.32 (dd, J = 7.6, 7.2 Hz, 2H), 7.24 (m, 2H), 7.10 (d, J = 7.2 Hz, 2H), 6.08 (s, 1H), 5.31 (dd, J = 11.9, 4.6 Hz, 1H), 4.34 (d, J = 13.1 Hz, 2H), 3.81 (s, 3H), 3.49 (ddd, J = 18.8, 12.0, 1.3 Hz, 1H), 3.38 (tt, J = 11.5, 3.8 Hz, 1H), 2.98 (m, 2H), 2.68 (ddd, J = 18.8, 4.6, 1.5 Hz, 1H), 1.86 (d, J = 11.9 Hz, 1H), 1.73 (d, J = 11.9 Hz, 1H), 1.45 (m, 2H) | 1.92 Method 1 | 366 |
| 13 | (S)-(1-(5- methoxypyrimidin- 2-yl)piperidin-4- yl)(5-phenyl-4,5- dihydro-1H- pyrazol-1- yl)methanone | 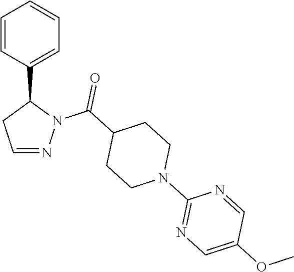 [SMILES: COc1cnc(N2CCC(C(=O)N3N=CC[C@H]3c3ccccc3)CC2)nc1] | ¹H NMR (400 MHz, DMSO-d6) δ ppm 8.19 (s, 2H), 7.32 (dd, J = 7.6, 7.2 Hz, 2H), 7.24 (m, 2H), 7.11 (d, J = 7.2 Hz, 2H), 5.31 (dd, J = 11.9, 4.6 Hz, 1H), 4.53 (d, J = 13.1 Hz, 2H), 3.76 (s, 3H), 3.49 (ddd, J = 18.8, 12.0, 1.3 Hz, 1H), 3.35 (m, 1H), 2.93 (m, 2H), 2.67 (ddd, J = 18.8, 4.6, 1.7 Hz, 1H), 1.85 (d, J = 12.3 Hz, 1H), 1.72 (d, J = 11.2 Hz, 1H), 1.46 (m, 2H) | 2.41 Method 1 | 366 |
| 14 | (S)-6-(4-(5-phenyl- 4,5-dihydro-1H- pyrazole-1- carbonyl)piperidin- 1-yl)pyrimidine-4- carbonitrile | 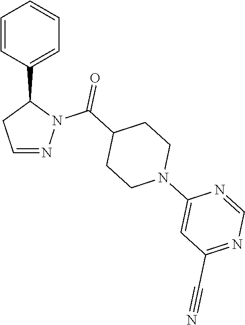 [SMILES: N#Cc1cc(N2CCC(C(=O)N3N=CC[C@H]3c3ccccc3)CC2)ncn1] | ¹H NMR (400 MHz, DMSO-d6) δ ppm 8.54 (s, 1H), 7.56 (s, 1H), 7.32 (dd, J = 7.6, 7.2 Hz, 2H), 7.24 (m, 2H), 7.11 (d, J = 7.2 Hz, 2H), 5.3 (dd, J = 11.9, 4.6 Hz, 1H), 4.0-4.9 (br. s, 2H), 3.50 (dd, J = 18.9, 11.8, 1.6 Hz, 1H), 3.43 (tt, J = 11.3, 3.7 Hz, 1H), 3.10 (br. s, 2H), 2.68 (ddd, J = 18.9, 4.6, 1.6 Hz, 1H), 1.93 (d, J = 12.9 Hz, 1H), 1.79 (d, J = 12.9 Hz, 1H), 1.49 (m, 2H) | 2.34 Method 1 | 361 |
| 15 | (S)-(1-(2- (methylamino)pyrimidin- 4-yl)piperidin-4-yl)(5- phenyl-4,5-dihydro-1 H-pyrazol-1- yl)methanone | 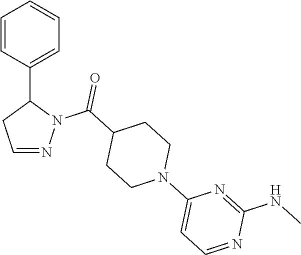 [SMILES: CNc1nccc(N2CCC(C(=O)N3N=CCC3c3ccccc3)CC2)n1] | ¹H NMR (400 MHz, DMSO-d6) δ ppm 7.77 (d, J = 6.0 Hz, 1H), 7.32 (dd, J = 7.6, 7.2 Hz, 2H), 7.24 (m, 2H) 7.10 (d, J = 7.2 Hz, 2H), 6.38 (br. s, 1H), 6.01 (d, J = 6.0Hz, 1H), 5.31 (dd, J = 12.0, 4.6 Hz, 1H), 4.33 (d, J = 12.5 Hz, 2H), 3.49 (ddd, J = 18.8, 12.0, 1.6 Hz, 1H), 3.36 (tt, J = 11.4, 3.8 Hz, 1H), 2.90 (m, 2H), 2.72 (d, J = 4.7 Hz, 3H), 2.67 (ddd, J = 18.8, 4.6, 1.6 Hz, 1H), 1.84 (d, J = 12.2 Hz, 1H), 1.72 (d, J = 12.2 Hz, 1H), 1.44 (m, 2H) | 1.58 Method 1 | 365 |
| 16 | (S)-(1-(4- (methylamino)pyrimidin- 2-yl)piperidin-4-yl)(5- phenyl-4,5-dihydro- 1H-pyrazol-1- yl)methanone | 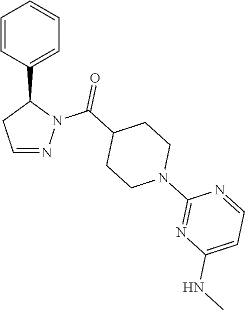 [SMILES: CNc1ccnc(N2CCC(C(=O)N3N=CC[C@H]3c3ccccc3)CC2)n1] | ¹H NMR (400 MHz, DMSO-d6) δ ppm 7.69 (br. s, 1H), 7.32 (dd, J = 7.6, 7.2 Hz, 2H), 7.24 (m, 2H), 7.11 (d, J = 7.2 Hz, 2H), 6.88 (br. s, 1H), 5.71 (d, J = 5.7 Hz, 1H), 5.31 (dd, J = 11.9, 4.5 Hz, 1H), 4.65 (d, J = 13.1 Hz, 2H), 3.49 (dd, J = 18.8, 12.0 Hz, 1H), 3.32 (m, 1H), 2.84 (m, 2H), 2.74 (d, J = 4.0 Hz, 3H), 2.67 (ddd, J = 18.8, 4.5, 1.3 Hz, 1H), 1.82 (d, J = 11.8 Hz, 1H), 1.70 (d, J = 11.8 Hz, 1H), 1.43 (m, 2H) | 1.57 Method 1 | 365 |
| 17 | (S)-(1-(2- methoxypyrimidin- 4-yl)piperidin-4- yl)(5-phenyl-4,5- dihydro-1H- pyrazol-1- yl)methanone | 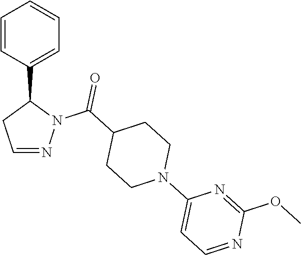 [SMILES: COc1nccc(N2CCC(C(=O)N3N=CC[C@H]3c3ccccc3)CC2)n1] | ¹H NMR (400 MHz, DMSO-d6) δ ppm 7.98 (d, J = 6.1 Hz, 1H), 7.32 (dd, J = 7.6, 7.0 Hz, 2H), 7.24 (m, 2H), 7.11 (m, 2H), 6.48 (d, J = 6.1 Hz, 1H), 5.31 (dd, J = 11.9, 4.6 Hz, 1H), 4.35 (s, 2H), 3.78 (s, 3H), 3.50 (ddd, J = 18.8, 12.0, 1.3 Hz, 1H), 3.39 (m, 1H), 3.01 (m, 2H), 2.68 (ddd, J = 18.8, 4.6, 1.7 Hz, 1H), 1.89 (d, J = 13.1 Hz, 1H), 1.76 (d, J = 11.4 Hz, 1H), 1.46 (m, 2H) | 1.56 Method 1 | 366 |
| 18 | (S)-4-(4-(5-phenyl- 4,5-dihydro-1H- pyrazole-1- carbonyl)piperidin- 1-yl)pyrimidine-2- carbonitrile | 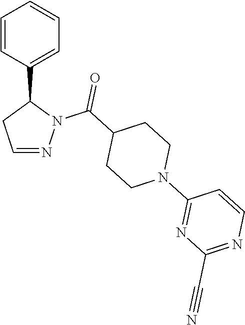 [SMILES: N#Cc1nccc(N2CCC(C(=O)N3N=CC[C@H]3c3ccccc3)CC2)n1] | ¹H NMR (400 MHz, DMSO-d6) δ ppm 8.23 (d, J = 6.5 Hz, 1H), 7.32 (dd, J = 7.6, 7.2 Hz, 2H), 7.24 (m, 2H), 7.11 (d, J = 6.5 Hz, 3H), 5.31 (dd, J = 11.9, 4.6 Hz, 1H), 4.36 (br. s, 2H), 3.50 (ddd, J = 18.9, 11.9, 1.2 Hz, 1H), 3.43 (tt, J = 11.4, 3.8 Hz, 1H), 3.12 (m, 2H), 2.68 (ddd, J = 18.9, 4.6, 1.7 Hz, 1H), 1.94 (d, J = 13.1 Hz, 1H), 1.81 (d, J = 13.1 Hz, 1H), 1.5 (m, 2H) | 2.42 Method 1 | 361 |
| 19 | (S)-2-(4-(5-phenyl- 4,5-dihydro-1H- pyrazole-1- carbonyl)piperidin- 1-yl)pyrimidin- 4(3H)-one | 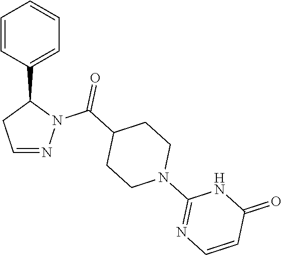 [SMILES: O=C(C1CCN(c2nccc(=O)[nH]2)CC1)N1N=CC[C@H]1c1ccccc1] | ¹H NMR (400 MHz, DMSO-d6) δ ppm 11.16/7.70 (br. s, 1H), 7.32 (dd, J = 7.6, 7.2 Hz, 2H), 7.24 (m, 2H), 7.10 (d, J = 7.2 Hz, 2H), 5.61 (br. s, 1H), 5.31 (dd, J = 12.0, 4.6 Hz, 1H), 4.39 (m, 2H), 3.49 (ddd, J = 18.9, 12.0, 1.3 Hz, 1H), 3.36 (m, 1H), 2.99 (m, 2H), 2.67 (ddd, J = 18.9, 4.6, 1.7 Hz, 1H), 1.85 (d, J = 13.1 Hz, 1H), 1.72 (d, J = 13.1 Hz, 1H), 1.47 (m, 2H) | 1.60 Method 1 | 352 |
| 20 | (S)-(1-(6- aminopyrimidin-4- yl)piperidin-4-yl)(5- phenyl-4,5-dihydro- 1H-pyrazol-1- yl)methanone | 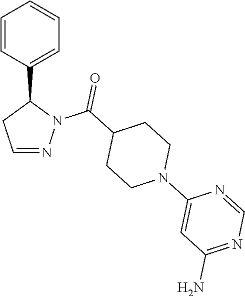 [SMILES: Nc1cc(N2CCC(C(=O)N3N=CC[C@H]3c3ccccc3)CC2)ncn1] | ¹H NMR (400 MHz, DMSO-d6) δ ppm 7.94 (s, 1H), 7.32 (dd, J = 7.6, 7.2 Hz, 2H), 7.23 (m, 2H), 7.1 (d, J = 7.2 Hz, 2H) 6.15 (s, 2H), 5.61 (s, 1H), 5.31 (dd, J = 11.9, 4.5 Hz, 1H), 4.19 (d, J = 12.9 Hz, 2H), 3.49 (dd, J = 18.4, 12.3 Hz, 1H), 3.34 (m, 1H), 2.87 (m, 2H), 2.67 (dd, J = 18.9, 3.1 Hz, 1H), 1.84 (d, J = 11.8 Hz, 1H), 1.71 (d, J = 11.8 Hz, 1H), 1.45 (m, 2H) | 1.48 Method 1 | 351 |
| 21 | (S)-(5-phenyl-4,5- dihydro-1H- pyrazol-1-yl)(1- (pyrazolo[1,5- a]pyrimidin-5- yl)piperidin-4- yl)methanone | 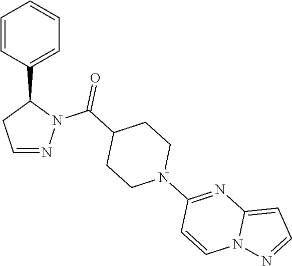 [SMILES: O=C(C1CCN(c2ccn3nccc3n2)CC1)N1N=CC[C@H]1c1ccccc1] | ¹H NMR (400 MHz, DMSO-d6) δ ppm 8.6 (d, J = 7.8 Hz, 1H), 7.83 (s, 1H), 7.32 (dd, J = 7.2*2 Hz, 2H), 7.23 (m, 2H), 7.11 (d, J = 7.4 Hz, 2H), 6.72 (d, J = 7.8 Hz, 1H), 6.01 (s, 1H), 5.31 (dd, J = 11.7, 4.3 Hz, 1H), 4.42 (d, J = 12.9 Hz, 2H), 3.49 (dd, J = 18.6, 12.0 Hz, 1H), 3.40 (t, J = 11.3 Hz, 1H), 3.06 (m, 2H), 2.68 (dd, J = 18.8, 3.4 Hz, 1H), 1.91 (d, J = 12.1 Hz, 1H), 1.78 (d, J = 12.1 Hz, 1H), 1.52 (m, 2H) | 2.17 Method 1 | 375 |
| 22 | (S)-(1- (imidazo[1,2- b]pyridazin-6- yl)piperidin-4-yl)(5- phenyl-4,5-dihydro- 1H-pyrazol-1- yl)methanone | 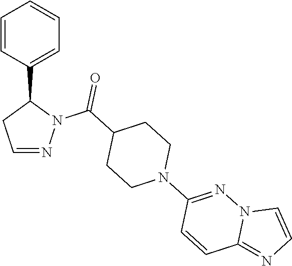 [SMILES: O=C(C1CCN(c2ccc3nccn3n2)CC1)N1N=CC[C@H]1c1ccccc1] | ¹H NMR (400 MHz, DMSO-d6) δ ppm 7.88 (br. s, 1H), 7.81 (d, J = 9.7 Hz, 1H), 7.47 (br. s, 1H), 7.31 (m, 2H), 7.25 (br. s, 2H), 7.14 (m, 3H), 5.32 (m, 1H), 4.16 (d, J = 12.3 Hz, 2H), 3.49 (dd, J = 18.4, 12.3 Hz, 1H), 3.36 (m, 1H), 2.99 (m, 2H), 2.68 (d, J = 18.4 Hz, 1H), 1.91 (d, J = 11.6 Hz, 1H), 1.78 (d, J = 11.6 Hz, 1H), 1.60 (m, 2H) | 1.58 Method 1 | 375 |
| 23 | (S)-(1-(9-methyl- 9H-purin-2- yl)piperidin-4-yl)(5- phenyl-4,5-dihydro- 1H-pyrazol-1- yl)methanone | 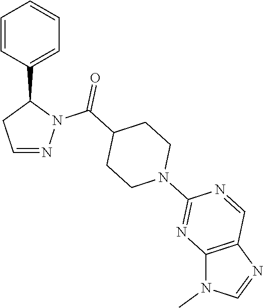 [SMILES: Cn1cnc2cnc(N3CCC(C(=O)N4N=CC[C@H]4c4ccccc4)CC3)nc21] | ¹H NMR (400 MHz, DMSO-d6) δ ppm 8.68 (s, 1H), 8.07 (s, 1H), 7.31 (dd, J = 7.4 Hz*2, 2H), 7.23 (m, 2H), 7.11 (d, J = 7.4 Hz, 2H), 5.31 (dd, J = 11.8, 4.4 Hz, 1H), 4.75 (d, J = 12.9, 2H), 3.65 (s, 3H), 3.49 (dd, J = 18.5, 12.1 Hz, 1H), 3.38 (m, 1H), 3.01 (m, 2H), 2.67 (dd, J = 18.9, 3.3 Hz, 1H), 1.90 (d, J = 12.0 Hz, 1H), 1.78 (d, J = 12.0 Hz, 1H), 1.52 (m, 2H) | 2.00 Method 1 | 390 |
| 24 | (S)-2-(4-(5-phenyl- 4,5-dihydro-1H- pyrazole-1- carbonyl)piperidin- 1-yl)-5H- pyrrolo[2,3- d]pyrimidin-6(7H)- one | 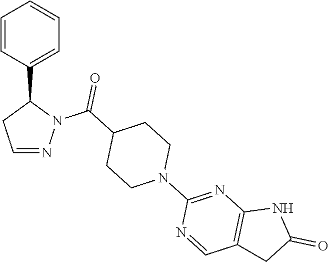 [SMILES: O=C1Cc2cnc(N3CCC(C(=O)N4N=CC[C@H]4c4ccccc4)CC3)nc2N1] | ¹H NMR (400 MHz, DMSO-d6) δ ppm 11.05 (s, 1H), 7.91 (s, 1H), 7.32 (dd, J = 7.4, 7.2 Hz, 2H), 7.24 (m, 2H), 7.10 (d, J = 7.2 Hz, 2H), 5.31 (dd, J = 11.8, 4.4 Hz, 1H), 4.60 (d, J = 12.9 Hz, 2H), 3.49 (dd, J = 18.8, 12.0 Hz, 1H), 3.39 (s, 2H), 3.37 (m, 1H), 2.96 (m, 2H), 2.67 (dd, J = 18.3 3.7 Hz, 1H), 1.85 (d, J = 11.4 Hz, 1H), 1.72 (d, J = 11.4 Hz, 1H), 1.44 (m, 2H) | 1.45 Method 1 | 391 |
| 25 | (S)-6-(4-(5-phenyl- 4,5-dihydro-1H- pyrazole-1- carbonyl)piperidin- 1-yl)pyridazine-3- carboxamide | 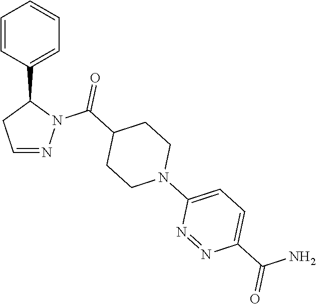 [SMILES: NC(=O)c1ccc(N2CCC(C(=O)N3N=CC[C@H]3c3ccccc3)CC2)nn1] | ¹H NMR (400 MHz, DMSO-d6) δ ppm 8.11 (br. s, 1H), 7.82 (d, J = 9.7 Hz, 1H), 7.51 (br. s, 1H), 7.34 (d, J = 9.5 Hz, 1H), 7.32 (dd, J = 7.6, 7.0 Hz, 2H), 7.24 (m, 2H), 7.11 (d, J = 7.0 Hz, 2H), 5.32 (dd, J = 11.8, 4.6 Hz, 1H), 4.50 (d, J = 11.0 Hz, 2H), 3.50 (ddd, J = 18.8, 12.0, 1.0 Hz, 1H), 3.44 (tt, J = 11.4, 3.8 Hz, 1H), 3.12 (m, 2H), 2.68 (ddd, J = 18.8, 4.4, 1.3 Hz, 1H), 1.94 (d, J = 11.9 Hz, 1H), 1.81 (d, J = 11.9 Hz, 1H), 1.55 (m, 2H) | 1.85 Method 1 | 379 |
| 26 | (S)-(5-phenyl-4,5- dihydro-1H- pyrazol-1-yl)(1-(6- (trifluoromethyl)pyridazin- 3-yl)piperidin-4- yl)methanone | 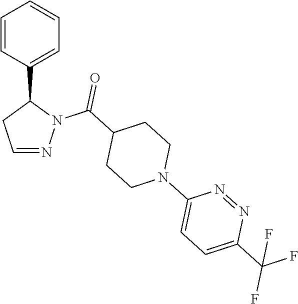 [SMILES: O=C(C1CCN(c2ccc(C(F)(F)F)nn2)CC1)N1N=CC[C@H]1c1ccccc1] | ¹H NMR (400 MHz, DMSO-d6) δ ppm 7.77 (d, J = 9.9 Hz, 1H), 7.42 (d, J = 9.7 Hz, 1H), 7.32 (dd, J = 7.6, 7.2 Hz, 2H), 7.24 (m, 2H), 7.11 (d, J = 7.11 Hz, 2H), 5.32 (dd, J = 11.9, 4.6 Hz, 1H), 4.51 (dd, J = 12.9, 2.7 Hz, 2H), 3.50 (ddd, J = 18.8, 11.8, 1.3 Hz, 1H), 3.45 (tt, J = 11.7, 3.8 Hz, 1H), 3.16 (m, 2H), 2.69 (ddd, J = 18.8, 4.6, 1.5 Hz, 1H), 1.95 (d, J = 12.8 Hz, 1H), 1.82 (d, J = 12.8 Hz, 1H), 1.55 (m, 2H) | 2.55 Method 1 | 404 |
| 27 | (S)-(1-(4-amino-5- fluoropyrimidin-2- yl)piperidin-4-yl)(5- phenyl-4,5-dihydro- 1H-pyrazol-1- yl)methanone | 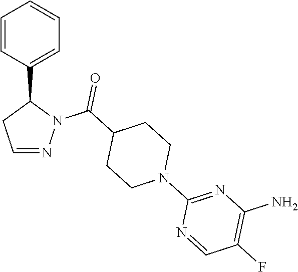 [SMILES: Nc1nc(N2CCC(C(=O)N3N=CC[C@H]3c3ccccc3)CC2)ncc1F] | ¹H NMR (400 MHz, DMSO-d6) δ ppm 7.78 (d, J = 3.6 Hz, 1H), 7.32 (dd, J = 7.4 Hz, 2H), 7.24 (m, 2H), 7.10 (d, J = 7.4 Hz, 2H), 6.80 (s, 2H), 5.30 (dd, J = 11.8, 4.6 Hz, 1H), 4.52 (d, J = 12.9 Hz, 2H), 3.49 (dd, J = 18.4, 12.3 Hz, 1H), 3.30 (tt, J = 11.6, 3.5 Hz, 1H), 2.83 (dt, J = 10.6, 7.6 Hz, 2H), 2.67 (dd, J = 18.8, 3.2 Hz, 1H), 1.80 (d, J = 12.0 Hz, 1H), 1.66 (d, J = 12.0 Hz, 1H), 1.42 (m, 2H) | 1.43 Method 1 | 369 |
| 28 | (S)-2-(4-(5-phenyl- 4,5-dihydro-1H- pyrazole-1- carbonyl)piperidin- 1-yl)pyrimidine-4- carboxamide | 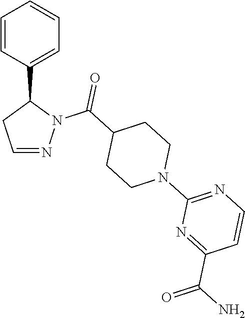 [SMILES: NC(=O)c1ccnc(N2CCC(C(=O)N3N=CC[C@H]3c3ccccc3)CC2)n1] | ¹H NMR (400 MHz, DMSO-d6) δ ppm 8.54 (d, J = 4.7 Hz, 1H), 8.16 (br. s, 1H), 7.72 (br. s, 1H), 7.32 (dd, J = 7.6, 7.2 Hz, 2H), 7.24 (m, 2H), 7.11 (d, J = 7.2 Hz, 2H), 7.07 (d, J = 4.7 Hz, 1H), 5.32 (dd, J = 11.9, 4.6 Hz, 1H), 4.80 (d, J = 13.1 Hz, 2H), 3.50 (ddd, J = 19.0, 12.0, 1.5 Hz, 1H), 3.39 (tt, J = 11.5, 3.7 Hz, 1H), 3.01 (m, 2H), 2.68 (ddd, J = 19.0, 4.7, 1.7 Hz, 1H), 1.9 (d, J = 11.6 Hz, 1H), 1.77 (d, J = 11.6 Hz, 1H), 1.50 (m, 2H) | 2.15 Method 1 | 379 |
| 29 | (S)-2-(4-(5-phenyl- 4,5-dihydro-1H- pyrazole-1- carbonyl)piperidin- 1-yl)pyrimidine-4- carboxylic acid | 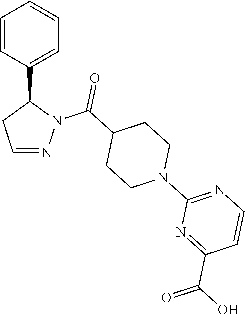 [SMILES: O=C(O)c1ccnc(N2CCC(C(=O)N3N=CC[C@H]3c3ccccc3)CC2)n1] | ¹H NMR (400 MHz, DMSO-d6) δ ppm 8.53 (d, J = 4.0 Hz, 1H), 7.32 (dd, J = 7.1, 6.8 Hz, 2H), 7.25 (m, 2H), 7.11 (d, J = 6.8 Hz, 2H), 7.03 (d, J = 4.0 Hz, 1H), 5.31 (m, 1H), 4.72 (d, J = 12.3 Hz, 2H), 3.52 (dd, J = 18.4, 12.1 Hz, 1H), 3.39 (m, 1H), 3.02 (m, 2H), 2.68 (d, J = 18.0 Hz, 1H), 1.90 (d, J = 11.6 Hz, 1H), 1.78 (d, J = 11.6 Hz, 1H), 1.47 (m, 2H) | 2.22 Method 1 | 380 |
| 30 | (S)-6-(4-(5-phenyl- 4,5-dihydro-1H- pyrazole-1- carbonyl)piperidin- 1-yl)nicotinamide | 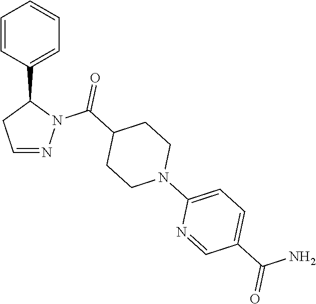 [SMILES: NC(=O)c1ccc(N2CCC(C(=O)N3N=CC[C@H]3c3ccccc3)CC2)nc1] | ¹H NMR (400 MHz, DMSO-d6) δ ppm 8.60 (br. s, 1H), 7.94 (d, J = 7.2 Hz, 1H), 7.74 (br. s, 1H), 7.32 (dd, J = 7.4, 7.0 Hz, 2H), 7.24 (m, 2H), 7.11 (d, J = 7.0 Hz, 3H), 6.84 (d, J = 8.9 Hz, 1H), 5.31 (dd, J = 11.8, 4.2 Hz, 1H), 4.42 (d, J = 12.9 Hz, 2H), 3.49 (dd, J = 18.6, 12.1 Hz, 1H), 3.39 (m, 1H), 2.99 (dt, J = 10.5, 9.5 Hz, 2H), 2.67 (dd, J = 18.7, 3.1 Hz, 1H), 1.88 (d, J = 12.0 Hz, 1H), 1.76 (d, J = 11.6 Hz, 1H), 1.50 (m, 2H) | 1.61 Method 1 | 378 |
| 31 | (S)-6-(4-(5-phenyl- 4,5-dihydro-1H- pyrazole-1- carbonyl)piperidin- 1-yl)pyrazine-2- carboxamide | 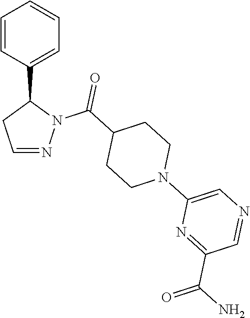 [SMILES: NC(=O)c1cncc(N2CCC(C(=O)N3N=CC[C@H]3c3ccccc3)CC2)n1] | ¹H NMR (400 MHz, DMSO-d6) δ ppm 8.48 (s, 1H), 8.31 (s, 1H), 8.04 (br. s, 1H), 7.64 (br. s, 1H), 7.32 (dd, J = 7.4, 7.2 Hz, 2H), 7.24 (m, 2H), 7.11 (d, J = 7.2 Hz, 2H), 5.32 (dd, J = 11.9, 4.5 Hz, 1H), 4.53 (d, J = 12.7 Hz, 2H), 3.50 (dd, J = 18.3, 12.4 Hz, 1H), 3.39 (m, 1H), 3.02 (m, 2H), 2.69 (dd, J = 18.3, 3.7 Hz, 1H), 1.92 (d, J = 12.0 Hz, 1H), 1.79 (d, J = 12.1 Hz, 1H), 1.54 (m, 2H) | 2.00 Method 1 | 379 |
| 32 | (S)-6-(4-(5-phenyl- 4,5-dihydro-1H- pyrazole-1- carbonyl)piperidin- 1-yl)pyrimidine-4- carboxamide | 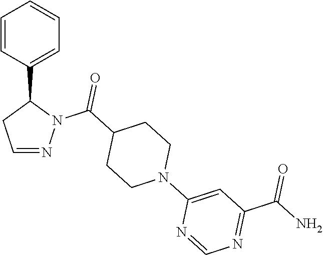 [SMILES: NC(=O)c1cc(N2CCC(C(=O)N3N=CC[C@H]3c3ccccc3)CC2)ncn1] | ¹H NMR (400 MHz, DMSO-d6) δ ppm 8.54 (s, 1H), 8.04 (br. s, 1H), 7.73 (br. s, 1H), 7.32 (m, 3H), 7.24 (m, 2H), 7.11 (d, J = 7.0 Hz, 2H), 5.32 (dd, J = 11.8, 4.6 Hz, 1H), 4.46 (br. s, 2H), 3.50 (ddd, J = 19.0, 12.0, 1.5 Hz, 1H), 3.43 (tt, J = 11.6, 3.8 Hz, 1H), 3.1 (m, 2H), 2.68 (ddd, J = 18.8, 4.6, 1.7 Hz, 1H), 1.93 (d, J = 13.9 Hz, 1H), 1.80 (d, J = 12.5 Hz, 1H), 1.49 (m, 2H) | 1.82 Method 1 | 379 |
| 33 | (S)-(1-(6-amino-2- methylpyrimidin-4- yl)piperidin-4-yl)(5- phenyl-4,5-dihydro- 1H-pyrazol-1- yl)methanone | 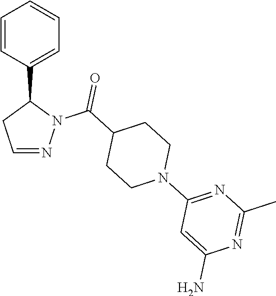 [SMILES: Cc1nc(N)cc(N2CCC(C(=O)N3N=CC[C@H]3c3ccccc3)CC2)n1] | ¹H NMR (400 MHz, DMSO-d6) δ ppm 7.32 (dd, J = 7.6, 7.2 Hz, 2H), 7.23 (m, 2H), 7.10 (d, J = 7.2 Hz, 2H), 6.05 (s, 2H), 5.45 (s, 1H), 5.31 (dd, J = 11.8, 4.6 Hz, 1H), 4.20 (d, J = 12.7 Hz, 2H), 3.49 (dd, J = 18.1, 12.6 Hz, 1H), 3.33 (m, 1H), 2.84 (m, 2H), 2.67 (ddd, J = 18.8, 4.6, 1.5 Hz, 1H), 2.15 (s, 3H), 1.83 (d, J = 12.0 Hz, 1H), 1.71 (d, J = 11.8 Hz, 1H), 1.45 (m, 2H) | 1.59 Method 1 | 365 |
| 34 | (S)-(1-(2-amino-6- methoxypyrimidin- 4-yl)piperidin-4- yl)(5-phenyl-4,5- dihydro-1H- pyrazol-1- yl)methanone | 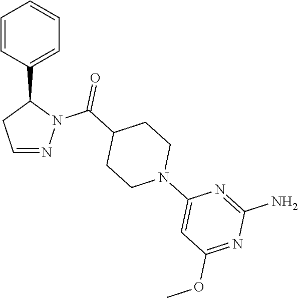 [SMILES: COc1cc(N2CCC(C(=O)N3N=CC[C@H]3c3ccccc3)CC2)nc(N)n1] | ¹H NMR (400 MHz, DMSO-d6) δ ppm 7.32 (dd, J = 7.4, 7.2 Hz, 2H), 7.24 (m, 2H), 7.10 (d, J = 7.2 Hz, 2H), 6.03 (s, 2H), 5.30 (dd, J = 11.8, 4.6 Hz, 1H), 5.33 (s, 1H), 4.25 (d, J = 12.1 Hz, 2H), 3.71 (s, 3H), 3.48 (dd, J = 18.6, 12.1 Hz, 1H), 3.36 (m, 1H), 2.85 (m, 2H), 2.67 (d, J = 18.7, 3.1 Hz, 1H), 1.80 (d, J = 11.8 Hz, 1H), 1.68 (d, J = 12.0 Hz, 1H), 1.42 (m, 2H) | 1.58 Method 1 | 381 |
| 35 | (S)-(1-(6-amino-2- methoxypyrimidin- 4-yl)piperidin-4- yl)(5-phenyl-4,5- dihydro-1H- pyrazol-1- yl)methanone | 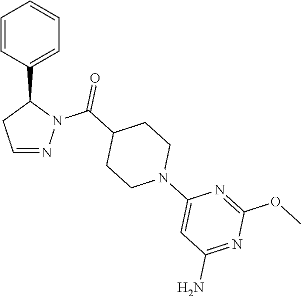 [SMILES: COc1nc(N)cc(N2CCC(C(=O)N3N=CC[C@H]3c3ccccc3)CC2)n1] | ¹H NMR (400 MHz, DMSO-d6) δ ppm 7.32 (dd, J = 7.6, 7.2 Hz, 2H), 7.23 (m, 2H), 7.10 (d, J = 7.2 Hz, 2H), 6.16 (s, 2H), 5.31 (s, 1H), 5.32 (dd, J = 11.8, 4.6 Hz, 1H), 4.16 (d, J = 12.9 Hz, 2H), 3.69 (s, 3H), 3.49 (ddd, J = 19.0, 7.0, 1.1 Hz, 1H), 3.32 (m, 1H), 2.87 (m, 2H), 2.67 (ddd, J = 19.0, 4.6, 1.3 Hz, 1H), 1.83 (d, J = 11.4 Hz, 1H), 1.70 (d, J = 11.6 Hz, 1H), 1.45 (m, 2H) | 1.66 Method 1 | 381 |
| 36 | (S)-N-(2-(4-(5- phenyl-4,5-dihydro- 1H-pyrazole-1- carbonyl)piperidin- 1-yl)pyrimidin-4- yl)acetamide | 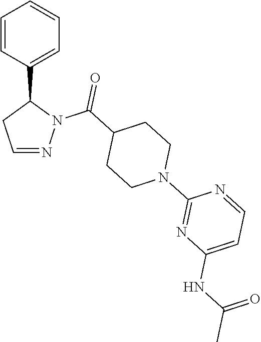 [SMILES: CC(=O)Nc1ccnc(N2CCC(C(=O)N3N=CC[C@H]3c3ccccc3)CC2)n1] | ¹H NMR (400 MHz, DMSO-d6) δ ppm 10.32 (s, 1H), 8.20 (d, J = 5.5 Hz, 1H), 7.32 (dd, J = 7.4 Hz*2, 2H), 7.22 (m, 3H), 7.11 (d, J = 7.4 Hz, 2H), 5.31 (dd, J = 11.9, 4.5 Hz, 1H), 4.65 (d, J = 12.7 Hz, 2H), 3.49 (dd, J = 18.5, 12.1 Hz, 1H), 3.37 (m, 1H), 2.95 (m, 2H), 2.68 (dd, J = 18.9, 3.3 Hz, 1H), 2.09 (s, 3H), 1.85 (d, J = 11.6 Hz, 1H), 1.75 (d, J = 12.0 Hz, 1H), 1.45 (m, 2H) | 1.73 Method 1 | 393 |
| 37 | (S)-6-(4-(5-phenyl- 4,5-dihydro-1H- pyrazole-1- carbonyl)piperidin- 1-yl)-1H- pyrazolo[3,4- d]pyrimidin-4(7H)- one | 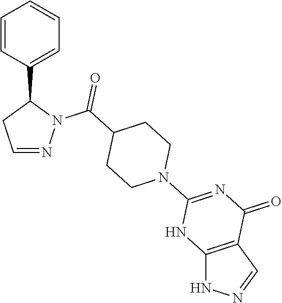 [SMILES: O=C(C1CCN(c2nc(=O)c3cn[nH]c3[nH]2)CC1)N1N=CC[C@H]1c1ccccc1] | ¹H NMR (400 MHz, DMSO-d6) δ ppm 12.90 (s, 1H), 10.83 (s, 1H), 7.76 (s, 1H), 7.32 (dd, J = 7.4, 7.2 Hz, 2H), 7.25 (m, 2H), 7.11 (d, J = 7.2 Hz, 2H), 5.31 (dd, J = 11.6, 4.2 Hz, 1H), 4.30 (d, J = 12.3 Hz, 2H), 3.49 (dd, J = 18.6, 12.0 Hz, 1H), 3.36 (m, 1H), 3.05 (m, 2H), 2.68 (dd, J = 18.7, 3.3 Hz, 1H), 1.87 (d, J = 11.6 Hz, 1H), 1.74 (d, J = 11.8 Hz, 1H), 1.53 (m, 2H) | 1.82 Method 1 | 392 |
| 38 | (S)-(5-phenyl-4,5- dihydro-1H- pyrazol-1-yl)(1-(6- phenylpyrazin-2- yl)piperidin-4- yl)methanone | 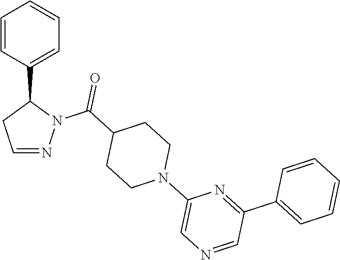 [SMILES: O=C(C1CCN(c2cncc(-c3ccccc3)n2)CC1)N1N=CC[C@H]1c1ccccc1] | ¹H NMR (400 MHz, DMSO-d6) δ ppm 8.42 (s, 1H), 8.30 (s, 1H), 8.07 (m, 2H), 7.48 (m, 3H), 7.32 (dd, J = 7.6, 7.2 Hz, 2H), 7.24 (m, 2H), 7.12 (d, J = 7.2 Hz, 2H), 5.32 (dd, J = 11.8, 4.6 Hz, 1H), 4.49 (d, J = 12.9 Hz, 2H), 3.51 (ddd, J = 19.0, 12.0, 1.3 Hz, 1H), 3.41 (tt, J = 11.5, 3.8 Hz, 1H), 3.06 (m, 2H), 2.69 (ddd, J = 19.0, 4.7, 1.7 Hz, 1H), 1.95 (d, J = 12.1 Hz, 1H), 1.82 (d, J = 12.1 Hz, 1H), 1.59 (m, 2H) | 2.84 Method 1 | 412 |
| 39 | (S)-(5-phenyl-4,5- dihydro-1H- pyrazol-1-yl)(1- (quinoxalin-2- yl)piperidin-4- yl)methanone | 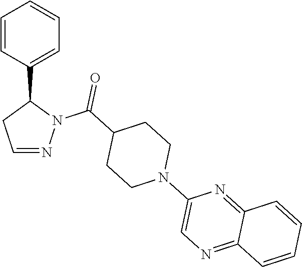 [SMILES: O=C(C1CCN(c2cnc3ccccc3n2)CC1)N1N=CC[C@H]1c1ccccc1] | ¹H NMR (400 MHz, DMSO-d6) δ ppm 8.83 (s, 1H), 7.81 (d, J = 8.2 Hz, 1H), 7.58 (m, 2H), 7.38 (m, 1H), 7.32 (dd, J = 7.8, 7.2 Hz, 2H), 7.24 (m, 2H), 7.12 (d, J = 7.2 Hz, 2H), 5.32 (dd, J = 11.9, 4.6 Hz, 1H), 4.61 (d, J = 13.3 Hz, 2H), 3.51 (ddd, J = 18.9, 12.0, 1.5 Hz, 1H), 3.44 (tt, J = 11.4, 3.8 Hz, 1H), 3.13 (m, 2H), 2.69 (ddd, J = 18.8, 4.7, 1.5 Hz, 1H), 1.96 (d, J = 11.4 Hz, 1H), 1.84 (d, J = 11.4 Hz, 1H), 1.60 (m, 2H) | 2.55 Method 1 | 386 |
| 40 | (S)-5-(4-(5-phenyl- 4,5-dihydro-1H- pyrazole-1- carbonyl)piperidin- 1-yl)pyrazine-2- carbonitrile | 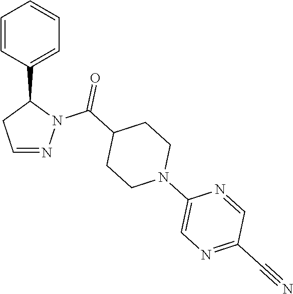 [SMILES: N#Cc1cnc(N2CCC(C(=O)N3N=CC[C@H]3c3ccccc3)CC2)cn1] | ¹H NMR (400 MHz, CDCl3) δ ppm 8.33 (d, J = 1.3 Hz, 1H), 8.13 (d, J = 1.3 Hz, 1H), 7.34 (dd, J = 7.6, 7.0 Hz, 2H), 7.28 (m, 1H), 7.16 (d, J = 7.0 Hz, 2H), 7.02 (t, J = 1.6 Hz, 1H), 5.39 (dd, J = 11.9, 4.8 Hz, 1H), 4.44 (m, 2H), 3.47 (ddd, J = 18.8, 12.0, 1.5 Hz, 1H), 3.48 (m, 1H), 3.23 (m, 2H), 2.86 (ddd, J = 18.8, 5.0, 1.7 Hz, 1H), 2.06 (m, 1H), 1.97 (m, 1H), 1.81 (m, 2H) | 2.44 Method 1 | 361 |
| 41 | (S)-(1-(6- aminopyrazin-2- yl)piperidin-4-yl)(5- phenyl-4,5-dihydro- 1H-pyrazol-1- yl)methanone | 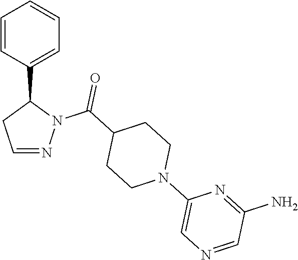 [SMILES: Nc1cncc(N2CCC(C(=O)N3N=CC[C@H]3c3ccccc3)CC2)n1] | ¹H NMR (400 MHz, DMSO-d6) δ ppm 7.35 (s, 1H), 7.32 (dd, J = 7.6, 7.2 Hz, 2H), 7.25 (m, 2H), 7.13 (s, 1H), 7.11 (d, J = 7.0 Hz, 2H), 5.92 (s, 2H), 5.31 (dd, J = 11.8, 4.6 Hz, 1H), 4.23 (d, J = 12.9 Hz, 2H), 3.49 (ddd, J = 18.8, 11.8, 1.3 Hz, 1H), 3.32 (m, 1H), 2.84 (m, 2H), 2.68 (ddd, J = 18.8, 4.6, 1.7 Hz, 1H), 1.84 (d, J = 11.8 Hz, 1H), 1.72 (d, J = 11.8 Hz, 1H), 1.49 (m, 2H) | 1.60 Method 1 | 351 |
| 42 | (S)-6-(4-(5-phenyl- 4,5-dihydro-1H- pyrazole-1- carbonyl)piperidin- 1-yl)pyridazine-3- carbonitrile | 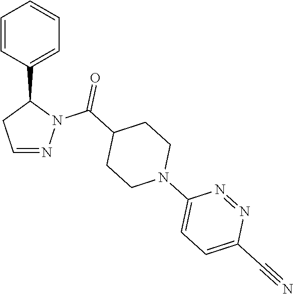 [SMILES: N#Cc1ccc(N2CCC(C(=O)N3N=CC[C@H]3c3ccccc3)CC2)nn1] | ¹H NMR (400 MHz, DMSO-d6) δ ppm 7.83 (d, J = 9.7 Hz, 1H), 7.36 (d, J = 9.7 Hz, 1H), 7.32 (dd, J = 7.6, 7.2 Hz, 2H), 7.24 (m, 2H), 7.11 (d, J = 7.2 Hz, 2H), 5.32 (dd, J = 11.8, 4.6 Hz, 1H), 4.52 (d, J = 12.0 Hz, 2H), 3.50 (ddd, J = 18.8, 12.0, 1.7 Hz, 1H), 3.46 (m, 1H), 3.19 (m, 2H), 2.69 (ddd, J = 18.9, 4.7, 1.7 Hz, 1H), 1.96 (d, J = 11.0 Hz, 1H), 1.82 (d, J = 11.0 Hz, 1H), 1.54 (m, 2H) | 2.28 Method | 361 |
| 43 | (S)-(1-(6- hydroxypyrimidin- 4-yl)piperidin-4- yl)(5-phenyl-4,5- dihydro-1H- pyrazol-1- yl)methanone | 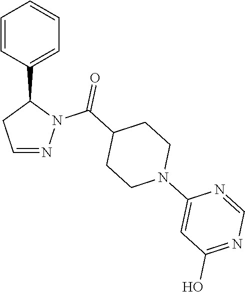 [SMILES: O=C(C1CCN(c2cc(O)ncn2)CC1)N1N=CC[C@H]1c1ccccc1] | ¹H NMR (400 MHz, DMSO-d6) δ ppm 11.57 (br. s, 1H), 7.88 (s, 1H), 7.32 (m, 2H), 7.24 (m, 2H), 7.10 (d, J = 7.2 Hz, 2H), 5.31 (dd, J = 12.0, 4.6 Hz, 1H), 5.26 (s, 1H), 4.21 (d, J = 12.9 Hz, 2H), 3.49 (ddd, J = 18.8, 12.0, 1.3 Hz, 1H), 3.35 (tt, J = 11.4, 3.8 Hz, 1H), 2.93 (m, 2H), 2.67 (ddd, J = 19.0, 4.7, 1.7 Hz, 1H), 1.85 (d, J = 11.4 Hz, 1H), 1.72 (d, J = 11.4 Hz, 1H), 1.45 (m, 2H) | 1.86 Method 1 | 352 |
| 44 | (S)-3-(4-(5-phenyl- 4,5-dihydro-1H- pyrazole-1- carbonyl)piperidin- 1-yl)pyrazine-2- carbonitrile | 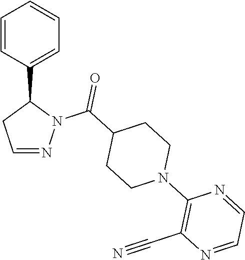 [SMILES: N#Cc1nccnc1N1CCC(C(=O)N2N=CC[C@H]2c2ccccc2)CC1] | ¹H NMR (400 MHz, DMSO-d6) δ ppm 8.43 (d, J = 2.3 Hz, 1H), 8.02 (m, 1H), 7.32 (dd, J = 7.6, 7.0 Hz, 2H), 7.24 (m, 2H), 7.12 (d, J = 7.0 Hz, 2H), 5.32 (dd, J = 12.0, 4.6 Hz, 1H), 4.36 (d, J = 13.3 Hz, 2H), 3.50 (ddd, J = 19.0, 12.0, 1.5 Hz, 1H), 3.43 (tt, J = 11.4, 4.0 Hz, 1H), 3.22 (m, 2H), 2.69 (ddd, J = 18.8, 4.6, 1.7 Hz, 1H), 1.97 (d, J = 11.0 Hz, 1H), 1.83 (d, J = 11.0 Hz, 1H), 1.64 (m, 2H) | 2.49 Method 1 | 361 |
| 45 | (S)-(1-(2- (methylthio)pyrimidin- 4-yl)piperidin-4- yl)(5-phenyl-4,5- dihydro-1H- pyrazol-1- yl)methanone | 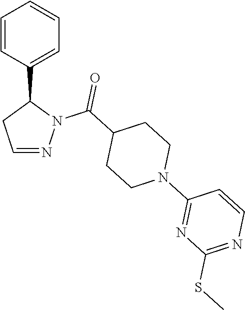 [SMILES: CSc1nccc(N2CCC(C(=O)N3N=CC[C@H]3c3ccccc3)CC2)n1] | ¹H NMR (400 MHz, DMSO-d6) δ ppm 7.99 (d, J = 6.3 Hz, 1H), 7.32 (dd, J = 7.6, 7.2 Hz, 2H), 7.23 (m, 2H), 7.11 (d, J = 7.2 Hz, 2H), 6.55 (d, J = 6.1 Hz, 1H), 5.31 (dd, J = 11.9 Hz, 4.5 Hz, 1H), 4.36 (br. s, 2H), 3.49 (dd, J = 18.5, 12.4 Hz, 1H), 3.40 (m, 1H), 3.02 (m, 2H), 2.68 (dd, J = 18.9, 3.1 Hz, 1H), 2.41 (s, 3H), 1.90 (d, J = 12.0 Hz, 1H), 1.77 (d, J = 12.0 Hz, 1H), 1.46 (m, 2H) | 1.66 Method 1 | 382 |
| 46 | (S)-(5-phenyl-4,5- dihydro-1H- pyrazol-1-yl)(1-(2- (trifluoromethyl)pyrimidin- 4-yl)piperidin-4- yl)methanone | 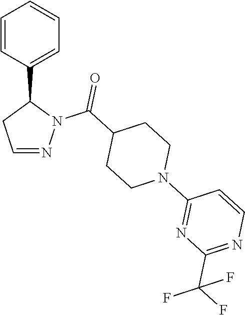 [SMILES: O=C(C1CCN(c2ccnc(C(F)(F)F)n2)CC1)N1N=CC[C@H]1c1ccccc1] | NMR (400 MHz, CDCl3) δ ppm 8.26 (d, J = 6.3 Hz, 1H), 7.34 (dd, J = 7.6, 7.0 Hz, 2H), 7.27 (m, 1H), 7.16 (d, J = 7.0 Hz, 2H), 7.02 (t, J = 1.5 Hz, 1H), 6.59 (d, J = 6.5 Hz, 1H), 5.38 (dd, J = 11.9, 4.8 Hz, 1H), 4.44 (br. s, 2H), 3.46 (m, 2H), 3.16 (m, 2H), 2.85 (ddd, J = 18.8, 12.0, 1.7 Hz, 1H), 2.06 (m, 1H), 1.95 (m, 1H), 1.79 (m, 2H) | 2.64 Method 1 | 404 |
| 47 | (S)-6-(4-(5-phenyl- 4,5-dihydro-1H- pyrazole-1- carbonyl)piperidin- 1-yl)pyrazine-2- carbonitrile | 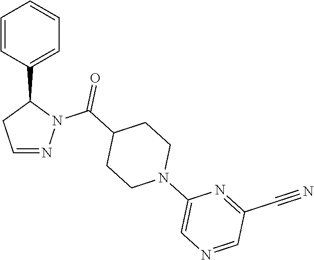 [SMILES: N#Cc1cncc(N2CCC(C(=O)N3N=CC[C@H]3c3ccccc3)CC2)n1] | ¹H NMR (400 MHz, DMSO-d6) δ ppm 8.65 (s, 1H), 8.26 (s, 1H), 7.32 (dd, J = 7.6, 7.2 Hz, 2H), 7.25 (m, 2H), 7.11 (d, J = 7.2 Hz, 2H) 5.32 (dd, J = 11.8, 4.6 Hz, 1H), 4.37 (d, J = 13.5 H, 2H), 3.50 (ddd, J = 18.8, 12.0, 1.3 Hz, 1H), 3.40 (tt, J = 11.2, 3.8 Hz, 1H), 3.09 (m, 2H), 2.68 (ddd, J = 18.8, 4.6, 1.7 Hz, 1H), 1.93 (d, J = 11.0 Hz, 1H), 1.80 (d, J = 11.2 Hz, 1H), 1.54 (m, 2H) | 2.50 Method 1 | 361 |
| 48 | (S)-(1-(6- methoxypyrazin-2- yl)piperidin-4-yl)(5- phenyl-4,5-dihydro- 1H-pyrazol-1- yl)methanone | 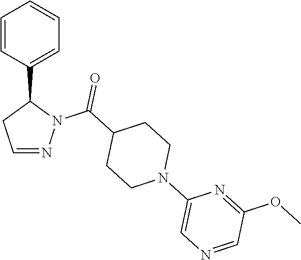 [SMILES: COc1cncc(N2CCC(C(=O)N3N=CC[C@H]3c3ccccc3)CC2)n1] | ¹H NMR (400 MHz, DMSO-d6) δ ppm 7.81 (s, 1H), 7.43 (s, 1H), 7.32 (dd, J = 7.6, 7.2 Hz, 2H), 7.24 (m, 2H), 7.11 (d, J = 7.0 Hz, 2H), 5.31 (dd, J = 11.9, 4.6 Hz, 1H), 4.33 (d, J = 13.0 Hz, 2H), 3.82 (s, 3H), 3.50 (ddd, J = 18.9, 12.0, 1.5 Hz, 1H), 3.39 (m, 1H), 2.98 (m, 2H), 2.68 (ddd, J = 18.9, 4.7, 1.7 Hz, 1H), 1.90 (d, J = 11.2 Hz, 1H), 1.78 (d, J = 11.2 Hz, 1H), 1.54 (m, 2H) | 2.42 Method 1 | 366 |
| 49 | (S)-(1-(6- methoxypyridazin- 3-yl)piperidin-4- yl)(5-phenyl-4,5- dihydro-1H- pyrazol-1- yl)methanone | 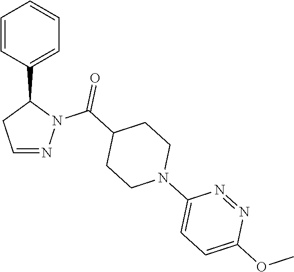 [SMILES: COc1ccc(N2CCC(C(=O)N3N=CC[C@H]3c3ccccc3)CC2)nn1] | ¹H NMR (400 MHz, DMSO-d6) δ ppm 7.39 (d, J = 9.7 Hz, 1H), 7.32 (dd, J = 7.6, 7.2 Hz, 2H), 7.24 (m, 2H), 7.11 (d, J = 7.2 Hz, 2H), 7.00 (d, J = 9.7 Hz, 1H), 5.31 (dd, J = 11.8, 4.6 Hz, 1H), 4.19 (d, J = 12.9 Hz, 2H), 3.89 (s, 3H), 3.49 (ddd, J = 18.8, 11.8, 1.2 Hz, 1H), 3.30 (m, 1H), 2.92 (m, 2H), 2.67 (ddd, J = 18.8, 4.6, 1.5 Hz, 1H), 1.86 (d, J = 12.2 Hz, 1H), 1.74 (d, J = 12.2 Hz, 1H), 1.57 (m, 2H) | 1.48 Method | 366 |
| 50 | (S)-4-(4-(5-phenyl- 4,5-dihydro-1H- pyrazole-1- carbonyl)piperidin- 1-yl)pyrimidine-5- carbonitrile | 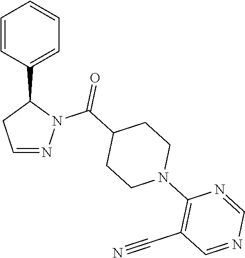 [SMILES: N#Cc1cncnc1N1CCC(C(=O)N2N=CC[C@H]2c2ccccc2)CC1] | ¹H NMR (400 MHz, DMSO-d6) δ ppm 8.67 (d, J = 6.1 Hz, 2H), 7.32 (J = 7.6, 7.0 Hz, 2H), 7.24 (m, 2H), 7.11 (d, J = 7.2 Hz, 2H), 5.32 (dd, J = 11.9, 4.6 Hz, 1H), 4.65 (d, J = 13.3 Hz, 2H), 3.50 (ddd, J = 19.0, 11.8, 1.3 Hz, 1H), 3.48 (m, 1H), 3.31 (m, 2H), 2.69 (ddd, J = 19.0, 4.7, 1.7 Hz, 1H), 1.99 (d, J = 13.2 Hz, 1H), 1.86 (d, J = 13.2 Hz 1H), 1.60 (m, 2H) | 2.20 Method 1 | 361 |
| 51 | (S)-(5-phenyl-4,5- dihydro-1H- pyrazol-1-yl)(1-(6- (trifluoromethyl)pyrimidin- 4-yl)piperidin-4- yl)methanone | 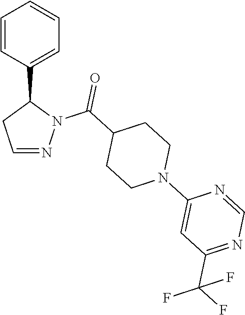 [SMILES: O=C(C1CCN(c2cc(C(F)(F)F)ncn2)CC1)N1N=CC[C@H]1c1ccccc1] | ¹H NMR (400 MHz, DMSO-d6) δ ppm 8.60 (s, 1H), 7.32 (dd, J = 7.6, 7.2 Hz, 2H), 7.24 (m, 3H), 7.11 (d, J = 7.2 Hz, 2H), 5.32 (dd, J = 11.9, 4.6 Hz, 1H), 4.36 (br. s, 2H), 3.50 (ddd, J = 19.0, 12.0, 1.5 Hz, 1H), 3.44 (tt, J = 11.2, 3.8 Hz, 1H), 3.13 (m, 2H), 2.68 (ddd, J = 19.0, 4.6, 1.5 Hz, 1H), 1.94 (d, J = 12.9 Hz, 1H), 1.81 (d, J = 13.0 Hz, 1H), 1.51 (m, 2H) | 2.58 Method 1 | 404 |
| 52 | (S)-(1-(1H- pyrazolo[3,4- d]pyrimidin-6- yl)piperidin-4-yl)(5- phenyl-4,5-dihydro- 1H-pyrazol-1- yl)methanone | 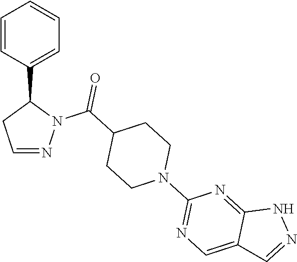 [SMILES: O=C(C1CCN(c2ncc3cn[nH]c3n2)CC1)N1N=CC[C@H]1c1ccccc1] | ¹H NMR (400 MHz, DMSO-d6) δ ppm 13.06 (s, 1H), 8.88 (s, 1H), 7.96 (s, 1H), 7.32 (dd, J = 7.6, 7.2 Hz, 2H), 7.23 (m, 2H), 7.11 (d, J = 7.2 Hz, 2H), 5.31 (dd, J = 11.8, 4.6 Hz, 1H), 4.74 (d, J = 13.1, Hz, 2H), 3.50 (ddd, J = 18.9, 12.0, 1.5 Hz, 1H), 3.41 (tt, J = 11.4, 3.6 Hz, 1H), 3.06 (m, 2H), 2.68 (ddd, J = 19.0, 4.7, 1.7 Hz, 1H), 1.90 (d, J = 12.3 Hz, 1H), 1.78 (d, J = 12.3 Hz, 1H), 1.50 (m, 2H) | 2.13 Method 1 | 376 |
| 53 | (S)-2-(4-(5-phenyl- 4,5-dihydro-1H- pyrazole-1- carbonyl)piperidin- 1-yl)thiazole-4- carbonitrile | 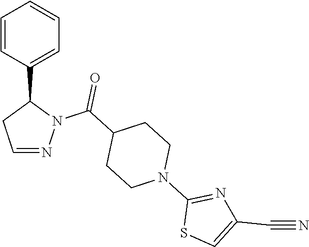 [SMILES: N#Cc1csc(N2CCC(C(=O)N3N=CC[C@H]3c3ccccc3)CC2)n1] | ¹H NMR (400 MHz, DMSO-d6) δ ppm 7.95 (s, 1H), 7.32 (dd, J = 7.6, 7.2 Hz, 2H), 7.24 (m, 2H), 7.11 (d, J = 7.2 Hz, 2H), 5.32 (dd, J = 12.0, 4.6 Hz, 1H), 3.90 (m, 2H), 3.50 (ddd, J = 19.0, 12.0, 1.5 Hz, 1H), 3.35 (tt, J = 11.6, 3.6 Hz, 1H), 3.19 (m, 2H), 2.68 (ddd, J18.8, 11.6, 1.5 Hz, 1H), 1.93 (d, J = 12.0 Hz, 1H), 1.81 (d, J = 12.0 Hz, 1H), 1.59 (m, 2H) | 2.52 Method 1 | 366 |
| 54 | (S)-N-methyl-2-(4- (5-phenyl-4,5- dihydro-1H- pyrazole-1- carbonyl)piperidin- 1-yl)thiazole-4- carboxamide | 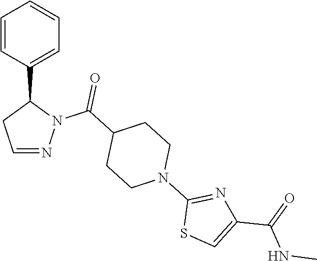 [SMILES: CNC(=O)c1csc(N2CCC(C(=O)N3N=CC[C@H]3c3ccccc3)CC2)n1] | ¹H NMR (400 MHz, DMSO-d6) δ ppm 8.01 (d, J = 4.4 Hz, 1H), 7.34 (s, 1H), 7.31 (d, J = 7.6 Hz, 2H), 7.24 (m, 2H), 7.12 (d, J = 7.2 Hz, 2H), 5.32 (dd, J = 11.8, 4.4 Hz, 1H), 4.01 (m, 2H), 3.50 (dd, J = 18.5, 12.2 Hz, 1H), 3.37 (m, 1H), 3.13 (m, 2H), 2.73 (d, J = 4.7 Hz, 3H), 2.69 (m, 1H), 1.91 (d, J = 12.3 Hz, 1H), 1.81 (d, J = 12.3 Hz, 1H), 1.60 (m, 2H) | 2.22 Method 1 | 398 |
| 55 | (S)-2-(4-(5-phenyl- 4,5-dihydro-1H- pyrazole-1- carbonyl)piperidin- 1-yl)thiazole-5- carboxamide | 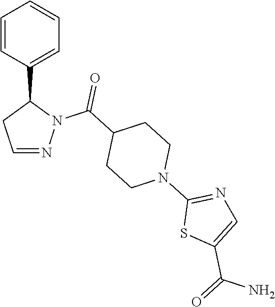 [SMILES: NC(=O)c1cnc(N2CCC(C(=O)N3N=CC[C@H]3c3ccccc3)CC2)s1] | ¹H NMR (400 MHz, DMSO-d6) δ ppm 7.77 (s, 1H), 7.67 (br. s, 1H), 7.32 (dd, J = 7.6, 7.2 Hz, 2H), 7.24 (m, 2H), 7.15 (br. s, 1H), 7.11 (d, J = 7.2 Hz, 2H), 5.32 (dd, J = 12.0, 4.7 Hz, 1H), 3.94 (d, J = 10.4 Hz, 2H), 3.50 (ddd, J = 19.0, 12.0, 1.5 Hz, 1H), 3.36 (m, 1H), 3.17 (m, 2H), 2.68 (ddd, J = 19.0, 4.7, 1.7 Hz, 1H), 1.92 (d, J = 12.0 Hz, 1H), 1.80 (d, J = 12.0 Hz, 1H), 1.59 (m, 2H) | 1.91 Method 1 | 384 |
| 56 | (S)-2-(4-(5-phenyl- 4,5-dihydro-1H- pyrazole-1- carbonyl)piperidin- 1-yl)thiazole-4- carboxamide | 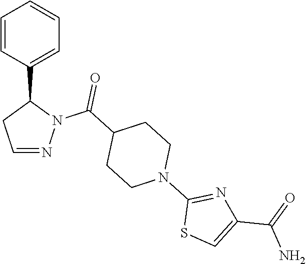 [SMILES: NC(=O)c1csc(N2CCC(C(=O)N3N=CC[C@H]3c3ccccc3)CC2)n1] | ¹H NMR (400 MHz, DMSO-d6) δ ppm 7.40 (d, J = 10.8 Hz, 2H), 7.37 (s, 1H), 7.33 (dd, J = 7.6, 7.2 Hz, 2H), 7.24 (m, 2H), 7.12 (d, J = 7.2 Hz, 2H), 5.32 (dd, J = 11.8, 4.6 Hz, 1H), 3.97 (m, 2H), 3.50 (ddd, J = 19.0, 12.0, 1.5 Hz, 1H), 3.34 (m, 1H), 3.13 (m, 2H), 2.69 (ddd, J = 19.0, 4.7, 1.7 Hz, 1H), 1.91 (d, J = 12.0 Hz, 1H), 1.80 (d, J = 12.0 Hz, 1H), 1.60 (m, 2H) | 2.11 Method 1 | 384 |
| 57 | (S)-(1-(5-phenyl- 1,3,4-oxadiazol-2- yl)piperidin-4-yl)(5- phenyl-4,5-dihydro- 1H-pyrazol-1- yl)methanone | 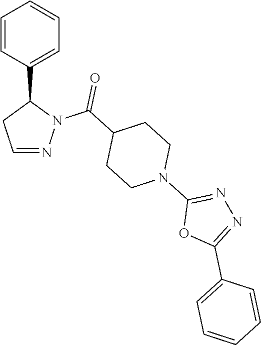 [SMILES: O=C(C1CCN(c2nnc(-c3ccccc3)o2)CC1)N1N=CC[C@H]1c1ccccc1] | ¹H NMR (400 MHz, DMSO-d6) δ ppm 7.88 (m, 2H), 7.53 (m, 3H), 7.33 (dd, J = 7.8, 7.0 Hz, 2H), 7.24 (m, 2H), 7.12 (d, J = 7.0 Hz, 2H), 5.33 (dd, J = 11.8, 4.6 Hz, 1H), 3.99 (m, 2H), 3.51 (ddd, J = 18.8, 11.8, 1.3 Hz, 1H), 3.35 (m, 1H), 3.21 (m, 2H), 2.69 (ddd, J = 18.8, 4.6, 1.7 Hz, 1H), 1.95 (d, J = 11.6 Hz, 1H), 1.83 (d, J = 11.6 Hz, 1H), 1.64 (m, 2H) | 2.49 Method 1 | 402 |
| 58 | (S)-(1-(4- ethoxypyrimidin-2- yl)piperidin-4-yl)(5- phenyl-4,5-dihydro- 1H-pyrazol-1- yl)methanone | 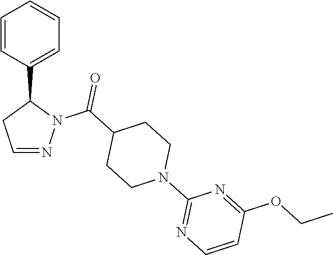 [SMILES: CCOc1ccnc(N2CCC(C(=O)N3N=CC[C@H]3c3ccccc3)CC2)n1] | ¹H NMR (400 MHz, DMSO-d6) δ ppm 8.06 (d, J = 5.7 Hz, 1H), 7.32 (dd, J = 7.6, 7.2 Hz, 2H), 7.25 (m, 2H), 7.11 (d, J = 7.2 Hz, 2H), 6.01 (d, J = 5.5 Hz, 1H), 5.31 (dd, J = 11.9, 4.5 Hz, 1H), 4.63 (d, J = 12.9 Hz, 2H), 4.29 (q, J = 7.0 Hz, 2H), 3.49 (ddd, J = 19.0, 12.0, 1.5 Hz, 1H), 3.37 (m, 1H), 2.96 (m, 2H), 2.68 (m, 1H), 1.87 (m, 1H), 1.74 (m, 1H), 1.45 (m, 2H), 1.29 (t, J = 7.0 Hz, 3H) | 1.84 Method 1 | 380 |
| 59 | (S)-(1-(6- (methylthio)pyrimidin- 4-yl)piperidin-4- yl)(5-phenyl-4,5- dihydro-1H- pyrazol-1- yl)methanone | 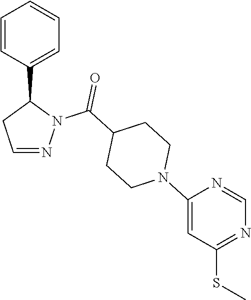 [SMILES: CSc1cc(N2CCC(C(=O)N3N=CC[C@H]3c3ccccc3)CC2)ncn1] | ¹H NMR (400 MHz, DMSO-d6) δ ppm 8.29 (d, J = 0.9 Hz, 1H), 7.32 (dd, J = 7.6, 7.2 Hz, 2H), 7.24 (m, 2H), 7.11 (d, J = 7.0 Hz, 2H), 6.62 (d, J = 0.8 Hz, 1H), 5.31 (dd, J = 11.8, 4.6 Hz, 1H), 4.37 (m, 2H), 3.49 (ddd, J = 18.8, 11.8, 1.3 Hz, 1H), 3.39(tt, J = 11.4, 3.8 Hz, 1H), 3.0 (m, 2H), 2.68 (ddd, J = 19.0, 4.7, 1.9 Hz, 1H), 2.46 (s, 3H), 1.88 (d, J = 11.9 Hz, 1H), 1.75 (d, J = 11.8 Hz, 1H), 1.46 (m, 2H) | 1.89 Method 1 | 382 |
| 60 | (S)-(1-(6-amino-2- (methylthio)pyrimidin- 4-yl)piperidin-4- yl)(5-phenyl-4,5- dihydro-1H- pyrazol-1- yl)methanone | 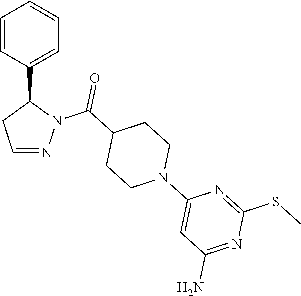 [SMILES: CSc1nc(N)cc(N2CCC(C(=O)N3N=CC[C@H]3c3ccccc3)CC2)n1] | ¹H NMR (400 MHz, DMSO-d6) δ ppm 7.32 (dd, J = 7.6, 7.2 Hz, 2H), 7.24 (m, 2H), 7.11 (d, J = 7.0 Hz, 2H), 6.21 (s, 2H), 5.36 (s, 1H), 5.31 (dd, J = 12.0, 4.6 Hz, 1H), 4.18 (d, J = 13.1 Hz, 2H), 3.52 (19.0, 12.0, 1.3 Hz, 1H), 3.45 (m, 1H), 2.88 (m, 2H), 2.67 (ddd, J = 18.8, 4.6, 1.7 Hz, 1H), 2.34 (s, 3H), 1.85 (d, J = 13.3 Hz, 1H), 1.72 (d, J = 13.3 Hz, 1H), 1.45 (m, 2H) | 1.72 Method 1 | 397 |
| 61 | (S)-(1-(6-amino-5- fluoropyrimidin-4- yl)piperidin-4-yl)(5- phenyl-4,5-dihydro- 1H-pyrazol-1- yl)methanone | 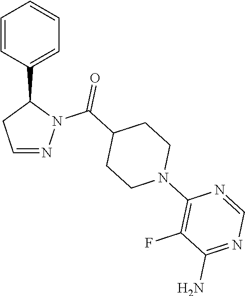 [SMILES: Nc1ncnc(N2CCC(C(=O)N3N=CC[C@H]3c3ccccc3)CC2)c1F] | ¹H NMR (400 MHz, DMSO-d6) δ ppm 7.76 (s, 1H), 7.32 (dd, J = 7.6, 7.2 Hz, 2H), 7.24 (m, 2H), 7.10 (d, J = 74.2 Hz, 2H), 6.53 (s, 2H), 5.31 (dd, J = 11.8, 4.6 Hz, 1H), 4.24 (d, J = 13.3 Hz, 2H), 3.50 (ddd, J = 18.8, 11.8, 1.3 Hz, 1H), 3.36 (tt, J = 11.4, 3.8 Hz, 1H), 3.01 (m, 2H), 2.67 (ddd, J = 18.8, 4.6, 1.7 Hz, 1H), 1.86 (d, J = 11.0Hz, 1H), 1.72 (d, J = 11.2 Hz, 1H), 1.54 (m, 2H) | 1.70 Method 1 | 369 |
| 62 | (S)-3-(1-(1- (pyrazolo[1,5- a]pyrimidin-5- yl)piperidine-4- carbonyl)-4,5- dihydro-1H- pyrazol-5- yl)benzonitrile | 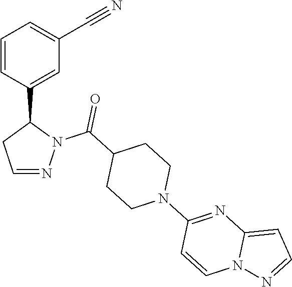 [SMILES: N#Cc1cccc([C@@H]2CC=NN2C(=O)C2CCN(c3ccn4nccc4n3)CC2)c1] | ¹H NMR (400 MHz, DMSO-d6) δ ppm 8.61 (d, J = 8.0 Hz, 1H), 7.83 (d, J = 1.9 Hz, 1H), 7.73 (d, J = 7.6 Hz, 1H), 7.60 (s, 1H), 7.55 (t, J = 7.7 Hz, 1H), 7.46 (d, J = 7.8 Hz, 1H), 7.28 (s, 1H), 6.72 (d, J = 7.8 Hz, 1H), 6.01 (s, 1H), 5.37 (dd, J = 12.0, 4.9 Hz, 1H), 4.42 (d, J = 13.1 Hz, 2H), 3.51 (dd, J = 18.5, 12.4 Hz, 1H), 3.59 (m, 1H), 3.05 (m, 2H), 2.76 (dd, J = 18.5, 4.1 Hz, 1H), 1.91 (d, J = 12.0 Hz, 1H), 1.79 (d, J = 12.0 Hz, 1H), 1.51 (m, 2H) | 2.10 Method 1 | 400 |
| 63 | (S)-5-(4-(5-phenyl- 4,5-dihydro-1H- pyrazole-1- carbonyl)piperidin- 1-yl)pyrazine-2- carboxamide | 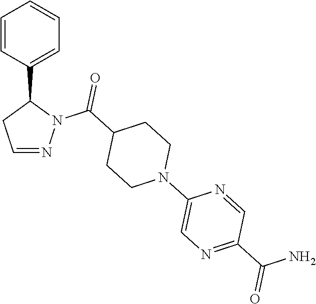 [SMILES: NC(=O)c1cnc(N2CCC(C(=O)N3N=CC[C@H]3c3ccccc3)CC2)cn1] | ¹H NMR (400 MHz, DMSO-d6) δ ppm 8.60 (d, J = 1.1 Hz, 1H), 8.27 (d, J = 1.1 Hz, 1H), 7.70 (s, 1H), 7.35 (s, 1H), 7.32 (dd, J = 7.6, 7.2 Hz, 2H), 7.23 (m, 2H), 7.11 (d, J = 7.2 Hz, 2H), 5.32 (dd, J = 11.9, 4.6 Hz, 1H), 4.48 (d, J = 11.2 Hz, 2H), 3.50 (J = 18.9, 12.0, 1.5 Hz, 1H), 3.42 (tt, J = 11.4, 3.8 Hz, 1H), 3.11 (m, 2H), 2.68 (ddd, J = 19.0, 4.6, 1.7 Hz, 1H), 1.93 (d, J = 12.9 Hz, 1H), 1.81 (d, J = 12.9 Hz, 1H), 1.55 (m, 2H) | 2.03 Method 1 | 379 |
| 64 | (S)-N-(6-(4-(5- phenyl-4,5-dihydro- 1H-pyrazole-1- carbonyl)piperidin- 1-yl)pyrazin-2- yl)acetamide | 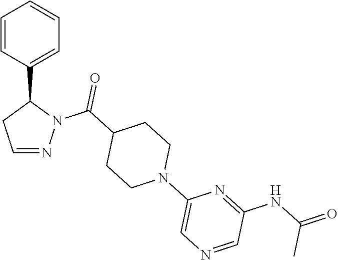 [SMILES: CC(=O)Nc1cncc(N2CCC(C(=O)N3N=CC[C@H]3c3ccccc3)CC2)n1] | ¹H NMR (400 MHz, DMSO-d6) δ ppm 10.18 (s, 1H), 8.50 (s, 1H), 7.99 (s, 1H), 7.32 (dd, J = 7.6, 7.2 Hz, 2H), 7.24 (m, 2H), 7.11 (d, J = 7.2 Hz, 2H), 5.31 (dd, J = 11.8, 4.6 Hz, 1H), 4.34 (d, J = 13.3 Hz, 2H), 3.50 (ddd, J = 18.8, 12.0, 1.3 Hz, 1H), 3.38 (tt, J = 11.4, 3.6 Hz, 1H), 2.98 (m, 2H), 2.68 (ddd, J = 19.0, 4.6, 1.7 Hz, 1H), 2.08 (s, 3H), 1.89 (d, J = 12.1 Hz, 1H), 1.77 (d, J = 12.0 Hz, 1H), 1.53 (m, 2H) | 2.05 Method 1 | 393 |
| 65 | (S)-ethyl 2-(4-(5- phenyl-4,5-dihydro- 1H-pyrazole-1- carbonyl)piperidin- 1-yl)oxazole-4- carboxylate | 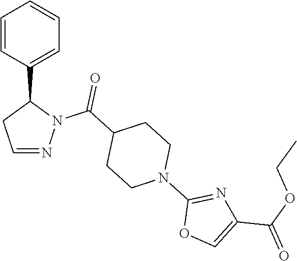 [SMILES: CCOC(=O)c1coc(N2CCC(C(=O)N3N=CC[C@H]3c3ccccc3)CC2)n1] | ¹H NMR (400 MHz, CDCl3) δ ppm 7.77 (s, 1H), 7.34 (m, 2H), 7.26 (m, 1H), 7.16 (m, 2H), 7.00 (t, J = 1.6 Hz, 1H), 5.38 (dd, J = 12.0, 4.9 Hz, 1H), 4.37 (q, J = 7.2 Hz, 2H), 4.18 (m, 2H), 3.45 (ddd, J = 18.8, 12.0, 1.5 Hz, 1H), 3.32 (m, 1H), 3.12 (m, 2H), 2.84 (ddd, J = 18.8, 4.9, 1.7 Hz, 1H), 1.85 (m, 4H), 1.37 (t, J = 7.0 Hz, 3H) | 2.42 Method 1 | 397 |
| 66 | (S)-ethyl 2-(4-(5- phenyl-4,5-dihydro- 1H-pyrazole-1- carbonyl)piperidin- 1-yl)oxazole-5- carboxylate | 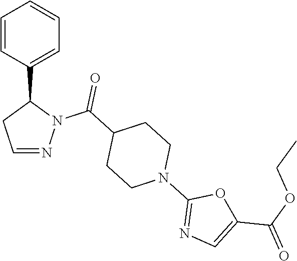 [SMILES: CCOC(=O)c1cnc(N2CCC(C(=O)N3N=CC[C@H]3c3ccccc3)CC2)o1] | ¹H NMR (400 MHz, CDCl3) δ ppm 7.54 (s, 1H), 7.34 (m, 2H), 7.27 (m, 1H), 7.16 (m, 2H), 7.01 (t, J = 1.5 Hz, 1H), 5.38 (dd, J = 12.0, 4.9 Hz, 1H), 4.32 (q, J = 7.2 Hz, 2H), 4.24 (m, 2H), 3.46 (ddd, J = 19.0, 12.0, 1.5 Hz, 1H), 3.36 (tt, J = 10.9, 3.9 Hz, 1H), 3.19 (m, 2H), 2.85 (ddd, J = 18.8, 12.0, 1.7 Hz, 1H), 1.87 (m, 4H), 1.36 (t, J = 7.2 Hz, 3H) | 2.41 Method 1 | 397 |
| 67 | (S)-(5-phenyl-4,5- dihydro-1H- pyrazol-1-yl)(1-(5- phenyloxazol-2- yl)piperidin-4- yl)methanone | 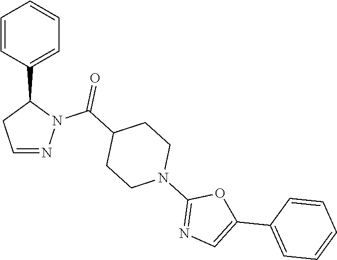 [SMILES: O=C(C1CCN(c2ncc(-c3ccccc3)o2)CC1)N1N=CC[C@H]1c1ccccc1] | ¹H NMR (400 MHz, CDCl3) δ ppm 7.49 (d, J = 7.40 Hz, 2H), 7.35 (m, 4H), 7.26 (m, 2H), 7.17 (d, J = 7.2 Hz, 2H), 7.06 (s, 1H), 7.01 (s, 1H), 5.39 (dd, J = 12.0, 4.7 Hz, 1H), 4.21 (m, 2H), 3.45 (ddd, J = 18.8, 12.0, 1.5 Hz, 1H), 5.35 (m, 1H), 3.15 (m, 2H), 2.85 (m, 2H), 2.85 (ddd, J = 18.8, 4.7, 1.5 Hz, 1H), 1.80-2.05 (m, 4H) | 2.51 Method 1- 20 V | 401 |
| 68 | (S)-6-(4-(5-(3- cyanophenyl)-4,5- dihydro-1H- pyrazole-1- carbonyl)piperidin- 1-yl)pyrimidine-4- carbonitrile | 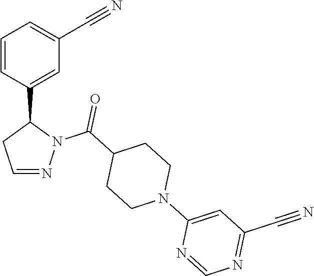 [SMILES: N#Cc1cccc([C@@H]2CC=NN2C(=O)C2CCN(c3cc(C#N)ncn3)CC2)c1] | ¹H NMR (400 MHz, DMSO-d6) δ ppm 8.54 (d, J = 1.1 Hz, 1H), 7.73 (m, 1H), 7.60 (s, 1H), 7.55 (m, 2H), 7.46 (m, 1H), 7.28 (s, 1H), 5.37 (dd, J = 12.0, 4.9 Hz, 1H), 4.42 (m, 2H), 3.51 (ddd, J = 19.0, 12.0, 1.3 Hz, 1H), 3.43 (tt, J = 11.4, 3.8 Hz, 1H), 3.13 (m, 2H), 2.76 (ddd, J = 19.0, 5.1, 1.7 Hz, 1H), 1.92 (d, J = 11.9 Hz, 1H), 1.81 (d, J = 11.9 Hz, 1H), 1.47 (m, 2H) | 2.24 Method 1 | 386 |
| 69 | (S)-3-(1-(1-(4- methoxypyrimidin- 2-yl)piperidine-4- carbonyl)-4,5- dihydro-1H- pyrazol-5- yl)benzonitrile | 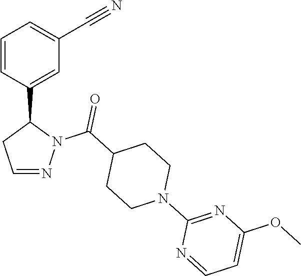 [SMILES: COc1ccnc(N2CCC(C(=O)N3N=CC[C@H]3c3cccc(C#N)c3)CC2)n1] | ¹H NMR (400 MHz, DMSO-d6) δ ppm 8.07 (d, J = 5.5 Hz, 1H), 7.73 (d, J = 7.6 Hz, 1H), 7.61 (s, 1H), 7.55 (t, J = 7.8 Hz, 1H), 7.45 (d, J = 8.0 Hz, 1H), 7.27 (s, 1H), 6.04 (d, J = 5.5 Hz, 1H), 5.37 (dd, J = 11.8, 4.9 Hz, 1H), 4.65 (d, J = 13.1 Hz, 2H), 3.82 (s, 3H), 3.51 (dd, J = 18.6, 12.5 Hz, 1H), 3.38 (m, 1H), 2.97 (m, 2H), 2.76 (dd, J = 18.4, 4.4, Hz, 1H), 1.87 (d, J = 12.1 Hz, 1H), 1.76 (d, J = 11.6 Hz, 1H), 1.45 (m, 2H) | 1.68 Method 1 | 391 |
| 70 | (S)-6-(4-(5-(3- cyanophenyl)-4,5- dihydro-1H- pyrazole-1- carbonyl)piperidin- 1-yl)pyrimidine-4- carboxamide | 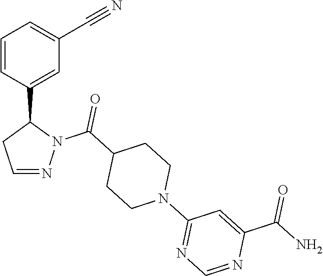 [SMILES: N#Cc1cccc([C@@H]2CC=NN2C(=O)C2CCN(c3cc(C(N)=O)ncn3)CC2)c1] | ¹H NMR (400 MHz, DMSO-d6) δ ppm 8.54 (s, 1H), 8.04 (s, 1H), 7.73 (m, 2H), 7.61 (s, 1H), 7.55 (t, J = 7.7 Hz, 1H), 7.46 (d, J = 8.0 Hz, 1H), 7.29 (m, 2H), 5.37 (dd, J = 12.0, 4.9 Hz, 1H), 4.46 (s, 2H), 3.51 (ddd, J = 19.0, 12.0, 1.3 Hz, 1H), 3.42 (tt, J = 11.4, 3.7 Hz, 1H), 3.10 (m, 2H), 2.76 (ddd, J = 19.0, 4.9, 1.5 Hz, 1H), 1.92 (d, J = 12.4 Hz, 1H), 1.81 (d, J = 12.6 Hz, 1H), 1.48 (m, 2H) | 1.77 Method 1 | 404 |
| 71 | (S)-2-(4-(5-(3- cyanophenyl)-4,5- dihydro-1H- pyrazole-1- carbonyl)piperidin- 1-yl)pyrimidine-4- carboxamide | 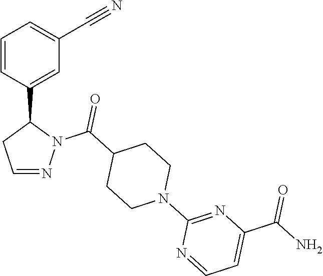 [SMILES: N#Cc1cccc([C@@H]2CC=NN2C(=O)C2CCN(c3nccc(C(N)=O)n3)CC2)c1] | ¹H NMR (400 MHz, DMSO-d6) δ ppm 8.54 (d, J = 4.7 Hz, 1H), 8.17 (br. s, 1H), 7.73 (m, 2H), 7.61 (s, 1H), 7.55 (t, J = 7.8 Hz, 1H), 7.46 (d, J = 8.0 Hz, 1H), 7.28 (s, 1H), 7.07 (d, J = 4.9 Hz, 1H), 5.38 (dd, J = 12.0, 4.9 Hz, 1H), 4.8 (d, J = 12.0 Hz, 2H), 3.51 (ddd, J = 19.0, 12.0, 1.0 Hz, 1H), 3.39 (m, 1H), 3.0 (m, 2H), 2.76 (ddd, J = 19.2, 5.1, 1.5 Hz, 1H), 1.90 (d, J = 12.0 Hz, 1H), 1.78 (d, J = 12.1 Hz, 1H), 1.47 (m, 2H) | 2.05 Method 1 | 404 |
| 72 | (S)-3-(1-(1-(4- amino-5- fluoropyrimidin-2- yl)piperidine-4- carbonyl)-4,5- dihydro-1H- pyrazol-5- yl)benzonitrile | 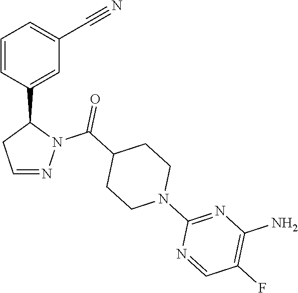 [SMILES: N#Cc1cccc([C@@H]2CC=NN2C(=O)C2CCN(c3ncc(F)c(N)n3)CC2)c1] | ¹H NMR (400 MHz, DMSO-d6) δ ppm 7.78 (d, J = 3.2 Hz, 1H), 7.73 (d, J = 7.6 Hz, 1H), 7.60 (s, 1H), 7.55 (t, J = 7.7 Hz, 1H), 7.45 (d, J = 7.8 Hz, 1H), 7.26 (s, 1H), 6.83 (s, 2H), 5.36 (dd, J = 11.9, 4.6 Hz, 1H), 4.51 (d, J = 12.7 Hz, 2H), 3.50 (dd, J = 18.7, 12.2 Hz, 1H), 3.32 (m, 1H), 2.83 (m, 2H), 2.75 (dd, J = 19.0, 4.2 Hz, 1H), 1.80 (d, J = 11.96 Hz, 1H), 1.70 (d, J = 11.9 Hz, 1H), 1.41 (m, 2H) | 1.39 Method 1 | 394 |
| 73 | (S)-3-(1-(1- (imidazo[1,2- b]pyridazin-6- yl)piperidine-4- carbonyl)-4,5- dihydro-1H- pyrazol-5- yl)benzonitrile | 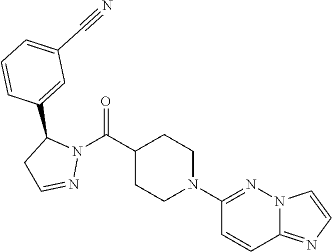 [SMILES: N#Cc1cccc([C@@H]2CC=NN2C(=O)C2CCN(c3ccc4nccn4n3)CC2)c1] | ¹H NMR (400 MHz, DMSO-d6) δ ppm 7.88 (s, 1H), 7.81 (d, J = 9.9 Hz, 1H), 7.73 (d, J = 7.6 Hz, 1H), 7.61 (s, 1H), 7.55 (dd, J = 7.8 Hz*2, 1H), 7.46 (m, 2H), 7.28 (s, 1H) 7.17 (d, J = 10.1 Hz, 1H), 5.38 (dd, J = 11.8, 4.7 Hz, 1H), 4.17 (d, J = 12.7 Hz, 2H), 3.51 (dd, J = 18.6, 12.1 Hz, 1H), 3.38 (m, 1H), 2.98 (m, 2H), 2.76 (dd, J = 19.1, 4.1 Hz, 1H), 1.91 (d, J = 12.2 Hz, 1H), 1.79 (d, J = 12.3 Hz, 1H), 1.59 (m, 2H) | 1.57 Method 1 | 400 |
| 74 | (S)-4-(4-(5-(3- cyanophenyl)-4,5- dihydro-1H- pyrazole-1- carbonyl)piperidin- 1-yl)pyrimidine-2- carbonitrile | 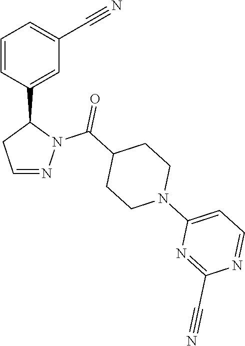 [SMILES: N#Cc1cccc([C@@H]2CC=NN2C(=O)C2CCN(c3ccnc(C#N)n3)CC2)c1] | ¹H NMR (400 MHz, DMSO-d6) δ ppm 8.23 (d, J = 6.3 Hz, 1H), 7.73 (d, J = 7.4 Hz, 1H), 7.61 (s, 1H), 7.55 (t, J = 7.6 Hz, 1H), 7.46 (d, J = 7.2 Hz, 1H), 7.28 (br. s, 1H), 7.11 (d, J = 6.3 Hz, 1H), 5.37 (dd, J = 11.7, 4.5 Hz, 1H), 4.37 (br. s, 2H), 3.51 (dd, J = 18.7, 12.2 Hz, 1H), 3.4 (m, 1H), 3.12 (m, 2H), 2.77 (dd, J = 18.6, 3.23 Hz, 1H), 1.94 (d, J = 11.8 Hz, 1H), 1.82 (d, J = 12.5 Hz, 1H), 1.48 (m, 2H) | 2.31 Method 1 | 386 |
| 75 | (S)-3-(1-(1-(2- methoxypyrimidin- 4-yl)piperidine-4- carbonyl)-4,5- dihydro-1H- pyrazol-5- yl)benzonitrile | 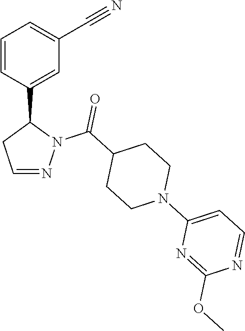 [SMILES: COc1nccc(N2CCC(C(=O)N3N=CC[C@H]3c3cccc(C#N)c3)CC2)n1] | ¹H NMR (400 MHz, DMSO-d6) δ ppm 7.98 (d, J = 6.1 Hz, 1H), 7.73 (d, J = 7.6 Hz, 1H), 7.60 (s, 1H), 7.55 (t, J = 7.8 Hz, 1H), 7.45 (d, J = 7.8 Hz, 1H), 7.27 (s, 1H), 6.49 (d, J = 6.1 Hz, 1H), 5.37 (dd, J = 12.0, 4.9 Hz, 1H), 4.35 (s, 2H), 3.78 (s, 3H), 3.51 (dd, J = 18.3, 12.6 Hz, 1H), 3.39 (m, 1H), 3.01 (m, 2H), 2.76 (ddd, J = 19.2, 4.9, 1.5 Hz, 1H), 1.89 (d, J = 11.8 Hz, 1H), 1.77 (d, J = 11.8 Hz, 1H), 1.45 (m, 2H) | 1.47 Method 1 | 391 |
| 76 | (S)-3-(1-(1-(1H- pyrazolo[3,4- d]pyrimidin-6- yl)piperidine-4- carbonyl)-4,5- dihydro-1H- pyrazol-5- yl)benzonitrile | 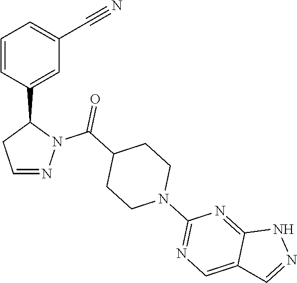 [SMILES: N#Cc1cccc([C@@H]2CC=NN2C(=O)C2CCN(c3ncc4cn[nH]c4n3)CC2)c1] | ¹H NMR (400 MHz, DMSO-d6) δ ppm 13.06 (s, 1H), 8.88 (s, 1H), 7.96 (s, 1H), 7.73 (d, J = 7.4 Hz, 1H), 7.60 (s, 1H), 7.55 (t, J = 7.8 Hz, 1H), 7.45 (d, J = 7.6 Hz, 1H), 7.27 (s, 1H), 5.37 (dd, J = 12.0, 4.7 Hz, 1H), 4.74 (d, J = 12.3 Hz, 2H), 3.51 (dd, J = 18.4, 12.1 Hz, 1H), 3.40 (m, 1H), 3.06 (m, 2H), 2.76 (dd, J = 18.6, 3.8 Hz, 1H), 1.90 (d, J = 12.1 Hz, 1H), 1.79 (d, J = 12.0 Hz, 1H), 1.48 (dd, J = 20.3, 8.7 Hz, 2H) | 2.05 Method 1 | 401 |

### Example 77

### (S)-(5-(3,5-difluorophenyl)-4,5-dihydro-1H-pyrazol-1-yl)(1-(5-methyl-1,3,4-oxadiazol-2-yl)piperidin-4-yl)methanone

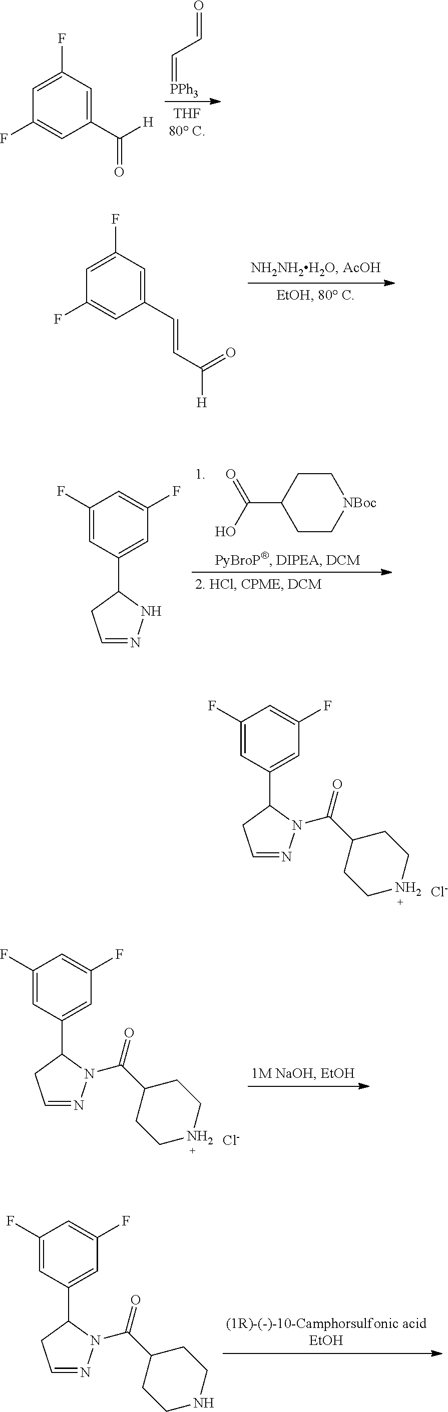

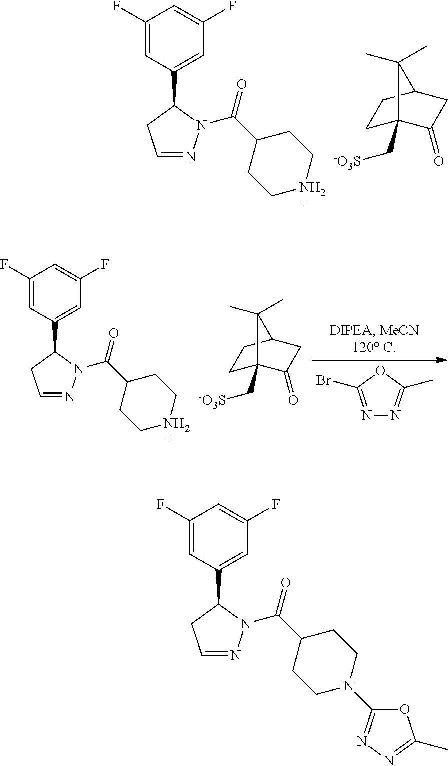

### Step 1

To a solution of 3,5-difluorobenzaldehyde (50 g, 352 mmol) in THF (300 mL) stirred under nitrogen at rt was added (triphenylphosphoranylidene)acetaldehyde (118 g, 387 mmol). The reaction mixture was stirred at 80° C. for 15 h and evaporated in vacuo. The residue was purified by normal phase column chromatography (CyH/EtOAc 100/0 to 90/10) to afford 3-(3,5-difluorophenyl)acrylaldehyde (25.6 g, 91 mmol, purity: 60%, recovery: 26%) as a yellow powder. LCMS (m/z) 169 (M+H)⁺, retention time: 2.28 min, LC/MS Method 1.

### Step 2

To a solution of hydrazine monohydrate (11.1 mL, 228 mmol) in ethanol (30 mL) was added acetic acid (14.8 mL, 259 mmol) at rt. The reaction mixture was heated to 45° C. and solid 3-(3,5-difluorophenyl)acrylaldehyde (25.6 g, 152 mmol) was added portion-wise during 20 min. The reaction vessel was sealed and heated to 80° C. for 21 h. The reaction mixture was concentrated in vacuo. The yellow residue was purified by normal phase column chromatography [CyH/(EtOAc/EtOH 3:1) 100/0 to 75/25] to afford 5-(3,5-difluorophenyl)-4,5-dihydro-1H-pyrazole (20 g, 110 mmol, purity: 63%, recovery: 72%) as an orange oil. LCMS (m/z) 183 (M+H)⁺, retention time: 1.89 min, LC/MS Method 1.

### Step 3

To a solution of 1-(tert-butoxycarbonyl)piperidine-4-carboxylic acid (25.2 g, 110 mmol) in DCM (300 mL) were added PyBroP® (53.7 g, 115 mmol) and DIPEA (21.09 mL, 121 mmol) at rt. After stirring for 5 min, 5-(3,5-difluorophenyl)-4,5-dihydro-1H-pyrazole (20 g, 110 mmol) was added. The reaction was stirred for 5 h and concentrated in vacuo. The residue was purified by normal phase column chromatography [CyH/(EtOAc/EtOH 3:1) 100/0 to 50/50] to provide tert-butyl 4-(5-(3,5-difluorophenyl)-4,5-dihydro-1H-pyrazole-1-carbonyl)piperidine-1-carboxylate. tert-Butyl 4-(5-(3,5-difluorophenyl)-4,5-dihydro-1H-pyrazole-1-carbonyl)piperidine-1-carboxylate was dissolved in DCM (500 mL) and a 3 M solution of HCl in CPME (91 mL, 274 mmol) was added at rt. The reaction was stirred at rt for 24 h. The precipitate was filtered off, washed with DCM (2×150 mL) and iPr₂O (3×200 mL) to give (5-(3,5-difluorophenyl)-4,5-dihydro-1H-pyrazol-1-yl)(piperidin-4-yl)methanone, hydrochloride (20 g, 60.6 mmol, purity: 90%, recovery: 55%) as a cream powder. LCMS (m/z) 294 (M+H)⁺, retention time: 1.17 min, LC/MS Method 1.

### Step 4

To a solution of (5-(3,5-difluorophenyl)-4,5-dihydro-1H-pyrazol-1-yl)(piperidin-4-yl)methanone, hydrochloride (20 g, 60.6 mmol) in EtOH (50 mL) was added a 1 M solution of sodium hydroxide (79 mL, 79 mmol). The reaction mixture was stirred at rt for 30 min. DCM (150 mL) was added and the two layers were separated. The aqueous layer was extracted with DCM (2×150 mL). The combined organic layers were dried over sodium sulfate, filtered, and evaporated in vacuo to give the free base as an oil (17.3 g). This residue was dissolved in EtOH (50 mL) and (1R)-(−)-10-camphorsulfonic acid (14.09 g, 06.6 mmol) was added at rt. The resulting suspension was heated at 60° C. for 30 min. The solution was then evaporated to dryness and the partially crystalline crude solid was suspended and slurried in ethanol (50 mL) to fully convert it to a crystalline form, and this suspension was evaporated to dryness to give a light orange crystalline solid. This solid was recrystallized from EtOH (300 mL) to afford (S)-(5-(3,5-difluorophenyl)-4,5-dihydro-1H-pyrazol-1-yl)(piperidin-4-yl)methanone, 1R-(−)-camphor-10-sulphonic acid salt (7 g, 13.3 mmol, purity: 100%, recovery: 22%) as a white powder. LCMS (m/z) 294 (M+H)⁺, retention time: 1.17 min, LC/MS Method 1. Chiral HPLC Method 1: 2.58 and 3.26 min, % ee=99.2%.

### Step 5

To a suspension of (S)-(5-(3,5-difluorophenyl)-4,5-dihydro-1H-pyrazol-1-yl)(piperidin-4-yl)methanone, 1R-(−)-camphor-10-sulphonic acid salt (350 mg, 0.666 mmol) in MeCN (10 mL) was added 2-bromo-5-methyl-1,3,4-oxadiazole (80 mg, 0.57 mmol) and DIPEA (0.291 mL, 1.66 mmol). The vessel was sealed, and heated with stirring at 120° C. for 2 h. The reaction mixture was evaporated in vacuo. This residue was purified by normal phase column chromatography [DCM:MeOH (100:0 to 95:5)]. A trituration with Et₂O afforded ((S)-(5-(3,5-difluorophenyl)-4,5-dihydro-1H-pyrazol-1-yl)(1-(5-methyl-1,3,4-oxadiazol-2-yl)piperidin-4-yl)methanone (110 mg, 0.293 mmol, purity: 100%, recovery: 44%) as a white foam. LCMS (m/z) 376 (M+H)⁺, retention time: 2.12 min, LC/MS Method 1. ¹H NMR (400 MHz, DMSO-d6) δ ppm 7.25 (s, 1H), 7.12 (tt, J=9.4, 2.2 Hz, 1H), 6.84 (d, J=7.3 Hz, 2H), 5.34 (dd, J=12.0, 4.9 Hz, 1H), 3.81 (m, 2H), 3.48 (ddd, J=19.0, 12.0, 1.3 Hz, 1H), 3.29 (m, 1H), 3.09 (m, 2H), 2.75 (ddd, J=19.0, 4.9, 1.5 Hz, 1H), 2.32 (s, 3H), 1.91 (d, J=11.8 Hz, 1H), 1.77 (d, J=11.8 Hz, 1H), 1.57 (m, 2H).

### Example 78

### (S)-6-(4-(5-(3,5-difluorophenyl)-4,5-dihydro-1H-pyrazole-1-carbonyl)piperidin-1-yl)pyrimidine-4-carbonitrile

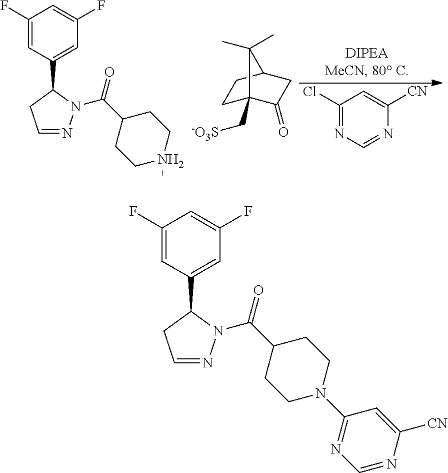

To a suspension of (S)-(5-(3,5-difluorophenyl)-4,5-dihydro-1H-pyrazol-1-yl)(piperidin-4-yl)methanone, 1R-(−)-camphor-10-sulphonic acid salt (300 mg, 0.57 mmol) in MeCN (30 mL) was added 6-chloropyrimidine-4-carbonitrile (80 mg, 0.57 mmol) and DIPEA (0.25 mL, 1.43 mmol) The vessel was sealed and heated at 80° C. for 2 h. The reaction mixture was evaporated in vacuo. This residue was purified by normal phase column chromatography [CyH/(EtOAc/EtOH 3:1) 100/0 to 70/30]. A trituration into iPr₂O afforded, after filtration, (S)-6-(4-(5-(3,5-difluorophenyl)-4,5-dihydro-1H-pyrazole-1-carbonyl)piperidin-1-yl)pyrimidine-4-carbonitrile (130 mg, 0.33 mmol, purity: 100%, recovery: 58%) as a light yellow powder. LCMS (m/z) 397 (M+H)⁺, retention time: 2.48 min, LC/MS Method 1. ¹H NMR (400 MHz, DMSO-d6) δ ppm 8.54 (s, 1H), 7.57 (s, 1H), 7.26 (s, 1H), 7.12 (tt, J=9.4, 2.1 Hz, 1H), 6.84 (d, J=6.5 Hz, 2H), 5.34 (dd, J=12.0, 4.9 Hz, 1H), 4.47 (br.s, 2H), 3.49 (ddd, J=19.0, 12.0, 1.0 Hz, 1H), 3.43 (tt, J=11.4, 3.7 Hz, 1H), 3.13 (br s, 2H), 2.75 (ddd, J=19.2, 4.9, 1.5 Hz, 1H), 1.95 (d, J=12.7 Hz, 1H), 1.81 (d, J=12.7 Hz, 1H), 1.48 (m, 2H).

Examples 79-107 were synthesized in an analogous manner to Examples 77 and 78. For step 5, DIPEA may be substituted for TEA and the temperature may vary from 80 to 150° C. For step 2, the combination of EtOH and AcOH at 80° C. may be substituted for Et₂O at rt. For step 3, PyBroP® may be substituted for HATU. For example 84, the starting product was 2-bromo-5-methyl-1,3,4-oxadiazole.

| | | | | LC: | |
|---|---|---|---|---|---|
| | | | | retention | |
| | | | | time | |
| | | | | (min); | |
| | | | | LC/MS | |
| | | | | Method of | MS |
| Ex. | Name | Structure | ¹H NMR | analysis | (M + H)⁺ |
| 79 | (S)-(5-(3,5- difluoro- phenyl)- 4,5-dihydro- 1H-pyrazol- 1-yl)(1-(4- methoxy- pyrimidin- 2-yl) piperidin- 4-yl) methanone | 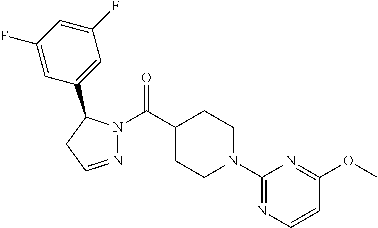 [SMILES: COc1ccnc(N2CCC(C(=O)N3N=CC[C@H]3c3cc(F)cc(F)c3)CC2)n1] | ¹H NMR (400 MHz, DMSO-d6) δ ppm 8.07 (d, J = 5.5 Hz, 1H), 7.25 (s, 1H), 7.12 (t, J = 9.3 Hz, 1H), 6.84 (d, J = 6.6 Hz, 2H), 6.04 (d, J = 5.5 Hz, 1H), 5.34 (dd, J = 11.9, 4.8 Hz, 1H), 4.66 (d, J = 12.9 Hz, 2H), 3.82 (s, 3H), 3.48 (dd, J = 18.8, 12.1 Hz, 1H), 3.38 (m, | 1.92 Method 1 | 402 |
| | | | 1H), 2.97 (m, 2H), | | |
| | | | 2.75 (dd, J = 18.8, | | |
| | | | 4.0 Hz, 1H), 1.89 | | |
| | | | (d, J = 12.1 Hz, | | |
| | | | 1H), 1.76 (d, J = | | |
| | | | 12.3 Hz, 1H), 1.47 | | |
| | | | (m, 2H) | | |
| 80 | (S)-2-(4- (5-(3,5- difluoro- phenyl)-4,5- dihydro-1H- pyrazole-1- carbonyl) piperidin- 1-yl)-5H- pyrrolo [2,3-d] pyrimidin- | 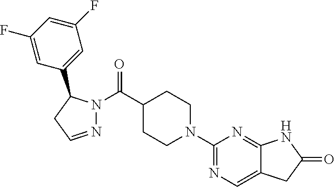 [SMILES: O=C1Cc2cnc(N3CCC(C(=O)N4N=CC[C@H]4c4cc(F)cc(F)c4)CC3)nc2N1] | ¹H NMR (400 MHz, DMSO-d6) δ ppm 11.05 (s, 1H), 7.92 (s, 1H), 7.25 (s, 1H), 7.12 (tt, J = 9.3, 2.1 Hz, 1H), 6.83 (d, J = 6.3 Hz, 2H), 5.34 (dd, J = 11.9, 4.8 Hz, 1H), 4.60 (d, J = 12.9 Hz, 2H), 3.48 (dd, J = 18.3, 12.4 Hz, 1H), 3.39 (s, | 1.59 Method 1 | 427 |
| | 6(7H)-one | | 2H), 3.37 (m, 1H), | | |
| | | | 2.96 (m, 2H), 2.75 | | |
| | | | (ddd, J = 19.0, 4.7, | | |
| | | | 1.3 Hz, 1H), 1.86 | | |
| | | | (d, J = 11.6 Hz, | | |
| | | | 1H), 1.73 (d, J = | | |
| | | | 11.6 Hz, 1H), 1.46 | | |
| | | | (m, 2H) | | |
| 81 | (S)-(1-(4- amino-5- fluoro- pyrimidin- 2-yl) piperidin- 4-yl)(5- (3,5- difluoro- phenyl)- 4,5- dihydro- 1H- pyrazol- 1-yl) | 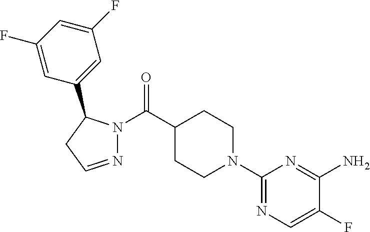 [SMILES: Nc1nc(N2CCC(C(=O)N3N=CC[C@H]3c3cc(F)cc(F)c3)CC2)ncc1F] | ¹H NMR (400 MHz, DMSO-d6) δ ppm 7.78 (d, J = 3.2 Hz, 1H), 7.24 (s, 1H), 7.12 (t, J = 9.1 Hz, 1H), 6.82 (m, 4H), 5.33 (dd, J = 11.8, 4.7 Hz, 1H), 4.52 (d, J = 12.7 Hz, 2H), 3.47 (dd, J = 18.6, 12.1 Hz, 1H), 3.29 (m, 1H), 2.84 (m, 2H), 2.74 (dd, J = 18.8, 4.0 Hz, 1H), 1.82 (d, J = 11.8 | 1.55 Method 1 | 405 |
| | methanone | | Hz, 1H), 1.69 (d, | | |
| | | | J = 12.1 Hz, 1H), | | |
| | | | 1.42 (m, 2H) | | |
| 82 | (S)-(5-(3,5- difluoro- phenyl)- 4,5-dihydro- 1H-pyrazol- 1-yl)(1-(2- (methylthio) pyrimidin- 4-yl) piperidin- 4-yl) methanone | 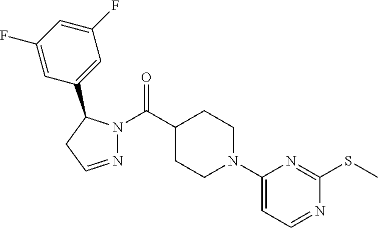 [SMILES: CSc1nccc(N2CCC(C(=O)N3N=CC[C@H]3c3cc(F)cc(F)c3)CC2)n1] | ¹H NMR (400 MHz, DMSO-d6) δ ppm 8.0 (d, J = 6.3 Hz, 1H), 7.26 (s, 1H), 7.12 (t, J = 9.3 Hz, 1H), 6.84 (d, J = 6.6 Hz, 2H), 6.56 (d, J = 6.3 Hz, 1H), 5.34 (dd, J = 11.8, 4.7 Hz, 1H), 4.38 (s, 2H), 3.48 (dd, J = 19.1, 12.2 Hz, 1H), 3.40 (m, 1H), 3.01 (m, 2H), 2.75 | 1.79 Method 1 | 418 |
| | | | (dd, J = 18.9 Hz, | | |
| | | | 1H), 2.41 (s, 3H), | | |
| | | | 1.91 (d, J = 12.1 | | |
| | | | Hz, 1H), 1.78 (d, | | |
| | | | J = 12.1 Hz, 1H), | | |
| | | | 1.47 (m, 2H) | | |
| 83 | (S)-2-(4- (5-(3,5- difluoro- phenyl)- 4,5-dihydro- 1H- pyrazole-1- carbonyl) piperidin- 1-yl) pyrimidine- 4-carbox- amide | 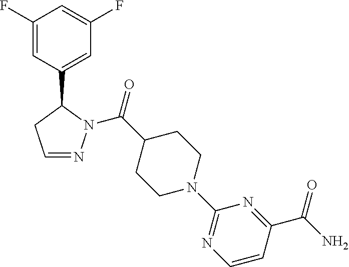 [SMILES: NC(=O)c1ccnc(N2CCC(C(=O)N3N=CC[C@H]3c3cc(F)cc(F)c3)CC2)n1] | ¹H NMR (400 MHz, DMSO-d6) δ ppm 8.54 (d, J = 4.9 Hz, 1H), 8.17 (s, 1H), 7.73 (s, 1H), 7.26 (s, 1H), 7.12 (tt, J = 9.3, 2.3 Hz, 1H), 7.07 (d, J = 4.9 Hz, 1H), 6.85 (m, 2H), 5.35 (dd, J = 12.0, 4.9 Hz, 1H), 4.81 (m, 2H), 3.49 (ddd, J = 19.0, 12.0, 1.3 Hz, 1H), 3.39 (tt, J = 11.5, 3.8 Hz, 1H), 3.01 (m, 2H), 2.75 | 2.26 Method 1 | 415 |
| | | | (ddd, J = 19.0, 4.9, | | |
| | | | 1.7 Hz, 1H), 1.92 | | |
| | | | (d, J = 12.7 Hz, | | |
| | | | 1H), 1.78 (d, J = | | |
| | | | 12.7 Hz, 1H), 1.49 | | |
| | | | (m, 2H) | | |
| 84 | (S)-(1-(2- amino- pyrimidin- 4-yl) piperidin- 4-yl)(5- (3,5- difluoro- phenyl)- 4,5- dihydro- 1H- pyrazol- 1-yl) methanone | 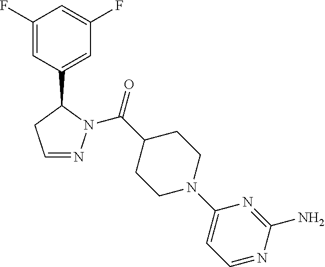 [SMILES: Nc1nccc(N2CCC(C(=O)N3N=CC[C@H]3c3cc(F)cc(F)c3)CC2)n1] | ¹H NMR (400 MHz, DMSO-d6) δ ppm 7.84 (d, J = 6.5 Hz, 1H), 7.00 (s, 1H), 6.71 (m, 3H), 5.34 (dd, J = 12.1, 5.0 Hz, 1H), 4.96 (br.s, 2H), 4.38 (d, J = 12.9 Hz, 2H), 3.46 (ddd, J = 18.8, 12.0, 1.3 Hz, 1H), 3.39 (m, 1H), 3.00 (m, 2H), 2.80 (ddd, J = 18.8, 4.9, 1.5 Hz, 1H), 1.70-2.00 (m, 4H) | 1.55 Method 1 | 387 |
| 85 | (S)-6-(4- (5-(3,5- difluoro- phenyl)- 4,5- dihydro-1H- pyrazole-1- carbonyl) piperidin- 1-yl) pyrimidine- 4- carboxamide | 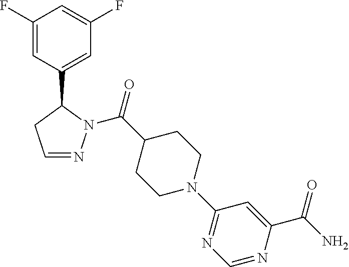 [SMILES: NC(=O)c1cc(N2CCC(C(=O)N3N=CC[C@H]3c3cc(F)cc(F)c3)CC2)ncn1] | ¹H NMR (400 MHz, DMSO-d6) δ ppm 8.54 (s, 1H), 8.05 (s, 1H), 7.74 (s, 1H), 7.31 (s, 1H), 7.26 (s, 1H), 7.12 (tt, J = 9.3, 2.3 Hz, 1H), 6.84 (d, J = 6.3 Hz, 2H), 5.34 (dd, J = 12.0, 4.9 Hz, 1H), 4.47 (s, 2H), 3.49 (ddd, J = 19.0, 12.2, 1.3 Hz, 1H), 3.42 (m, 1H), 3.10 (m, 2H), 2.75 (ddd, J = 19.0, | 1.98 Method 1 | 415 |
| | | | 4.9, 1.5 Hz, 1H), | | |
| | | | 1.95 (m, 1H), | | |
| | | | 1.81 (d, J = 12.4 | | |
| | | | Hz, 1H), 1.50 | | |
| | | | (m, 2H) | | |
| 86 | (S)-(1-(1H- pyrazolo [3,4-d] pyrimidin- 6-yl) piperidin- 4-yl)(5- (3,5- difluoro- phenyl)- 4,5- dihydro- 1H- pyrazol- 1-yl) methanone | 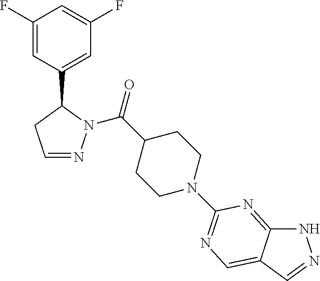 [SMILES: O=C(C1CCN(c2ncc3cn[nH]c3n2)CC1)N1N=CC[C@H]1c1cc(F)cc(F)c1] | ¹H NMR (400 MHz, DMSO-d6) δ ppm 13.07 (s, 1H), 8.88 (s, 1H), 7.97 (s, 1H), 7.26 (s, 1H), 7.12 (m, 1H), 6.84 (d, 2H, J = 6.5 Hz), 5.34 (dd, 1H, J = 11.9, 4.8 Hz), 4.75 (d, 2H, J = 12.9 Hz), 3.46 (dd, 1H, J = 19.0, 12.7 Hz), 3.40 (m, 1H), 3.06 (m, 2H), 2.75 (dd, 1H, J = 19.0, 4.7 Hz), 1.92 (d, 1H, J = 11.2 Hz), 1.78 (d, 1H, J = 11.2 | 2.26 Method 1 | 412 |
| | | | Hz), 1.51 (m, 2H) | | |
| 87 | (S)-(5- (3,5- difluoro- phenyl)- 4,5- dihydro- 1H- pyrazol-1- yl)(1- (imidazo [1,2-b] pyridazin- 6-yl) piperidin- 4-yl) methanone | 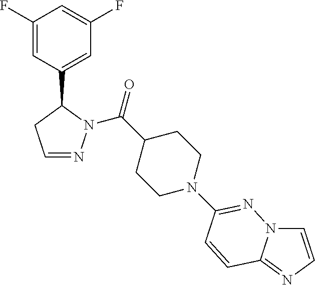 [SMILES: O=C(C1CCN(c2ccc3nccn3n2)CC1)N1N=CC[C@H]1c1cc(F)cc(F)c1] | ¹H NMR (400 MHz, DMSO-d6) δ ppm 7.89 (s, 1H), 7.82 (d, J = 10.1 Hz, 1H), 7.47 (s, 1H), 7.26 (s, 1H), 7.17 (d, J = 10.1 Hz, 1H), 7.12 (m, 1H), 6.85 (d, J = 6.5 Hz, 2H), 5.35 (dd, J = 11.9, 4.8 Hz, 1H), 4.17 (d, J = 11.8 Hz, 2H), 3.49 (dd, J = 18.6, 12.3 Hz, 1H), 3.38 (m, 1H), 3.0 (m, 2H), 2.75 (dd, J = 18.9, 3.9 Hz, 1H), 1.93 (d, | 1.74 Method 1 | 411 |
| | | | J = 12.1 Hz, 1H), | | |
| | | | 1.79 (d, J = 12.0 | | |
| | | | Hz, 1H), 1.61 (m, | | |
| | | | 2H) | | |
| 88 | (S)-4-(4- (5-(3,5- difluoro- phenyl)- 4,5- dihydro- 1H- pyrazole-1- carbonyl) piperidin-1- yl) pyrimidine- 5- carbonitrile | 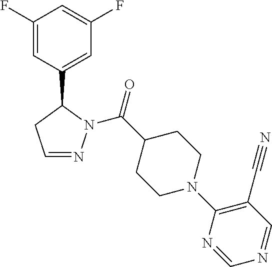 [SMILES: N#Cc1cncnc1N1CCC(C(=O)N2N=CC[C@H]2c2cc(F)cc(F)c2)CC1] | ¹H NMR (400 MHz, DMSO-d6) δ ppm 8.67 (d, J = 6.5 Hz, 2H), 7.26 (s, 1H), 7.12 (t, J = 9.1 Hz, 1H), 6.85 (d, J = 6.6 Hz, 2H), 5.35 (dd, J = 11.8, 4.7 Hz, 1H), 4.65 (d, J = 12.7 Hz, 2H), 3.49 (m, 2H), 3.31 (m, 2H), 2.74 (dd, J = 18.8, 3.8 Hz, 1H), 2.01 (d, J = 12.5 Hz, 1H), 1.86 (d, J = 12.1 Hz, 1H), 1.61 (m, 2H) | 2.34 Method 1 | 397 |
| 89 | (S)-4-(4- (5-(3,5- difluoro- phenyl)- 4,5- dihydro- 1H- pyrazole-1- carbonyl) piperidin- 1-yl) pyrimidine- 2- carbonitrile | 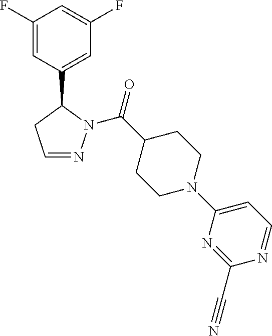 [SMILES: N#Cc1nccc(N2CCC(C(=O)N3N=CC[C@H]3c3cc(F)cc(F)c3)CC2)n1] | ¹H NMR (400 MHz, DMSO-d6) δ ppm 8.24 (s, 1H), 7.26 (s, 1H), 7.13 (m, 2H), 6.84 (d, J = 6.6 Hz, 2H), 5.34 (dd, J = 12.0, 4.7 Hz, 1H), 4.37 (br.s, 2H), 3.46 (m, 2H), 3.11 (m, 2H), 2.75 (dd, J = 18.9, 3.9 Hz, 1H), 1.97 (d, J = 12.2 Hz, 1H), 1.82 (d, J = 12.0 Hz, 1H), 1.50 (m, 2H) | 2.52 Method 1 | 397 |
| 90 | (S)-(5- (3,5- difluoro- phenyl)- 4,5- dihydro- 1H-pyrazol- 1-yl)(1- (pyrazolo [1,5-a] pyrimidin- 5-yl) piperidin- 4-yl) methanone | 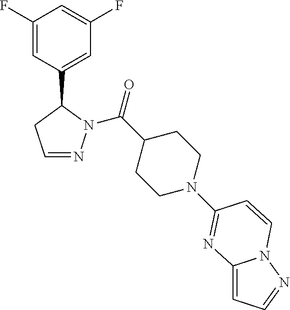 [SMILES: O=C(C1CCN(c2ccn3nccc3n2)CC1)N1N=CC[C@H]1c1cc(F)cc(F)c1] | ¹H NMR (400 MHz, DMSO-d6) δ ppm 8.61 (d, J = 8.0 Hz, 1H), 7.84 (s, 1H), 7.26 (s, 1H), 7.12 (t, J = 9.3 Hz, 1H), 6.84 (d, J = 6.6 Hz, 2H), 6.73 (d, J = 7.8 Hz, 1H), 6.02 (s, 1H), 5.34 (dd, J = 11.8, 4.7 Hz, 1H), 4.43 (d, J = 12.9 Hz, 2H), 3.49 (dd, J = 19.0, 12.3 Hz, 1H), 3.40 (m, 1H), 3.05 (m, 2H), 2.75 (dd, J = 18.8, 4.0 Hz, 1H), 1.93 (d, J = 12.0 Hz, | 2.32 Method 1 | 411 |
| | | | 1H), 1.79 (d, J = | | |
| | | | 12.1 Hz, 1H), | | |
| | | | 1.52 (m, 2H) | | |
| 91 | (S)-(5- (3,5- difluoro- phenyl)- 4,5- dihydro- 1H-pyrazol- 1-yl)(1-(6- methoxy- pyrimidin- 4-yl) piperidin- 4-yl) methanone | 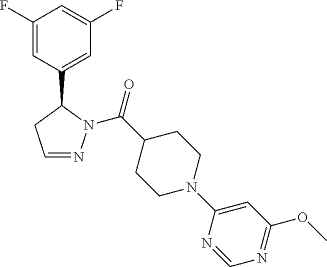 [SMILES: COc1cc(N2CCC(C(=O)N3N=CC[C@H]3c3cc(F)cc(F)c3)CC2)ncn1] | ¹H NMR (400 MHz, DMSO-d6) δ ppm 8.23 (s, 1H), 7.25 (s, 1H), 7.12 (tt, J = 9.3, 2.1 Hz, 1H), 6.84 (d, J = 6.5 Hz, 2H), 6.09 (s, 1H), 5.34 (dd, J = 11.9, 4.8 Hz, 1H), 4.35 (d, J = 12.7 Hz, 2H), 3.81 (s, 3H), 3.48 (dd, J = 18.8, 12.0 Hz, 1H), 3.38 (m, 1H), 2.97 (m, 2H), 2.75 (ddd, J = 19.0, 4.7, 1.3 Hz, 1H), | 2.10 Method 1 | 402 |
| | | | 1.88 (d, J = 11.6 | | |
| | | | Hz, 1H), 1.74 (d, | | |
| | | | J = 11.4 Hz, 1H), | | |
| | | | 1.45 (m, 2H) | | |
| 92 | (S)-2-(4- (5-(3,5- difluoro- phenyl)- 4,5- dihydro- 1H- pyrazole-1- carbonyl) piperidin- 1-yl) pyrimidine- 5- carbonitrile | 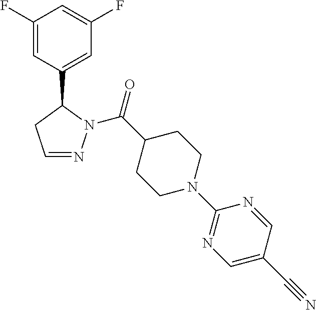 [SMILES: N#Cc1cnc(N2CCC(C(=O)N3N=CC[C@H]3c3cc(F)cc(F)c3)CC2)nc1] | ¹H NMR (400 MHz, DMSO-d6) δ ppm 8.74 (s, 2H), 7.26 (s, 1H), 7.12 (m, 1H), 6.34 (d, J = 6.3 Hz, 2H), 5.34 (dd, J = 12.0, 4.9 Hz, 1H), 4.7 (d, J = 13.1 Hz, 2H), 3.49 (dd, J = 18.9, 12.0, 1H), 3.43 (tt, J = 11.3, 3.8 Hz, 1H), 3.16 (m, 2H), 2.75 (ddd, J = 18.9, 4.7, 1.3 Hz, 1H), 1.96 (d, J = 11.4 Hz, 1H), 1.83 (d, J = 11.8 Hz, 1H), 1.48 (m, 2H) | 2.62 Method 1 | 397 |
| 93 | (S)-(1-(4- amino- pyrimidin- 2-yl) piperidin- 4-yl)(5- (3,5- difluoro- phenyl)- 4,5- dihydro- 1H-pyrazol- 1-yl) methanone | 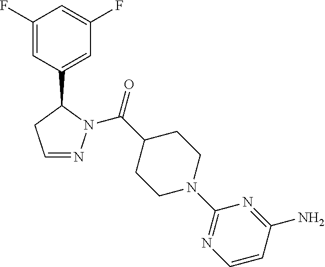 [SMILES: Nc1ccnc(N2CCC(C(=O)N3N=CC[C@H]3c3cc(F)cc(F)c3)CC2)n1] | ¹H NMR (400 MHz, DMSO-d6) δ ppm 7.73 (d, J = 5.7 Hz, 1H), 7.24 (s, 1H), 7.12 (tt, J = 9.3, 2.28 Hz, 1H), 6.83 (m, 2H), 6.36 (s, 2H), 5.70 (d, J = 5.5 Hz, 1H), 5.34 (dd, J = 12.0, 4.9 Hz, 1H), 4.62 (d, J = 13.1 Hz, 2H), 3.48 (ddd, J = 18.9, 12.0, 1.3 Hz, 1H), 3.31 (m, 1H), 2.82 (m, 2H), 2.74 (ddd, | 1.49 Method 1 | 387 |
| | | | J = 18.9 4.9, 1.7 | | |
| | | | Hz, 1H), 1.81 | | |
| | | | (d, J = 10.8 Hz, | | |
| | | | 1H), 1.69 (d, J = | | |
| | | | 11.2 Hz, 1H), | | |
| | | | 1.42 (m, 2H) | | |
| 94 | (S)-6-(4- (5-(3,5- difluoro- phenyl)- 4,5- dihydro- 1H- pyrazole-1- carbonyl) piperidin- 1-yl) pyridazine- 3- carbonitrile | 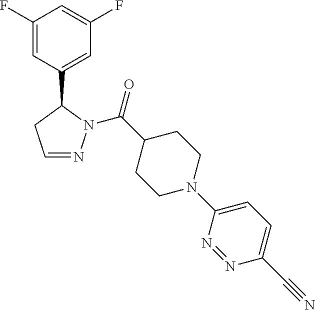 [SMILES: N#Cc1ccc(N2CCC(C(=O)N3N=CC[C@H]3c3cc(F)cc(F)c3)CC2)nn1] | ¹H NMR (400 MHz, DMSO-d6) δ ppm 7.84 (d, J = 9.7 Hz, 1H), 7.37 (d, J = 9.7 Hz, 1H), 7.27 (s, 1H), 7.12 (t, J = 9.2 Hz, 1H), 6.85 (d, J = 6.5 Hz, 2H), 5.35 (dd, J = 11.9, 4.8 Hz, 1H), 4.53 (br.s, 2H), 3.48 (m, 2H), 3.19 (dt, J = 12.1, 10.4 Hz, 2H), 2.75 (dd, J = 18.8, 4.0 Hz, 1H), 1.97 (d, J = Hz, 1H), 1.83 (d, J = 11.8 Hz, 1H), 1.55 (m, 2H) | 2.40 Method 1 | 397 |
| 95 | (S)-5-(4- (5-(3,5- difluoro- phenyl)- 4,5- dihydro- 1H- pyrazole-1- carbonyl) piperidin- 1-yl) pyrazine-2- carbonitrile | 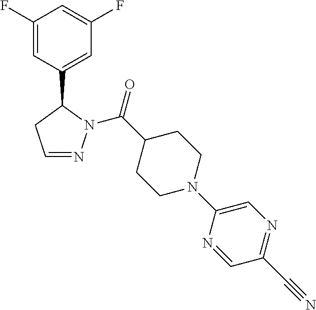 [SMILES: N#Cc1cnc(N2CCC(C(=O)N3N=CC[C@H]3c3cc(F)cc(F)c3)CC2)cn1] | ¹H NMR (400 MHz, DMSO-d6) δ ppm 8.53 (s, 1H), 8.44 (s, 1H), 7.26 (s, 1H), 7.12 (t, J = 9.2 Hz, 1H), 6.84 (d, J = 6.6 Hz, 2H), 5.34 (dd, J = 11.8, 4.9 Hz, 1H), 4.50 (d, J = 13.1 Hz, 2H), 3.45 (m, 2H), 3.17 (dt, J = 11.9, 10.4 Hz, 2H), 2.75 (dd, J = 18.9, 3.7 Hz, 1H), 1.97 (d, J = 12.1 Hz, 1H), 1.83 (d, J = 12.0 Hz, 1H), 1.54 (m, 2H) | 2.54 Method 1 | 397 |
| 96 | (S)-2-(4- (5-(3,5- difluoro- phenyl)- 4,5- dihydro- 1H- pyrazole-1- carbonyl) piperidin- 1-yl) pyrimidine- 5- carboxamide | 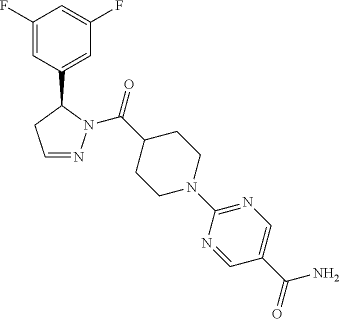 [SMILES: NC(=O)c1cnc(N2CCC(C(=O)N3N=CC[C@H]3c3cc(F)cc(F)c3)CC2)nc1] | ¹H NMR (400 MHz, DMSO-d6) δ ppm 8.77 (s, 2H), 7.83 (br.s, 1H), 7.26 (s, 2H), 7.12 (tt, J = 9.3, 2.2 Hz, 1H), 6.84 (m, 2H), 5.34 (dd, J = 12.0, 4.9 Hz, 1H), 4.73 (d, 13.1 Hz, 2H), 3.49 (ddd, J = 18.8, 12.0, 1.1 Hz, 1H), 3.41 (m, 1H) 3.08 (dt, J = 12.7, 9.5 Hz, 2H), 2.75 (ddd, J = 19.0, 4.9, 1.5 Hz, 1H), 1.93 (d, J = 11.4 Hz, 1H), 1.80 (d, J = 11.6 Hz, 1H), 1.47 (m, 2H) | 2.10 Method 1 | 415 |
| 97 | (S)-2-(4- (5-(3,5- difluoro- phenyl)- 4,5- dihydro- 1H- pyrazole-1- carbonyl) piperidin- 1-yl) pyrimidin- 4(3H)-one | 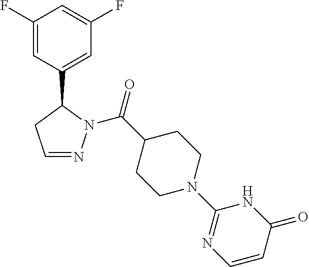 [SMILES: O=C(C1CCN(c2nccc(=O)[nH]2)CC1)N1N=CC[C@H]1c1cc(F)cc(F)c1] | ¹H NMR (400 MHz, DMSO-d6) δ ppm 11.16 (s, 1H), 7.69 (br.s, 1H), 7.25 (s, 1H), 7.12 (tt, J = 9.0, 2.2 Hz, 1H), 6.84 (d, J = 6.3 Hz, 2H), 5.61 (br.s, 1H), 5.34 (dd, J = 12.0, 4.9 Hz, 1H), 4.39 (br.s, 2H), 3.48 (ddd, J = 19.0, 12.1, 1.2 Hz, 1H), 3.36 (m, 1H), 2.98 (m, 2H), 2.74 (ddd, J = 18.9, 4.9, 1.5 Hz, 1H), 1.87 (d, J = | 1.73 Method 1 | 388 |
| | | | 11.4 Hz, 1H), 1.73 | | |
| | | | (d, J = 11.4 Hz, | | |
| | | | 1H), 1.48 (m, 2H) | | |
| 98 | (S)-(1-(6- amino- pyrimidin- 4-yl) piperidin- 4-yl)(5- (3,5- difluoro- phenyl)- 4,5- dihydro- 1H- pyrazol- 1-yl) methanone | 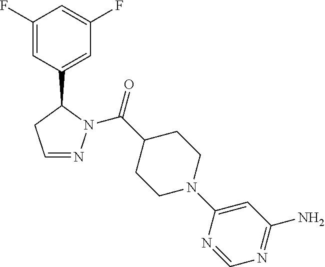 [SMILES: Nc1cc(N2CCC(C(=O)N3N=CC[C@H]3c3cc(F)cc(F)c3)CC2)ncn1] | ¹H NMR (400 MHz, DMSO-d6) δ ppm 7.94 (s, 1H), 7.24 (s, 1H), 7.12 (t, J = 9.2 Hz, 1H), 6.83 (d, J = 6.5 Hz, 2H), 6.15 (s, 2H), 5.61 (s, 1H), 5.34 (dd, J = 12.0, 4.7 Hz, 1H), 4.2 (d, J = 12.7 Hz, 2H), 3.48 (dd, J = 18.6, 12.1 Hz, 1H), 3.33 (m, 1H) 2.87 (dt, J = 12.1, 10.6 Hz, 2H), 2.74 (dd, J = 19.1, 4.0 Hz, 1H), | 1.59 Method 1 | 387 |
| | | | 1.85 (d, J = 12.0 | | |
| | | | Hz, 1H), 1.72 (d, | | |
| | | | J = 12.1 Hz, 1H), | | |
| | | | 1.45 (m, 2H) | | |
| 99 | (S)-(5- (3,5- difluoro- phenyl)- 4,5- dihydro- 1H- pyrazol-1- yl)(1-(2- methoxy- pyrimidin- 4-yl) piperidin- 4-yl) methanone | 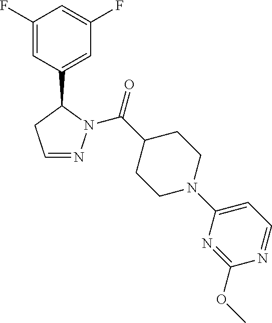 [SMILES: COc1nccc(N2CCC(C(=O)N3N=CC[C@H]3c3cc(F)cc(F)c3)CC2)n1] | ¹H NMR (400 MHz, DMSO-d6) δ ppm 7.98 (d, J = 5.9 Hz, 1H), 7.25 (s, 1H), 7.12 (t, J = 9.1 Hz, 1H), 6.84 (d, J = 6.5 Hz, 2H), 6.48 (d, J = 5.9 Hz, 1H), 5.34 (dd, J = 11.8, 4.6 Hz, 1H), 4.36 (br.s, 2H), 3.78 (s, 3H), 3.48 (dd, J = 18.9, 12.2 Hz, 1H), 3.38 (m, 1H), 3.01 (dt, J = 11.6, 10.4 Hz, 2H), 2.75 (dd, J = 18.9, 3.9 Hz, 1H), 1.90 (d, J = 12.6 Hz, 1H), 1.77 (d, J = 12.7 Hz, | 1.63 Method 1 | 402 |
| | | | 1H), 1.46 (m, 2H) | | |
| 100 | (S)-(5- (3,5- difluoro- phenyl)- 4,5- dihydro- 1H- pyrazol- 1-yl)(1-(2- (methyl- amino) pyrimidin- 4-yl) piperidin- 4-yl) methanone | 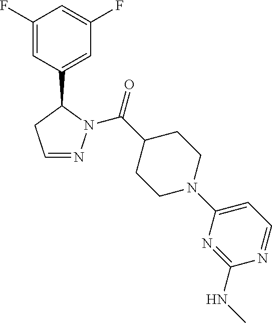 [SMILES: CNc1nccc(N2CCC(C(=O)N3N=CC[C@H]3c3cc(F)cc(F)c3)CC2)n1] | ¹H NMR (400 MHz, DMSO-d6) δ ppm 7.78 (d, J = 5.1 Hz, 1H), 7.24 (s, 1H), 7.12 (m, 1H), 6.83 (d, J = 6.5 Hz, 2H), 6.42 (br.s, 1H), 6.03 (d, J = 5.7 Hz, 1H), 5.34 (dd, J = 11.7, 4.5 Hz, 1H), 4.34 (d, J = 10.4 Hz, 2H), 3.48 (dd, J = 18.8, 12.1 Hz, 1H), 3.37 (m, 1H), 2.91 (dt, J = 11.6, 10.4 Hz, 2H), 2.75 (m, 1H), 2.72 (d, J = 4.7 Hz, 3H), 1.86 (d, J = 10.6 Hz, 1H), 1.73 (d, J = | 1.69 Method 1 | 401 |
| | | | 12.0 Hz, 1H), 1.44 | | |
| | | | (m, 2H) | | |
| 101 | (S)-(5- (2,5- difluoro- phenyl)- 4,5- dihydro- 1H- pyrazol-1- yl)(1-(5- methoxy- pyrimidin- 2-yl) piperidin- 4-yl) methanone | 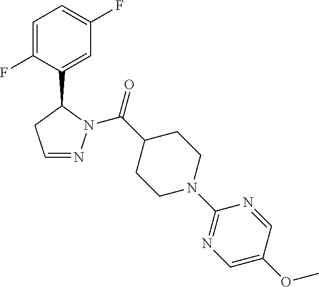 [SMILES: COc1cnc(N2CCC(C(=O)N3N=CC[C@H]3c3cc(F)ccc3F)CC2)nc1] | ¹H NMR (400 MHz, DMSO-d6) δ ppm 8.19 (s, 2H), 7.27 (s, 1H), 7.25 (m, 1H), 7.15 (m, 1H), 6.86 (m, 1H), 5.41 (dd, J = 12.1, 5.3 Hz, 1H, 4.53 (d, J = 12.9 Hz, 2H), 3.76 (s, 3H), 3.51 (dd, J = 18.3, 12.8 Hz, 1H), 3.36 (m, 1H), 2.93 (m, 2H), 2.77 (dd, J = 18.8, 4.6 Hz, 1H), 1.85 (d, J = 11.6 Hz, 1H), 1.72 (d, J = 12.0 Hz, 1H), 1.45 | 2.52 Method 1 | 402 |
| | | | (m, 2H) | | |
| 102 | (S)-2-(4- (5-(2,5- difluoro- phenyl)- 4,5- dihydro- 1H- pyrazole-1- carbonyl) piperidin- 1-yl) pyrimidine- 4- carboxamide | 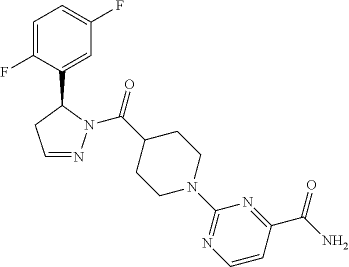 [SMILES: NC(=O)c1ccnc(N2CCC(C(=O)N3N=CC[C@H]3c3cc(F)ccc3F)CC2)n1] | ¹H NMR (400 MHz, DMSO-d6) δ ppm 8.54 (d, J = 4.7 Hz, 1H), 8.17 (s, 1H), 7.72 (s, 1H), 7.29 (s, 1H), 7.25 (dd, J = 9.5, 4.6 Hz, 1H), 7.16 (m, 1H), 7.07 (d, J = 4.7 Hz, 1H), 6.87 (m, 1H), 5.43 (dd, J = 12.0, 5.3 Hz, 1H), 4.80 (d, J = 12.1 Hz, 2H), 3.52 (ddd, J = 18.8, 12.8, 1.1 Hz, 1H), 3.38 (tt, J = 11.6, 3.6 Hz, | 2.23 Method 1 | 415 |
| | | | 1H), 3.00 (m, 2H), | | |
| | | | 2.78 (dd, J = 18.5, | | |
| | | | 4.6 Hz, 1H), 1.90 | | |
| | | | (d, J = 12.5 Hz, | | |
| | | | 1H), 1.77 (d, J = | | |
| | | | 12.6 Hz, 1H), | | |
| | | | 1.47 (m, 2H) | | |
| 103 | (S)-5-(4- (5-(2,5- difluoro- phenyl)- 4,5- dihydro- 1H- pyrazole-1- carbonyl) piperidin- 1-yl) pyrazine- 2- carbonitrile | 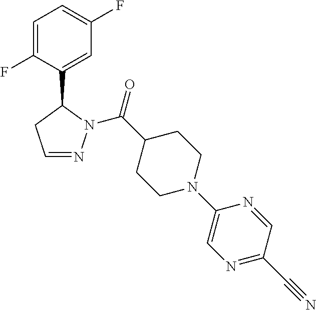 [SMILES: N#Cc1cnc(N2CCC(C(=O)N3N=CC[C@H]3c3cc(F)ccc3F)CC2)cn1] | ¹H NMR (400 MHz, DMSO-d6) δ ppm 8.53 (d, J = 1.3 Hz, 1H), 8.43 (d, J = 1.3 Hz, 1H), 7.26 (m, 2H), 7.16 (m, 1H), 6.88 (m, 1H), 5.42 (dd, J = 12.1, 5.3 Hz, 1H), 4.49 (d, J = 10.8 Hz, 2H), 3.52 (ddd, J = 18.8, 12.1, 1.1 Hz, 1H), 3.43 (tt, J = 11.3, 3.8 Hz, 1H), 3.17 (m, 2H), 2.78 (dd, J = 18.6, 4.7 Hz, 1H), 1.95 (d, J = 13.1 Hz, 1H), 1.82 (d, J = 12.9 Hz, 1H), 1.53 (m, 2H) | 2.49 Method 1 | 397 |
| 104 | (S)-6-(4- (5-(2,5- difluoro- phenyl)- 4,5- dihydro- 1H- pyrazole-1- carbonyl) piperidin- 1-yl) pyrimidine- 4- carbonitrile | 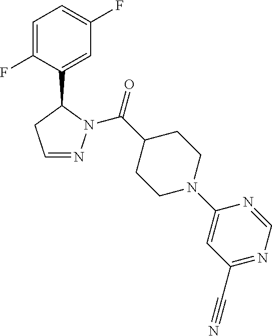 [SMILES: N#Cc1cc(N2CCC(C(=O)N3N=CC[C@H]3c3cc(F)ccc3F)CC2)ncn1] | ¹H NMR (400 MHz, DMSO-d6) δ ppm 8.54 (s, 1H), 7.56 (s, 1H), 7.29 (s, 1H), 7.25 (m, 1H), 7.16 (m, 1H), 6.87 (m, 1H), 5.42 (dd, J = 12.1, 5.3 Hz, 1H), 4.20-4.80 (br.s, 2H), 3.52 (dd, J = 18.5, 12.6 Hz, 1H), 3.42 (m, 1H), 3.13 (m, 2H), 2.78 (dd, J = 18.4, 4.7 Hz, 1H), 1.93 (d, J = 11.6 Hz, 1H), 1.80 (d, J = 11.2 Hz, 1H), 1.50 (m, 2H) | 2.41 Method 1 | 397 |
| 105 | (S)-(5- (2,5- difluoro- phenyl)- 4,5- dihydro- 1H- pyrazol-1- yl)(1-(6- methoxy- pyrimidin- 4-yl) piperidin- 4-yl) methanone | 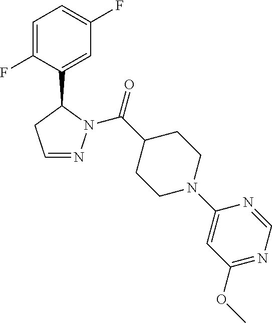 [SMILES: COc1cc(N2CCC(C(=O)N3N=CC[C@H]3c3cc(F)ccc3F)CC2)ncn1] | ¹H NMR (400 MHz, DMSO-d6) δ ppm 8.22 (s, 1H), 7.28 (s 1H), 7.25 (m, 1H), 7.15 (m, 1H), 6.86 (m, 1H), 6.08 (s, 1H), 5.41 (dd, J = 12.1, 5.3 Hz, 1H), 4.34 (m, 2H), 3.81 (s, 3H), 3.51 (ddd, J = 18.8, 5.1, 1.3 Hz, 1H), 3.36 (tt, J = 11.6, 3.8 Hz, 1H), 2.97 (m, 2H), 2.77 (ddd, J = 19.0, 5.3, 1.1 Hz, 1H), 1.86 (d, J = 12.9 Hz, 1H), 1.74 (d, J = 13.3 Hz, 1H), 1.44 (m, 2H) | 2.07 Method 1 | 402 |
| 106 | (S)-ethyl 2-(4-(5- (3,5- difluoro- phenyl)- 4,5- dihydro- 1H- pyrazole-1- carbonyl) piperidin- 1-yl) oxazole-4- carboxylate | 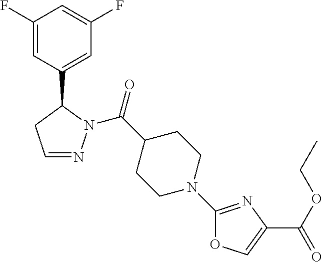 [SMILES: CCOC(=O)c1coc(N2CCC(C(=O)N3N=CC[C@H]3c3cc(F)cc(F)c3)CC2)n1] | ¹H NMR (400 MHz, CDCl3) δ ppm 7.76 (s, 1H), 6.99 (t, J = 1.6 Hz, 1H), 6.69 (m, 3H), 5.32 (dd, J = 12.1, 4.7 Hz, 1H), 4.35 (q, J = 7.2 Hz, 2H), 4.17 (m, 2H), 3.45 (ddd, J = 20.3, 12.0, 1.5 Hz, 1H), 3.31 (tt, J = 11.2, 3.9 Hz, 1H), 3.11 (m, 2H), 2.80 (ddd, J = 19.0, 5.1, 1.7 Hz, 1H), 1.75- | 2.53 Method 1 | 433 |
| | | | 2.00 (m, 4H), 1.36 | | |
| | | | (t, J = 7.1 Hz, 3H) | | |
| 107 | (S)-ethyl 2-(4-(5- (3,5- difluoro- phenyl)- 4,5- dihydro- 1H- pyrazole-1- carbonyl) piperidin- 1-yl) oxazole-5- carboxylate | 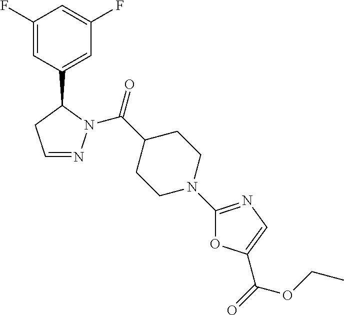 [SMILES: CCOC(=O)c1cnc(N2CCC(C(=O)N3N=CC[C@H]3c3cc(F)cc(F)c3)CC2)o1] | ¹H NMR (400 MHz, CDCl3) δ ppm 7.54 (s, 1H), 7.01 (s, 1H), 6.71 (m, 3H), 5.34 (dd, J = 12.0, 5.1 Hz, 1H), 4.33 (q, J = 7.2 Hz, 2H), 4.29 (m, 2H), 3.46 (ddd, J = 19.0, 12.0, 1.3 Hz, 1H), 3.36 (tt, J = 11.1, 3.8 Hz, 1H), 3.20 (m, 2H), 2.81 (ddd, J = 18.8, 4.9, 1.5 Hz, 1H), 1.75-2.05 (m, 4H), 1.36 (t, J = 7.0 Hz, 3H) | 2.53 Method 1 | 433 |

### Example 108

### (S)-6-(4-(5-(5-fluoropyridin-3-yl)-4,5-dihydro-1H-pyrazole-1-carbonyl)piperidin-1-yl)pyrimidine-4-carboxamide

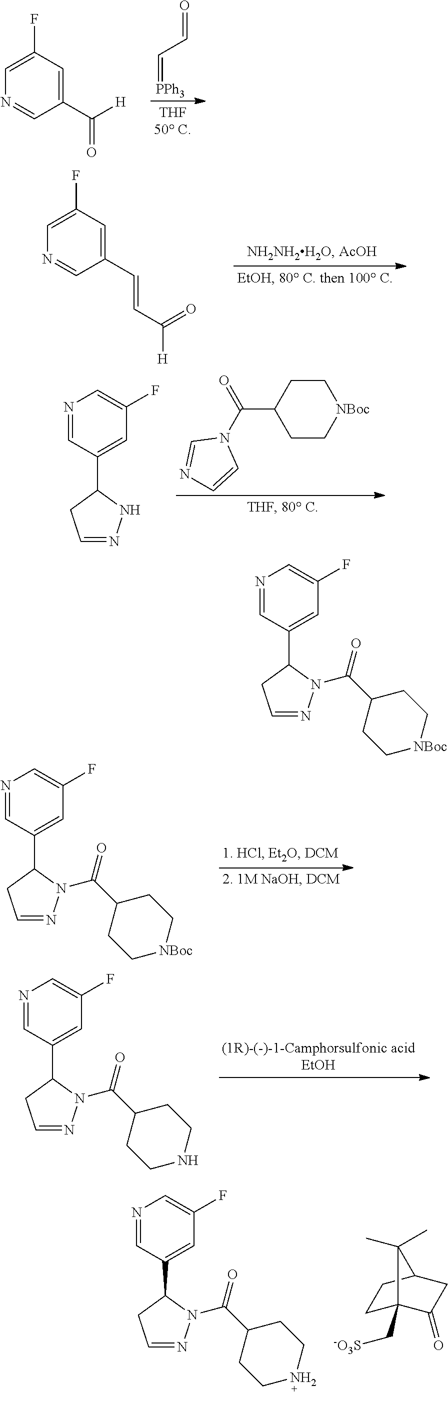

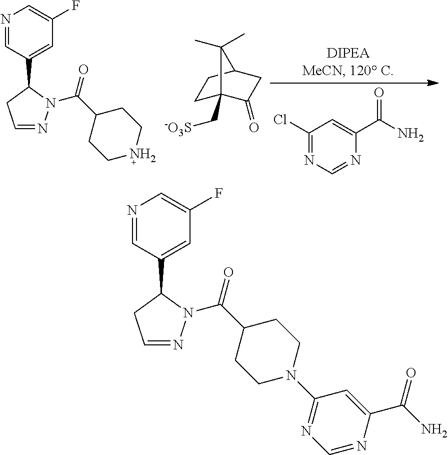

### Step 1

To a solution of 5-fluoronicotinaldehyde (43.8 g, 343 mmol) in THF (300 mL) stirred under argon at rt was added solid 2-(triphenylphosphoranylidene)acetaldehyde (112 g, 360 mmol) portion-wise. The reaction mixture was stirred at 50° C. overnight. The reaction mixture was evaporated in vacuo to give a brown solid. The crude solid was treated with toluene (100 mL) which led to precipitation of triphenylphosphine oxide, which was filtered off and discarded. The resulting filtrate was evaporated to dryness and retreated with toluene (50 mL) which led to more precipitation of triphenylphosphine oxide, which was filtered off and discarded. The resulting filtrate was evaporated to dryness to give an orange oil which crystallized. The residue was recrystallized twice successively in the minimum amount of hot EtOH to afford 3-(5-fluoropyridin-3-yl)acrylaldehyde (26.6 g, 176 mmol, purity: 91%, 9% of triphenylphosphine oxide, recovery: 51%) as light brown crystals. LCMS (m/z) 152 (M+H)⁺, retention time: 1.45 min, Method 1. The filtrate was evaporated and solubilized in hot EtOH (25 mL) and crystallized at rt to afford, after filtration, 3-(5-fluoropyridin-3-yl)acrylaldehyde (8.7 g, 57.4 mmol, purity: 98%, 2% of triphenylphosphine oxide, recovery: 17%) as light brown crystals. LCMS (m/z) 152 (M+H)⁺, retention time: 1.47 min, LC/MS Method 1. ¹H NMR (400 MHz, CDCl₃) δ ppm 9.77 (d, J=7.4 Hz, 1H), 8.61 (s, 1H), 8.55 (d, J=2.7 Hz, 1H), 7.60 (dt, J=8.9, 2.2 Hz, 1H), 7.51 (d, J=15.9 Hz, 1H), 6.79 (dd, J=7.4 Hz, 1H).

### Step 2

To a solution of hydrazine monohydrate (13.5 mL, 278 mmol) in EtOH (300 mL) stirred at 0° C. was added neat acetic acid (17.2 mL, 301 mmol). The reaction mixture was then heated at 45° C. and solid 3-(5-fluoropyridin-3-yl)acrylaldehyde (35 g, 232 mmol) was added portion-wise. The reaction mixture was stirred at 80° C. overnight. The reaction mixture was heated at 100° C. for a further 5 h. The reaction mixture was evaporated in vacuo to give an orange oil. This was treated with water (200 mL) and saturated solution of NaHCO₃ was then added until pH 7 was obtained. This aqueous layer was extracted with DCM (2×300 mL). The combined organic layers were washed with brine, dried over Na₂SO₄, and evaporated in vacuo to afford 3-(4,5-dihydro-1H-pyrazol-5-yl)-5-fluoropyridine (36.8 g, 203 mmol, purity: 91%, 9% of triphenylphosphine oxide, recovery: 88%) as an orange oil with a solid. LCMS (m/z) 166 (M+H)⁺, retention time: 0.88 min, LC/MS Method 1. This solid was used in next reaction without further purification. ¹H NMR (400 MHz, CDCl₃) δ ppm 8.42 (m, 2H), 7.51 (dt, J=9.2, 2.1 Hz, 1H), 6.86 (s, 1H), 4.82 (t, J=10 Hz, 1H), 3.22 (ddd, J=17, 10.8, 1.7 Hz, 1H), 2.68 (ddd, J=17.1, 10, 1.5 Hz, 1H).

### Step 3

To a solution of 3-(4,5-dihydro-1H-pyrazol-5-yl)-5-fluoropyridine (33.5 g, 203 mmol) in THF (500 mL) stirred under nitrogen at rt was added neat tert-butyl 4-(1H-imidazole-1-carbonyl)piperidine-1-carboxylate (56.7 g, 203 mmol) portion-wise. The reaction mixture was stirred at 80° C. for 72 h. DCM (1 L) was added, and the mixture was washed successively with 0.5 M HCl (600 mL), a saturated solution of NaHCO₃ (600 mL), and brine (600 mL). The organic layer was dried over Na₂SO₄ and evaporated in vacuo to give tert-butyl 4-(5-(5-fluoropyridin-3-yl)-4,5-dihydro-1H-pyrazole-1-carbonyl)piperidine-1-carboxylate (60.72 g, 161 mmol, purity: 96%, recovery: 79%) as an orange oil. This oil was used in next reaction without further purification. LCMS (m/z) 377 (M+H)⁺, retention time: 2.30 min, LC/MS Method 1. ¹H NMR (400 MHz, CDCl₃) δ ppm 8.46 (d, J=2.7 Hz, 1H), 8.29 (m, 1H), 7.46 (dt, J=9.8 and 2.3 Hz, 1H), 7.27 (s, 1H), 5.40 (dd, J=12.0 and 5.3 Hz, 1H), 4.96 (dd, J=12.0 and 5.1 Hz, 1H), 4.02-3.80 (m, 4H), 3.51 (ddd, J=19.0, 12.0 and 1.5 Hz, 1H), 3.23 (tt, J=15.0 and 3.6 Hz, 1H), 2.83 (ddd, J=19.0, 5.2 and 1.7 Hz, 1H), 1.91-1.62 (m, 4H), 0.94 (s, 9H).

### Step 4

To a solution of tert-butyl 4-(5-(5-fluoropyridin-3-yl)-4,5-dihydro-1H-pyrazole-1-carbonyl)piperidine-1-carboxylate (60 g, 159 mmol) in DCM (300 mL) stirred under nitrogen at rt was added a solution of 2M HCl in Et₂O (300 mL, 600 mmol) portion-wise. The reaction mixture was stirred at rt overnight. The solid was filtered, washed with acetonitrile, followed by Et₂O to give a brown solid. A solution of 1M NaOH (335 mL, 335 mmol) and DCM (400 mL) were added. The mixture was stirred until complete dissolution of the solid. After separation, the aqueous layer was extracted with DCM (400 mL). The combined organic layers were washed with brine, dried over Na₂SO₄, and evaporated in vacuo to afford (5-(5-fluoropyridin-3-yl)-4,5-dihydro-1H-pyrazol-1-yl)(piperidin-4-yl)methanone (29.6 g, 107 mmol, purity: 100%, recovery: 67%) as a dark brown oil. This oil was used in next reaction without further purification. LCMS (m/z) 277 (M+H)⁺, retention time: 0.37 min, LC/MS Method 2. ¹H NMR (400 MHz, DMSO-d6) δ ppm 8.46 (d, J=2.7 Hz, 1H), 8.28 (s, 1H), 7.45 (dt, J=9.7, 2.3 Hz, 1H), 7.23 (s, 1H), 5.40 (dd, J=12.0, 5.1 Hz, 1H), 3.50 (ddd, J=19.0, 12.0, 1.4 Hz, 1H), 3.13 (tt, J=11.7, 3.7 Hz, 2H), 2.95 (m, 2H), 2.82 (ddd, J=19.0, 5.1, 1.7 Hz, 1H), 2.51 (m, 2H), 1.70 (m, 1H), 1.60 (m, 1H), 1.41 (m, 2H).

### Step 5

To a solution of (5-(5-fluoropyridin-3-yl)-4,5-dihydro-1H-pyrazol-1-yl)(piperidin-4-yl)methanone (30 g, 109 mmol) in EtOH (100 mL) stirred at rt was added solid (1R)-(−)-10-camphorsulfonic acid (22.7 g, 96 mmol). The mixture was heated at reflux. EtOH was added portion-wise until complete dissolution (190 mL) and the mixture was allowed to cool to rt. After 15 h, the solid was filtered off, washed with EtOH, and dried at 50° C. in vacuo to form crude product as cream crystals. Recrystallization from hot ethanol (250 mL), followed by filtratin and ethanol wash afforded partially racemic product with 54 enantiomeric excess by chiral HPLC analysis. A second recrystallization from hot ethanol (250 mL) was done and the product was filtered off when the temperature had dropped to 34° C. The product was washed with EtOH and dried to afford (S)-(5-(5-fluoropyridin-3-yl)-4,5-dihydro-1H-pyrazol-1-yl)(piperidin-4-yl)methanone, (1R)-(−)-10-camphorsulphonic acid salt (10 g, 19.7 mmol, purity: 100%, recovery: 18%). LCMS (m/z) 277 (M+H)⁺, retention time: 0.38 min, LC/MS Method 2. Chiral HPLC Method 2: 10.6 and 12.8 min, % ee=97.9%. A second batch subsequently crystallized out from the filtrate on standing and this was filtered off to yield more product (3.46 g, 6.8 mmol, purity: 100%, recovery: 6%). Chiral HPLC Method 2: 10.7 and 12.8 min, % ee=98.6%. ¹H NMR (400 MHz, DMSO-d6) δ ppm 8.49 (d, J=2.7 Hz, 1H), 8.46 (br s, 2H), 8.29 (m, 1H), 7.49 (dt, J=9.6, 2.1 Hz, 1H), 7.31 (s, 1H), 5.41 (dd, J=9.6, 2.1 Hz, 1H), 3.53 (ddd, J=19.0, 12.0, 1.4 Hz, 1H), 3.38-3.24 (m, 3H), 3.04-2.94 (m, 2H), 2.90 (d, J=14.6 Hz, 1H), 2.85 (ddd, J=19.1, 5.2, 1.5 Hz, 1H), 2.70-2.61 (m, 1H), 2.40 (d, J=14.6 Hz, 1H), 2.25 (dt, J=18.1, 3.9 Hz, 1H), 2.02-1.92 (m, 2H), 1.90-1.83 (m, 2H), 1.80 (d, J=18 Hz, 1H), 1.35-1.23 (m, 2H), 1.04 (s, 3H), 0.74 (s, 3H).

### Step 6

To a suspension of (S)-(5-(5-fluoropyridin-3-yl)-4,5-dihydro-1H-pyrazol-1-yl)(piperidin-4-yl)methanone, 1R-(−)-camphor-10-sulphonic acid salt (500 mg, 0.983 mmol) in MeCN (10 mL) was added 6-chloropyrimidine-4-carboxamide (155 mg, 0.98 mmol) and DIPEA (0.429 mL, 2.46 mmol). The reaction was sealed and stirred at 120° C. for 2 h. The reaction mixture was evaporated in vacuo. The residue was purified by normal phase column chromatography (DCM/MeOH 100/0 to 95/5). The appropriate fractions were combined, concentrated in vacuo, and recrystallized from MeCN to afford (S)-6-(4-(5-(5-fluoropyridin-3-yl)-4,5-dihydro-1H-pyrazole-1-carbonyl)piperidin-1-yl)pyrimidine-4-carboxamide (200 mg, 0.503 mmol, purity: 100%, recovery: 51%) as a white powder. LCMS (m/z) 398 (M+H)⁺, retention time: 1.52 min, LC/MS Method 1. ¹H NMR (400 MHz, DMSO-d6) δ ppm 8.54 (s, 1H), 8.47 (d, J=2.6 Hz, 1H), 8.29 (s, 1H), 8.05 (s, 1H), 7.74 (s, 1H), 7.48 (dd, J=9.8, 1.8 Hz, 1H), 7.30 (s, 2H), 5.41 (dd, J=12.2, 5.2 Hz, 1H), 4.45 (m, 2H), 3.53 (ddd, J=19.0, 12.1, 1.1 Hz, 1H), 3.41 (m, 1H), 3.09 (m, 2H), 2.84 (dd, J=19.0, 5.2, 1.5 Hz, 1H), 1.91 (d, J=12.0 Hz, 1H), 1.82 (d, J=12.2 Hz, 1H), 1.47 (sextuplet of d, J=12.5, 3.6 Hz, 2H).

### Step 7a

### Compound A—Form 1: Small Scale Purification

0.8 g of (S)-6-(4-(5-(5-fluoropyridin-3-yl)-4,5-dihydro-1H-pyrazole-1-carbonyl)piperidin-1-yl)pyrimidine-4-carboxamide was mixed at 1000 rpm in 20 mL ACN at 25° C. for approximately 2 hours. The suspension was then filtered under vacuum. The mother liquor was collected. 0.5 g (S)-6-(4-(5-(5-fluoropyridin-3-yl)-4,5-dihydro-1H-pyrazole-1-carbonyl)piperidin-1-yl)pyrimidine-4-carboxamide was suspended in the mother liquor. The suspension was mixed at 25° C. for approximately 5 days. Then the suspension was filtered with a centrifuge with a rotation speed of 14000 RPM for 2×1 min. The resulting wet cake was dried in a vacuum oven at approximately 50° C. for approximately 4 hours and was further dried under vacuum without heating for approximately 2 days to afford crystalline (S)-6-(4-(5-(5-fluoropyridin-3-yl)-4,5-dihydro-1H-pyrazole-1-carbonyl)piperidin-1-yl)pyrimidine-4-carboxamide (Compound A—Form 1).

The X-ray powder diffraction (XRPD) pattern of this material (Compound A—Form 1) is shown in FIG. 13. A summary of the diffraction angles and d-spacings is given in Table I below. The XRPD analysis was conducted on a Rigaku X-ray powder diffractometer, model Miniflex II using Rigaku MiniFlex II SC (Scintillator Counter) detector. The acquisition conditions included: Cu K_{α} radiation, generator tension: 30 kV, generator current: 15 mA, start angle: 2.0° 2θ, end angle: 40.0° 2θ, step width: 0.040° 2θ per step, count time: 0.5 seconds. The sample was prepared by packing a few milligrams of material on a zero background sample holder, gently flattened using a glass slide resulting in a thin layer of powder.

TABLE I

| Diff. Angle [°2θ] | d-spacing [Å] |
|---|---|
| 13.2 | 6.7 |
| 18.5 | 4.8 |
| 21.7 | 4.1 |
| 22.7 | 3.9 |
| 26.3 | 3.4 |
| 27.7 | 3.2 |

The differential scanning calorimetry (DSC) thermogram of the title compound was recorded on a TA Q2000 Differential Scanning calorimeter and is shown in FIG. 14. The experiment was conducted using a heating rate of 10° C./min in a non-hermetically sealed aluminum pan. The DSC thermogram of Compound A—Form 1 exhibits an endotherm with an onset temperature at about 195° C. A person skilled in the art would recognize that the onset temperature of the endotherm may vary depending on the experimental conditions.

### Step 7b

### Compound A—Form 1: Scale-Up Purification

3.5 g of (S)-6-(4-(5-(5-fluoropyridin-3-yl)-4,5-dihydro-1H-pyrazole-1-carbonyl)piperidin-1-yl)pyrimidine-4-carboxamide was mixed at 1000 rpm in 25 mL ACN at 25° C. for approximately 5 days. Then the suspension was filtered under vacuum. The mother liquor was utilized to rinse the cake. The resulting wet cake was dried in a vacuum oven at approximately 50° C. for approximately 15 hours to afford crystalline (S)-6-(4-(5-(5-fluoropyridin-3-yl)-4,5-dihydro-1H-pyrazole-1-carbonyl)piperidin-1-yl)pyrimidine-4-carboxamide (Compound A—Form 1).

The X-ray powder diffraction (XRPD) pattern of this material (Compound A—Form 1) is concordant with that of the crystalline (S)-6-(4-(5-(5-fluoropyridin-3-yl)-4,5-dihydro-1H-pyrazole-1-carbonyl)piperidin-1-yl)pyrimidine-4-carboxamide (Compound A—Form 1) obtained in Step 7a (small scale purification).

### Step 7c

### Compound A—Form 2

0.2 g of (S)-6-(4-(5-(5-fluoropyridin-3-yl)-4,5-dihydro-1H-pyrazole-1-carbonyl)piperidin-1-yl)pyrimidine-4-carboxamide was dissolved in 0.5 ml DMSO (2.5 volumes) at 40° C. The solution was then filtered through 0.2-micron syringe filter. The filtrate was seeded with 1 mg of Form 2 seeds. The seeded solution was cooled down to 5° C. and maintained at 5° C. for three days. The solids were isolated by filtration. Filter cake was washed with ˜1 ml MTBE (methyl t-butyl ether) and dried under vacuum at ambient temperature for no less than 16 hours.

### Form 2 Seed Preparation

0.3 g of (S)-6-(4-(5-(5-fluoropyridin-3-yl)-4,5-dihydro-1H-pyrazole-1-carbonyl)piperidin-1-yl)pyrimidine-4-carboxamide was added in 0.4 ml of DMSO, which is pre-heated to 40° C. to prepare a saturated solution. The solution was filtered through 0.2 micron syringe filter to remove the un-dissolved solid. Filtrate was rapidly cooled down to 4° C. and held at 4° C. for five days. Solids were harvested via filtration. The filter cake was rinsed with MTBE to remove DMSO residual. The rinse filter cake was dried under vacuum at ambient temperature for no less than 16 hours.

The X-ray powder diffraction (XRPD) pattern of this material (Form 2) is shown in FIG. 15. A summary of the diffraction angles and d-spacings is given in Table II below. The XRPD analysis was conducted on a Panalytical X-ray powder diffractometer. The acquisition conditions included: Cu K_{α} radiation, generator tension: 40 kV, generator current: 40 mA, start angle: 2.0° 2θ, end angle: 40.0° 2θ, step width: 0.0167° 2θ per step, 32 second per step. The sample was prepared by packing ˜10 mg of material on a zero-background silicone wafer holder, gently flattened using a glass slide resulting in a thin layer of powder.

| Diffraction Position. [°2θ] | d-spacing [Å] |
|---|---|
| 8.3935 | 10.53458 |
| 14.6901 | 6.03027 |
| 14.8705 | 5.95751 |
| 15.253 | 5.80899 |
| 18.8439 | 4.70935 |
| 19.1686 | 4.6303 |
| 20.8422 | 4.26212 |
| 25.2576 | 3.52616 |

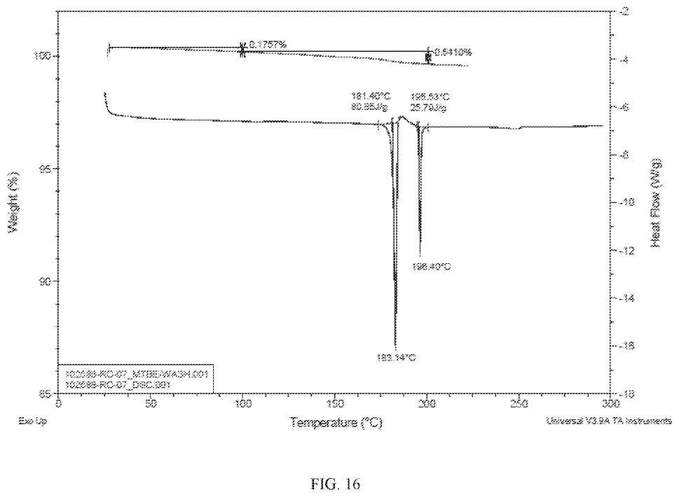

The differential scanning calorimetry (DSC) thermogram of the title compound Form 2 was recorded on a TA Q2000 Differential Scanning calorimeter and is shown in FIG. 16. The analysis was conducted using a heating rate of 10° C./min in a non-hermetically crimped aluminum pan. The DSC thermogram of Compound A—Form 2 exhibits the first major endotherm with an onset temperature at about 181.4° C., followed by a minor endotherm with an onset temperature at 195.5° C. A person skilled in the art would recognize that the onset temperature of the endotherm may vary depending on the experimental conditions.

Examples 109-117 were synthesized using steps 1-6 in an analogous manner. For step 6, DIPEA may be substituted for TEA.

| | | | | LC: | |
|---|---|---|---|---|---|
| | | | | retention | |
| | | | | time | |
| | | | | (min); | |
| | | | | LC/MS | |
| | | | | Method of | MS |
| Ex. | Name | Structure | ¹H NMR | analysis | (M + H)⁺ |
| 109 | (S)-2-(4- (5-(5- fluoro- pyridin- 3-yl)-4,5- dihydro- 1H- pyrazole-1- carbonyl) piperidin- 1-yl) pyrimidine- 4- carboxamide | 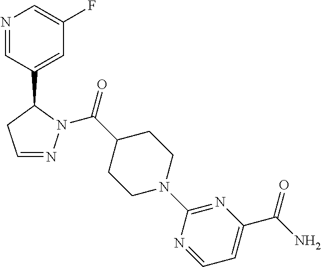 [SMILES: NC(=O)c1ccnc(N2CCC(C(=O)N3N=CC[C@H]3c3cncc(F)c3)CC2)n1] | ¹H NMR (400 MHz, DMSO-d6) δ ppm 8.54 (d, J = 4.7 Hz, 1H), 8.47 (d, J = 2.7 Hz, 1H), 8.29 (s, 1H), 8.17 (s, 1H), 7.73 (s, 1H), 7.47 (m, 1H), 7.29 (s, 1H), 7.07 (d, J = 4.7 Hz, 1H), 5.42 (dd, J = 12.0, 5.1 Hz, 1H), 4.80 (d, J = 12.5 Hz, 2H), 3.53 (ddd, J = 19.0, 12.0, 1.3 Hz, 1H), 3.38 (tt, J = 11.6, 3.6 | 1.82 Method 1 | 398 |
| | | | Hz, 1H), 3.00 (m, | | |
| | | | 2H), 2.85 (ddd, | | |
| | | | J = 19.0, 5.1, 1.7 | | |
| | | | Hz, 1H), 1.89 (d, | | |
| | | | J = 11.4 Hz, 1H), | | |
| | | | 1.78 (d, J = 11.2 | | |
| | | | Hz, 1H), 1.47 | | |
| | | | (m, 2H) | | |
| 110 | (S)-(5-(5- fluoro- pyridin-3- yl)-4,5- dihydro- 1H- pyrazol-1- yl)(1-(2- methoxy- pyrimidin- 4-yl) piperidin- 4-yl) methanone | 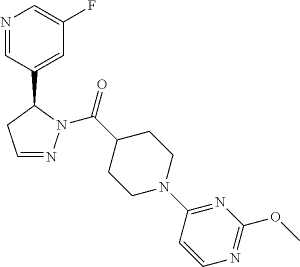 [SMILES: COc1nccc(N2CCC(C(=O)N3N=CC[C@H]3c3cncc(F)c3)CC2)n1] | ¹H NMR (400 MHz, DMSO-d6) δ ppm 8.47 (d, J = 2.7 Hz, 1H), 8.29 (s, 1H), 7.98 (d, J = 6.1 Hz, 1H), 7.47 (m, 1H), 7.29 (s, 1H), 6.47 (d, J = 6.1 Hz, 1H), 5.41 (dd, J = 12.0, 5.1 Hz, 1H), 4.35 (s, 2H), 3.77 (s, 3H), 3.52 (dd, J = 19.0, 12.0, 1.3 Hz, 1H), 3.38 (tt, J = 11.4, 3.7 Hz, 1H), 3.00 (m, 2H), 2.84 (ddd, J = 19.0, 5.1, | 1.30 Method 1 | 385 |
| | | | 1.5 Hz, 1H), 1.88 | | |
| | | | (d, J = 11.2 Hz, | | |
| | | | 1H), 1.77 (d, J = | | |
| | | | 11.2 Hz, 1H), 1.45 | | |
| | | | (m, 2H) | | |
| 111 | (S)-6-(4- (5-(5- fluoro- pyridin- 3-yl)-4,5- dihydro- 1H- pyrazole-1- carbonyl) piperidin- 1-yl) pyrimidine- 4- carbonitrile | 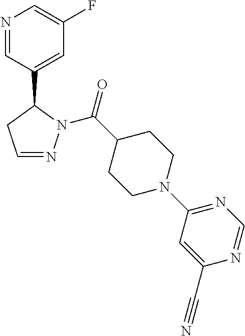 [SMILES: N#Cc1cc(N2CCC(C(=O)N3N=CC[C@H]3c3cncc(F)c3)CC2)ncn1] | ¹H NMR (400 MHz, DMSO-d6) δ ppm 8.54 (s, 1H), 8.48 (d, J = 2.7 Hz, 1H), 8.29 (s, 1H), 7.56 (s, 1H), 7.47 (d, J = 9.7 Hz, 1H), 7.30 (s, 1H), 5.41 (dd, J = 12.0, 5.1 Hz, 1H), 4-5.00 (m, 2H), 3.53 (ddd, J = 19.0, 12.0, 1.3 Hz, 1H), 3.42 (tt, J = 11.3, 3.6 Hz, 1H), 3.12 (s, 2H), 2.85 (ddd, J = 19.1, 5.3, 1.5 Hz, 1H), 1.92 (d, J = 11.8 Hz, 1H), 1.81 (d, J = 11.6 Hz, 1H), 1.49 (m, 2H) | 1.98 Method 1 | 380 |
| 112 | (S)-(1-(4- amino-5- fluoro- pyrimidin- 2-yl) piperidin- 4-yl)(5- (5-fluoro- pyridin- 3-yl)-4,5- dihydro- 1H- pyrazol- 1-yl) | 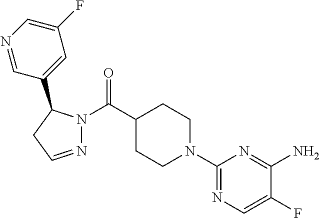 [SMILES: Nc1nc(N2CCC(C(=O)N3N=CC[C@H]3c3cncc(F)c3)CC2)ncc1F] | ¹H NMR (400 MHz, DMSO-d6) δ ppm 8.48 (d, J = 2.3 Hz, 1H), 8.28 (s, 1H), 7.78 (d, J = 3.4 Hz, 1H), 7.46 (d, J = 9.7 Hz, 1H), 7.27 (s, 1H), 6.80 (s, 2H), 5.40 (dd, J = 12.0, 5.1 Hz, 1H), 4.51 (d, J = 11.8 Hz, 2H), 3.51 (dd, J = 18.8, 12.3 Hz, 1H), 3.29 (m, 1H), | 1.84 Method 1 | 388 |
| | methanone | | 2.83 (dd, J = 18.6, | | |
| | | | 4.9 Hz, 3H), 1.79 | | |
| | | | (d, J = 12.0 Hz, | | |
| | | | 1H), 1.70 (d, J = | | |
| | | | 12.0 Hz, 1H), 1.40 | | |
| | | | (m, 2H) | | |
| 113 | (S)-(5-(5- fluoro- pyridin- 3-yl)-4,5- dihydro- 1H- pyrazol-1- yl)(1-(6- methoxy- pyrimidin- 4-yl) piperidin- 4-yl) methanone | 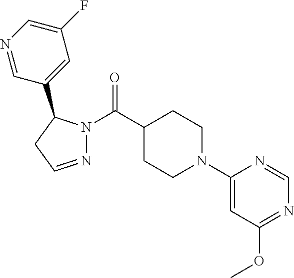 [SMILES: COc1cc(N2CCC(C(=O)N3N=CC[C@H]3c3cncc(F)c3)CC2)ncn1] | ¹H NMR (400 MHz, DMSO-d6) δ ppm 8.47 (d, J = 2.5 Hz, 1H), 8.29 (s, 1H), 8.22 (s, 1H), 7.47 (d, J = 9.7 Hz, 1H), 7.29 (s, 1H), 6.08 (s, 1H), 5.41 (dd, J = 12.0, 5.1 Hz, 1H), 4.33 (m, 2H), 3.81 (s, 3H), 3.52 (dd, J = 18.6, 12.3 Hz, 1H), 3.36 (m, 1H), 2.96 (m, 2H), 2.84 (dd, J = 19.0, 4.0 Hz, 1H), 1.85 (d, J = 12.1 Hz, | 2.63 Method 1 | 385 |
| | | | 1H), 1.74 (d, J = | | |
| | | | 12.0 Hz, 1H), 1.44 | | |
| | | | (m, 2H) | | |
| 114 | (S)-(5-(5- fluoro- pyridin-3- yl)-4,5- dihydro- 1H- pyrazol-1- yl)(1-(2- (methylthio) pyrimidin- 4-yl) piperidin- 4-yl) methanone | 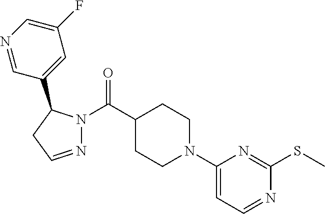 [SMILES: CSc1nccc(N2CCC(C(=O)N3N=CC[C@H]3c3cncc(F)c3)CC2)n1] | ¹H NMR (400 MHz, DMSO-d6) δ ppm 8.47 (d, J = 2.5 Hz, 1H), 8.29 (s, 1H), 7.99 (d, J = 6.1 Hz, 1H), 7.47 (d, J = 9.5 Hz, 1H), 7.29 (s, 1H), 6.54 (d, J = 6.1 Hz, 1H), 5.41 (dd, J = 12.0, 5.1 Hz, 1H), 4.36 (s, 2H), 3.52 (dd, J = 18.5, 12.4 Hz, 1H), 3.38 (m, 1H), | 2Method- hode 2 | 401 |
| | | | 3.01 (m, 2H), 2.84 | | |
| | | | (dd, J = 18.4, 4.6 | | |
| | | | Hz, 1H), 2.41 (s, | | |
| | | | 3H), 1.89 (d, J = | | |
| | | | 12.0 Hz, 1H), 1.78 | | |
| | | | (d, J = 12.0 Hz, | | |
| | | | 1H), 1.45 (m, 2H) | | |
| 115 | (S)-4-(4- (5-(5- fluoro- pyridin-3- yl)-4,5- dihydro- 1H- pyrazole-1- carbonyl) piperidin- 1-yl) pyrimidine- 2- | 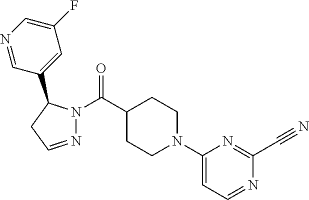 [SMILES: N#Cc1nccc(N2CCC(C(=O)N3N=CC[C@H]3c3cncc(F)c3)CC2)n1] | ¹H NMR (400 MHz, DMSO-d6) δ ppm 8.47 (d, J = 2.7 Hz, 1H), 8.29 (s, 1H), 8.23 (d, J = 6.5 Hz, 1H), 7.48 (m, 1H), 7.30 (s, 1H), 7.11 (d, J = 6.5 Hz, 1H), 5.41 (dd, J = 12.0, 5.3 Hz, 1H), 4.37 (s, 2H), 3.53 (ddd, J = 19.0, 13.2, 1.0 Hz, 1H), 3.41 (tt, | 3.65 Method 2 | 380 |
| | carbonitrile | | J = 11.2, 3.7 Hz, | | |
| | | | 1H), 3.11 (m, 2H), | | |
| | | | 2.85 (ddd, J = 19.0, | | |
| | | | 5.1, 1.5 Hz, 1H), | | |
| | | | 1.93 (d, J = 11.8 | | |
| | | | Hz, 1H), 1.82 (d, | | |
| | | | J = 11.8 Hz, 1H), | | |
| | | | 1.49 (m, 2H) | | |
| 116 | (S)-(5-(5- fluoro- pyridin-3- yl)-4,5- dihydro- 1H- pyrazol- 1-yl)(1- (pyrazolo [1,5-a] pyrimidin- 5-yl) piperidin- 4-yl) methanone | 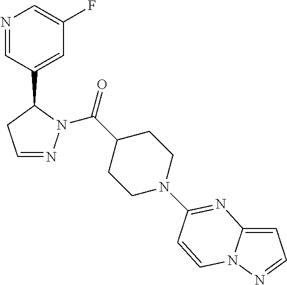 [SMILES: O=C(C1CCN(c2ccn3nccc3n2)CC1)N1N=CC[C@H]1c1cncc(F)c1] | ¹H NMR (400 MHz, DMSO-d6) δ ppm 8.61 (d, J = 9.0 Hz, 1H), 8.47 (d, J = 2.7 Hz, 1H), 8.29 (s, 1H), 7.83 (d, J = 2.1 Hz, 1H), 7.47 (dt, J = 9.6, 2.2 Hz, 1H), 7.29 (s, 1H), 6.72 (d, J = 7.8 Hz, 1H), 6.01 (d, J = 2.1 Hz, 1H), 5.41 (dd, J = 12.0, 5.1 Hz, 1H), 4.42 (m, 2H), 3.52 (ddd, J = 19.0, 12.0, 1.3 Hz, 1H), 3.39 (tt, J = 11.5, 3.8 Hz, | 3.16 Method 2 | 394 |
| | | | 1H), 3.05 (m, 2H), | | |
| | | | 2.85 (ddd, J = 19.0, | | |
| | | | 5.3 1.7 Hz, 1H), | | |
| | | | 1.90 (d, J = 12.96 | | |
| | | | Hz, 1H), 1.79 (d, | | |
| | | | J = 13.0 Hz, 1H), | | |
| | | | 1.50 (m, 2H) | | |
| 117 | (S)-(5-(5- fluoro- pyridin-3- yl)-4,5- dihydro- 1H- pyrazol-1- yl)(1- (imidazo [1,2-b] pyridazin- 6-yl) piperidin- 4-yl) | 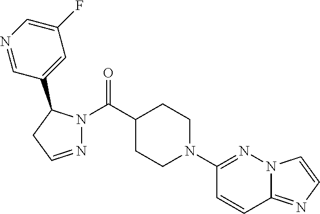 [SMILES: O=C(C1CCN(c2ccc3nccn3n2)CC1)N1N=CC[C@H]1c1cncc(F)c1] | ¹H NMR (400 MHz, DMSO-d6) δ ppm 8.47 (d, J = 2.7 Hz, 1H), 8.29 (s, 1H), 7.88 (s, 1H), 7.81 (d, J = 10.1 Hz, 1H), 7.48 (m, 2H), 7.29 (s, 1H), 7.16 (d, J = 9.9 Hz, 1H), 5.41 ((dd, J = 12.0, 5.1 Hz, 1H), 4.16 (d, J = 13.1 Hz, 2H), 3.52 (ddd, J = 19.0, 12.0, 1.3 | 2.16 Method 2 | 394 |
| | methanone | | Hz, 1H), 3.35 (m, | | |
| | | | 1H), 2.99 (m, 2H), | | |
| | | | 2.84 (ddd, J = 19.0, | | |
| | | | 5.1, 1.3 Hz, 1H), | | |
| | | | 1.90 (d, J = 11.8 | | |
| | | | Hz, 1H), 1.79 (d, | | |
| | | | J = 11.6 Hz, 1H), | | |
| | | | 1.60 (m, 2H) | | |

### Example 118

### (S)—N-(2-(4-(5-(2,5-difluorophenyl)-4,5-dihydro-1H-pyrazole-1-carbonyl)piperidin-1-yl)pyrimidin-4-yl)acetamide

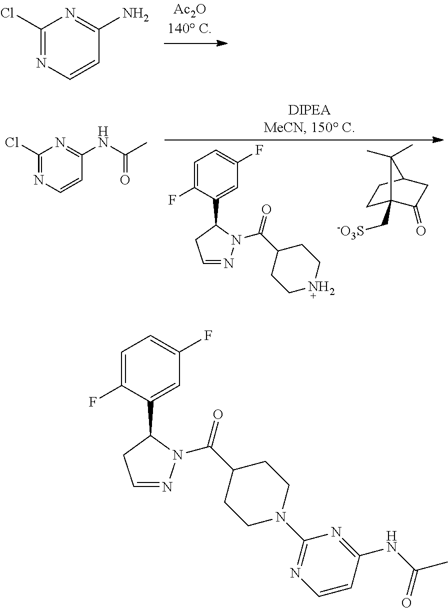

### Step 1

4-Amino-2-chloropyrimidine (1.3 g, 10.03 mmol) was suspended in Ac₂O (9.47 mL, 100 mmol) then heated at 140° C. for 3 h. The reaction was cooled to rt then Et₂O (50 mL) was added and the resulting precipitate was filtered off. This solid was purified by normal phase column chromatography [CyH/(EtOAc/EtOH 3:1) 90/10 to 50/50] to afford N-(2-chloropyrimidin-4-yl)acetamide (500 mg, 2.91 mmol, purity: 100%, recovery: 29%) as a white powder. LCMS (m/z) 172 and 174 (M+H)⁺, retention time: 1.29 min, LC/MS Method 1.

### Step 2

To a suspension of (S)-(5-(2,5-difluorophenyl)-4,5-dihydro-1H-pyrazol-1-yl)(piperidin-4-yl)methanone, 1R-(−)-camphor-10-sulphonic acid salt (250 mg, 0.476 mmol) in MeCN (2.5 mL) was added N-(2-chloropyrimidin-4-yl)acetamide (90 mg, 0.523 mmol) and DIPEA (0.25 mL, 1.43 mmol). The vessel was sealed, and heated with stirring at 150° C. for 24 h. The reaction mixture was evaporated in vacuo. This residue was purified by normal phase column chromatography [CyH/(EtOAc/EtOH 3:1) 90/10 to 50/50] to afford (S)—N-(2-(4-(5-(2,5-difluorophenyl)-4,5-dihydro-1H-pyrazole-1-carbonyl)piperidin-1-yl)pyrimidin-4-yl)acetamide (100 mg, 0.23 mmol, purity: 100%, recovery: 49%) as a cream powder. LCMS (m/z) 429 (M+H)⁺, retention time: 1.80 min, LC/MS Method 1. ¹H NMR (400 MHz, DMSO-d6) δ ppm 10.30 (s, 1H), 8.20 (d, J=5.5 Hz, 1H), 7.28 (s, 1H), 7.26 (m, 1H), 7.21 (d, J=5.5 Hz, 1H), 7.16 (m, 1H), 6.87 (m, 1H), 5.42 (dd, J=12.1, 5.3 Hz, 1H), 4.65 (d, J=12.7 Hz, 2H), 3.52 (ddd, J=18.8, 12.2, 1.1 Hz, 1H), 3.36 (tt, J=11.4, 3.6 Hz, 1H), 2.95 (m, 2H), 2.77 (dd, J=18.5, 436 Hz, 1H), 2.08 (s, 3H), 1.85 (d, J=12.2 Hz, 1H), 1.75 (d, J=12.7 Hz, 1H), 1.44 (m, 2H).

Examples 119 and 120 were synthesized in an analogous manner.

| | | | | LC: | |
|---|---|---|---|---|---|
| | | | | retention | |
| | | | | time | |
| | | | | (min); | |
| | | | | LC/MS | |
| | | | | Method of | MS |
| Ex. | Name | Structure | ¹H NMR | analysis | (M + H)⁺ |
| 119 | (S)-N- (6-(4-(5- phenyl- 4,5- dihydro- 1H- pyrazole-1- carbonyl) piperidin- 1-yl) pyrimidin- 4-yl) acetamide | 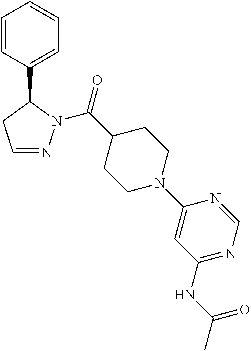 [SMILES: CC(=O)Nc1cc(N2CCC(C(=O)N3N=CC[C@H]3c3ccccc3)CC2)ncn1] | ¹H NMR (400 MHz, DMSO-d6) δ ppm 10.41 (s, 1H), 8.26 (s, 1H), 7.41 (s, 1H), 7.32 (dd, J = 7.6, 7.2 Hz, 2H), 7.23 (m, 2H), 7.11 (d, J = 7.2 Hz, 2H), 5.31 (dd, J = 11.8, 4.4 Hz, 1H), 4.28 (d, J = 11.2 Hz, 2H), 3.50 (dd, J = 18.4, 12.5 Hz, 1H), 3.40 (tt, J = 11.4, 3.6 Hz, 1H), 3.03 (m, 2H), 2.68 (dd, J = 18.9, 3.1 Hz, 1H), 2.07 (s, 3H), 1.90 (d, J = 11.8 Hz, 1H), 1.78 (d, J = 12.0 Hz, 1H), 1.48 (m, 2H) | 1.65 Method 1 | 393 |
| 120 | (S)-N-(6- (4-(5-(3,5- difluoro- phenyl)- 4,5- dihydro- 1H- pyrazole- 1- carbonyl) piperidin- 1-yl) pyrimidin- 4-yl) acetamide | 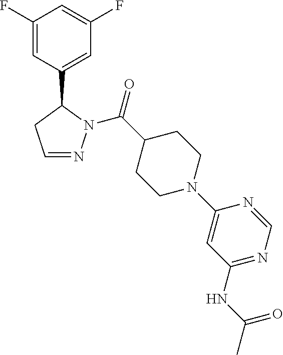 [SMILES: CC(=O)Nc1cc(N2CCC(C(=O)N3N=CC[C@H]3c3cc(F)cc(F)c3)CC2)ncn1] | ¹H NMR (400 MHz, DMSO-d6) δ ppm 10.41 (s, 1H), 8.26, (s, 1H), 7.41 (s, 1H), 7.25 (s, 1H), 7.12 (tt, J = 9.3, 2.3 Hz, 1H), 6.84 (m, 2H), 5.34 (dd, J = 12.0, 4.9 Hz, 1H), 4.28 (m, 2H), 3.48 (ddd, J = 19.0, 12.0, 1.3 Hz, 1H), 3.40 (tt, J = 11.4, 3.8 Hz, 1H), 3.02 (m, 2H), 2.75 (ddd, J = 19.2, 5.1, 1.7 Hz, 1H), 2.08 (s, 3H), 1.92 (d, J = 12.2 Hz, 1H), 1.79 (d, J = 11.8 Hz, 1H), 1.47 (m, 2H) | 1.78 Method 1 | 429 |

### Example 121

### (S)-(1-(5-fluoro-4-(4-methylpiperazin-1-yl)pyrimidin-2-yl)piperidin-4-yl)(5-phenyl-4,5-dihydro-1H-pyrazol-1-yl)methanone

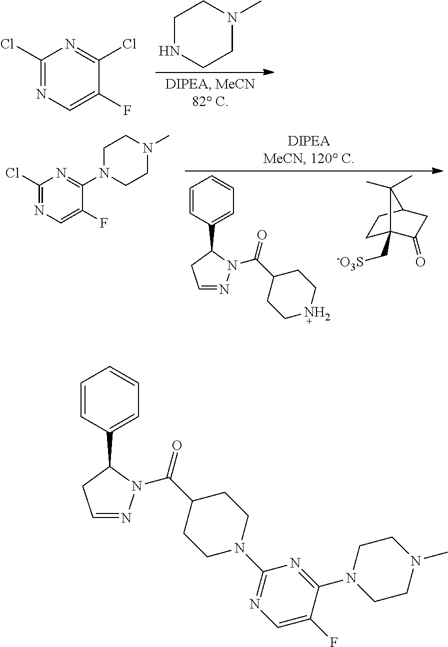

### Step 1

To a solution of 2,4-dichloro-5-fluoropyrimidine (1.12 g, 6.7 mmol) in MeCN (70 mL) stirred at rt was added 1-methylpiperazine (0.86 mL, 7.4 mmol) and DIPEA (1.8 mL, 10.1 mmol) in one charge. The reaction mixture was stirred at 82° C. for 15 h and then evaporated in vacuo to give an orange solid. The solid was dissolved in EtOAc (100 mL) and washed successively with a saturated solution of ammonium chloride in water (20 mL) and water (20 mL). After separation, the aqueous layer was extracted with EtOAc (2×50 mL). The combined organic layers were washed with brine, dried over sodium sulfate, and evaporated in vacuo to give an orange solid. The aqueous layer was extracted again with DCM (3×100 mL). The combined organic layers were washed with brine, dried over sodium sulfate, and evaporated in vacuo to afford an orange solid. The combined residues were purified by normal phase column chromatography [CyH/(EtOH/EtOAc 4:1) 100/0 to 40/60] to afford 2-chloro-5-fluoro-4-(4-methylpiperazin-1-yl)pyrimidine (540 mg, 2.3 mmol, purity: 100%, recovery: 35%) as an orange solid. ¹H NMR (400 MHz, DMSO-d6) δ ppm 8.20 (d, J=6.4 Hz, 1H), 3.71 (t, J=5.0 Hz, 4H), 2.41 (t, J=5.0 Hz, 4H), 2.21 (s, 3H).

### Step 2

To a suspension of (S)-(5-phenyl-4,5-dihydro-1H-pyrazol-1-yl)(piperidin-4-yl)methanone, (1R)-10-camphorsulphonate salt (200 mg, 0.41 mmol) in MeCN (2.5 mL) was added 2-chloro-5-fluoro-4-(4-methylpiperazin-1-yl)pyrimidine (104 mg, 0.45 mmol) and DIPEA (0.18 mL, 1.02 mmol). The vessel was sealed and heated with stirring at 120° C. for 72 h. The reaction mixture was evaporated in vacuo. The crude was purified by column chromatography on NH₂ silica [(EtOH/EtOAc 4:1)/CyH 0-25%] to afforded (S)-(1-(5-fluoro-4-(4-methylpiperazin-1-yl)pyrimidin-2-yl)piperidin-4-yl)(5-phenyl-4,5-dihydro-1H-pyrazol-1-yl)methanone (76 mg, 0.17 mmol, purity: 95%, recovery: 41%) as a light yellow residue. LCMS (m/z) 452 (M+H)⁺, retention time: 1.28 min, LC/MS Method 1. ¹H NMR (400 MHz, DMSO-d6) δ ppm 7.89 (d, J=6.6 Hz, 1H), 7.32 (dd, J=7.6, 7.2 Hz, 2H), 7.23 (m, 2H), 7.10 (d, J=7.2 Hz, 2H), 5.30 (dd, J=11.8, 4.6 Hz, 1H), 4.50 (d, J=13.1 Hz, 2H), 3.60 (m, 4H), 3.48 (ddd, J=18.8, 12.0, 1.1 Hz, 1H), 3.31 (tt, J=11.5, 3.6 Hz, 1H), 2.87 (m, 2H), 2.67 (J=18.8, 4.6, 1.5 Hz, 1H), 2.37 (m, 4H), 2.19 (s, 3H), 1.83 (d, J=12.8 Hz, 1H), 1.70 (d, J=12.8 Hz, 1H), 1.44 (m, 2H).

### Example 122

### (S)-(1-(2-(dimethylamino)pyrimidin-4-yl)piperidin-4-yl)(5-phenyl-4,5-dihydro-1H-pyrazol-1-yl)methanone

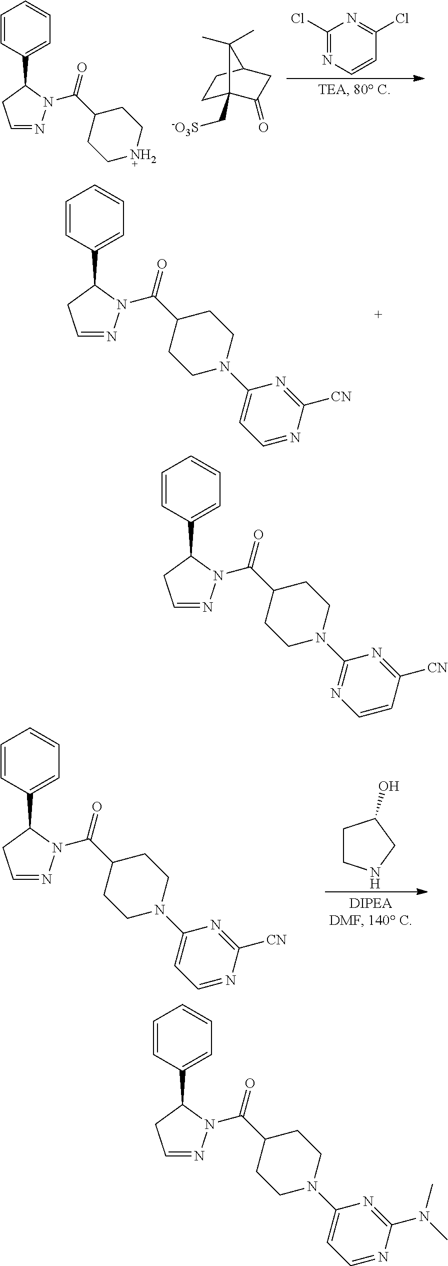

### Step 1

To a suspension of (S)-(5-phenyl-4,5-dihydro-1H-pyrazol-1-yl)(piperidin-4-yl)methanone, (1R)-10-camphorsulphonic acid salt (3 g, 6.14 mmol), 2,4-dichloropyrimidine (1.1 g, 7.4 mmol) in MeCN (31 mL) stirred at rt was added TEA (2.1 mL, 15.4 mmol). The reaction vessel was sealed and the reaction mixture was stirred at 80° C. for 15 h. The reaction mixture was evaporated in vacuo to give a pink oil. This residue was purified by normal phase column chromatography [CyH/(EtOH/EtOAc 1:4) 0-70%] to afford (S)-(1-(4-chloropyrimidin-2-yl)piperidin-4-yl)(5-phenyl-4,5-dihydro-1H-pyrazol-1-yl)methanone (0.288 g, 0.78 mmol, purity: 100%, recovery: 13%) as a light yellow solid and (S)-(1-(2-chloropyrimidin-4-yl)piperidin-4-yl)(5-phenyl-4,5-dihydro-1H-pyrazol-1-yl)methanone (2 g, 5.41 mmol, purity: 100%, recovery: 88%) as a colorless oil. LCMS of the major product (m/z) 370 and 372 (M+H)⁺, retention time: 2.35 min, LC/MS Method 1.

### Step 2

To a solution of (S)-(1-(2-chloropyrimidin-4-yl)piperidin-4-yl)(5-phenyl-4,5-dihydro-1H-pyrazol-1-yl)methanone (200 mg, 0.54 mmol) and DIPEA (0.14 mL, 0.81 mmol) in DMF (5 mL) stirred at rt was added (S)-pyrrolidin-3-ol (56.5 mg, 0.65 mmol) in one charge. The reaction mixture was stirred at 140° C. for 72 h and evaporated in vacuo to give a black oil. This residue was purified by 2 successive column chromatographies (MeOH/DCM 0-5%+1% TEA and NH₂ silica [(EtOH/EtOAc 4:1)/CyH 0-60%] to afford (S)-(1-(2-(dimethylamino)pyrimidin-4-yl)piperidin-4-yl)(5-phenyl-4,5-dihydro-1H-pyrazol-1-yl)methanone (25 mg, 0.07 mmol, purity: 100%, recovery: 12%) as a white solid. This was an unintended product, arising from reaction of (S)-(1-(2-chloropyrimidin-4-yl)piperidin-4-yl)(5-phenyl-4,5-dihydro-1H-pyrazol-1-yl)methanone with dimethylamine (from the decomposition of DMF). LCMS (m/z) 379 (M+H)⁺, retention time: 1.62 min, LC/MS Method 1. ¹H NMR (400 MHz, DMSO-d6) δ ppm 7.84 (d, J=5.9 Hz, 1H), 7.32 (dd, J=7.6, 7.2 Hz, 2H), 7.24 (m, 2H), 7.10 (d, J=7.2 Hz, 2H), 6.03 (d, J=5.9 Hz, 1H), 5.31 (dd, J=11.9, 4.6 Hz, 1H), 4.35 (m, 2H), 3.49 (ddd, J=19.0, 12.0, 1.5 Hz, 1H), 3.37 (tt, J=11.6, 3.8 Hz, 1H), 3.03 (s, 6H), 2.92 (m, 2H), 2.67 (ddd, J=19.0, 4.7, 1.9 Hz, 1H), 1.86 (d, J=12.9 Hz, 1H), 1.73 (d, J=12.9 Hz, 1H), 1.45 (m, 2H).

### Example 123

### (S)-2-(4-(5-(2,5-difluorophenyl)-4,5-dihydro-1H-pyrazole-1-carbonyl)piperidin-1-yl)pyrimidine-5-carboxamide

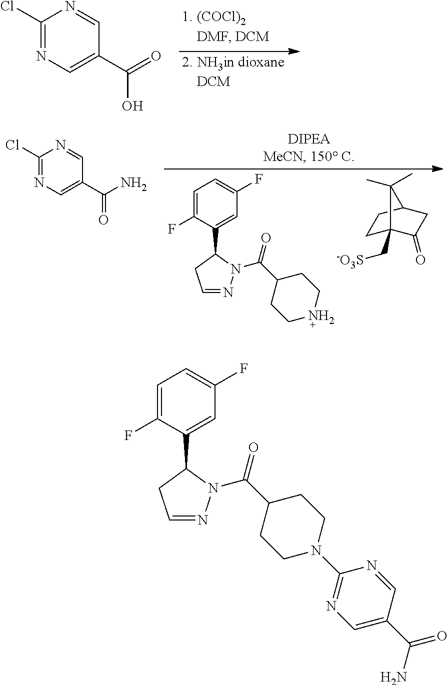

### Step 1

To a suspension of 2-chloropyrimidine-5-carboxylic acid (2 g, 12.6 mmol) in DCM (100 mL) and a few drops of DMF was added a 2 M solution of oxalyl chloride in DCM (6.3 mL, 12.6 mmol) at 0° C. The reaction mixture was stirred at rt for 1 h and evaporated in vacuo. To a 0.5 M solution of ammonia in dioxane (25.2 mL, 12.6 mmol) was added a solution of the acyl chloride in DCM at 0° C. The reaction mixture was stirred at rt for 18 h. The reaction mixture was evaporated in vacuo and water was added (300 mL). The resulting precipitate was filtered and washed with iPr₂O (2×50 mL) to give 2-chloropyrimidine-5-carboxamide (1.2 g, 7.6 mmol, purity: 82%, recovery: 60%) as a cream-colored powder. LCMS (m/z) 158 and 160 (M+H)⁺, retention time: 0.76 min, LC/MS Method 1.

### Step 2

To a suspension of (S)-(5-(2,5-difluorophenyl)-4,5-dihydro-1H-pyrazol-1-yl)(piperidin-4-yl)methanone, 1R-(−)-camphor-10-sulphonic acid salt (250 mg, 0.48 mmol) in MeCN (2.5 mL) was added 2-chloropyrimidine-5-carboxamide (82 mg, 0.52 mmol) and DIPEA (0.25 mL, 1.4 mmol). The vessel was sealed, and heated with stirring at 150° C. for 24 h. The reaction mixture was evaporated in vacuo. The crude was purified by recrystallization in MeCN to afford (S)-2-(4-(5-(2,5-difluorophenyl)-4,5-dihydro-1H-pyrazole-1-carbonyl)piperidin-1-yl)pyrimidine-5-carboxamide (116 mg, 0.28 mmol, purity: 100%, recovery: 59%) as a cream-colored powder. LCMS (m/z) 415 (M+H)⁺, retention time: 2.08 min, LC/MS Method 1. ¹H NMR (400 MHz, DMSO-d6) δ ppm 8.76 (s, 2H), 7.82 (s, 2H), 7.28 (s, 1H), 7.25 (m, 1H), 7.15 (m, 1H), 6.87 (m, 1H), 5.42 (dd, J=12.1, 5.2 Hz, 1H), 4.72 (d, J=12.9 Hz, 2H), 3.52 (dd, J=18.3, 12.4 Hz, 1H), 3.40 (tt, J=11.4, 3.6 Hz, 1H), 3.08 (m, 2H), 2.78 (dd, J=18.9, 4.6 Hz, 1H), 1.91 (d, J=12.0 Hz, 1H), 1.80 (d, J=13.7 Hz, 1H), 1.47 (m, 2H).

### Example 124

### (S)-5-chloro-2-(4-(5-(5-fluoropyridin-3-yl)-4,5-dihydro-1H-pyrazole-1-carbonyl)piperidin-1-yl)pyrimidine-4-carboxamide

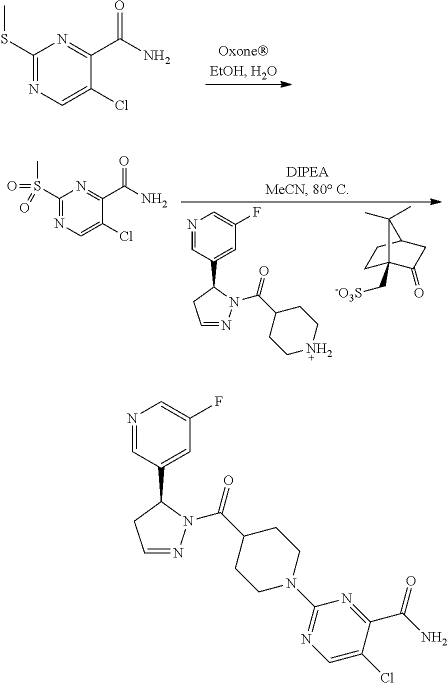

### Step 1

To a suspension of 5-chloro-2-(methylthio)pyrimidine-4-carboxamide (500 mg, 2.46 mmol) in EtOH (10 mL) and water (10 mL) stirred at rt was added oxone (3.02 g, 4.91 mmol) portion wise. The reaction mixture was stirred at rt for 3 h. Water (10 mL) and EtOAc (50 mL) were added. After separation, the aqueous layer was extracted with EtOAc (2×50 mL). The combined organic layers were washed with brine, dried over sodium sulfate, and evaporated in vacuo to afford 5-chloro-2-(methylsulfonyl)pyrimidine-4-carboxamide (350 mg, 1.5 mmol, purity: 100%, recovery: 61%) as a white solid. LCMS (m/z) 236 and 238 (M+H)⁺, retention time: 0.69 min, LC/MS Method 1.

### Step 2

To a suspension of (S)-(5-(5-fluoropyridin-3-yl)-4,5-dihydro-1H-pyrazol-1-yl)(piperidin-4-yl)methanone, (1R)-10-camphorsulphonate salt (200 mg, 0.394 mmol) in MeCN (5 mL) was added DIPEA (0.17 mL, 0.985 mmol), followed by 2 5-chloro-2-(methylsulfonyl)pyrimidine-4-carboxamide (102 mg, 0.433 mmol). The vessel was sealed, and heated with stirring at 80° C. for 16 h. The reaction mixture was evaporated in vacuo. Two successive purifications by column chromatography [CyH/(EtOH/EtOAc 4:1) 100/0 to 25/75] and (Et₂O/Acetone 60/40) were necessary to give the intended product as a yellow oil. Treatment of the oil with diethyl ether afforded, after filtration, (S)-5-chloro-2-(4-(5-(5-fluoropyridin-3-yl)-4,5-dihydro-1H-pyrazole-1-carbonyl)piperidin-1-yl)pyrimidine-4-carboxamide (40 mg, 0.093 mmol, 24%) as a yellow solid. LCMS (m/z) 432 and 434 (M+H)⁺, retention time: 1.95 min, LC/MS Method 1. ¹H NMR (400 MHz, DMSO-d6) δ ppm 8.47 (d, J=2.8 Hz, 1H), 8.46 (s, 1H), 8.29 (s, 1H), 8.07 (s, 1H), 7.78 (s, 1H), 7.47 (m, 1H), 7.29 (s, 1H), 5.41 (dd, J=11.8, 5.0 Hz, 1H), 4.60 (d, J=13.0 Hz, 2H), 3.52 (ddd, J=19.0, 12.0, 1.4 Hz, 1H), 3.38 (m, 1H), 3.03 (t, J=12.8 Hz, 2H), 2.85 (ddd, J=19.2, 5.2, 1.6 Hz, 1H), 1.89 (d, J=13.5 Hz, 1H), 1.78 (d, J=12.4 Hz, 1H), 1.46 (m, 2H).

### Example 125

### (S)—N-cyclopropyl-2-(4-(5-phenyl-4,5-dihydro-1H-pyrazole-1-carbonyl)piperidin-1-yl)pyrimidine-4-carboxamide

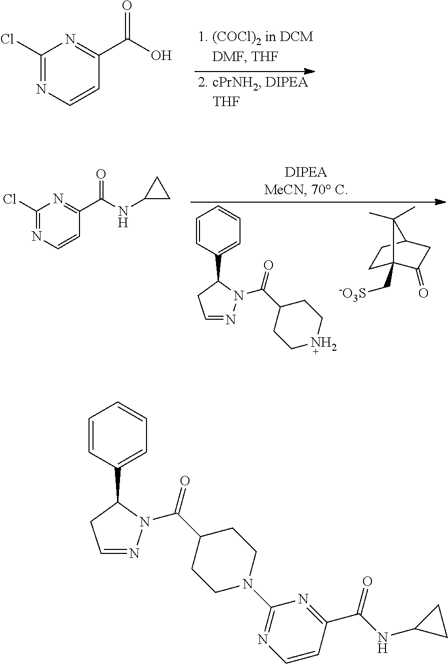

### Step 1

To a solution of 2-chloropyrimidine-4-carboxylic acid (500 mg, 3.15 mmol) in THF (30 mL) stirred at rt was added a 2 M solution of oxalyl chloride in DCM (4.7 mL, 9.40 mmol) portion-wise. Three drops of DMF were added to the mixture. The reaction mixture was stirred at rt for 30 min and evaporated in vacuo at rt for 30 min. THF (15 mL) and DCM (15 mL) were added to the residue and the mixture was evaporated in vacuo at 40° C. for 1 h. THF (30 mL) was added to the residue. Cyclopropylamine (1.4 mL, 19.0 mmol) and DIPEA (2.4 mL, 13.7 mmol) were added and the mixture was stirred at rt for 1 h. A saturated solution sodium bicarbonate solution (50 mL) and DCM (50 mL) were added. After separation, the organic layer was dried over sodium sulfate and evaporated in vacuo. The residue was purified by normal phase column chromatography (CyH/EtOAc 100/0 to 80/20) to afford 2-chloro-N-cyclopropylpyrimidine-4-carboxamide (390 mg, 1.88 mmol, purity: 100%, recovery: 59%) as a white foam. LCMS (m/z) 198 and 200 (M+H)⁺, retention time: 1.64 min, LC/MS Method 1.

### Step 2

To a suspension of (S)-(5-phenyl-4,5-dihydro-1H-pyrazol-1-yl)(piperidin-4-yl)methanone, (1R)-10-camphorsulphonic acid salt (300 mg, 0.61 mmol) and 2-chloro-N-cyclopropylpyrimidine-4-carboxamide (121 mg, 0.61 mmol) in MeCN (2 mL) stirred at rt was added neat DIPEA (0.38 mL, 2.15 mmol) in one charge. The reaction vessel was sealed and the reaction mixture was stirred at 70° C. for 1 h. The reaction mixture was evaporated in vacuo. The residue was purified by normal phase column chromatography (CyH/EtOAc 100/0 to 50/50). A trituration into iPr₂O afforded, after filtration, (S)—N-cyclopropyl-2-(4-(5-phenyl-4,5-dihydro-1H-pyrazole-1-carbonyl)piperidin-1-yl)pyrimidine-4-carboxamide (90 mg, 0.20 mmol, purity: 100%, recovery: 33%) as an off-white powder. LCMS (m/z) 419 (M+H)⁺, retention time: 3.44 min, LC/MS Method 1. ¹H NMR (400 MHz, DMSO-d6) δ ppm 8.56 (d, J=4.2 Hz, 1H), 8.53 (d, J=4.7 Hz, 1H), 7.32 (dd, J=7.6, 7.2 Hz, 2H), 7.24 (m, 2H), 7.11 (d, J=7.2 Hz, 2H), 7.05 (d, J=4.7 Hz, 1H), 5.32 (dd, J=12.0, 4.6 Hz, 1H), 4.8 (d, 12.7 Hz, 2H), 3.50 (ddd, J=18.8, 11.8, 1.3 Hz, 1H), 3.88 (m, 1H), 3.00 (m, 2H), 2.81 (m, 1H), 2.68 (ddd, 18.8, 4.6, 1.7 Hz, 1H), 1.91 (d, 11.4 Hz, 1H), 1.78 (d, 11.4 Hz, 1H), 1.48 (m, 2H), 0.69 (m, 4H).

Example 126 was synthesized in an analogous manner.

| | | | | LC: | |
|---|---|---|---|---|---|
| | | | | retention | |
| | | | | time | |
| | | | | (min); | |
| | | | | LC/MS | |
| | | | | Method of | MS |
| Ex. | Name | Structure | ¹H NMR | analysis | (M + H)⁺ |
| 126 | (S)-N-(2- hydroxy- ethyl)-2- (4-(5- phenyl- 4,5- dihydro- 1H- pyrazole-1- carbonyl) piperidin- 1-yl) pyrimidine- | 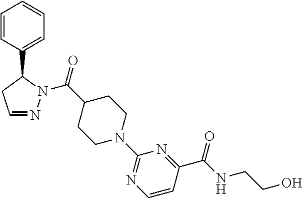 [SMILES: O=C(NCCO)c1ccnc(N2CCC(C(=O)N3N=CC[C@H]3c3ccccc3)CC2)n1] | ¹H NMR (400 MHz, DMSO-d6) δ ppm 8.66 (s, 1H), 8.55 (d, 1H, J = 4.4 Hz), 7.32 (m, 2H), 7.25 (m, 2H), 7.10 (m, 3H), 5.32 (dd, 1H, J = 11.4, 3.8 Hz), 4.79 (m, 2H), 3.50 (m, 3H), 3.36 (m, 3H), 3.03 (m, 2H), 2.69 (d, 1H, J = 18.8 Hz), 1.92 (d, | 2.08 Method 1 | 423 |
| | 4- | | 1H, J = 11.6 Hz), | | |
| | carboxamide | | 1.80 (d, 1H, J = | | |
| | | | 12.1 Hz), 1.50 | | |
| | | | (m, 2H) | | |

### Example 127

### (S)-(1-(5-fluoro-4-(2-morpholinoethoxy)pyrimidin-2-yl)piperidin-4-yl)(5-phenyl-4,5-dihydro-1H-pyrazol-1-yl)methanone

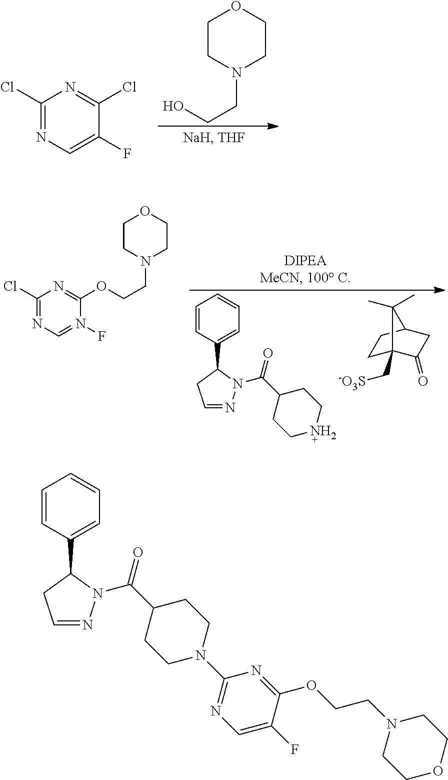

### Step 1

To a solution of 2-morpholinoethanol (0.870 mL, 7.2 mmol) in THF (60 mL) stirred under nitrogen at 0° C. was added 60% NaH in mineral oil (0.311 g, 7.8 mmol) portion-wise. The reaction mixture was allowed to warm to rt and stirred for further 20 min. The reaction mixture was cooled to 0° C. and 2,4-dichloro-5-fluoropyrimidine (1 g, 6 mmol) was added portion wise. The reaction mixture was allowed to warm to rt and stirred for 2.5 h. EtOAc (100 mL) and water (50 mL) were added. After separation, the aqueous layer was extracted with EtOAc (2×50 mL). The combined organic layers were washed with brine, dried over sodium sulfate and evaporated in vacuo to afford a cream-colored oil. This residue was purified by normal phase column chromatography [CyH/(EtOH/EtOAc 4:1) 100/0 to 80/20] to afford 4-(2-((2-chloro-5-fluoropyrimidin-4-yl)oxy)ethyl)morpholine (1.29 g, 4.9 mmol, purity: 100%, recovery: 82%) as a colorless oil. LCMS (m/z) 262 and 264 (M+H)⁺, retention time: 0.39 min, LC/MS Method 1.

### Step 2

To a suspension of (S)-(5-phenyl-4,5-dihydro-1H-pyrazol-1-yl)(piperidin-4-yl)methanone, (1R)-10-camphorsulphonate salt (200 mg, 0.41 mmol) in MeCN (5 mL) was added 4-(2-((2-chloro-5-fluoropyrimidin-4-yl)oxy)ethyl)morpholine (118 mg, 0.45 mmol) and DIPEA (0.18 mL, 1.02 mmol). The vessel was sealed, and heated with stirring at 100° C. for 16 h. The reaction mixture was evaporated in vacuo to give a brown oil. Purification of the crude by normal phase column chromatography [CyH/(EtOH/EtOAc) 80/20 to 70/30] afforded (S)-(1-(5-fluoro-4-(2-morpholinoethoxy)pyrimidin-2-yl)piperidin-4-yl)(5-phenyl-4,5-dihydro-1H-pyrazol-1-yl)methanone (89 mg, 0.18 mmol, purity: 100%, recovery: 45%) as a yellow resin. LCMS (m/z) 483 (M+H)⁺, retention time: 1.63 min, LC/MS Method 1. ¹H NMR (400 MHz, DMSO-d6) δ ppm 8.15 (d, J=3.0 Hz, 1H), 7.32 (dd, J=7.6, 7.4 Hz, 2H), 7.24 (m, 2H), 7.11 (d, J=7.4 Hz, 2H), 5.31 (dd, J=11.8, 4.6 Hz, 1H), 4.53 (d, J=12.9 Hz, 2H), 4.47 (t, J=5.8 Hz, 2H), 3.55 (t, J=4.6 Hz, 4H), 3.46 (m, 1H), 3.35 (m, 1H), 2.97 (m, 2H), 2.69 (t, J=5.8 Hz, 2H), 2.66 (m, 1H), 2.45 (m, 4H), 1.88 (d, J=11.8 Hz, 1H), 1.74 (d, J=11.8 Hz, 1H), 1.47 (m, 2H).

### Example 128

### (S)-(1-(5-hydroxypyrimidin-2-yl)piperidin-4-yl)(5-phenyl-4,5-dihydro-1H-pyrazol-1-yl)methanone

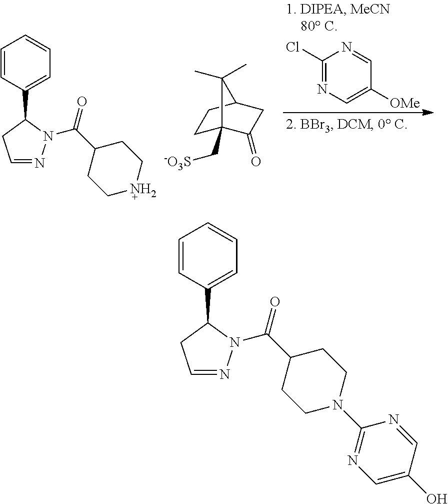

To a suspension of (S)-(5-phenyl-4,5-dihydro-1H-pyrazol-1-yl)(piperidin-4-yl)methanone, 1R-(−)-camphor-10-sulphonic acid salt (600 mg, 1.2 mmol) in MeCN (10 mL) were added 2-chloro-5-methoxypyrimidine (177 mg, 1.2 mmol) and DIPEA (0.54 mL, 3.1 mmol). The reaction vessel was sealed, stirred at 150° C. for 2 h and then concentrated in vacuo. This residue was dissolved in DCM (100 mL) and boron tribromide 1 M solution in DCM (3.1 mL, 3.1 mmol) was added dropwise. The mixture was stirred at 0° C. for 2 h. Water (300 mL) and DCM (100 mL) were added. After separation, the aqueous layer was washed with DCM (100 mL). The combined organic layers were dried over sodium sulfate, filtered, and evaporated in vacuo. The residue was purified by normal phase column chromatography [CyH/(EtOH/EtOAc 4:1) 100/0 to 70/30]. A trituration into iPr₂O afforded, after filtration, (S)-(1-(5-hydroxypyrimidin-2-yl)piperidin-4-yl)(5-phenyl-4,5-dihydro-1H-pyrazol-1-yl)methanone (30 mg, 0.09 mmol, purity: 100%, recovery: 7%) as a cream-colored powder. LCMS (m/z) 352 (M+H)⁺, retention time: 2.04 min, LC/MS Method 1. ¹H NMR (400 MHz, DMSO-d6) δ ppm 9.17 (s, 1H), 8.01 (s, 2H), 7.32 (dd, J=7.6, 7.2 Hz, 2H), 7.23 (m, 2H), 7.10 (d, J=7.4 Hz, 2H), 5.30 (dd, J=12.0, 4.6 Hz, 1H), 4.49 (d, J=12.7 Hz, 2H), 3.49 (dd, J=18.3, 12.4 Hz, 1H), 3.32 (m, 1H), 2.87 (m, 2H), 2.67 (dd, J=18.9, 4.6 Hz, 1H), 1.83 (d, J=11.8 Hz, 1H), 1.70 (d, J=12.1 Hz, 1H), 1.46 (m, 2H).

### Example 129

### (S)-(1-(6-(5-methyl-1,3,4-oxadiazol-2-yl)pyrimidin-4-yl)piperidin-4-yl)(5-phenyl-4,5-dihydro-1H-pyrazol-1-yl)methanone

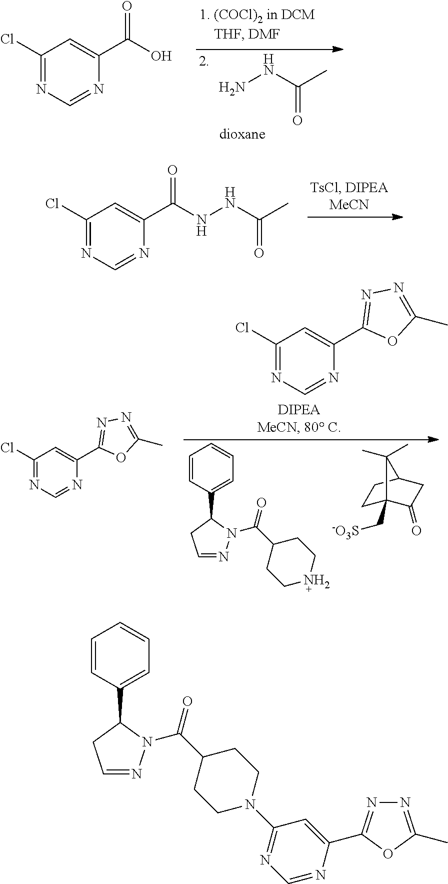

### Step 1

To a solution of 6-chloropyrimidine-4-carboxylic acid (1 g, 6.31 mmol) and DMF (9.8 μL, 0.126 mmol) in THF (60 mL) stirred under nitrogen at 0° C. was added oxalyl chloride 2 M solution in DCM (4.73 mL, 9.46 mmol) dropwise. The reaction mixture was allowed to warm to rt and stirred for further 1 h. The reaction mixture was evaporated in vacuo to give a brown oil. This residue was dissolved in 1,4-dioxane (60 mL) and acetohydrazide (0.935 g, 12.6 mmol) added in one charge. The reaction mixture was allowed to warm to rt and stirred for further 1 h. The reaction mixture was evaporated in vacuo to afford N′-acetyl-6-chloropyrimidine-4-carbohydrazide (1.94 g, 9 mmol, purity: 37%, recovery: 143%) as an off-white solid, which was taken on to the next step without further purification. LCMS (m/z) 215 and 217 (M+H)⁺, retention time: 1.38 min, LC/MS Method 2.

### Step 2

To a suspension of N′-acetyl-6-chloropyrimidine-4-carbohydrazide (500 mg, 2.3 mmol) in MeCN (25 mL) stirred under nitrogen at 0° C. was added neat DIPEA (0.814 mL, 4.66 mmol) dropwise followed by solid TsCl (1.333 g, 7 mmol) in one charge. The reaction mixture was allowed to warm to rt and stirred for further 15 min. The reaction mixture was evaporated in vacuo to give an orange oil. This residue was purified by normal phase column chromatography [CyH/(EtOH/EtOAc 4:1) 100/0 to 75/25%] to afford 2-(6-chloropyrimidin-4-yl)-5-methyl-1,3,4-oxadiazole (376 mg, 1.9 mmol, purity: 62%, recovery: 82%) as an off-white solid. LCMS (m/z) 197 and 199 (M+H)⁺, retention time: 1.99 min, LC/MS Method 2.

### Step 3

To a suspension of (S)-(5-phenyl-4,5-dihydro-1H-pyrazol-1-yl)(piperidin-4-yl)methanone, (1R)-10-camphorsulphonate salt (200 mg, 0.41 mmol) in MeCN (5 mL) was added 2-(6-chloropyrimidin-4-yl)-5-methyl-1,3,4-oxadiazole (89 mg, 0.450 mmol) and DIPEA (0.18 mL, 1.02 mmol). The vessel was sealed, and heated with stirring at 80° C. for 16 h. The reaction mixture was evaporated in vacuo to give an orange oil. Purification of the crude by normal phase column chromatography [CyH/(EtOH/EtOAc) 100/0 to 50/50] gave (S)-(1-(6-(5-methyl-1,3,4-oxadiazol-2-yl)pyrimidin-4-yl)piperidin-4-yl)(5-phenyl-4,5-dihydro-1H-pyrazol-1-yl)methanone as a off-white foam. Treatment with iPr₂O led to precipitation of the product, which afforded, after filtration, (S)-(1-(6-(5-methyl-1,3,4-oxadiazol-2-yl)pyrimidin-4-yl)piperidin-4-yl)(5-phenyl-4,5-dihydro-1H-pyrazol-1-yl)methanone (162 mg, 0.39 mmol, purity: 100%; recovery: 95%) as an off-white solid. LCMS (m/z) 418 (M+H)⁺, retention time: 2.06 min, LC/MS Method 1. ¹H NMR (400 MHz, DMSO-d6) δ ppm 8.62 (d, J=0.9 Hz, 1H), 7.43 (s, 1H), 7.32 (dd, J=7.7, 7.3 Hz, 2H), 7.23 (m, 2H), 7.11 (d, J=7.3, 2H), 5.32 (dd, J=11.9, 4.4 Hz, 1H), 4.49 (s, 2H), 3.50 (ddd, J=18.9, 12.0, 1.4 Hz, 1H), 3.44 (m, 1H), 3.13 (m, 2H), 2.69 (ddd, J=18.9, 4.6, 1.7 Hz, 1H), 2.61 (s, 3H), 1.95 (d, J=13.1 Hz, 1H), 1.82 (d, J=13.1, 1H), 1.51 (m, 2H).

### Example 130

### (S)-(1-(4-(hydroxymethyl)pyrimidin-2-yl)piperidin-4-yl)(5-phenyl-4,5-dihydro-1H-pyrazol-1-yl)methanone

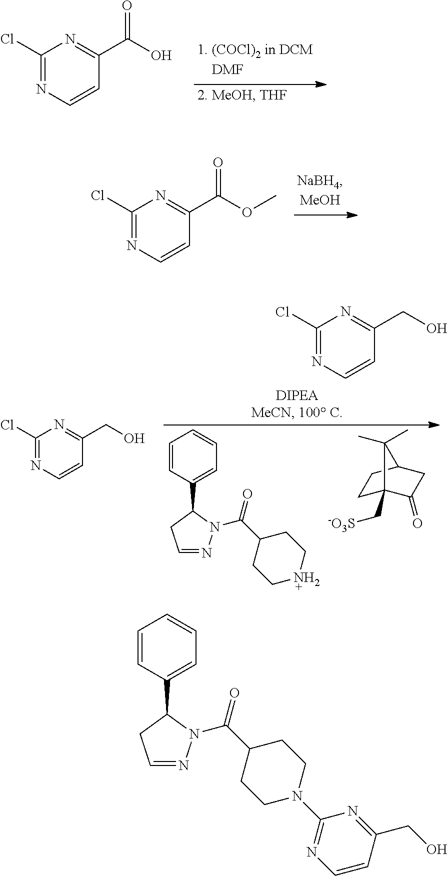

### Step 1

To a suspension of 2-chloropyrimidine-4-carboxylic acid (500 mg, 3.15 mmol) and DMF (0.024 mL, 0.32 mmol) in DCM (30 mL) stirred under nitrogen at 0° C. was added a 2 M solution of oxalyl chloride (1.74 mL, 3.47 mmol) in DCM dropwise. The reaction mixture was allowed to warm to rt and stirred for further 3 h at rt. The reaction mixture was evaporated in vacuo to give a brown oil. This residue was dissolved in THF (20 mL) and MeOH was added (0.14 mL, 3.5 mmol) dropwise. The reaction mixture was stirred at rt under nitrogen for 1 h. The reaction mixture was evaporated in vacuo to afford methyl 2-chloropyrimidine-4-carboxylate (780 mg, 4.5 mmol, purity: 80%, recovery: 143%) as a brown oil. The compound was used in the next step without purification. LCMS (m/z) 173 and 175 (M+H)⁺, retention time: 1.86 min, LC/MS Method 2.

### Step 2

To a solution of methyl 2-chloropyrimidine-4-carboxylate (750 mg, 4.35 mmol) in methanol (20 mL) stirred under nitrogen at 0° C. was added sodium borohydride (329 mg, 8.7 mmol) portion-wise. The reaction mixture was allowed to warm to rt and stirred at rt for 2 h. A saturated solution of ammonium chloride in water (40 mL) and EtOAc (40 mL) were added.

After separation, the aqueous layer was extracted with EtOAc (2×40 mL). The combined organic layers were washed with brine, dried over sodium sulfate, and evaporated in vacuo to afford a brown oil. This residue was purified by normal phase column chromatography [(EtOH/EtOAc 4:1)/CyH 0-40%] to afford (2-chloropyrimidin-4-yl)methanol (187 mg, 1.3 mmol, purity: 42%, recovery: 30%) as a light yellow solid. LCMS (m/z) 145 and 147 (M+H)⁺, retention time: 1.26 min, LC/MS Method 1.

### Step 3

To a suspension of (S)-(5-phenyl-4,5-dihydro-1H-pyrazol-1-yl)(piperidin-4-yl)methanone, (1R)-10-camphorsulphonate salt (200 mg, 0.41 mmol) in MeCN (5 mL) was added 2 (2-chloropyrimidin-4-yl)methanol (65 mg, 0.45 mmol) and DIPEA (0.18 mL, 1.02 mmol). The vessel was sealed and heated with stirring at 100° C. for 16 h. The reaction mixture was evaporated in vacuo to give a brown oil. Purification of the crude by normal phase column chromatography [CyH/(EtOH/EtOAc) 100/0 to 80/20] afforded a yellow oil. A precipitation into diisopropyl ether afforded, after filtration, (S)-(1-(4-(hydroxymethyl)pyrimidin-2-yl)piperidin-4-yl)(5-phenyl-4,5-dihydro-1H-pyrazol-1-yl)methanone (80 mg, 0.22 mmol, purity: 100%; recovery: 54%) as an off-white solid. LCMS (m/z) 366 (M+H)⁺, retention time: 1.86 min, LC/MS Method 1. ¹H NMR (400 MHz, DMSO-d6) δ ppm 8.32 (d, J=5.0, 1H), 7.32 (dd, J=7.3, 7.6 Hz, 2H) 7.24 (m, 2H), 7.11 (d, J=7.3 Hz, 2H), 6.69 (d, J=5.0, 1H), 5.39 (t, J=5.9 Hz, 1H), 5.31 (dd, J=11.8, 4.6 Hz, 1H), 4.66 (d, J=13.0, 2H), 4.34 (d, J=5.9, 2H), 3.49 (ddd, J=18.8, 11.8, 1.5 Hz, 1H), 3.37 (m, 1H), 2.95 (m, 2H), 2.67 (ddd, 18.9, 4.7, 1.7 Hz, 1H), 1.86 (d, J=12.5, 1H), 1.74 (d, J=13.0, 1H), 1.44 (m, 2H).

### Example 131

### (S)-2-(4-(5-phenyl-4,5-dihydro-1H-pyrazole-1-carbonyl)piperidin-1-yl)pyrimidine-5-carboxamide

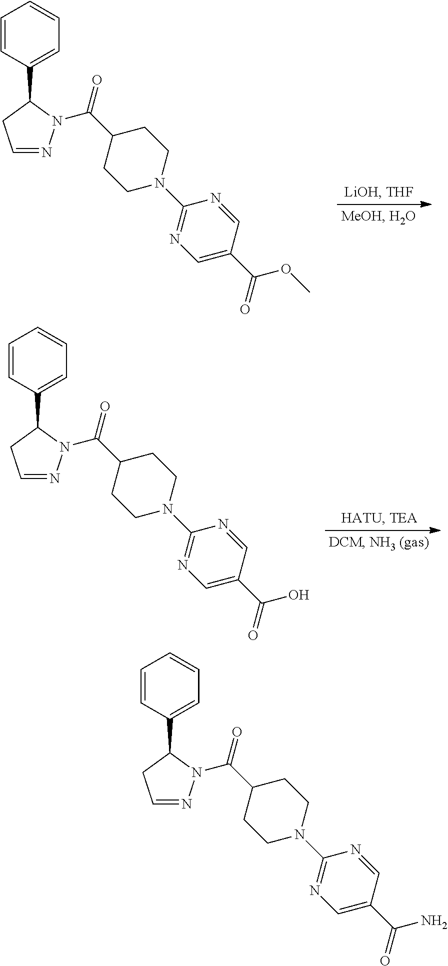 [SMILES: COC(=O)c1cnc(N2CCC(C(=O)N3N=CC[C@H]3c3ccccc3)CC2)nc1 ; O=C(O)c1cnc(N2CCC(C(=O)N3N=CC[C@H]3c3ccccc3)CC2)nc1 ; NC(=O)c1cnc(N2CCC(C(=O)N3N=CC[C@H]3c3ccccc3)CC2)nc1]

### Step 1

To a solution of (S)-methyl 2-(4-(5-phenyl-4,5-dihydro-1H-pyrazole-1-carbonyl)piperidin-1-yl)pyrimidine-5-carboxylate (700 mg, 1.78 mmol), MeOH (20 mL), THF (20 mL), and water (10 mL) was added LiOH (47 mg, 1.96 mmol). The reaction mixture was stirred at 50° C. for 3 h. The reaction mixture was evaporated in vacuo. Water (10 mL) was added and the pH was adjusted to 3 by addition of AcOH. The resulting precipitate was filtered off and dissolved in DCM (10 mL). The solution was washed with water (10 mL). The aqueous layer was extracted with DCM (10 mL). The combined organic layers were dried over sodium sulfate, filtered, and evaporated in vacuo. The residue was triturated in Et₂O to give (S)-2-(4-(5-phenyl-4,5-dihydro-1H-pyrazole-1-carbonyl)piperidin-1-yl)pyrimidine-5-carboxylic acid (590 mg, 1.56 mmol, purity: 100%, recovery: 87%) as a white powder. LCMS (m/z) 380 (M+H)⁺, retention time: 2.27 min, LC/MS Method 1.

### Step 2

To a solution of (S)-2-(4-(5-phenyl-4,5-dihydro-1H-pyrazole-1-carbonyl)piperidin-1-yl)pyrimidine-5-carboxylic acid (250 mg, 0.659 mmol) in DCM (50 mL) was added Et₃N (0.138 mL, 1 mmol) and HATU (263 mg, 0.692 mmol). Ammonia gas was bubbled in the reaction mixture for 5 min. The reaction mixture was stirred for 15 h. The reaction mixture was evaporated in vacuo. The residue was dissolved in DCM (200 mL) and washed successively with water (2×150 mL), a saturated solution of sodium bicarbonate (150 mL) and brine (150 mL). The organic layer was dried over sodium sulfate, filtered, and concentrated in vacuo. The residue was purified by normal phase column chromatography (DCM/MeOH 100/0 to 95/5 to give (S)-2-(4-(5-phenyl-4,5-dihydro-1H-pyrazole-1-carbonyl)piperidin-1-yl)pyrimidine-5-carboxamide. A trituration into iPr₂O afforded, after filtration, (S)-2-(4-(5-phenyl-4,5-dihydro-1H-pyrazole-1-carbonyl)piperidin-1-yl)pyrimidine-5-carboxamide (85 mg, 0.23 mmol, purity: 100%, recovery: 34%) as a white powder. LCMS (m/z) 379 (M+H)⁺, retention time: 1.98 min, LC/MS Method 1. ¹H NMR (400 MHz, DMSO-d6) δ ppm 8.76 (s, 2H), 7.82 (s, 1H), 7.32 (dd, J=7.6, 7.2 Hz, 2H), 7.23 (m, 3H), 7.11 (d, J=7.2 Hz, 2H), 5.31 (dd, J=11.8, 4.4 Hz, 1H), 4.72 (d, J=12.7 Hz, 2H), 3.50 (dd, J=18.8, 11.9 Hz, 1H), 3.40 (m, 1H), 3.08 (m, 2H), 2.68 ((dd, J=18.8, 3.2 Hz, 1H), 1.98 (d, J=12.3 Hz, 1H), 1.79 (d, J=12.3 Hz, 1H), 1.47 (m, 2H).

### Example 132

### (S)-2-(4-(5-(3,5-difluorophenyl)-4,5-dihydro-1H-pyrazole-1-carbonyl)piperidin-1-yl)oxazole-5-carboxamide

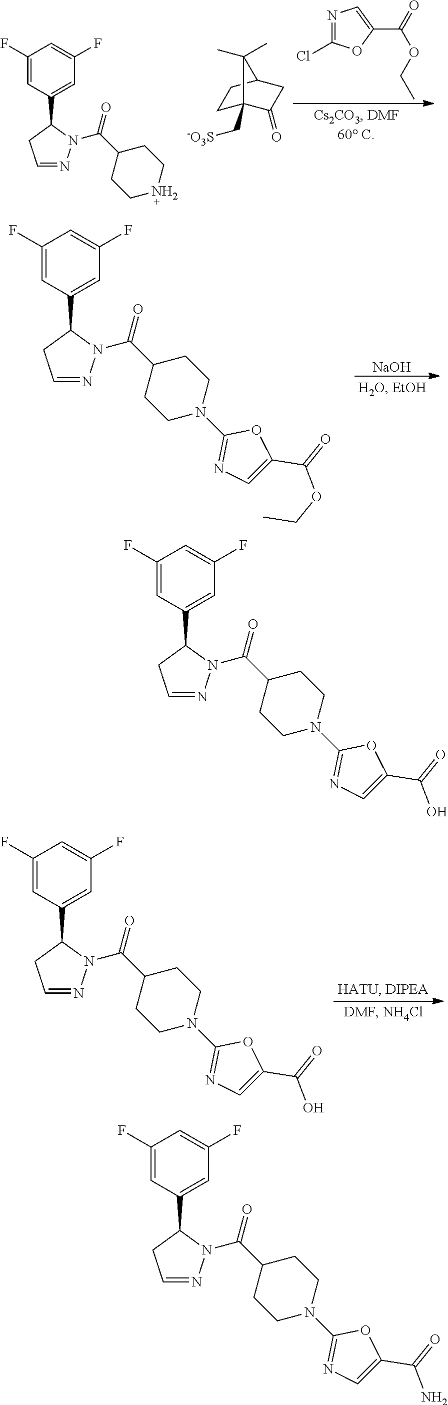

### Step 1

To a solution of (S)-(5-(3,5-difluorophenyl)-4,5-dihydro-1H-pyrazol-1-yl)(piperidin-4-yl)methanone, (1R)-10-camphorsulphonate salt (500 mg, 1.7 mmol) and cesium carbonate (1.11 g, 3.4 mmol) in DMF (2 mL) was added ethyl 2-chlorooxazole-5-carboxylate (359 mg, 2.1 mmol). The reaction mixture was stirred 60° C. for 3 h and concentrated in vacuo. The residue was dissolved in EtOAc (20 mL) which was then washed with water (10 mL). After separation, the organic layer was washed with brine (10 mL), dried over sodium sulfate, and concentrated in vacuo. The residue was precipitated in hexanes to afford (S)-ethyl 2-(4-(5-(3,5-difluorophenyl)-4,5-dihydro-1H-pyrazole-1-carbonyl)piperidin-1-yl)oxazole-5-carboxylate (477 mg, 1.1 mmol, purity: 100%, recovery: 65%) as a white solid. LCMS (m/z) 433 (M+H)⁺, retention time: 2.53 min, LC/MS Method 1.

### Step 2

To a solution of (S)-ethyl 2-(4-(5-(3,5-difluorophenyl)-4,5-dihydro-1H-pyrazole-1-carbonyl)piperidin-1-yl)oxazole-5-carboxylate (447 mg, 1.0 mmol) in EtOH (10 mL) a 1 M solution of sodium hydroxide (2.1 mL, 2.1 mmol) was added. The reaction mixture was stirred rt for 3 h and concentrated in vacuo. The residue was partitioned between water (10 mL) and Et₂O (10 mL). The aqueous phase separated and acidified with 1 M HCl solution to pH=1 and extracted with EtOAc (20 mL). The EtOAc solution was separated, dried over sodium sulfate, and concentrated in vacuo. Trituration in iPr₂O led to precipitation and afforded (S)-2-(4-(5-(3,5-difluorophenyl)-4,5-dihydro-1H-pyrazole-1-carbonyl)piperidin-1-yl)oxazole-5-carboxylic acid (380 mg, 0.94 mmol, purity; 100%, recovery: 91%) as a off white solid. LCMS (m/z) 405 (M+H)⁺, retention time: 2.08 min, LC/MS Method 1.

### Step 3

To a solution of (S)-2-(4-(5-(3,5-difluorophenyl)-4,5-dihydro-1H-pyrazole-1-carbonyl)piperidin-1-yl)oxazole-5-carboxylic acid (340 mg, 0.84 mmol), HATU (480 mg, 1.26 mmol), NH₄Cl (450 mg, 8.41 mmol) in DMF (3 mL) was added DIPEA (1304 mg, 10.1 mmol). The reaction was stirred at rt for 48 h and concentrated in vacuo. The residue was partitioned between water (20 mL) and EtOAc (20 mL). After separation, the organic layer was washed with brine (10 mL), dried over sodium sulfate, filtered, and concentrated in vacuo to afford (S)-2-(4-(5-(3,5-difluorophenyl)-4,5-dihydro-1H-pyrazole-1-carbonyl)piperidin-1-yl)oxazole-5-carboxamide (322 mg, 0.8 mmol, purity: 100%, recovery: 95%) as a cream-colored solid. LCMS (m/z) 404 (M+H)⁺, retention time: 1.95 min, LC/MS Method 1.

### Example 133

### (S)-2-(4-(5-(3,5-difluorophenyl)-4,5-dihydro-1H-pyrazole-1-carbonyl)piperidin-1-yl)oxazole-5-carbonitrile

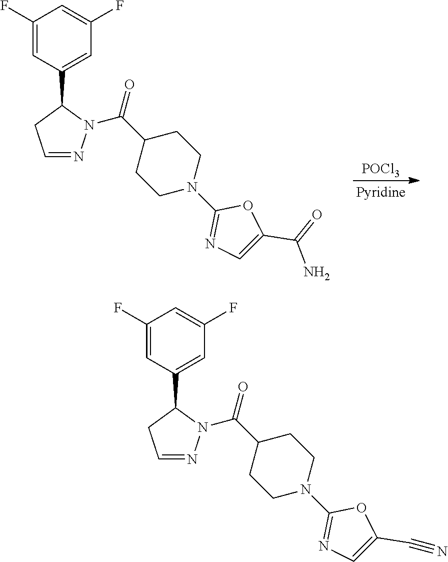 [SMILES: NC(=O)c1cnc(N2CCC(C(=O)N3N=CC[C@H]3c3cc(F)cc(F)c3)CC2)o1 ; "POCl3" ; N#Cc1cnc(N2CCC(C(=O)N3N=CC[C@H]3c3cc(F)cc(F)c3)CC2)o1]

To a solution of (S)-2-(4-(5-(3,5-difluorophenyl)-4,5-dihydro-1H-pyrazole-1-carbonyl)piperidin-1-yl)oxazole-5-carboxamide (300 mg, 0.744 mmol) in pyridine (3 mL) at rt was added phosphoryl trichloride (114 mg, 0.74 mmol). The reaction was stirred at rt for 2 h and concentrated in vacuo. The residue was partitioned between water (20 mL) and EtOAc (20 mL). After separation, the organic layer was washed with brine (10 mL), dried over sodium sulfate, filtered, and concentrated in vacuo to afford (S)-2-(4-(5-(3,5-difluorophenyl)-4,5-dihydro-1H-pyrazole-1-carbonyl)piperidin-1-yl)oxazole-5-carbonitrile (160 mg, 0.42 mmol, purity: 100%, recovery: 56%) as a off-white solid. LCMS (m/z) 386 (M+H)⁺, retention time: 2.50 min, LC/MS Method 1. ¹H NMR (400 MHz, DMSO) δ ppm 7.97 (s, 1H), 7.26 (m, 1H), 7.12 (tt, J=9.4, 2.3 Hz, 1H), 6.85 (m, 2H), 5.34 (dd, J=12.0, 4.9 Hz, 1H), 4.5 (d, J=13.1 Hz, 2H), 3.49 (ddd, J=19.0, 12.0, 1.5 Hz, 1H), 3.33 (tt, J=11.4, 3.6 Hz, 1H), 3.20 (m, 2H), 2.75 (ddd, J=19.0, 5.1, 1.7 Hz, 1H), 1.95 (d, J=13.3 Hz, 1H), 1.81 (d, J=13.3 Hz, 1H), 1.56 (m, 2H).

Examples 134-138 were synthesized in an analogous manner to Example 132 and 133.

| | | | | LC: | |
|---|---|---|---|---|---|
| | | | | retention | |
| | | | | time | |
| | | | | (min); | |
| | | | | LC/MS | |
| | | | | Method of | MS |
| Ex. | Name | Structure | ¹H NMR | analysis | (M + H)⁺ |
| 134 | (S)-2-(4- (5-(3,5- difluoro- phenyl)- 4,5- dihydro- 1H- pyrazole-1- carbonyl) piperidin- 1-yl) oxazole- 4- carboxamide | 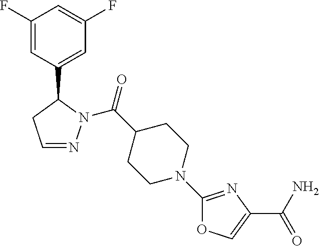 [SMILES: NC(=O)c1coc(N2CCC(C(=O)N3N=CC[C@H]3c3cc(F)cc(F)c3)CC2)n1] | ¹H NMR (400 MHz, CDCl₃) δ ppm 7.87 (s, 1H), 7.19 (t, J = 1.5 Hz, 1H), 6.84 (m, 3H), 5.37 (dd, J = 11.8, 4.9 Hz, 1H), 4.13 (d, J = 13.1 Hz, 2H), 3.54 (19.1, 12.0, 1.5 Hz, 1H) 3.47 (tt, J = 11.6, 3.7 Hz, 1H), 3.14 (m, 2H), 2.82 (ddd, J = 19.0, 4.9, 1.7 Hz, 1H), 2.01 (m, 1H), | 2.11 Method 1 | 404 |
| | | | 1.91 (m, 1H), 1.72 | | |
| | | | (m, 2H) | | |
| 135 | (S)-2-(4- (5-(3,5- difluoro- phenyl)- 4,5- dihydro- 1H- pyrazole-1- carbonyl) piperidin- 1-yl) oxazole-4- carbonitrile | 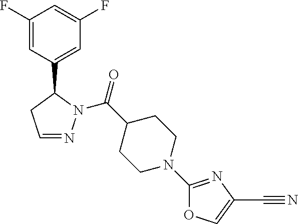 [SMILES: N#Cc1coc(N2CCC(C(=O)N3N=CC[C@H]3c3cc(F)cc(F)c3)CC2)n1] | ¹H NMR (400 MHz, CDCl₃) δ ppm 7.69 (s, 1H), 7.01 (s, 1H), 6.71 (m, 3H), 5.33 (dd, J = 12.0, 4.9 Hz, 1H), 4.11 (m, 2H), 3.47 (ddd, J = 18.8, 12.0, 1.5 Hz, 1H), 3.34 (tt, J = 11.2, 3.8 Hz, 1H), 3.14 (m, 2H), 2.81 (ddd, J = 19.0, 5.1, 1.7 Hz, 1H), 1.75-2.05 (m, 4H) | 2.50 Method 1 | 386 |
| 136 | (S)-2-(4- (5-phenyl- 4,5- dihydro- 1H- pyrazole-1- carbonyl) piperidin- 1-yl) oxazole- 4- carboxamide | 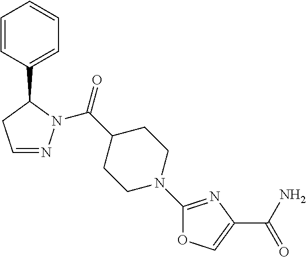 [SMILES: NC(=O)c1coc(N2CCC(C(=O)N3N=CC[C@H]3c3ccccc3)CC2)n1] | ¹H NMR (400 MHz, CDCl₃) δ ppm 7.75 (s, 1H), 7.31 (m, 2H), 7.25 (m, 1H), 7.14 (m, 2H), 7.01 (t, J = 1.6 Hz, 1H), 5.35 (dd, J = 11.9, 4.8 Hz, 1H), 4.06 (m, 2H), 3.44 (ddd, J = 8.8, 12.0, 1.5 Hz, 1H), 3.32 (tt, J = 11.2, 3.7 Hz, 1H), 3.09 (m, 2H), 2.82 (ddd, J = 18.8, 4.9, 1.7 Hz, 1H), 1.83 (m, 4H) | 1.99 Method 1 | 368 |
| 137 | (S)-2-(4- (5-phenyl- 4,5- dihydro- 2H- pyrazole-1- carbonyl) piperidin- 1-yl) oxazole- 4- carbonitrile | 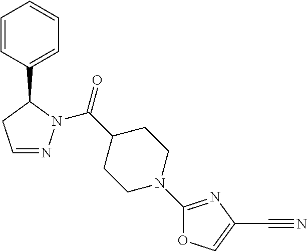 [SMILES: N#Cc1coc(N2CCC(C(=O)N3N=CC[C@H]3c3ccccc3)CC2)n1] | ¹H NMR (400 MHz, CDCl₃) δ ppm 7.67 (s, 1H), 7.37- 7.31 (m, 2H), 7.30- 7.24 (m, 1H), 7.19- 7.14 (m, 2H), 7.01 (t, J = 1.6 Hz, 1H) 5.38 (dd, J = 5.0 and 11.9 Hz, 1H), 4.14-4.04 (m, 2H), 3.46 (ddd, J = 1.5, 12.0 and 18.8 Hz, 1H), 3.39-3.30 (m, 1H), 3.20-3.09 (m, 2H), 2.85 (ddd, J = 1.7, 5.0 and | 2.38 Method 1 | 350 |
| | | | 18.9 Hz, 1H), 2.04- | | |
| | | | 1.96 (m, 1H), 1.95- | | |
| | | | 1.73 (m, 3H) | | |
| 138 | (S)-2-(4- (5-phenyl- 4,5- dihydro- 1H- pyrazole-1- carbonyl) piperidin- 1-yl) oxazole- 5- carbonitrile | 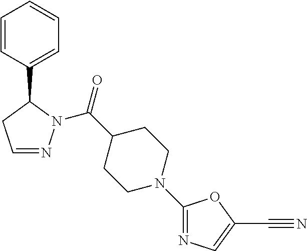 [SMILES: N#Cc1cnc(N2CCC(C(=O)N3N=CC[C@H]3c3ccccc3)CC2)o1] | ¹H NMR (400 MHz, CDCl₃) δ ppm 7.47 (s, 1H), 7.34 (dd, J = 7.6, 7.02 Hz, 2H), 7.28 (m, 1H), 7.16 (d, J = 7.0 Hz, 2H), 7.02 (s, 1H), 5.38 (dd, J = 12.0, 4.9 Hz, 1H), 4.18 (tt, J = 12.7, 3.6 Hz, 2H), 3.47 (ddd, J = 19.0, 12.0, 1.5 Hz, 1H), 3.38 (tt, J = 10.8, 4.0 Hz, 1H), 3.22 (m, 2H), 2.86 (ddd, | 2.39 Method 1 | 350 |
| | | | J = 19.0, 4.9, 1.7 | | |
| | | | Hz, 1H), 1.75-2.05 | | |
| | | | (m, 4H) | | |

### Example 139

### (S)-(1-(7H-purin-2-yl)piperidin-4-yl)(5-(3,5-difluorophenyl)-4,5-dihydro-1H-pyrazol-1-yl)methanone

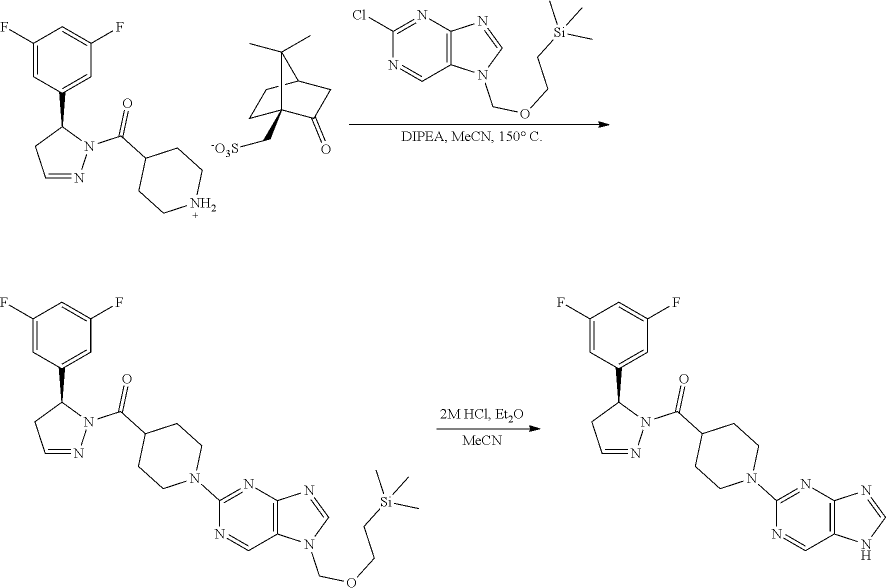

### Step 1

To a suspension of (S)-(5-(3,5-difluorophenyl)-4,5-dihydro-1H-pyrazol-1-yl)(piperidin-4-yl)methanone, 1R-(−)-camphor-10-sulphonic acid salt (450 mg, 0.86 mmol) in MeCN (10 mL) were added 2-chloro-7-((2-(trimethylsilyl)ethoxy)methyl)-7H-purine (244 mg, 0.86 mmol) and DIPEA (0.224 mL, 1.28 mmol). The reaction vessel was sealed and heated at 150° C. for 72 h. The reaction mixture was evaporated in vacuo. The residue was purified by normal phase column chromatography [CyH/(EtOAc/EtOH 3:1) 100/0 to 70/30] (S)-(5-(3,5-difluorophenyl)-4,5-dihydro-1H-pyrazol-1-yl)(1-(7-((2-(trimethylsilyl)ethoxy)methyl)-7H-purin-2-yl)piperidin-4-yl)methanone. Trituration in iPr₂O afforded, after filtration, (S)-(5-(3,5-difluorophenyl)-4,5-dihydro-1H-pyrazol-1-yl)(1-(7-((2-(trimethylsilyl)ethoxy)methyl)-7H-purin-2-yl)piperidin-4-yl)methanone (280 mg, 0.52 mmol, purity: 100%, recovery: 60%) as a white powder. LCMS (m/z) 542 (M+H)⁺, retention time: 2.89 min, LC/MS Method 1.

### Step 2

To a suspension of (S)-(5-(3,5-difluorophenyl)-4,5-dihydro-1H-pyrazol-1-yl)(1-(7-((2-(trimethylsilyl)ethoxy)methyl)-7H-purin-2-yl)piperidin-4-yl)methanone (280 mg, 0.52 mmol) in MeCN (10 mL) was added HCl as a 2 M solution in Et₂O (0.258 mL, 0.517 mmol). The reaction mixture was stirred at rt for 24 h and evaporated in vacuo. The crude was dissolved in water (50 mL) and the pH adjusted to 9 with addition of 1 M sodium hydroxide solution. This aqueous phase was extracted with DCM (2×60 mL). The combined organic layers were dried over sodium sulfate, filtered, and evaporated in vacuo. The residue was purified by normal phase column chromatography [CyH/(EtOAc/EtOH 3:1) 100/0 to 70/30] to (S)-(1-(7H-purin-2-yl)piperidin-4-yl)(5-(3,5-difluorophenyl)-4,5-dihydro-1H-pyrazol-1-yl)methanone. Trituration in Et₂O afforded, after filtration, (S)-(1-(7H-purin-2-yl)piperidin-4-yl)(5-(3,5-difluorophenyl)-4,5-dihydro-1H-pyrazol-1-yl)methanone (40 mg, 0.01 mmol, purity: 100%, recovery: 19%) as a cream powder. LCMS (m/z) 412 (M+H)⁺, retention time: 1.90 min, LC/MS Method 1. ¹H NMR (400 MHz, DMSO-d6) δ ppm 12.7 (s, 1H), 8.70 (s, 1H), 8.13 (s, 1H), 7.25 (s, 1H), 7.12 (t, J=9.3 Hz, 1H), 6.84 (d, J=6.6 Hz, 2H), 5.34 (dd, J=11.9, 4.8 Hz, 1H), 4.69 (d, J=13.1 Hz, 2H), 3.48 (dd, J=18.6, 12.3 Hz, 1H), 3.38 (m, 1H), 3.00 (m, 2H), 2.75 (dd, J=18.4, 4.2 Hz, 1H), 1.90 (d, J=12.5 Hz, 1H), 1.75 (d, J=12.0 Hz, 1H), 1.50 (m, 2H).

### Example 140

### (S)-(1-(4-(4,5-dihydro-1H-imidazol-2-yl)pyrimidin-2-yl)piperidin-4-yl)(5-phenyl-4,5-dihydro-1H-pyrazol-1-yl)methanone

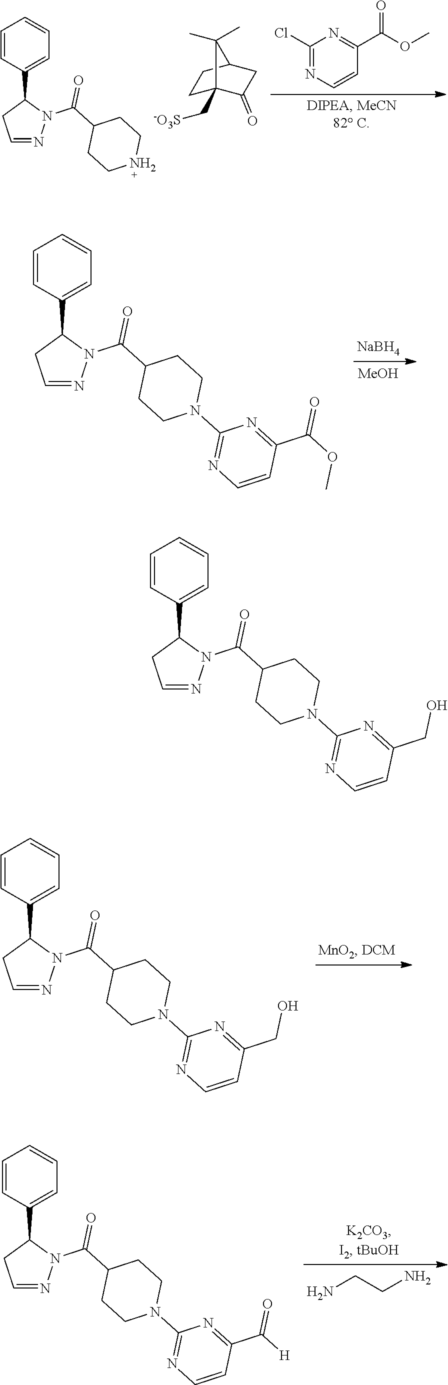

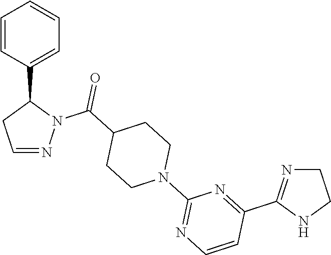 [SMILES: O=C(C1CCN(c2nccc(C3=NCCN3)n2)CC1)N1N=CC[C@H]1c1ccccc1]

### Step 1

To a suspension of (S)-(5-phenyl-4,5-dihydro-1H-pyrazol-1-yl)(piperidin-4-yl)methanone, (1R)-10-camphorsulphonic acid salt (2.6 g, 5.3 mmol) and methyl 2-chloropyrimidine-4-carboxylate (1.0 g, 5.85 mmol) in MeCN (50 mL) stirred at rt was added neat DIPEA (2.3 mL, 13.3 mmol) in one charge. The reaction mixture was stirred at 82° C. for 3 h and evaporated in vacuo to give a brown oil. This residue was purified by normal phase column chromatography (CyH/EtOAc 100/0 to 65/35) to afford (S)-methyl 2-(4-(5-phenyl-4,5-dihydro-1H-pyrazole-1-carbonyl)piperidin-1-yl)pyrimidine-4-carboxylate (2 g, 5.2 mmol, purity: 100%, recovery: 97%) as a yellow oil. LCMS (m/z) 394 (M+H)⁺, retention time: 2.51 min, LC/MS Method 1.

### Step 2

To a solution of (S)-methyl 2-(4-(5-phenyl-4,5-dihydro-1H-pyrazole-1-carbonyl)piperidin-1-yl)pyrimidine-4-carboxylate (500 mg, 1.27 mmol) in MeOH (5 mL) stirred under nitrogen at 0° C. was added sodium borohydride (96 mg, 2.54 mmol) in one charge. The reaction mixture was allowed to warm to rt and stirred for 3 h under nitrogen. A second batch of sodium borohydride (24 mg, 0.64 mmol) was added to the reaction mixture and stirred at rt for a further 2 h. A third batch of sodium borohydride (24.04 mg, 0.635 mmol) was added to the reaction mixture and stirred at rt for a further 15 h. The reaction mixture was evaporated in vacuo to give a white solid. DCM (10 mL) and a 3M solution of citric acid (1.5 mL) were added to the crude solid. After separation, the aqueous layer was extracted with DCM (2×10 mL). The combined organic layers were washed with brine (10 mL), dried over sodium sulfate, and evaporated in vacuo to give an off-white oil. This residue was purified by column chromatography [CyH/(EtOH/EtOAc 4:1) 100/0 to 60/40] to afford (S)-(1-(4-(hydroxymethyl)pyrimidin-2-yl)piperidin-4-yl)(5-phenyl-4,5-dihydro-1H-pyrazol-1-yl)methanone (455 mg, 1.25 mmol, purity: 100%, recovery: 98%) as an off-white sticky oil. LCMS (m/z) 366 (M+H)⁺, retention time: 1.86 min, LC/MS Method 1.

### Step 3

To a solution of (S)-(1-(4-(hydroxymethyl)pyrimidin-2-yl)piperidin-4-yl)(5-phenyl-4,5-dihydro-1H-pyrazol-1-yl)methanone (500 mg, 1.37 mmol) in DCM (10 mL) stirred under nitrogen at rt was added manganese dioxide (1.78 g, 20.5 mmol) in one charge. The reaction mixture was stirred at rt for 1 h under nitrogen. A second batch of manganese dioxide (595 mg, 6.84 mmol) was added to reaction mixture and stirred a further 1 h at rt. A third batch of manganese dioxide (595 mg, 6.84 mmol) was added to reaction mixture and stirred a further 30 min at rt. The reaction mixture was filtered through celite three times to afford, after evaporation in vacuo, (S)-2-(4-(5-phenyl-4,5-dihydro-1H-pyrazole-1-carbonyl)piperidin-1-yl)pyrimidine-4-carbaldehyde (312 mg, 0.86 mmol, purity: 55%, recovery: 63%) as a yellow oil. This product was used in the next step without purification. LCMS (m/z) 364 (M+H)⁺, retention time: 2.56 min, LC/MS Method 1.

### Step 4

To a solution of (S)-2-(4-(5-phenyl-4,5-dihydro-1H-pyrazole-1-carbonyl)piperidin-1-yl)pyrimidine-4-carbaldehyde (250 mg, 0.69 mmol) in tBuOH (7 mL) stirred under nitrogen at rt was added ethane-1,2-diamine (0.051 mL, 0.76 mmol) in one charge. The reaction mixture was stirred at rt for 1.5 h under nitrogen and potassium carbonate (285 mg, 2.06 mmol), followed by diiodine (218 mg, 0.86 mmol), were added to the reaction mixture. The reaction mixture was stirred at 70° C. under nitrogen for 3 h. The reaction mixture was cooled to rt and quenched with a saturated solution of sodium sulfite (10 mL) and the mixture was stirred until reaction had lightened to a consistent color. DCM (25 mL) was added. After separation, the aqueous layer was extracted with DCM (2×20 mL). The combined organic layers were successively washed with a saturated solution of sodium bicarbonate (25 mL) and brine (20 mL), dried over sodium sulfate, and evaporated in vacuo to afford a yellow oil. This residue was purified by normal phase column chromatography (MeOH/DCM 100/0 to 95/5+1% TEA) to give a yellow oil. Trituration in iPr₂O afforded, after evaporation, (S)-(1-(4-(4,5-dihydro-1Himidazol-2-yl)pyrimidin-2-yl)piperidin-4-yl)(5-phenyl-4,5-dihydro-1H-pyrazol-1-yl)methanone (277 mg, 0.687 mmol, 100%) as a light yellow solid. LCMS (m/z) 404 (M+H)⁺, retention time: 1.69 min, LC/MS Method 1. ¹H NMR (400 MHz, DMSO-d6) δ ppm 8.41 (d, J=4.9 Hz, 1H), 7.32 (dd, J=7.6, 7.3 Hz, 2H), 7.24 (m, 2H), 7.11 (d, J=7.7 Hz, 2H), 7.06 (d, J=4.8 Hz, 1H), 6.98 (s, 1H), 5.32 (dd, J=12.0, 4.5 Hz, 1H), 4.77 (d, J=13.0, 2H), 3.84 (s, 2H), 3.50 (ddd, J=18.9, 11.8, 1.5 Hz, 1H), 3.39 (m, 3H), 3.00 (m, 2H), 2.68 (ddd, J=18.8, 4.7, 1.6 Hz, 1H), 1.89 (d, J=12.2, 1H), 1.77 (d, J=13.4 Hz, 1H), 1.47 (m, 2H).

### Example 141

### (S)-(4-methylpiperazin-1-yl)(2-(4-(5-phenyl-4,5-dihydro-1H-pyrazole-1-carbonyl)piperidin-1-yl)pyrimidin-4-yl)methanone

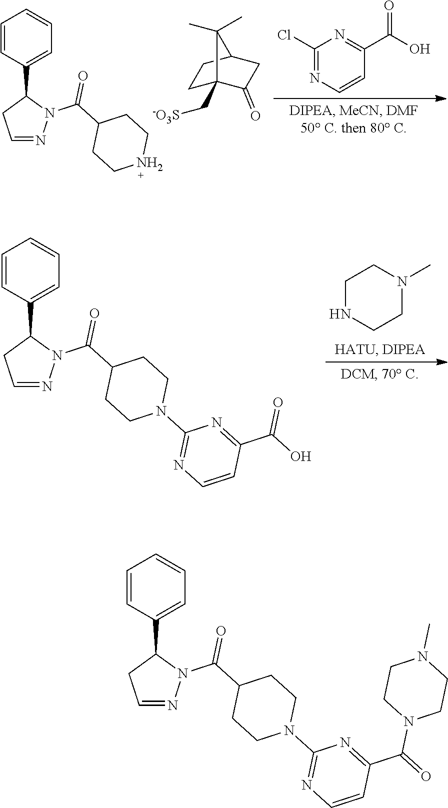

### Step 1

To a suspension of (S)-(5-phenyl-4,5-dihydro-1H-pyrazol-1-yl)(piperidin-4-yl)methanone, (1R)-10-camphorsulphonic acid salt (2 g, 4.1 mmol) and 2-chloropyrimidine-4-carboxylic acid (720 mg, 4.54 mmol) in MeCN (30 mL) stirred at rt was added neat DIPEA (2 mL, 11.5 mmol) in one charge. The reaction mixture was stirred at rt for 30 min and at 50° C. for 30 min. DIPEA (0.7 mL, 4 mmol) was added and the reaction mixture was stirred at 50° C. for 3.25 hours. The reaction mixture was evaporated in vacuo. DMF (40 mL) and DIPEA (2.7 mL, 15.5 mmol) were added to the residue and the reaction mixture was stirred at 50° C. for 16 h and at 80° C. for 24 h. Water (100 mL) and DCM (50 mL) were added. After separation, the aqueous layer was extracted with DCM (50 mL). The combined organic layers were dried over sodium sulfate and evaporated in vacuo to provide (S)-2-(4-(5-phenyl-4,5-dihydro-1H-pyrazole-1-carbonyl)piperidin-1-yl)pyrimidine-4-carboxylic acid (1.34 g, 3.2 mmol, purity: 91%, recovery: 78%) as an orange oil. LCMS (m/z) 380 (M+H)⁺, retention time: 2.22 min, LC/MS Method 1.

### Step 2

To a solution of 1-methylpiperazine (0.27 mL, 2.4 mmol) and (S)-2-(4-(5-phenyl-4,5-dihydro-1H-pyrazole-1-carbonyl)piperidin-1-yl)pyrimidine-4-carboxylic acid (150 mg, 0.4 mmol) in DCM (2 mL) stirred at rt were added neat DIPEA (0.24 mL, 1.37 mmol) and HATU (180 mg, 0.47 mmol) in one charge. The reaction vessel was sealed and stirred at 70° C. for 4 h. Water (50 mL) and EtOAc (100 mL) were added. After separation, the organic layer was dried over sodium sulfate and evaporated in vacuo. The residue purified by normal phase column chromatography (DCM/MeOH 100/0 to 96/4) to give (S)-(4-methylpiperazin-1-yl)(2-(4-(5-phenyl-4,5-dihydro-1H-pyrazole-1-carbonyl)piperidin-1-yl)pyrimidin-4-yl)methanone. A trituration in iPr₂O afforded, after filtration, (S)-(4-methylpiperazin-1-yl)(2-(4-(5-phenyl-4,5-dihydro-1H-pyrazole-1-carbonyl)piperidin-1-yl)pyrimidin-4-yl)methanone (35 mg, 0.07 mmol, purity: 100%, recovery: 18%) as an off-white powder. LCMS (m/z) 462 (M+H)⁺, retention time: 1.61 min, LC/MS Method 1. ¹H NMR (400 MHz, DMSO-d6) δ ppm 8.46 (d, J=4.9 Hz, 1H), 7.32 (t, J=7.4 Hz, 2H), 7.23 (m, 2H), 7.11 (d, J=7.2 Hz, 2H), 6.65 (d, J=4.7 Hz, 1H), 5.31 (dd, J=11.9, 4.5 Hz, 1H), 4.62 (d, J=12.7 Hz, 2H), 3.60 (m, 2H), 3.49 (ddd, J=18.8, 12.0, 1.1 Hz, 1H), 3.38 (m, 3H), 3.02 (m, 2H), 2.68 (ddd, J=18.8, 4.5, 1.4 Hz, 1H), 2.40 (s, 2H), 2.32 (s, 2H), 2.22 (s, 3H), 1.89 (d, J=11.8 Hz, 1H), 1.77 (d, J=11.6 Hz, 1H), 1.46 (m, 2H).

Examples 142-147 were synthesized in an analogous manner. For step 1, DMF may be substituted for DCM.

| | | | | LC: | |
|---|---|---|---|---|---|
| | | | | retention | |
| | | | | time | |
| | | | | (min); | |
| | | | | LC/MS | |
| | | | | Method of | MS |
| Ex. | Name | Structure | ¹H NMR | analysis | (M + H)⁺ |
| 142 | (S)-N,N- diethyl- 2-(4-(5- phenyl- 4,5- dihydro- 1H- pyrazole-1- carbonyl) piperidin- 1-yl) pyrimidine- 4- carboxamide | 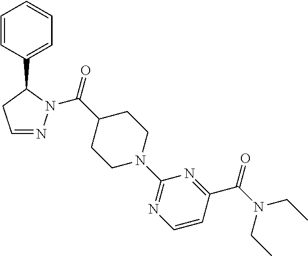 [SMILES: CCN(CC)C(=O)c1ccnc(N2CCC(C(=O)N3N=CC[C@H]3c3ccccc3)CC2)n1] | ¹H NMR (400 MHz, DMSO-d6) δ ppm 8.45 (d, 1H, J = 4.9 Hz), 7.32 (dd, 2H, J = 7.6, 7.2 Hz), 7.24 (m, 2H), 7.10 (d, 2H, J = 7.2 Hz), 6.61 (d, 1H, J = 4.9 Hz), 5.31 (dd, 1H, 11.8, 4.6 Hz), 4.64 (d, 2H, J = 12.7 Hz), 3.49 (ddd, 1H, J = 18.8, 11.8, 1.2 Hz), 3.40 (m, 3H), 3.19 (q, 2H, J = 7.0 Hz), 3.02 (m, 2H), 2.67 | 2.46 Method 1 | 435 |
| | | | (ddd, 1H, J = 18.8, | | |
| | | | 4.6, 1.5 Hz), 1.89 | | |
| | | | (d, 1H, J = 11.4 Hz, | | |
| | | | 1.77 (d, 1H, J = 11.4 | | |
| | | | Hz), 1.45 (m, 2H), | | |
| | | | 1.13 (t, 3H, J = 7.0 | | |
| | | | Hz), 1.10 (t, 3H, | | |
| | | | J = 7.0 Hz) | | |
| 143 | (S)-N′,N′- dimethyl- 2-(4-(5- phenyl- 4,5- dihydro- 1H- pyrazole-1- carbonyl) piperidin- 1-yl) pyrimidine- 4-carbo- hydrazide | 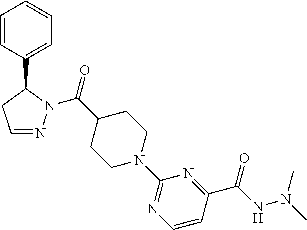 [SMILES: CN(C)NC(=O)c1ccnc(N2CCC(C(=O)N3N=CC[C@H]3c3ccccc3)CC2)n1] | ¹H NMR (400 MHz, DMSO-d6) δ ppm 9.47 (s, 1H), 8.53 (d, J = 4.7 Hz, 1H), 7.32 (t, J = 7.4 Hz, 2H), 7.24 (m, 2H), 7.1 (d, J = 7.2 Hz, 2H), 7.03 (d, J = 4.7 Hz, 1H), 5.32 (dd, J = 11.8, 4.6 Hz, 1H), 4.79 (d, J = 12.5, 2H), 3.5 (dd, J = 18.4, 12.3 Hz, 1H), 3.39 (m, 1H), 3.01 (q, J = 10.7 | 2.21 Method 1 | 422 |
| | | | Hz, 2H), 2.69 (dd, | | |
| | | | J = 19.0, 3.2 Hz, | | |
| | | | 1H), 2.60 (s, 3H), | | |
| | | | 2.60 (s, 3 + 3H), | | |
| | | | 1.93 (d, J = 10.8 | | |
| | | | Hz, 1H), 1.80 (d, | | |
| | | | J = 12.7 Hz, 1H), | | |
| | | | 1.49 (m, 2H) | | |
| 144 | (S)-N-(1- acetyl- piperidin- 4-yl)-2- (4-(5- phenyl- 4,5- dihydro-1H- pyraz ole-1- carbonyl) piperidin- | 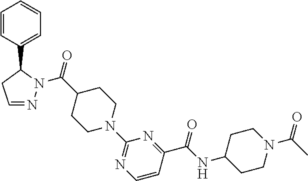 [SMILES: CC(=O)N1CCC(NC(=O)c2ccnc(N3CCC(C(=O)N4N=CC[C@H]4c4ccccc4)CC3)n2)CC1] | ¹H NMR (400 MHz, DMSO-d6) δ ppm 8.54 (d, J = 4.9 Hz, 1H), 8.40 (d, J = 8.7 Hz, 1H), 7.32 (t, J = 7.4 Hz, 2H), 7.11 (d, J = 7.4 Hz, 2H), 7.08 (d, J = 4.7 Hz, 1H), 5.32 (dd, J = 11.8, 4.5 Hz, 1H), 4.79 (d, J = 12.2 Hz, 2H), 4.38 | 2.23 Method 1 | 504 |
| | 1-yl) | | (d, J = 13.1 Hz, | | |
| | pyrimidine- | | 1H, 4.04 (m, 1H), | | |
| | 4- | | 3.83 (d, J = 13.5 | | |
| | carboxamide | | Hz, 1H), 3.50 | | |
| | | | (dd, J = 18.4, 12.3 | | |
| | | | Hz, 1H), 3.39 (m, | | |
| | | | 1H), 3.13 (m, 1H), | | |
| | | | 3.02 (q, J = 11.5 | | |
| | | | Hz, 2H), 2.69 (dd, | | |
| | | | J = 18.7, 2.9 Hz, | | |
| | | | 1H), 2.61 (d, J = | | |
| | | | 12.5 Hz, 1H), 2.02 | | |
| | | | (s, 1H), 1.93 (d, | | |
| | | | J = 12.3 Hz, 1H), | | |
| | | | 1.77 (m, 3H), 1.54 | | |
| | | | (m, 4H) | | |
| 145 | (S)-(1-(4- (morpholine- 4- carbonyl) pyrimidin- 2-yl) piperidin- 4-yl)(5- phenyl- 4,5- dihydro- 1H- pyrazol- 1-yl) methanone | 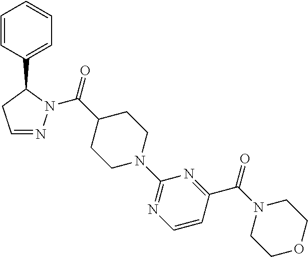 [SMILES: O=C(c1ccnc(N2CCC(C(=O)N3N=CC[C@H]3c3ccccc3)CC2)n1)N1CCOCC1] | ¹H NMR (400 MHz, DMSO-d6) δ ppm 8.47 (d, J = 4.7 Hz, 1H), 7.32 (t, J = 7.4 Hz, 2H), 7.23 (m, 2H), 7.11 (d, J = 7.2 Hz, 2H), 6.68 (d, J = 4.9 Hz, 1H), 5.31 (dd, J = 11.8, 4.6 Hz, 1H), 4.62 (d, J = 12.7 Hz, 2H), 3.63 (m, 4H), 3.55 (t, J = 4.8 Hz, 2H), 3.49 (ddd, J = 19.0, 12.0, 1.5), 3.38 (m, 3H), 3.02 | 2.21 Method 1 | 449 |
| | | | (m, 2H), 2.68 | | |
| | | | (ddd, J = 19.0, 4.5, | | |
| | | | 1.7), 1.89 (d, J = | | |
| | | | 11.6 Hz, 1H), 1.77 | | |
| | | | (d, J = 11.4 Hz, | | |
| | | | 1H), 1.46 (m, 2H) | | |
| 146 | (S)-(5- phenyl- 4,5- dihydro- 1H- pyrazol-1- yl)(1-(4- (piperazine- 1-carbonyl) pyrimidin- 2-yl) piperidin- 4-yl) methanone | 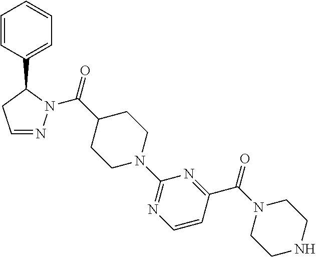 [SMILES: O=C(c1ccnc(N2CCC(C(=O)N3N=CC[C@H]3c3ccccc3)CC2)n1)N1CCNCC1] | ¹H NMR (400 MHz, DMSO-d6) δ ppm 8.46 (d, J = 4.7 Hz, 1H) 7.32 (dd, J = 7.6, 7.2 Hz, 2H), 7.23 (m, 2H), 7.11 (d, J = 7.2 Hz, 2H), 6.65 (d, J = 4.7 Hz, 1H), 5.31 (dd, J = 11.8, 4.6 Hz, 1H), 4.62 (dd, J = 12.7 Hz, 2H), 3.43 (m, 7H), 3.01 (m, 2H), 2.78 (m, 2H), 2.67 (m, 3H), 1.89 (d, J = 11.4 Hz, 1H), | 1.61 Method 1 | 448 |
| | | | 1.77 (d, J = 11.4 | | |
| | | | Hz, 1H), 1.46 (m, | | |
| | | | 2H) | | |

### Example 147

### (S)-2-(4-(5-phenyl-4,5-dihydro-1H-pyrazole-1-carbonyl)piperidin-1-yl)-N-(piperidin-4-yl)pyrimidine-4-carboxamide

Example 147 was synthesized in an analogous manner to Example 141 with an additional deprotection step.

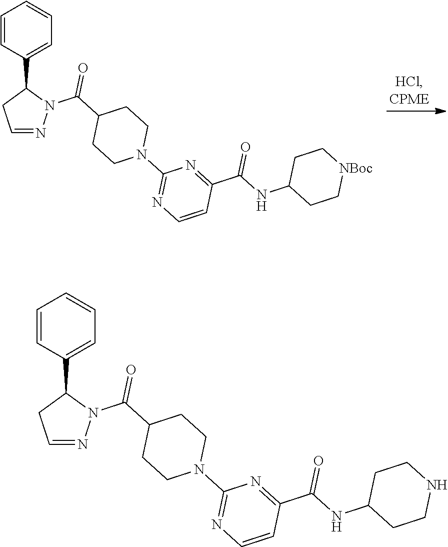 [SMILES: CC(C)(C)OC(=O)N1CCC(NC(=O)c2ccnc(N3CCC(C(=O)N4N=CC[C@H]4c4ccccc4)CC3)n2)CC1 ; O=C(NC1CCNCC1)c1ccnc(N2CCC(C(=O)N3N=CC[C@H]3c3ccccc3)CC2)n1]

To (S)-tert-butyl 4-(2-(4-(5-phenyl-4,5-dihydro-1H-pyrazole-1-carbonyl)piperidin-1-yl)pyrimidine-4-carboxamido)piperidine-1-carboxylate (330 mg, 0.59 mmol) was added 3M HCl solution in CPME (3 mL, 9 mmol). The reaction mixture was stirred at rt for 18 h. A saturated solution of sodium bicarbonate (50 mL) and EtOAc (50 mL) were added. After separation, the aqueous phase was extracted with EtOAc (2×50 mL). The combined organic layers were dried over sodium sulfate and evaporated in vacuo. The residue was purified by normal phase column chromatography (DCM/MeOH 100/0 to 80/20) to give (S)-2-(4-(5-phenyl-4,5-dihydro-1H-pyrazole-1-carbonyl)piperidin-1-yl)-N-(piperidin-4-yl)pyrimidine-4-carboxamide. Trituration in iPr₂O afforded, after filtration, (S)-2-(4-(5-phenyl-4,5-dihydro-1H-pyrazole-1-carbonyl)piperidin-1-yl)-N-(piperidin-4-yl)pyrimidine-4-carboxamide (5 mg, 10.3 μmol, purity: 100%, recovery: 2%) as a white powder. LCMS (m/z) 462 (M+H)⁺, retention time: 1.72 min, LC/MS Method 1. ¹H NMR (400 MHz, DMSO-d6) δ ppm 8.73 (s, 1H), 8.58 (d, J=8.0 Hz, 1H), 8.55 (d, J=4.7 Hz, 1H), 7.32 (dd, J=7.6, 7.2 Hz, 2H), 7.24 (m, 2H), 7.12 (d, J=7.2 Hz, 2H), 7.06 (d, J=4.9 Hz, 1H), 5.32 (dd, J=12.0, 4.6 Hz, 1H), 4.81 (d, J=12.7 Hz, 2H), 4.02 (m, 1H), 3.50 (ddd, J=18.8, 11.8, 1.2 Hz, 1H), 3.40 (m, 1H), 3.29 (m, 2H), 3.00 (m, 4H), 2.68 (ddd, J=18.8, 4.4, 1.5 Hz, 1H), 1.86 (m, 6H), 1.50 (m, 2H).

### Example 148

### (S)-(1-(4-(2H-tetrazol-5-yl)pyrimidin-2-yl)piperidin-4-yl)(5-phenyl-4,5-dihydro-1H-pyrazol-1-yl)methanone

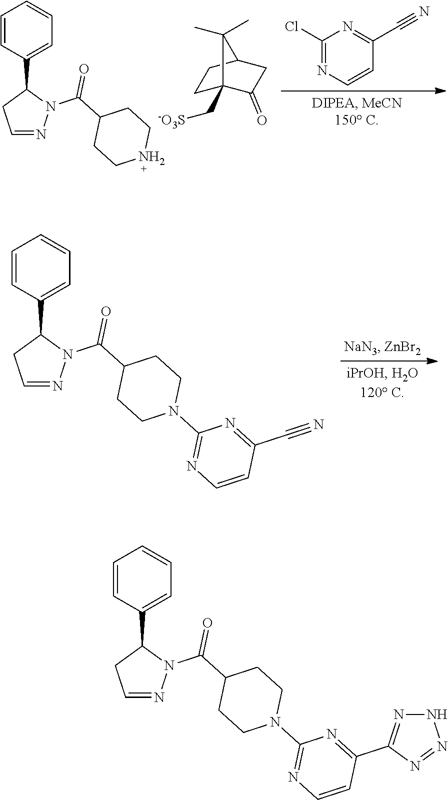

### Step 1

To a suspension of (S)-(5-phenyl-4,5-dihydro-1H-pyrazol-1-yl)(piperidin-4-yl)methanone, 1R-(−)-camphor-10-sulphonic acid salt (980 mg, 2 mmol) in MeCN (5 mL) was added DIPEA (1 mL, 6 mmol) and 2-chloropyrimidine-4-carbonitrile (279 mg, 2 mmol). The reaction vessel was sealed and heated to 150° C. for 2 h. The reaction mixture was cooled to rt and filtration of the resulting precipitate afforded (S)-2-(4-(5-phenyl-4,5-dihydro-1H-pyrazole-1-carbonyl)piperidin-1-yl)pyrimidine-4-carbonitrile (374 mg, 1.04 mmol, 100%, purity: 52%) as a white powder. LCMS (m/z) 361 (M+H)⁺, retention time: 2.66 min, LC/MS Method 1.

### Step 2

To a suspension of (S)-2-(4-(5-phenyl-4,5-dihydro-1H-pyrazole-1-carbonyl)piperidin-1-yl)pyrimidine-4-carbonitrile (374 mg, 1.04 mmol) in water (3 mL) were added sodium azide (74.2 mg, 1.1 mmol) and zinc bromide (234 mg, 1.04 mmol). The reaction vessel was sealed and heated to 120° C. for 1 h. Isopropanol (3 mL) was added and the reaction mixture was stirred at 120° C. for 4 h. A 6 M solution of HCl in water (3 mL) was added and the reaction was stirred for 1 h. The residue was partitioned between water (20 mL) and EtOAc (20 mL). After separation, the organic layer was washed with brine (10 mL), dried over sodium sulfate, filtered, and concentrated in vacuo. A trituration in Et₂O afforded, after filtration, (S)-(1-(4-(2H-tetrazol-5-yl)pyrimidin-2-yl)piperidin-4-yl)(5-phenyl-4,5-dihydro-1H-pyrazol-1-yl)methanone (256 mg, 0.64 mmol, purity: 100%, recovery: 61%) as a white powder. LCMS (m/z) 404 (M+H)⁺, retention time: 2.29 min, LC/MS Method 1. ¹H NMR (400 MHz, DMSO-d6) δ ppm 8.61 (d, J=4.93 Hz, 1H), 7.27 (m, 5H), 7.11 (m, 2H), 5.32 (dd, J=11.77, 4.55 Hz, 1H), 4.85 (d, J=11.58 Hz, 2H), 3.45 (m, 3H), 3.08 (m, 2H), 2.69 (ddd, J=18.93, 4.60, 1.71 Hz, 1H), 1.93 (m, 1H), 1.82 (d, J=11.2 Hz, 1H), 1.52 (m, 2H).

Example 149 was synthesized in an analogous manner.

| | | | | LC: | |
|---|---|---|---|---|---|
| | | | | retention | |
| | | | | time | |
| | | | | (min); | |
| | | | | LC/MS | |
| | | | | Method of | MS |
| Ex. | Name | Structure | ¹H NMR | analysis | (M + H)⁺ |
| 149 | (S)-(1-(4- (2H- tetrazol- 5-yl) pyrimidin- 2-yl) piperidin- 4-yl)(5- (5-fluoro- pyridin- 3-yl)-4,5- dihydro- 1H- pyrazol- 1-yl) | 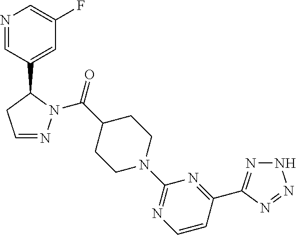 [SMILES: O=C(C1CCN(c2nccc(-c3nn[nH]n3)n2)CC1)N1N=CC[C@H]1c1cncc(F)c1] | ¹H NMR (400 MHz, DMSO-d6) δ ppm 8.59 (d, J = 4.9 Hz, 1 H), 8.47 (d, J = 2.7 Hz, 1 H), 8.30 (s, 1 H), 7.48 (d, J = 9.5 Hz, 1 H), 7.25-7.35 (m, 2 H), 5.42 (dd, J = 12.0, 5.1 Hz, 1 H), 4.84 (d, J = 11.2 Hz, 2 H), 3.37- 3.67 (m, 2 H), 2.96- 3.14 (m, 2 H), 2.85 (dd, J = 19.1, 3.7 | 1.95 Method 1 | 423 |
| | methanone | | Hz, 1 H), 1.88-1.97 | | |
| | | | (m, 1 H), 1.82 (d, | | |
| | | | J = 11.6 Hz, 1 H), | | |
| | | | 1.39-1.65 (m, 3 H). | | |

### Example 150

### 3-(5-fluoro-2-(4-((S)-5-phenyl-4,5-dihydro-1H-pyrazole-1-carbonyl)piperidin-1-yl)pyrimidin-4-yl)pyrrolidin-2-one

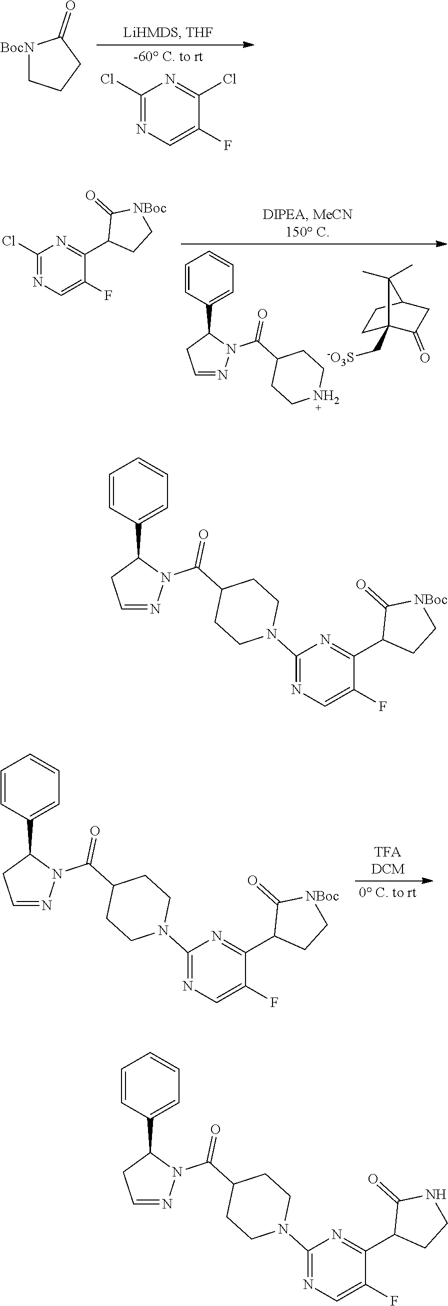

### Step 1

To a solution of 1-(tert-butoxycarbonyl)-2-pyrrolidinone (1.9 mL, 11 mmol) in THF (20 mL) was added dropwise a 1 M solution of lithium bis(trimethylsilyl) amide in THF (11.5 mL, 11.5 mmol) at −78° C. The reaction mixture was stirred at −60° C. for 45 min and then a solution of 2,4-dichloro-5-fluoropyrimidine (1.67 g, 10 mmol) in THF (10 mL) was added. The reaction mixture was allowed to warm to rt and stirred at rt for 15 h. The reaction mixture was partitioned between EtOAc (100 mL) and 1M HCl solution (50 mL). After separation, the organic layer was washed successively with a saturated solution of sodium bicarbonate (30 mL) and brine (30 mL), dried over sodium sulfate, filtered, and concentrated in vacuo. The residue was purified by normal phase column chromatography (CyH/EtOAc 95/5 to 60/40) to afford tert-butyl 3-(2-chloro-5-fluoropyrimidin-4-yl)-2-oxopyrrolidine-1-carboxylate (2.12 g, 6.7 mmol, purity: 89%, recovery: 67%) as a brown oil. LCMS (m/z) 314 (M−H)⁻, retention time: 2.38 min, LC/MS Method 1.

### Step 2

To a suspension of (S)-(5-phenyl-4,5-dihydro-1H-pyrazol-1-yl)(piperidin-4-yl)methanone, 1R-(−)-camphor-10-sulphonic acid salt (980 mg, 2 mmol) in MeCN (5 mL) was added DIPEA (1 mL, 6 mmol) then tert-butyl 3-(2-chloro-5-fluoropyrimidin-4-yl)-2-oxopyrrolidine-1-carboxylate (695 mg, 2.2 mmol). The reaction vessel was sealed, heated to 150° C. for 2 h, and evaporated in vacuo. The residue was purified by normal phase column chromatography (CyH/EtOAc 80/20 to 50/50) to afford tert-butyl 3-(5-fluoro-2-(4-((S)-5-phenyl-4,5-dihydro-1H-pyrazole-1-carbonyl)piperidin-1-yl)pyrimidin-4-yl)-2-oxopyrrolidine-1-carboxylate (990 mg, 1.85 mmol, purity: 83%, recovery: 92%) as yellow oil. LCMS (m/z) 537 (M+H)⁺, retention time: 2.77 min, LC/MS Method 1.

### Step 3

To a solution of tert-butyl 3-(5-fluoro-2-(4-((S)-5-phenyl-4,5-dihydro-1H-pyrazole-1-carbonyl)piperidin-1-yl)pyrimidin-4-yl)-2-oxopyrrolidine-1-carboxylate (990 mg, 1.85 mmol) in DCM (15 mL) was added TFA (1.42 mL, 18.5 mmol) at 0° C. The reaction mixture was stirred at rt for 72 h. The reaction mixture was partitioned between DCM (50 mL) and saturated sodium bicarbonate solution (50 mL). After separation, the organic layer was washed with brine (30 mL), dried over sodium sulfate, filtered, and concentrated in vacuo. Trituration in iPr₂O afforded, after filtration, (5-fluoro-2-(4-((S)-5-phenyl-4,5-dihydro-1H-pyrazole-1-carbonyl)piperidin-1-yl)pyrimidin-4-yl)pyrrolidin-2-one (490 mg, 1.12 mmol, purity: 100%, recovery: 61%) as white powder. LCMS (m/z) 437 (M+H)⁺, retention time: 2.27 min, LC/MS Method 1. ¹H NMR (400 MHz, DMSO-d6) δ ppm 8.35 (d, J=1.71 Hz, 1H) 7.91 (s, 1H), 7.27 (m, 4H), 7.11 (d, J=7.21 Hz, 2H), 5.31 (dd, J=11.77, 4.55 Hz, 1H), 4.56 (d, J=12.91 Hz, 2H), 3.84 (t, 1H), 3.41 (m, 4H), 2.98 (m, 2H), 2.68 (m, 1H), 2.39 (m, 2H), 1.87 (d, J=11.77 Hz, 1H), 1.75 (d, J=11.96 Hz, 1H), 1.47 (m, 2H).

Example 151 was synthesized in an analogous manner.

| | | | | LC: | |
|---|---|---|---|---|---|
| | | | | retention | |
| | | | | time | |
| | | | | (min); | |
| | | | | LC/MS | |
| | | | | Method of | MS |
| Ex. | Name | Structure | ¹H NMR | analysis | (M + H)⁺ |
| 151 | 3-(5- fluoro- 2-(4- ((S)-5-(5- fluoro- pyridin- 3-yl)-4,5- dihydro- 1H- pyrazole-1- carbonyl) piperidin- 1-yl) pyrimidin- 4-yl) pyrrolidin- | 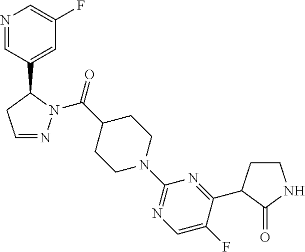 [SMILES: O=C1NCCC1c1nc(N2CCC(C(=O)N3N=CC[C@H]3c3cncc(F)c3)CC2)ncc1F] | ¹H NMR (400 MHz, DMSO-d6) δ ppm 8.47 (d, J = 2.7 Hz, 1H), 8.35 (d, J = 1.7 Hz, 1H), 8.29 (s, 1H), 7.91 (s, 1H), 7.47 (d, J = 9.7 Hz, 1H), 7.28 (s, 1H), 5.41 (dd, J = 12.0, 5.1 Hz, 1H), 4.55 (d, J = 12.9 Hz, 2H), 3.84 (t, J = 8.7 Hz, 1H), 3.52 (m, 1H), 3.36 (m, 2H), 2.96 (t, J = | 1.94 Method 1 | 456 |
| | 2-one | | 12.62 Hz, 2H), | | |
| | | | 2.84 ( ddd, J = | | |
| | | | 19.0, 5.1, 1.5 Hz, | | |
| | | | 1H), 2.68 (br. s, | | |
| | | | 1H), 2.39 (m, 2H), | | |
| | | | 1.88 (m, 1H), 1.76 | | |
| | | | (d, J = 11.8 Hz, | | |
| | | | 1H), 1.45 (m, 2H) | | |

### Example 152

### (S)-2-((2-(4-(5-phenyl-4,5-dihydro-1H-pyrazole-1-carbonyl)piperidin-1-yl)pyrimidin-4-yl)oxy)acetic acid

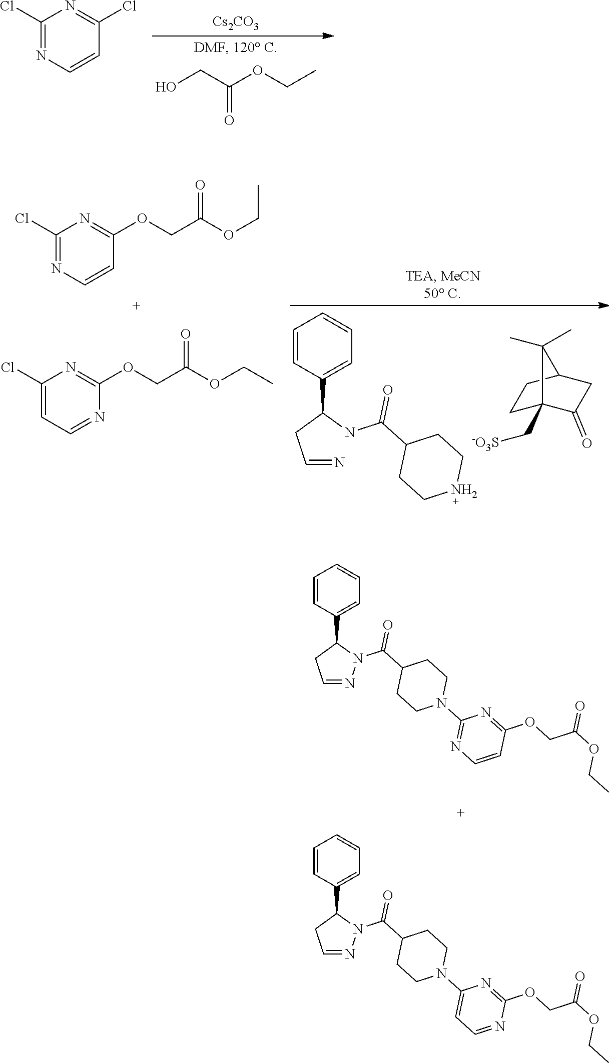

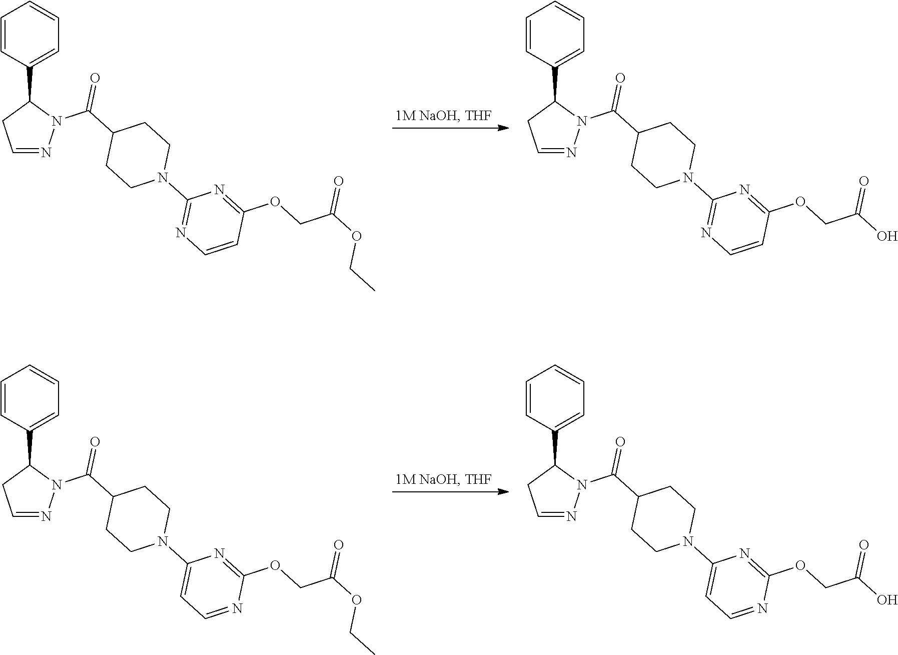 [SMILES: CCOC(=O)COc1ccnc(N2CCC(C(=O)N3N=CC[C@H]3c3ccccc3)CC2)n1 ; O=C(O)COc1ccnc(N2CCC(C(=O)N3N=CC[C@H]3c3ccccc3)CC2)n1 ; CCOC(=O)COc1nccc(N2CCC(C(=O)N3N=CC[C@H]3c3ccccc3)CC2)n1 ; O=C(O)COc1nccc(N2CCC(C(=O)N3N=CC[C@H]3c3ccccc3)CC2)n1]

### Step 1

To a suspension of 2,4-dichloropyrimidine (1 g, 6.71 mmol) and ethyl 2-hydroxyacetate (0.95 mL, 10.1 mmol) in DMF (15 mL) stirred at rt was added solid cesium carbonate (2.19 g, 6.7 mmol) in one charge. The reaction vessel was sealed. The reaction mixture was stirred at 120° C. for 5 min and evaporated in vacuo. The residue was dissolved in DCM (50 mL), filtered, and evaporated in vacuo to give a yellow oil. This residue was purified by normal phase column chromatography (CyH/EtOAc 100/0 to 60/40) to afford an inseparable mixture of ethyl 2-((2-chloropyrimidin-4-yl)oxy)acetate and ethyl 2-((4-chloropyrimidin-2-yl)oxy)acetate (ratio 2:1) (854 mg, 3.94 mmol, purity: 100%, recovery: 59%) as a colorless oil. LCMS (m/z) 217 (M+H)⁺, retention time: 2.03 min, LC/MS Method 1.

### Step 2

To a suspension of (S)-(5-phenyl-4,5-dihydro-1H-pyrazol-1-yl)(piperidin-4-yl)methanone, (1R)-10-camphorsulphonic acid salt (849 mg, 1.74 mmol), 2:1 mixture of ethyl 2-((2-chloropyrimidin-4-yl)oxy)acetate and ethyl 2-((4-chloropyrimidin-2-yl)oxy)acetate (546 mg, 2.53 mmol) in MeCN (8 mL) stirred at rt was added neat TEA (0.6 mL, 4.3 mmol). The reaction vessel was sealed. The reaction mixture was stirred at 50° C. for 15 h and evaporated in vacuo to give a pink oil. This residue was purified by normal phase column chromatography[CyH(EtOH/EtOAc 1:4) 100/0 to 30/70] to afford (S)-ethyl 2-((4-(4-(5-phenyl-4,5-dihydro-1H-pyrazole-1-carbonyl)piperidin-1-yl)pyrimidin-2-yl)oxy)acetate (502 mg, 1.2 mmol, purity: 88%, recovery: 66%) as a colorless oil and (S)-ethyl 2-((2-(4-(5-phenyl-4,5-dihydro-1H-pyrazole-1-carbonyl)piperidin-1-yl)pyrimidin-4-yl)oxy)acetate (278 mg, 0.64 mmol, purity: 100%, recovery: 37%) as a colorless oil. LCMS of the major isomer (m/z) 438 (M+H)⁺, retention time: 2.10 min, Method 1. LCMS of the minor isomer (m/z) 438 (M+H)⁺, retention time: 1.73 min, LC/MS Method 1.

### Step 3

To a solution of (S)-ethyl 2-((2-(4-(5-phenyl-4,5-dihydro-1H-pyrazole-1-carbonyl)piperidin-1-yl)pyrimidin-4-yl)oxy)acetate (270 mg, 0.62 mmol) in THF (3 mL) stirred at rt was added a 1 M solution of sodium hydroxide in water (1.85 mL, 1.85 mmol) dropwise. The reaction mixture was stirred at rt for 15 h. 1 M HCl solution (3 mL), EtOAc (15 mL), and water (10 mL) were added. After separation, the aqueous layer was extracted with EtOAc (2×15 mL). The combined organic layers were washed with brine (15 mL), dried over sodium sulfate, and evaporated in vacuo to afford (S)-2-((2-(4-(5-phenyl-4,5-dihydro-1H-pyrazole-1-carbonyl)piperidin-1-yl)pyrimidin-4-yl)oxy)acetic acid (104 mg, 0.25 mmol, purity: 100%, recovery: 41%) as a white solid. LCMS (m/z) 410 (M+H)⁺, retention time: 1.51 min, LC/MS Method 1. ¹H NMR (400 MHz, DMSO-d6) δ ppm 12.78 (s, 1H), 7.97 (s, 1H), 7.32 (dd, J=7.6, 7.2 Hz, 2H), 7.24 (m, 2H), 7.11 (d, J=7.2 Hz, 2H), 6.50 (d, J=6.1 Hz, 1H), 5.31 (dd, J=12.0, 4.6 Hz, 1H), 4.68 (s, 2H), 4.33 (m, 2H), 3.50 (ddd, J=18.8, 12.0, 1.3 Hz, 1H), 3.40 (tt, J=11.5, 3.8 Hz, 1H), 3.02 (m, 2H), 2.68 (ddd, J=19.0, 4.7, 1.7 Hz, 1H), 1.88 (d, J=13.0 Hz, 1H), 1.76 (d, J=13.0 Hz, 1H), 1.45 (m, 2H).

Example 153 was synthesized in an analogous manner.

| | | | | LC: | |
|---|---|---|---|---|---|
| | | | | retention | |
| | | | | time | |
| | | | | (min); | |
| | | | | LC/MS | |
| | | | | Method of | MS |
| Ex. | Name | Structure | ¹H NMR | analysis | (M + H)⁺ |
| 153 | (S)-2- ((4-(4-(5- phenyl- 4,5- dihydro- 1H- pyrazole-1- carbonyl) piperidin- 1-yl) pyrimidin- 2-yl)oxy) acetic acid | 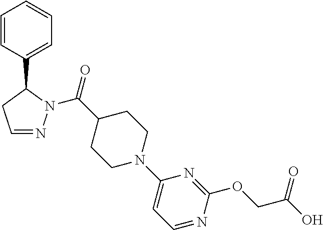 [SMILES: O=C(O)COc1nccc(N2CCC(C(=O)N3N=CC[C@H]3c3ccccc3)CC2)n1] | ¹H NMR (400 MHz, DMSO-d6) δ ppm 12.91 (s, 1H), 8.11 (d, J = 5.5 Hz, 1H), 7.32 (dd, J = 7.6, 7.2 Hz, 2H), 7.24 (m, 2H), 7.11 (m, 2H), 6.12 (d, J = 5.5 Hz, 1H), 5.31 (dd, J = 11.9, 4.6 Hz, 1H), 4.75 (s, 2H), 4.57 (d, J = 12.7 Hz, 2H), 3.49 (ddd, J = 18.8, 11.8, 1.3 Hz, 1H), | 1.61 Method 1 | 410 |
| | | | 3.38 (tt, J = 11.6, | | |
| | | | 3.8 Hz, 1H), 2.95 | | |
| | | | (m, 2H), 2.68 | | |
| | | | (ddd, J = 18.8, | | |
| | | | 4.6, 1.7 Hz, 1H), | | |
| | | | 1.84 (d, J = 12.9 | | |
| | | | Hz, 1H), 1.73 | | |
| | | | (d, J = 12.9 Hz, | | |
| | | | 1H), 1.45 (m, 2H) | | |

### Example 154

### (S)-(1-(4-((2H-tetrazol-5-yl)methoxy)-5-fluoropyrimidin-2-yl)piperidin-4-yl)(5-phenyl-4,5-dihydro-1H-pyrazol-1-yl)methanone

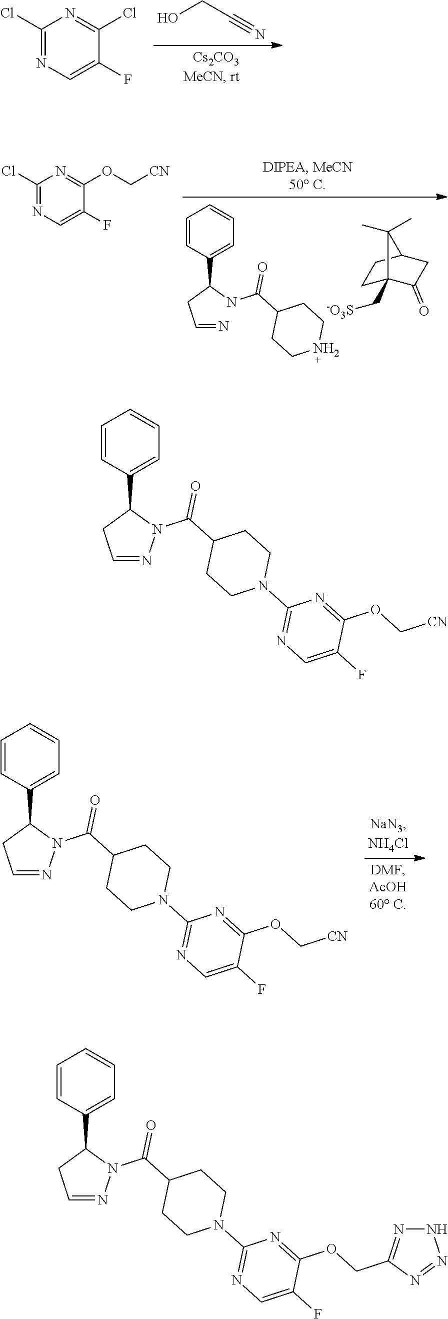

### Step 1

To a suspension of 2,4-dichloro-5-fluoropyrimidine (500 mg, 3 mmol) in MeCN (40 mL) stirred at rt was added a 70% solution of 2-hydroxyacetonitrile in water (0.227 mL, 3 mmol) and cesium carbonate (1.95 g, 6 mmol). The reaction mixture was stirred at rt for 1.5 h and filtered. The filtrate was evaporated in vacuo. The residue was purified by normal phase column chromatography (CyH/DCM 100/0 to 50/50) to afford 2-((2-chloro-5-fluoropyrimidin-4-yl)oxy)acetonitrile (280 mg, 1.42 mmol, purity: 100%, recovery: 47%) as a white powder. LCMS (m/z) 188 (M+H)⁺, retention time: 1.78 min, LC/MS Method 1.

### Step 2

To a suspension of (S)-(5-phenyl-4,5-dihydro-1H-pyrazol-1-yl)(piperidin-4-yl)methanone, (1R)-10-camphorsulphonic acid salt (700 mg, 1.43 mmol) and 2-((2-chloro-5-fluoropyrimidin-4-yl)oxy)acetonitrile (270 mg, 1.44 mmol) in MeCN (2 mL) stirred at rt was added neat DIPEA (0.25 mL, 1.44 mmol) in one charge. The reaction vessel was sealed and the reaction mixture was stirred at 50° C. for 42 h. The reaction mixture was evaporated in vacuo. The residue was purified by normal phase column chromatography (CyH/EtOAc 100/0 to 50/50) to afford (S)-2-((5-fluoro-2-(4-(5-phenyl-4,5-dihydro-1H-pyrazole-1-carbonyl)piperidin-1-yl)pyrimidin-4-yl)oxy)acetonitrile (460 mg, 1.07 mmol, purity: 79%, recovery: 74%) as a white powder. LCMS (m/z) 409 (M+H)⁺, retention time: 2.60 min, LC/MS Method 1.

### Step 3

To a solution of (S)-2-((5-fluoro-2-(4-(5-phenyl-4,5-dihydro-1H-pyrazole-1-carbonyl)piperidin-1-yl)pyrimidin-4-yl)oxy)acetonitrile (200 mg, 0.49 mmol) and ammonium chloride (144 mg, 2.69 mmol) in DMF (1.6 mL) and acetic acid (0.02 mL) stirred at rt was added sodium azide (96 mg, 1.47 mmol) in one charge. The reaction mixture was stirred at 60° C. for 68 h. To the reaction mixture was cooled to 0° C. Water (10 mL), 1 M HCl solution (0.5 mL), DCM (10 mL), and EtOH (10 mL) were added and the resulting mixture evaporated in vacuo. Water (20 mL) and a 1 M solution of HCl in water (0.5 mL) were added to the residue. The resulting solid was dissolved in DCM (50 mL) and water (30 mL). The aqueous phase was acidified with 1 M HCl solution to pH 1. After separation, the organic phase was dried over sodium sulfate, filtered, and evaporated in vacuo. The residue was added purified by normal phase column chromatography (DCM/EtOH 100/0 to 95/5) to afford (S)-(1-(4-((2H-tetrazol-5-yl)methoxy)-5-fluoropyrimidin-2-yl)piperidin-4-yl)(5-phenyl-4,5-dihydro-1H-pyrazol-1-yl)methanone (25 mg, 0.05 mmol, purity: 100%, recovery: 11%) as an off white powder. LCMS (m/z) 452 (M+H)⁺, retention time: 2.29 min, LC/MS Method 1. ¹H NMR (400 MHz, DMSO-d6) δ ppm 8.24 (d, J=3.0 Hz, 1H), 7.33 (dd, J=7.6, 7.2 Hz, 2H), 7.24 (m, 2H), 7.10 (d, J=7.2 Hz, 2H), 5.77 (s, 2H), 5.31 (dd, J=12.0, 4.6 Hz, 1H), 4.42 (d, J=12.9 Hz, 2H), 3.50 (ddd, J=18.8, 12.0, 1.2 Hz, 1H), 3.33 (tt, J=11.5, 3.6 Hz, 1H), 2.93 (m, 2H), 2.67 (ddd, J=18.8, 4.6, 1.5 Hz, 1H), 1.82 (d, J=11.4 Hz, 1H), 1.67 (d, J=11.6 Hz, 1H), 1.35 (m, 2H). Proton of the tetrazole not visible.

### Example 155

### (S)-(1-(5-fluoro-4-(2-hydroxyethoxy)pyrimidin-2-yl)piperidin-4-yl)(5-phenyl-4,5-dihydro-1H-pyrazol-1-yl)methanone

Example 155 was synthesized in an analogous manner as Example 152 with a deprotection step.

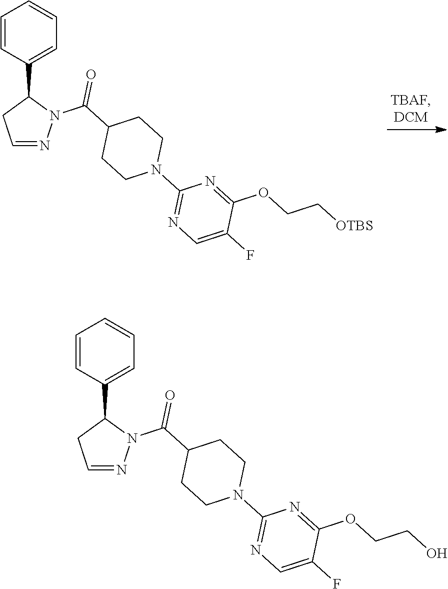 [SMILES: CC(C)(C)[Si](C)(C)OCCOc1nc(N2CCC(C(=O)N3N=CC[C@H]3c3ccccc3)CC2)ncc1F ; O=C(C1CCN(c2ncc(F)c(OCCO)n2)CC1)N1N=CC[C@H]1c1ccccc1]

To a solution of (S)-(1-(4-(2-((tert-butyldimethylsilyl)oxy)ethoxy)-5-fluoropyrimidin-2-yl)piperidin-4-yl)(5-phenyl-4,5-dihydro-1H-pyrazol-1-yl)methanone (240 mg, 0.46 mmol) in DCM (5 mL) stirred at 0° C. was added a 1 M solution of TBAF in THF (0.46 mL, 0.46 mmol) in one charge. The reaction mixture was stirred at rt for 20 minutes. A second batch of 1 M solution of TBAF in THF (1.4 mL, 1.4 mmol) was added at rt and the mixture was stirred at rt for a further 2.5 h. The solvent was evaporated in vacuo. The residue was purified by normal phase column chromatography (DCM/MeOH 100/0 to 85/15) to afford (S)-(1-(5-fluoro-4-(2-hydroxyethoxy)pyrimidin-2-yl)piperidin-4-yl)(5-phenyl-4,5-dihydro-1H-pyrazol-1-yl)methanone (20 mg, 0.05 mmol, purity: 100%, recovery: 10%) as a off-white powder. LCMS (m/z) 414 (M+H)⁺, retention time: 2.24 min, LC/MS Method 1. ¹H NMR (400 MHz, DMSO-d6) δ ppm 8.23 (d, J=3.0 Hz, 1H), 7.32 (t, J=7.1, 7.2 Hz, 2H), 7.24 (t, J=7.9 Hz, 2H), 7.11 (d, J=7.2 Hz, 2H), 5.31 (dd, J=11.6, 4.4 Hz, 1H), 4.42 (d, J=4.6, 11.9 Hz, 2H), 3.49 (ddd, J=1.1, 12.0, 18.7 Hz, 1H), 3.33 (tt, J=3.6, 11.3 Hz, 1H), 2.98-2.86 (m, 2H), 2.67 (ddd, J=1.5, 4.6, 19.0 Hz, 1H), 2.55-2.48 (m, 3H), 2.09 (s, 1H), 1.82 (d, J=11.4 Hz, 1H), 1.67 (d, J=11.8 Hz, 1H), 1.48-1.25 (m, 2H). Proton of the alcohol not visible.

### Example 156

### (S)-(1-(6-ethynylpyrimidin-4-yl)piperidin-4-yl)(5-phenyl-4,5-dihydro-1H-pyrazol-1-yl)methanone

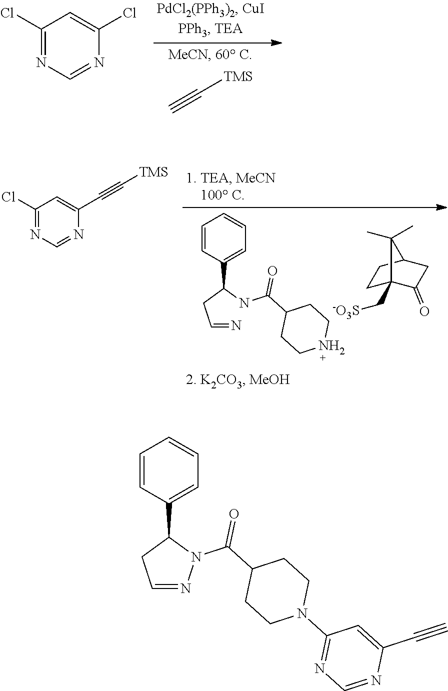

### Step 1

To a suspension of ethynyltrimethylsilane (2.9 mL, 20.1 mmol), 4,6-dichloropyrimidine (2 g, 13.4 mmol), copper(I) iodide (0.26 g, 1.34 mmol), triphenylphosphine (0.35 g, 1.34 mmol) and TEA (3.7 mL, 26.8 mmol) in MeCN (13 mL) stirred under argon at rt was added PdCl₂(PPh₃)₂ (0.24 g, 0.34 mmol) in one charge. The reaction mixture was stirred at 60° C. for 2 h. Water (100 mL) and EtOAc (200 mL) were added. After separation, the aqueous layer was extracted with EtOAc (2×100 mL). The combined organic layers were washed with brine, dried over sodium sulfate and evaporated in vacuo to give a black oil. This residue was purified by normal phase column chromatography (CyH/EtOAc 100/0-90/10) to afford 4-chloro-6-((trimethylsilyl)ethynyl)pyrimidine (707 mg, 3.35 mmol, purity: 63%, recovery: 25%) as a brown solid. LCMS (m/z) 211 (M+H)⁺, retention time: 2.94 min, LC/MS Method 1.

### Step 2

To a suspension of (S)-(5-phenyl-4,5-dihydro-1H-pyrazol-1-yl)(piperidin-4-yl)methanone, (1R)-10-camphorsulphonic acid salt (220 mg, 0.450 mmol) and 4-chloro-6-((trimethylsilypethynyl)pyrimidine (114 mg, 0.540 mmol) in MeCN (4 mL) stirred at rt was added TEA (0.188 mL, 1.35 mmol). The reaction vessel was sealed and microwaved at 100° C. for 20 min. The reaction mixture was evaporated in vacuo to give a brown oil. This residue was dissolved in MeOH (4 mL) and potassium carbonate (187 mg, 1.35 mmol) was added. The reaction mixture was stirred 2 h at rt, filtered over celite, and evaporated in vacuo to give an orange solid. Water (25 mL) and EtOAc (25 mL) were added. After separation, the aqueous layer was extracted with EtOAc (2×25 mL). The combined organic layers were washed with brine, dried over sodium sulfate and evaporated in vacuo to give an orange oil. This residue was purified by normal phase column chromatography [CyH/(EtOH/EtOAc 1:4) 100/0 to 60/40] to afford (S)-(1-(6-ethynylpyrimidin-4-yl)piperidin-4-yl)(5-phenyl-4,5-dihydro-1H-pyrazol-1-yl)methanone (60 mg, 0.17 mmol, purity: 100%, recovery: 37%) as a cream-colored solid. LCMS (m/z) 360 (M+H)⁺, retention time: 1.75 min, LC/MS Method 1. ¹H NMR (400 MHz, DMSO-d6) δ ppm 8.42 (d, J=1.1 Hz, 1H), 7.32 (dd, J=7.6, 7.2 Hz, 2H), 7.24 (m, 2H), 7.10 (d, J=7.2 Hz, 2H), 7.02 (d, J=1.0 Hz, 1H), 5.31 (dd, J=11.8, 4.6 Hz, 1H), 4.42 (s, 1H), 4.39 (br.s, 2H), 3.50 (ddd, J=19.0, 12.0, 1.5 Hz, 1H), 3.40 (tt, J=11.3, 3.7 Hz, 1H), 3.04 (m, 2H), 2.68 (ddd, J=18.8, 4.6, 1.7 Hz, 1H), 1.90 (d, J=12.0 Hz, 1H), 1.77 (d, J=12.0 Hz, 1H), 1.47 (m, 2H).

Examples 157 and 158 were synthesized in an analogous manner.

| | | | | LC: | |
|---|---|---|---|---|---|
| | | | | retention | |
| | | | | time | |
| | | | | (min); | |
| | | | | LC/MS | |
| | | | | Method of | MS |
| Ex. | Name | Structure | ¹H NMR | analysis | (M + H)⁺ |
| 157 | (S)-(5-(3,5- difluoro- phenyl)- 4,5- dihydro- 1H- pyrazol-1- yl)(1-(6- ethynyl- pyrimidin- 4-yl) piperidin- 4-yl) methanone | 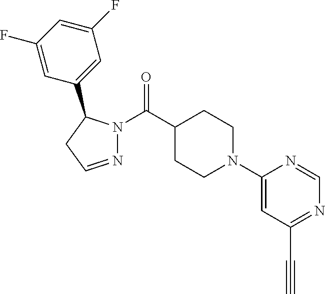 [SMILES: C#Cc1cc(N2CCC(C(=O)N3N=CC[C@H]3c3cc(F)cc(F)c3)CC2)ncn1] | ¹H NMR (400 MHz, DMSO-d6) δ ppm 8.42 (s, 1H), 7.25 (s, 1H), 7.12 (t, J = 9.1 Hz, 1H), 7.03 (s, 1H), 6.84 (d, J = 6.5 Hz, 2H), 5.34 (dd, J = 11.8, 4.6 Hz, 1H), 4.42 (s, 1H), 4.41 (m, 2H), 3.48 (dd, J = 18.8, 12.3 Hz, 1H), 3.40 (m, 1H), 3.03 (m, 2H), 2.75 (dd, J = 18.9, 3.9 Hz, 1H), 1.91 (d, J = 11.8 Hz, 1H), 1.77 (d, J = 11.8 Hz, 1H), 1.46 (m, 2H) | 1.92 Method 1 | 396 |
| 158 | (S)-3-(1- (1-(6- ethynyl- pyrimidin- 4-yl) piperidine- 4- carbonyl)- 4,5- dihydro- 1H- pyrazol- 5-yl) benzonitrile | 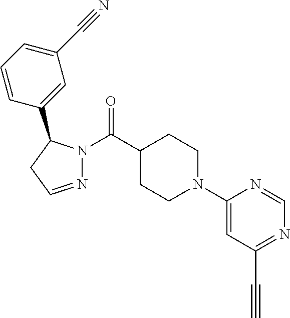 [SMILES: C#Cc1cc(N2CCC(C(=O)N3N=CC[C@H]3c3cccc(C#N)c3)CC2)ncn1] | ¹H NMR (400 MHz, DMSO-d6) δ ppm 8.42 (s, 1H), 7.73 (d, J = 7.6 Hz, 1H), 7.60 (s, 1H), 7.55 (dd, J = 7.8 Hz, 1H), 7.45 (d, J = 7.8 Hz, 1H), 7.27 (s, 1H), 7.02 (s, 1H), 5.37 (dd, J = 12.0, 5.1 Hz, 1H), 4.42 (s, 1H), 4.40 (m, 2H), 3.51 (dd, J = 18.4, 12.7 Hz, 1H), 3.38 (m, 1H), 3.03 (m, 2H), 2.76 (dd, J = 19.0, 4.9, 1.3 Hz, 1H), 1.89 (d, J = 12.1 Hz, 1H), 1.78 (d J = | 1.83 Method 1 | 385 |
| | | | 12.0 Hz, 1H), 1.45 | | |
| | | | (m, 2H) | | |

### Example 159

### (S)-5-fluoro-6-(4-(5-phenyl-4,5-dihydro-1H-pyrazole-1-carbonyl)piperidin-1-yl)pyrimidine-4-carboxylic acid

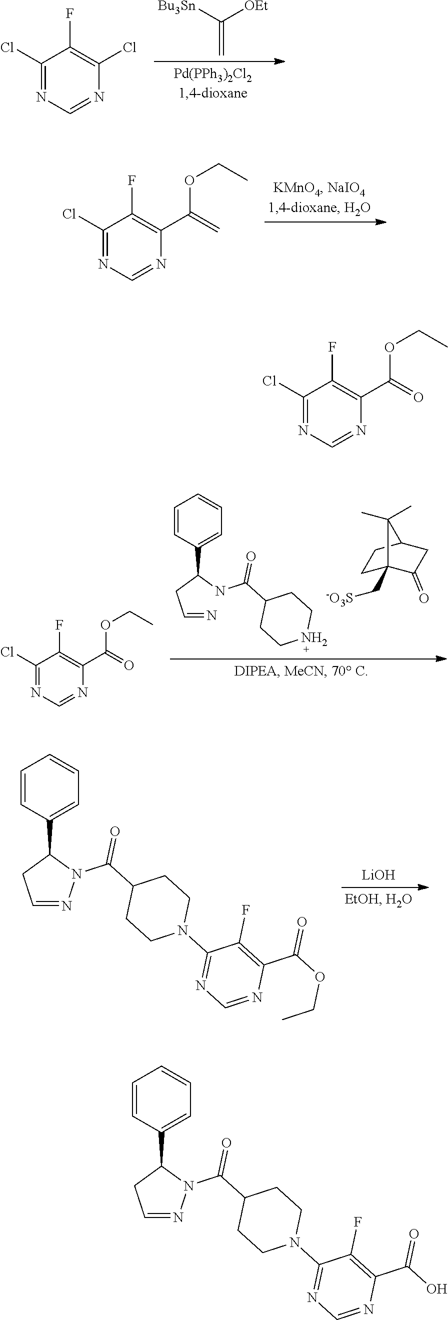

Step 1 To a solution of Pd(PPh₃)₂Cl₂ (4.20 g, 5.99 mmol) in 1,4-dioxane (200 mL) stirred at rt was added tributyl(1-ethoxyvinyl)stannane (20.2 mL, 60 mmol) and 4,6-dichloro-5-fluoropyrimidine (10 g, 60 mmol) in one charge. Nitrogen was bubbled in the reaction mixture for 10 min. The reaction mixture was then stirred at rt for 18 h under nitrogen. The solvent was evaporated in vacuo. Water (500 mL) and Et₂O (500 mL) were added. After separation, the organic layer was dried over sodium sulfate and evaporated in vacuo. The residue was purified by normal phase column chromatography (CyH/DCM 100/0 to 40/60) to afford 4-chloro-6-(1-ethoxyvinyl)-5-fluoropyrimidine (5 g, 23.44 mmol, purity: 100%, recovery: 39%) as an orange oil. LCMS (m/z) 203 and 205 (M+H)⁺, retention time: 2.49 min, LC/MS Method 1.

### Step 2

To a solution of 4-chloro-6-(1-ethoxyvinyl)-5-fluoropyrimidine (2.5 g, 12.34 mmol) in 1,4-dioxane (150 mL) stirred at 0° C. was added sodium periodate (5.28 g, 24.68 mmol) in water (40 mL) in one charge followed by potassium permanganate (0.975 g, 6.17 mmol) portion-wise. The reaction mixture was stirred at rt for 15 h. The residue was filtered and the solid was rinsed with DCM (150 mL) and MeOH (50 mL). Brine (200 mL) was added to the filtrate. After separation, the aqueous layer was extracted with DCM (200 mL). The combined organic layers were dried over sodium sulfate and evaporated in vacuo. The residue was purified by normal phase column chromatography (CyH/DCM 90/10) to afford ethyl 6-chloro-5-fluoropyrimidine-4-carboxylate (480 mg, 2.23 mmol, purity: 100%, recovery: 8%) as a colorless oil. LCMS (m/z) 205 and 207 (M+H)⁺, retention time: 2.06 min, LC/MS Method 1.

### Step 3

To a suspension of ethyl 6-chloro-5-fluoropyrimidine-4-carboxylate (0.9 g, 4.4 mmol) and (S)-(5-phenyl-4,5-dihydro-1H-pyrazol-1-yl)(piperidin-4-yl)methanone camphor-10-sulphonic acid salt (2 g, 4.1 mmol) in MeCN (20 mL) was added DIPEA (1.8 mL, 10.3 mmol). The reaction vessel was sealed and then stirred at 80° C. for 30 min. The reaction mixture was evaporated in vacuo. The residue was purified by normal phase column chromatography [CyH/(EtOAc/EtOH 3:1) 100/0 to 85/15] to afford (S)-ethyl 5-fluoro-6-(4-(5-phenyl-4,5-dihydro-1H-pyrazole-1-carbonyl)piperidin-1-yl)pyrimidine-4-carboxylate (1.78 g, 4.0 mmol, 97%) as a light yellow oil. LCMS (m/z) 426 (M+H)⁺, retention time: 2.5 min, LC/MS Method 1.

### Step 4

To a solution of (S)-ethyl 5-fluoro-6-(4-(5-phenyl-4,5-dihydro-1H-pyrazole-1-carbonyl)piperidin-1-yl)pyrimidine-4-carboxylate (1.78 g, 4.2 mmol) in EtOH (30 mL) stirred at 0° C. was added LiOH (470 mg, 6.28 mmol) in water (10 mL) portion-wise. The reaction was stirred at rt for 30 min. The reaction mixture was evaporated in vacuo. Water was added (250 mL) and the pH adjusted to 3 with 1N HCl solution. DCM was added (150 mL). After separation, the aqueous layer was extracted with DCM (150 mL). The combined organic layers were dried over sodium sulfate and evaporated to give (S)-5-fluoro-6-(4-(5-phenyl-4,5-dihydro-1H-pyrazole-1-carbonyl)piperidin-1-yl)pyrimidine-4-carboxylic acid (1.35 g, 3.23 mmol, 77%) as an off-white powder. LCMS (m/z) 398 (M+H)⁺, retention time: 1.77 min, LC/MS Method 1. ¹H NMR (400 MHz, DMSO-d6) δ ppm: 13.87 (br s, 1H), 8.36 (d, J=2.7 Hz, 1H), 7.32 (t, J=7.4 Hz, 2H), 7.24 (m, 2H), 7.11 (d, J=7.0 Hz, 2H), 5.32 (dd, J=11.8, 4.6 Hz, 1H), 4.42 (d, J=13.5 Hz, 2H), 3.50 (dd, J=11.8 Hz, 1.4 Hz, 1H), 3.44 (m, 1H), 3.22 (m, 2H), 2.68 (ddd, J=19.0, 4.6, 1.7, 1H), 1.95 (m, 1H), 1.81 (d, J=13.0 Hz, 1H), 1.60 (sextuplet of d, J=4.0, 11.5 Hz, 2H).

### Example 160

### (S)-5-fluoro-6-(4-(5-(5-fluoropyridin-3-yl)-4,5-dihydro-1H-pyrazole-1-carbonyl)piperidin-1-yl)pyrimidine-4-carboxamide

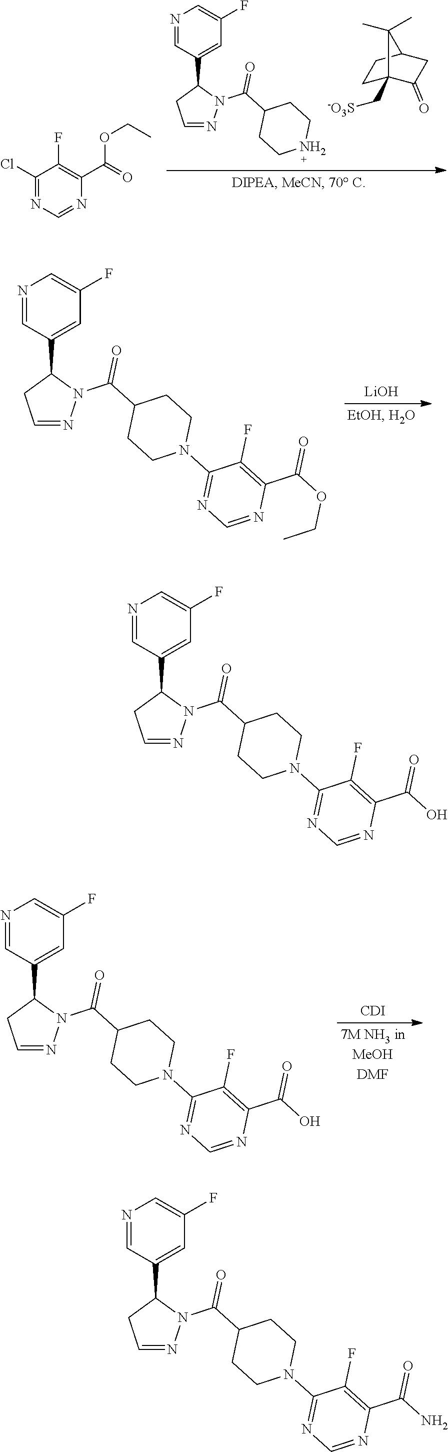

### Step 1

To a suspension of (S)-(5-(5-fluoropyridin-3-yl)-4,5-dihydro-1H-pyrazol-1-yl)(piperidin-4-yl)methanone, 1R-(−)-camphor-10-sulphonic acid salt (240 mg, 0.47 mmol) in MeCN (10 mL) were added ethyl 6-chloro-5-fluoropyrimidine-4-carboxylate (97 mg, 0.47 mmol) and DIPEA (0.21 mL, 1.2 mmol). The mixture was then stirred at 70° C. for 2 h. The reaction mixture was evaporated in vacuo. Water (150 mL) and DCM (150 mL) were added. After separation, the aqueous layer was extracted with DCM (150 mL). The combined organic layers were dried over sodium sulfate and evaporated in vacuo. The residue was purified by normal phase column chromatography [CyH/(EtOAc/EtOH 3:1) 100/0 to 85/15] to afford (S)-ethyl 5-fluoro-6-(4-(5-(5-fluoropyridin-3-yl)-4,5-dihydro-1H-pyrazole-1-carbonyl)piperidin-1-yl)pyrimidine-4-carboxylate (250 mg, 0.56 mmol, purity: 80%, recovery: 100%) as a yellow oil. LCMS (m/z) 445 (M+H)⁺, retention time: 2.14 min, LC/MS Method 1.

### Step 2

To a solution of (S)-ethyl 5-fluoro-6-(4-(5-(5-fluoropyridin-3-yl)-4,5-dihydro-1H-pyrazole-1-carbonyl)piperidin-1-yl)pyrimidine-4-carboxylate (250 mg, 0.56 mmol) in EtOH (15 mL) and water (5 mL) stirred at 0° C. was added LiOH (20 mg, 0.84 mmol) portion-wise. The reaction was stirred at rt for 2 h. The reaction mixture was evaporated in vacuo. Water was added (100 mL) and the pH adjusted to 2 with AcOH. DCM was added (100 mL). After separation, the aqueous layer was extracted with DCM (100 mL). The combined organic layers were dried over sodium sulfate and evaporated to give (S)-5-fluoro-6-(4-(5-(5-fluoropyridin-3-yl)-4,5-dihydro-1H-pyrazole-1-carbonyl)piperidin-1-yl)pyrimidine-4-carboxylic acid (120 mg, 0.288 mmol, purity: 100%, recovery: 51%) as a yellow gum. LCMS (m/z) 417 (M+H)⁺, retention time: 1.46 min, LC/MS Method 1.

### Step 3

To a solution of (S)-5-fluoro-6-(4-(5-(5-fluoropyridin-3-yl)-4,5-dihydro-1H-pyrazole-1-carbonyl)piperidin-1-yl)pyrimidine-4-carboxylic acid (100 mg, 0.24 mmol) in DMF (5 mL) were added CDI (38.9 mg, 0.24 mmol) and a 7 M solution of ammonia in MeOH (0.274 mL, 1.92 mmol). The reaction mixture was stirred for 3 h at rt and evaporated in vacuo. Water (100 mL) and DCM (100 mL) were added. After separation, the aqueous layer was extracted with DCM (100 mL). The combined organic layers were dried over sodium sulfate and evaporated in vacuo. The residue was purified by normal phase column chromatography (DCM/MeOH 100/0 to 95/5) to afford (S)-5-fluoro-6-(4-(5-(5-fluoropyridin-3-yl)-4,5-dihydro-1H-pyrazole-1-carbonyl)piperidin-1-yl)pyrimidine-4-carboxamide. Trituration in Et₂O afforded, after filtration, (S)-5-fluoro-6-(4-(5-(5-fluoropyridin-3-yl)-4,5-dihydro-1H-pyrazole-1-carbonyl)piperidin-1-yl)pyrimidine-4-carboxamide (14 mg, 0.03 mmol, purity: 100%, recovery: 14%) as a white powder. LCMS (m/z) 416 (M+H)⁺, retention time: 1.71 min, LC/MS Method 1. ¹H NMR (400 MHz, DMSO-d6) δ ppm: 8.47 (d, J=2.5 Hz, 1H), 8.34 (d, J=2.5 Hz, 1H), 8.29 (s, 1H), 8.00 (s, 1H), 7.75 (s, 1H), 7.47 (d, J=8.2 Hz, 1H), 7.29 (s, 1H), 5.41 (dd, J=11.8, 5.1 Hz, 1H), 4.41 (d, J=12.9 Hz, 2H), 3.52 (dd, J=18.9, 12.2 Hz, 1H), 3.41 (m, 1H), 3.21 (t, J=13.0 Hz, 2H), 2.84 (ddd, J=19.0, 5.0, 1.2 Hz, 1H), 1.93 (d, J=12.3 Hz, 1H), 1.81 (d, J=12.2 Hz, 1H), 1.57 (m, 2H).

Examples 161 and 162 were synthesized in an analogous manner to Example 160 from 2,4-dichloro-5-fluoropyrimidine. Examples 163-165 were synthesized in an analogous manner to Example 160.

| | | | | LC: | |
|---|---|---|---|---|---|
| | | | | retention | |
| | | | | time | |
| | | | | (min); | |
| | | | | LC/MS | |
| | | | | Method of | MS |
| Ex. | Name | Structure | ¹H NMR | analysis | (M + H)⁺ |
| 161 | (S)-5- fluoro-2- (4-(5-(5- fluoro- pyridin- 3-yl)-4,5- dihydro- 1H- pyrazole-1- carbonyl) piperidin- 1-yl) pyrimidine- 4- carboxamide | 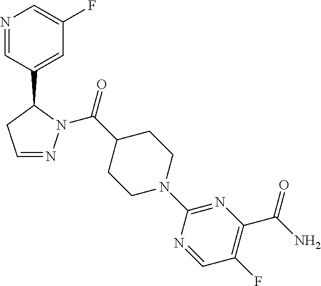 [SMILES: NC(=O)c1nc(N2CCC(C(=O)N3N=CC[C@H]3c3cncc(F)c3)CC2)ncc1F] | ¹H NMR (400 MHz, DMSO-d6) δ ppm 8.54 (d, J = 2.5 Hz, 1H), 8.45 (d, J = 2.5 Hz, 1H), 8.29 (s, 1H), 8.12 (br s, 1H), 7.81 (br s, 1H), 7.47 (d, J = 9.5 Hz, 1H), 7.29 (s, 1H), 5.41 (dd, J = 11.8, 5.1 Hz, 1H), 4.63 (d, J = 13.3 Hz, 2H), 3.52 (dd, J = 18.2, 12.2 Hz, 1H), 3.38 (m, 1H), 3.00 (m, 1H), 2.84 (dd, J = 18.8, 5.1 Hz, 1H), 1.88 (d, J = 12.5 Hz, | 1.74 Method 1 | 416 |
| | | | 1H), 1.77 (d, J = | | |
| | | | 12.5 Hz, 1H), 1.46 | | |
| | | | (m, 2H) | | |
| 162 | (S)-5- fluoro- 2-(4-(5- phenyl- 4,5- dihydro- 1H- pyrazole- 1- carbonyl) piperidin- 1-yl) pyrimidine- 4- carboxamide | 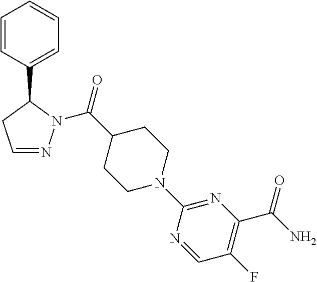 [SMILES: NC(=O)c1nc(N2CCC(C(=O)N3N=CC[C@H]3c3ccccc3)CC2)ncc1F] | ¹H NMR (400 MHz, DMSO-d6) δ ppm 8.53 (d, J = 2.5 Hz, 1H), 8.11 (s, 1H), 7.80 (s, 1H), 7.32 (dd, J = 7.6, 7.2 Hz, 2H), 7.23 (m, 2H), 7.11 (d, J = 7.0 Hz, 2H), 5.31 (dd, J = 12.0, 4.6 Hz, 1H), 4.63 (d, J = 13.3 Hz, 2H), 3.50 (m, 1H), 3.42 (m, 1H), 3.01 (m, 2H), 2.68 (ddd, J = 17.3, 4.6, 1.5 Hz, 1H), 1.90 (d, J = 12.3 Hz, 1H), 1.76 (d, J = | 2.20 Method 1 | 397 |
| | | | 12.0 Hz, 1H), 1.50 | | |
| | | | (m, 2H) | | |
| 163 | (S)-N,N- diethyl- 5-fluoro- 6-(4-(5- phenyl- 4,5- dihydro- 1H- pyrazole- 1- carbonyl) piperidin- 1-yl) pyrimidine- 4- carboxamide | 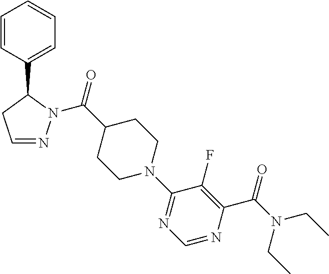 [SMILES: CCN(CC)C(=O)c1ncnc(N2CCC(C(=O)N3N=CC[C@H]3c3ccccc3)CC2)c1F] | ¹H NMR (400 MHz, DMSO-d6) δ ppm 8.34 (d, J = 3.0 Hz, 1H), 7.32 (t, J = 7.6 Hz, 2H), 7.24 (m, 2H), 7.11 (d, J = 7.2 Hz, 2H), 5.32 (dd, J = 11.8, 4.6 Hz, 1H), 4.43 (d, J = 13.3 Hz, 2H), 3.50 (dd, J = 18.8, 11.8 Hz, 1H), 3.43 (q, J = 7.1 Hz, 2H), 3.43 (m, 1H), 3.21 (m, 2H), 3.15 (q, J = 7.0 Hz, 2H), 2.68 (ddd, J = 19.0, 4.4, 1.1 Hz), | 1.79 Method 1 | 418 |
| | | | 1.94 (d, J = 11.6 | | |
| | | | Hz, 1H), 1.81 (d, | | |
| | | | J = 11.2 Hz, 1H), | | |
| | | | 1.59 (m, 2H), 1.13 | | |
| | | | (t, J = 7.1 Hz, 3 H), | | |
| | | | 1.04 (t, J = 6.9 Hz, | | |
| | | | 3H) | | |
| 164 | (S)-6-(4- (5-(3,5- difluoro- phenyl)- 4,5- dihydro- 1H- pyrazole- 1- carbonyl) piperidin- 1-yl)-5- fluoro- pyrimidine- 4- carboxylic acid | 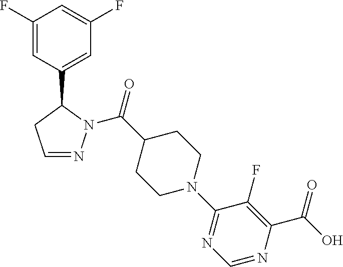 [SMILES: O=C(O)c1ncnc(N2CCC(C(=O)N3N=CC[C@H]3c3cc(F)cc(F)c3)CC2)c1F] | ¹H NMR (400 MHz, DMSO-d6) δ ppm 13.89 (s, 1H), 8.36 (s, 1H), 7.26 (s, 1H), 7.12 (m, 1H), 6.84 (d, J = 6.5 Hz, 2H), 5.34 (dd, J = 11.8, 4.6 Hz, 1H), 4.42 (d, J = 12.7 Hz, 2H), 3.47 (m, 2H), 3.23 (m, 2H), 2.73 (dd, J = 19.1, 3.8 Hz, 1H), 1.96 (d, J = 12.3 Hz, 1H), 1.82 (d, J = 11.0 Hz, 1H), 1.61 (m, 2H) | 1.91 Method 1 | 434 |
| 165 | (S)-6-(4- (5-(3,5- difluoro- phenyl)- 4,5- dihydro- 1H- pyrazole- 1- carbonyl) piperidin- 1-yl)-5- fluoro- pyrimidine- 4- carboxamide | 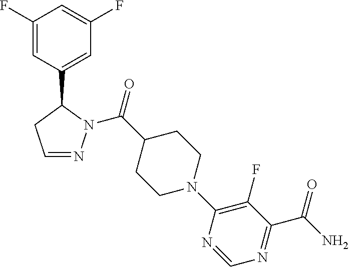 [SMILES: NC(=O)c1ncnc(N2CCC(C(=O)N3N=CC[C@H]3c3cc(F)cc(F)c3)CC2)c1F] | ¹H NMR (400 MHz, DMSO-d6) δ ppm 8.34 (d, J = 2.5 Hz, 1H), 8.01 (s, 1H), 7.75 (s, 1H), 7.26 (s, 1H), 7.12 (t, J = 9.3 Hz, 1H), 6.84 (d, J = 6.5 Hz, 2H), 5.34 (dd, J = 11.9, 4.8 Hz, 1H), 4.42 (d, J = 12.7 Hz, 2H), 3.48 (m, 2H), 3.21 (m, 2H), 2.75 (dd, J = 18.9, 4.8 Hz, 1H), 1.96 (d, J = 11.8 Hz, 1H), 1.81 (d, J = 11.6 | 2.15 Method 1 | 433 |
| | | | Hz, 1H), 1.59 (m, | | |
| | | | 2H) | | |

### Example 166

### (R)-3-(5-fluoro-2-(4-((S)-5-(5-fluoropyridin-3-yl)-4,5-dihydro-1H-pyrazole-1-carbonyl)piperidin-1-yl)pyrimidin-4-yl)pyrrolidin-2-one

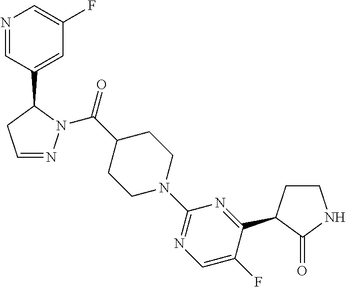 [SMILES: O=C1NCC[C@@H]1c1nc(N2CCC(C(=O)N3N=CC[C@H]3c3cncc(F)c3)CC2)ncc1F]

Example 151 was purified by chiral HPLC (conditions: on CHIRALCEL® OD-H, 5 μm, I.D. was using 20 mm×250 mm, Heptane/EtOH 60/40+0.1% diethylamine, at a flow rate of 19 mL/min) to provide 3-(5-fluoro-2-(4-((S)-5-(5-fluoropyridin-3-yl)-4,5-dihydro-1H-pyrazole-1-carbonyl)piperidin-1-yl)pyrimidin-4-yl)pyrrolidin-2-one. A trituration into iPr₂O afforded, after filtration, 3-(5-fluoro-2-(4-((S)-5-(5-fluoropyridin-3-yl)-4,5-dihydro-1H-pyrazole-1-carbonyl)piperidin-1-yl)pyrimidin-4-yl)pyrrolidin-2-one (105 mg, 0.23 mmol, purity: 100%, recovery: 26%). LCMS (m/z) 456 (M+H)⁺, retention time: 1.99 min, LC/MS Method 1. ¹H NMR (400 MHz, DMSO-d6) δ ppm: 8.47 (d, J=2.8 Hz, 1H), 8.35 (d, J=1.9 Hz, 1H), 8.29 (s, 1H), 7.91 (s, 1H), 7.47 (dt, J=2.1, 9.7 Hz, 1H), 7.28 (s, 1H), 5.41 (dd, J=5.1, 12.0 Hz, 1H), 4.55 (dd, J=2.7, 13.3 Hz, 2H), 3.84 (t, J=8.7 Hz, 1H), 3.52 (ddd, J=1.5, 12.1, 19.0 Hz, 1H), 3.42-3.28 (m, 3H), 3.04-2.91 (m, 2H), 2.84 (ddd, J=1.6, 5.2, 19.0 Hz, 1H), 2.46-2.30 (m, 2H), 1.90-1.82 (m, 1H), 1.80-1.72 (m, 1H), 1.44 (sextuplet of d, J=4.4, 12.5 Hz, 2H).

### Example 167

### (S)-2-((5-fluoro-2-(4-(5-phenyl-4,5-dihydro-1H-pyrazole-1-carbonyl)piperidin-1-yl)pyrimidin-4-yl)oxy)acetamide

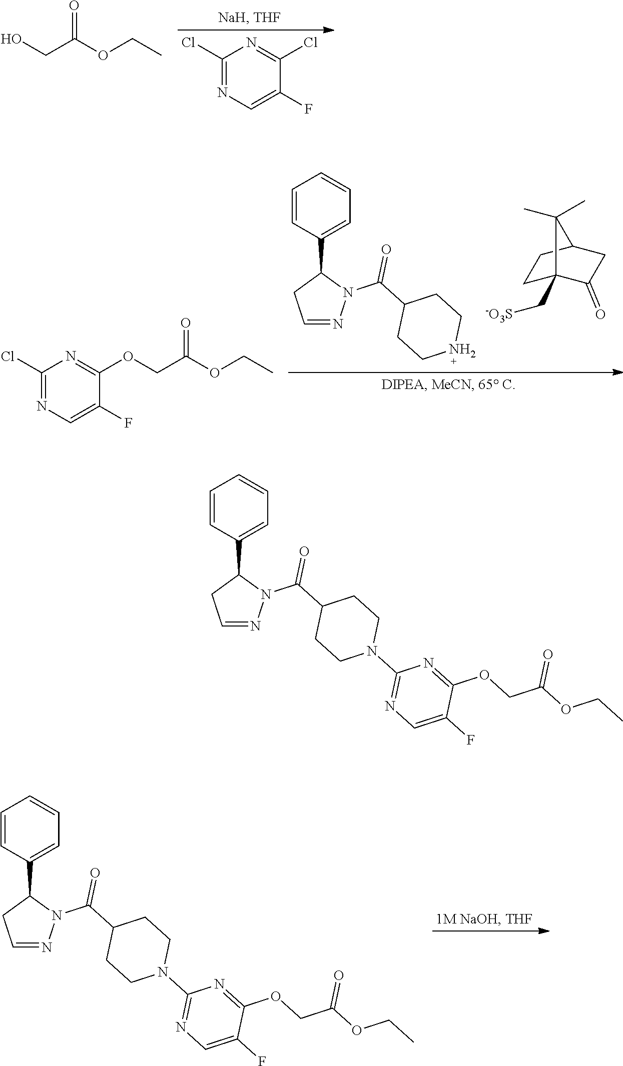

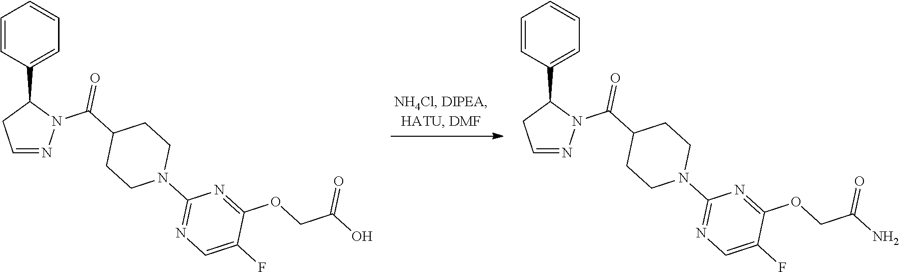 [SMILES: O=C(O)COc1nc(N2CCC(C(=O)N3N=CC[C@H]3c3ccccc3)CC2)ncc1F ; NC(=O)COc1nc(N2CCC(C(=O)N3N=CC[C@H]3c3ccccc3)CC2)ncc1F]

### Step 1

To a solution of ethyl 2-hydroxyacetate (0.6 ml, 6.34 mmol) in THF (40 ml) stirred at 0° C. was added 60% NaH in mineral oil (0.3 g, 7.5 mmol) portion-wise. The reaction mixture was allowed to warm to rt and stirred for 15 min. The reaction mixture was then cooled to 0° C. and 2,4-dichloro-5-fluoropyrimidine (1 g, 6 mmol) was added portion-wise. The reaction mixture was allowed to warm to rt and was stirred for 1 h. Water (50 mL) was added. A 1 M solution of HCl in water was added until pH 1. The aqueous layer was extracted with DCM (50 mL). The organic layer was dried over sodium sulphate and evaporated in vacuo. The residue was purified by normal phase column chromatography (CyH/EtOAc 100/0 to 90/10) to afford ethyl 2-((2-chloro-5-fluoropyrimidin-4-yl)oxy)acetate (1.22 g, 4.92 mmol, purity: 100%, recovery: 82%) as a colourless oil which solidified. LCMS (m/z) 158 and 160 (M+H)⁺, retention time: 1.07 min, LC/MS Method 1.

### Step 2

To a suspension of (S)-(5-phenyl-4,5-dihydro-1H-pyrazol-1-yl)(piperidin-4-yl)methanone, (1R)-10-camphorsulphonic acid salt (1.14 g, 2.3 mmol) and ethyl 2-((2-chloro-5-fluoropyrimidin-4-yl)oxy)acetate (547 mg, 2.3 mmol) in MeCN (2 mL) stirred at rt was added DIPEA (0.41 mL, 2.3 mmol) in one charge. The reaction vessel was sealed and the reaction mixture was stirred at 65° C. for 19 h. The reaction mixture was evaporated in vacuo. The residue was purified by normal phase column chromatography (CyH/EtOAc 100/0 to 75/25) to afford (S)-ethyl 2-((5-fluoro-2-(4-(5-phenyl-4,5-dihydro-1H-pyrazole-1-carbonyl)piperidin-1-yl)pyrimidin-4-yl)oxy)acetate (480 mg, 1 mmol, purity: 100%, recovery: 43%) as a yellow oil. LCMS (m/z) 456 (M+H)⁺, retention time: 2.68 min, LC/MS Method 1.

### Step 3

To a solution of (S)-ethyl 2-((5-fluoro-2-(4-(5-phenyl-4,5-dihydro-1H-pyrazole-1-carbonyl)piperidin-1-yl)pyrimidin-4-yl)oxy)acetate (385 mg, 0.85 mmol) in THF (8 mL) stirred at 0° C. was added a 1 M solution of sodium hydroxide in water (4.23 mL, 4.23 mmol) dropwise. The reaction mixture was allowed to warm to rt and stirred for 2 h. A 1 M solution of HCl in water (4.2 mL) was added until pH 4, followed by EtOAc (20 mL). After separation, the aqueous layer was extracted with EtOAc (2×10 mL). The combined organic layers were washed with brine, dried over sodium sulphate, and evaporated in vacuo to afford (S)-2-((5-fluoro-2-(4-(5-phenyl-4,5-dihydro-1H-pyrazole-1-carbonyl)piperidin-1-yl)pyrimidin-4-yl)oxy)acetic acid (361 mg, 0.85 mmol, purity: 90%, recovery: 100%) as a white foam. LCMS (m/z) 428 (M+H)⁺, retention time: 3.62 min, LC/MS Method 2.

### Step 4

To a solution of (S)-2-((5-fluoro-2-(4-(5-phenyl-4,5-dihydro-1H-pyrazole-1-carbonyl)piperidin-1-yl)pyrimidin-4-yl)oxy)acetic acid (361 mg, 0.85 mmol), ammonium chloride (49.7 mg, 0.93 mmol) and DIPEA (0.325 mL, 1.86 mmol) in DMF (5 mL) stirred at rt was added HATU (385 mg, 1.01 mmol) in one charge. The reaction mixture was stirred at rt for 16 h and was evaporated in vacuo to afford a yellow oil. EtOAc (10 mL) and a saturated solution of ammonium chloride in water (10 mL) were added. After separation, the organic layer was washed successively with a saturated solution of sodium bicarbonate in water (10 mL), with brine, dried over sodium sulphate, and evaporated in vacuo to afford a yellow oil. The residue was purified by normal phase column chromatography [CyH(EtOH/EtOAc 4:1)/CyH 100/0 to %] to afford (S)-2-((5-fluoro-2-(4-(5-phenyl-4,5-dihydro-1H-pyrazole-1-carbonyl)piperidin-1-yl)pyrimidin-4-yl)oxy)acetamide as a white foam with DMF as impurity. EtOAc (20 mL) and brine (10 mL) were added to this residue. After separation, the organic layer was washed with brine to remove DMF (4×10 mL). The organic layer was dried over sodium sulphate and evaporated in vacuo to afford a colourless oil. The residue was purified by normal phase column chromatography (DCM/MeOH 100/0 to 95/5) to afford (S)-2-((5-fluoro-2-(4-(5-phenyl-4,5-dihydro-1H-pyrazole-1-carbonyl)piperidin-1-yl)pyrimidin-4-yl)oxy)acetamide as a white foam. A precipitation into iPr₂O afforded, after filtration, (S)-2-((5-fluoro-2-(4-(5-phenyl-4,5-dihydro-1H-pyrazole-1-carbonyl)piperidin-1-yl)pyrimidin-4-yl)oxy)acetamide (329 mg, 0.77 mmol, purity: 100%, recovery: 91%) as a white solid. LCMS (m/z) 427 (M+H)⁺, retention time: 2.12 min, LC/MS Method 1. ¹H NMR (400 MHz, DMSO-d6) δ ppm: 8.19 (d, J=3.2 Hz, 1H), 7.53 (br s, 1H), 7.32 (t, J=7.2 Hz, 2H), 7.27-7.20 (m, 3H), 7.11 (m, 2H), 5.31 (dd, J=4.6, 11.8 Hz, 1H), 4.72 (s, 2H), 4.49 (m, 2H), 3.49 (ddd, J=1.5, 2.2, 19.1 Hz, 1H), 3.35 (tt, J=3.8, 12.0 Hz, 1H), 2.95 (qd, J=2.7, 12.5 Hz, 2H), 2.72-2.63 (m, 1H), 1.88-1.79 (m, 1H), 1.76-1.68 (m, 1H), 1.53-1.35 (m, 2H).

### Example 168

### (1-(4-(morpholin-3-yl)pyrimidin-2-yl)piperidin-4-yl)((S)-5-phenyl-4,5-dihydro-1H-pyrazol-1-yl)methanone

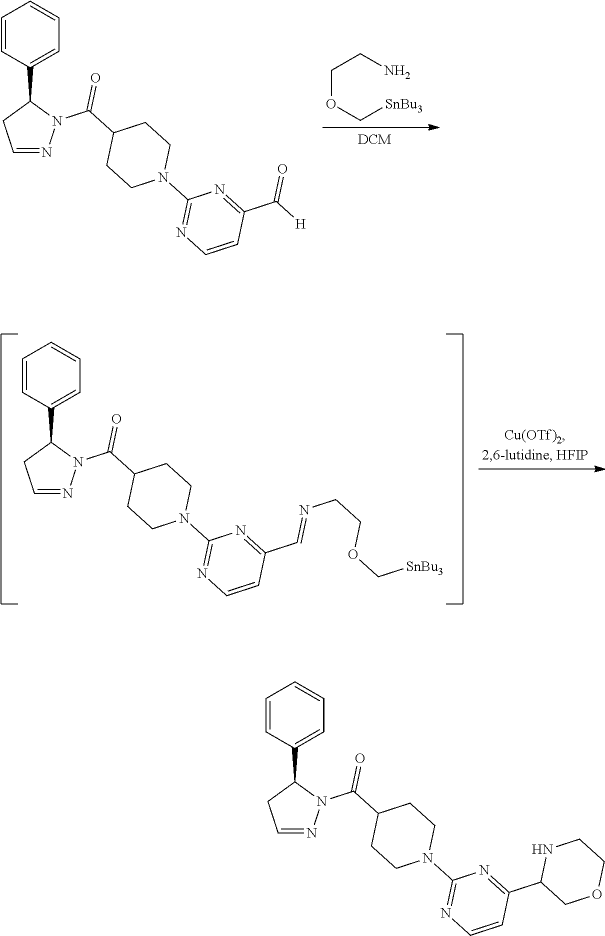

To neat 2-((tributylstannyl)methoxy)ethanamine (230 mg, 0.63 mmol) under nitrogen at rt was added (S)-2-(4-(5-phenyl-4,5-dihydro-1H-pyrazole-1-carbonyl)piperidin-1-yl)pyrimidine-4-carbaldehyde (230 mg, 0.63 mmol) in DCM (4 mL) in one charge. The reaction mixture was stirred at rt for 2.5 h under nitrogen. The reaction mixture was filtered on celite and the filtrate was evaporated in vacuo to give the corresponding imine as a yellow oil. Separately, to a suspension of copper (II) trifluoromethanesulfonate (229 mg, 0.63 mmol) in HFIP (2.5 mL) stirred under nitrogen at rt was added neat 2,6-lutidine (0.074 mL, 0.63 mmol) in one charge. The reaction mixture was stirred at rt for 1 h 30 min under nitrogen and was added a solution of the crude imine in DCM (10 mL) in one charge. The reaction mixture was stirred at rt for 15 h. A 10% solution of ammonia in water (6 mL) was added and stirred vigorously for 15 min. After separation, the aqueous layer was extracted with DCM (3×4 mL). The combined organic layers were washed with water (3×6 mL) and brine, dried over sodium sulphate, and evaporated in vacuo to give a brown oil. The residue was purified by 2 successive normal phase column chromatographies (MeOH/DCM 0-5%) and (MeOH/DCM 3%) to afford (1-(4-(morpholin-3-yl)pyrimidin-2-yl)piperidin-4-yl)((S)-5-phenyl-4,5-dihydro-1H-pyrazol-1-yl)methanone as a yellow oil. A precipitation into iPr₂O afforded, after filtration, (1-(4-(morpholin-3-yl)pyrimidin-2-yl)piperidin-4-yl)((S)-5-phenyl-4,5-dihydro-1H-pyrazol-1-yl)methanone (46 mg, 0.11 mmol, 17%) as a light yellow solid. LCMS (m/z) 421 (M+H)⁺, retention time: 1.52 min, LC/MS Method 1. ¹H NMR (400 MHz, DMSO-d6): δ ppm 8.30 (d, J=4.9 Hz, 1H), 7.32 (dd, J=7.6, 7.2 Hz, 2H), 7.24 (m, 2H), 7.11 (d, J=7.2 Hz, 2H), 6.68 (d, J=4.9 Hz, 1H), 5.31 (dd, J=11.8, 4.6 Hz, 1H), 4.68 (d, J=13.3 Hz, 2H), 3.89 (dd, J=10.6, 3.0 Hz, 1H), 3.69 (m, 2H), 3.50 (ddd, J=19.0, 12.0, 1.3 Hz, 1H), 3.40 (m, 2H), 3.27 (t, J=10.1 Hz, 1H), 2.96 (dq, J=12.5, 2.3 Hz, 2H), 2.84 (m, 2H), 2.68 (dd, J=18.8, 4.6, 1.7 Hz, 1H), 1.87 (d, J=11.6 Hz, 1H), 1.75 (d, J=12.0 Hz, 1H), 1.45 (m, 2H).

### Example 169

### (S)-2-(5-fluoro-2-(4-(5-phenyl-4,5-dihydro-1H-pyrazole-1-carbonyl)piperidin-1-yl)pyrimidin-4-yl)acetamide

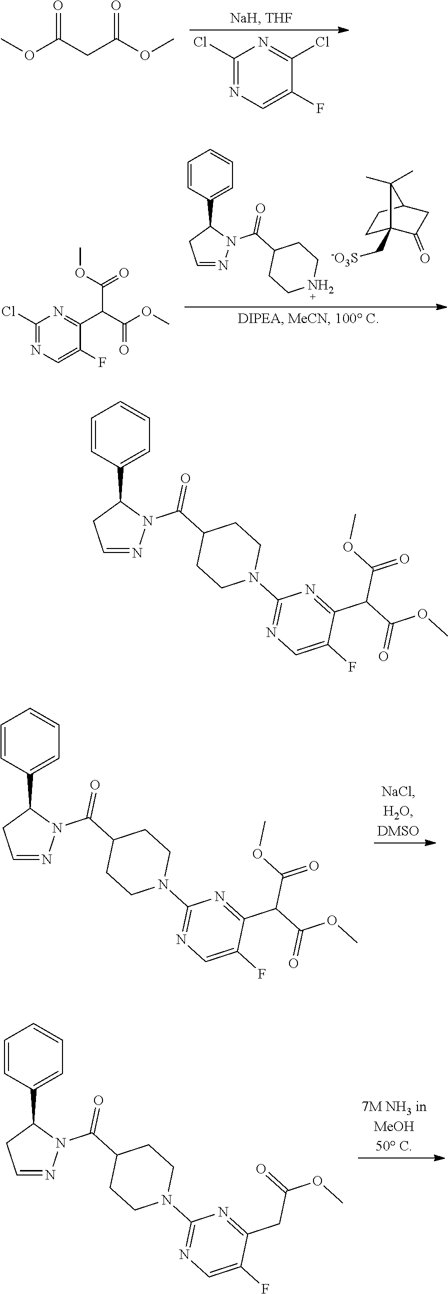

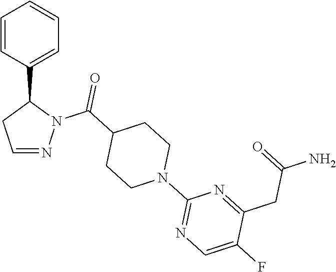 [SMILES: NC(=O)Cc1nc(N2CCC(C(=O)N3N=CC[C@H]3c3ccccc3)CC2)ncc1F]

### Step 1

To a solution of dimethyl malonate (0.787 ml, 6.9 mmol) in THF (40 ml) stirred at −10° C. was added 60% NaH in mineral oil (0.563 g, 14.1 mmol) portion-wise. The reaction mixture was stirred at −10° C. for 15 min and was added a solution of 2,4-dichloro-5-fluoropyrimidine (1 g, 6 mmol) in THF (5 ml) dropwise. The reaction mixture was stirred at −10° C. for 30 min. Water (50 mL) was added and the mixture was allowed to warm to rt. DCM (50 mL) was added. After separation, the aqueous layer was extracted with DCM (2×50 mL). The organic layer was washed with brine, dried over sodium sulphate, and evaporated in vacuo to afford an orange oil. The residue was purified by normal phase column chromatography [CyH(EtOH/EtOAc 4:1) 100/0 to 75/25] to afford dimethyl 2-(2-chloro-5-fluoropyrimidin-4-yl)malonate (1.45 g, 5.53 mmol, purity: 100%, recovery: 92%) as a colourless oil. LCMS (m/z) 263 and 265 (M+H)⁺, retention time: 2.04 min, LC/MS Method 1.

### Step 2

To a suspension of (S)-(5-phenyl-4,5-dihydro-1H-pyrazol-1-yl)(piperidin-4-yl)methanone, (1R)-10-camphorsulphonic acid salt (500 mg, 1.02 mmol) and dimethyl 2-(2-chloro-5-fluoropyrimidin-4-yl)malonate (403 mg, 1.54 mmol) in MeCN (10 mL) stirred at rt was added DIPEA (0.447 mL, 2.56 mmol) in one charge. The reaction mixture was stirred at 100° C. for 1.5 h and was evaporated to give an orange oil. The residue was purified by normal phase column chromatography [CyH(EtOH/EtOAc 4:1) 100/0 to 75/25] to afford (S)-dimethyl 2-(5-fluoro-2-(4-(5-phenyl-4,5-dihydro-1H-pyrazole-1-carbonyl)piperidin-1-yl)pyrimidin-4-yl)malonate (400 mg, 0.83 mmol, purity: 100%, recovery: 81%) as a yellow residue. LCMS (m/z) 484 (M+H)⁺, retention time: 2.67 min, LC/MS Method 1.

### Step 3

To a solution of (S)-dimethyl 2-(5-fluoro-2-(4-(5-phenyl-4,5-dihydro-1H-pyrazole-1-carbonyl)piperidin-1-yl)pyrimidin-4-yl)malonate (390 mg, 0.81 mmol) in DMSO (3.5 mL) and water (120 μl) stirred at rt was added sodium chloride (47.1 mg, 0.81 mmol). The reaction mixture was stirred at 150° C. for 3 h. EtOAc (15 mL) and water (10 mL) were added. After separation, the organic layer was washed successively with water (2×10 mL) and brine, dried over sodium sulphate, and evaporated in vacuo to afford a yellow oil. The residue was purified by normal phase column chromatography [CyH(EtOH/EtOAc 4:1)/CyH 100/0 to 75/25] to afford (S)-methyl 2-(5-fluoro-2-(4-(5-phenyl-4,5-dihydro-1H-pyrazole-1-carbonyl)piperidin-1-yl)pyrimidin-4-yl)acetate (242 mg, 0.57 mmol, purity: 100%, recovery: 71%) as a light yellow oil. LCMS (m/z) 426 (M+H)⁺, retention time: 2.60 min, LC/MS Method 1.

### Step 4

To neat (S)-methyl 2-(5-fluoro-2-(4-(5-phenyl-4,5-dihydro-1H-pyrazole-1-carbonyl)piperidin-1-yl)pyrimidin-4-yl)acetate (237 mg, 0.56 mmol) under nitrogen at rt was added a 7 M solution of ammonia in methanol (8 mL, 55.7 mmol). The reaction mixture was stirred at 50° C. for 15 h and at 80° C. for 4 days. The reaction mixture was evaporated in vacuo to give an orange foam. The residue was purified twice by normal phase column chromatography [CyH(EtOH/EtOAc 4:1) 100/0 to 50/50] to afford (S)-2-(5-fluoro-2-(4-(5-phenyl-4,5-dihydro-1H-pyrazole-1-carbonyl)piperidin-1-yl)pyrimidin-4-yl)acetamide as a yellow oil. A precipitation into iPr₂O afforded, after filtration, (S)-2-(5-fluoro-2-(4-(5-phenyl-4,5-dihydro-1H-pyrazole-1-carbonyl)piperidin-1-yl)pyrimidin-4-yl)acetamide (111 mg, 0.27 mmol, purity: 100%, recovery: 49%) as a yellow solid. LCMS (m/z) 411 (M+H)⁺, retention time: 2.04 min, LC/MS Method 1. ¹H NMR (400 MHz, DMSO-d6): δ ppm 8.30 (d, J=1.5 Hz, 1H), 7.55 (s, 1H), 7.32 (dd, J=7.6, 7.2 Hz, 2H), 7.24 (m, 2H), 7.10 (d, J=7.4 Hz, 3H), 5.31 (dd, J=11.8, 4.7 Hz, 1H), 4.57 (d, J=12.8 Hz, 2H), 3.50 (d, J=1.3 Hz, 2H), 3.49 (m, 1H), 3.35 (m, 1H), 2.96 (m, 2H), 2.68 (ddd, J=18.8, 4.5, 1.5 Hz, 1H), 1.87 (d, J=12.2 Hz, 1H), 1.75 (d, J=11.9, 1H), 1.46 (m, 2H).

### Pharmaceutical Compositions

EXAMPLE A—An ointment is prepared by combining 20% (w/w) of the compound of Example 20, 32, 71, 77, 78, 85, 108, 109, 131, or 159; and 80% (w/w) of petrolatum. The mixture is passed through a roller mill until a uniform consistency is obtained.

EXAMPLE B—Aerosol Spray: A solution is prepared from the following components: [Ingredient (Amount (w/w))]: Compound of Example 20, 32, 71, 77, 78, 85, 108, 109, 131, or 159 (1.00); propylene glycol (5.00); golysorbate 80 (1.00); ethanol (78.00); and purified water (15.00). The solution is placed in a conventional aerosol container, a valve mechanism is attached, and the container is charged with nitrogen to 100 psig.

EXAMPLE C—Tablets are prepared using conventional methods and are formulated as follows: [Ingredient (Amount per tablet)]: Compound of Example 20, 32, 71, 77, 78, 85, 108, 109, 131, or 159 (5 mg); microcrystalline cellulose (100 mg); lactose (100 mg); sodium starch glycollate (30 mg); and magnesium stearate (2 mg).

EXAMPLE D—Capsules are prepared using conventional methods and are formulated as follows: [Ingredient (Amount per tablet)]: Compound of Example 20, 32, 71, 77, 78, 85, 108, 109, 131, or 159 (15 mg); dried starch (178 mg); and magnesium stearate (2 mg).

### Biological Assays

Biological In Vitro Assay

A fluorescent polarization based binding assay was used to assess the activity of the compounds of this invention, the details of which are disclosed in International Patent Appln. No. PCT/IB2014/059004, now, International Patent Appln. Pub. No. WO2014/125444.

The pIC₅₀s are averaged to determine a mean value, for a minimum of 2 experiments.

As determined using the above method, the compounds of Examples 1-169 exhibited a pIC₅₀ between approximately 6.0 and 9.0.

For example, the compounds of Examples 2-4, 7, 9-22, 24, 26-28, 30-42, 44-54, 56-65, 67-85, 87-109, 112-118, 120, 123, 124, 126-130, 133-142, 144-148, 150, 154-158, 161, 162, 164-167, and 169 exhibited a pIC₅₀ between approximately 7.5 and 9.0.

The compounds of Examples 12-15, 17, 18, 21, 22, 27, 28, 32, 36, 39, 40, 44-46, 50, 52, 53, 58, 59, 61, 64, 69, 78-83, 85, 87, 90-92, 95, 98, 99, 101-106, 127, 133, 135, 137, 138, 140, 142, 148, 150, 154, 156, 157, 162, 165, and 169 exhibited a pIC₅₀ between approximately 8.0 and 9.0.

For instance, the compounds of Examples 20, 32, 71, 77, 78, 85, 108, 109, 131, and 159 inhibited RIP1 kinase in the above method with a pIC₅₀ of approximately 7.8, 8, 7.8, 7.9, 8, 8, 7.5, 7.6, 7.4, and 7.3 respectively.

Biological In Vitro Cell Assay

The efficacy of RIP1 inhibitors can be tested in mice in vitro using a human monocytic leukemia U937 or mouse L929 fibrosarcoma cells in a necroptosis assay. As determined using the method described in S. He et al., Cell, 137(6):1100-1111 (2009) and International Patent Appln. No. PCT/IB2014/059004, now, International Patent Appln. Pub. No. WO2014/125444, the compounds of Examples 1-29, 31-89, 91-165, 168, and 169 exhibited a pIC₅₀ between approximately 5.0 and 9.0.

For instance, the compounds of Examples 1-5, 7, 9, 11-22, 24, 26-28, 31-36, 38-42, 44-54, 56-61, 64, 67-74, 76-89, 91-106, 108, 109, 111, 112, 115-118, 123-130, 133-135, 137-142, 144-147, 150, 151, 155-162, 164, 165, and 169 inhibited necrosis in U937 cells in the above method with a pIC₅₀ between approximately 7.0 and 9.0.

For instance, the compounds of Examples 3-5, 12, 14, 17, 18, 21, 22, 27, 28, 32, 36, 39, 44, 45, 52, 53, 58, 78, 79, 81-83, 85-89, 92, 95, 99, 102-105, 118, 124, 133, 135, 137, 139, 156, 157, 161, 162, 164, and 165 inhibited necrosis in U937 cells in the above method with a pIC₅₀ between approximately 8.0 and 9.0.

For instance, the compounds of of Examples 20, 32, 71, 77, 78, 85, 108, 109, 131, and 159 inhibited necrosis in U937 cells in the above method with a mean pIC₅₀ of approximately 7.5, 8.3, 7.8, 7.2, 8.4, 8.3, 7.3, 7.9, 6.8, and 7.9 respectively.

For instance, the compounds of Examples 1, 7, 9, 11-18, 20-22, 24, 26-28, 31-34, 36, 40-42, 44-46, 49-54, 56-60, 63, 67-74, 76-78, 80-89, 91, 93-105, 108, 109, 113, 115-118, 123-129, 131, 133, 135, 138-140, 142, 144, 145, 150, 155, 156, 159-162, 164, and 165 inhibited necrosis in L929 cells in the above method with a pIC50 between approximately 5.0 and 9.0.

For instance, the compounds of Examples 1, 12, 14, 17, 18, 20-22, 24, 27, 28, 32, 36, 40, 44-46, 50-53, 56, 58, 59, 71, 78, 80-89, 91, 95, 98, 99, 101-105, 109, 118, 133, 135, 138-140, 155, 156, 159, 161, 162, 164, and 165 inhibited necrosis in L929 cells in the above method with a pIC50 between approximately 6.0 and 9.0.

For instance, the compounds of Examples 1, 14, 27, 28, 36, 52, 78, 81, 83, 86, 89, 102, 104, 118, 156, and 162 inhibited necrosis in L929 cells in the above method with a pIC50 between approximately 7.0 and 9.0.

For instance, the compounds of Examples 20, 32, 71, 77, 78, 85, 108, 109, 131, and 159 inhibited necrosis in L929 cells in the above method with a pIC₅₀ of approximately 6, 6.5, 6.1, 5.5, 7.5, 6.5, 5, 6.6, 5.4, and 6.6 respectively. Viability was measured by quantitating cellular levels of ATP using the Cell Titer-Glo kit. All data are means±standard deviation of the mean.

Biological In Vivo Assay

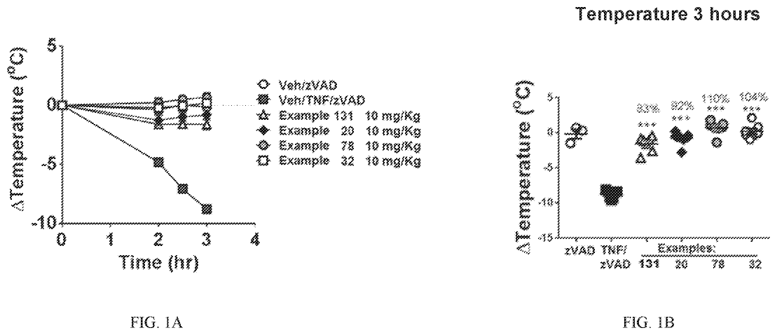

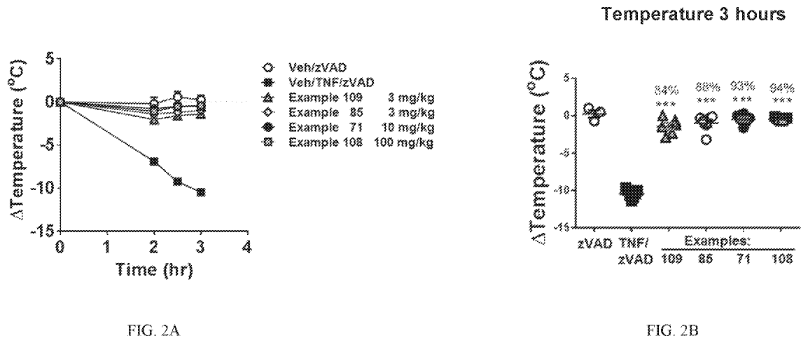

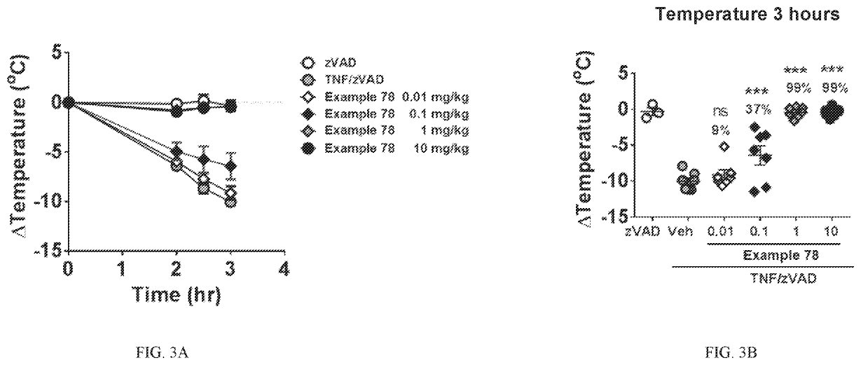

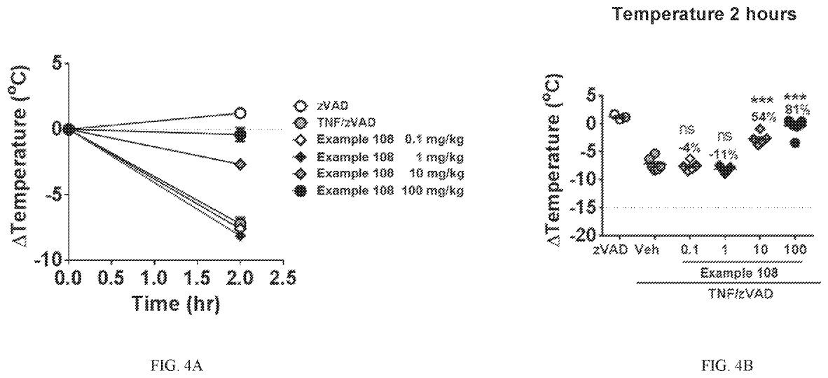

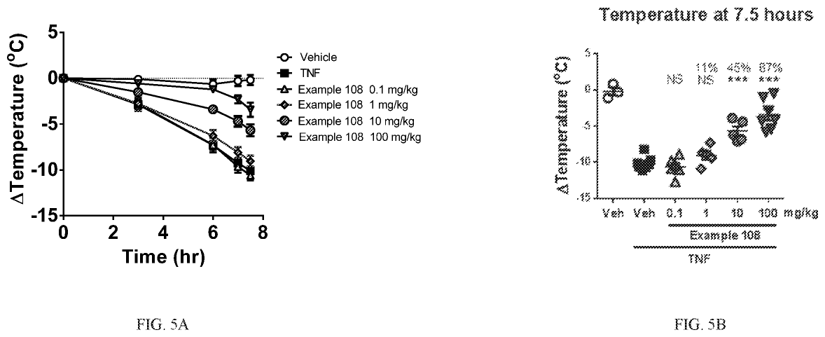

The efficacy of RIP1 inhibitors can be tested in mice in vivo using a TNF-driven systemic inflammatory response syndrome model (L. Duprez et al. Immunity 35(6):908-918, (2011)) using TNF plus the caspase inhibitor zVAD or TNF alone. The TNF/zVAD model is terminated at ˜3 hrs and the TNF alone model is terminated at ˜8 hrs (under IACUC guidelines for temperature loss). TNF (or TNF/zVAD) induced manifestations include temperature loss, the production of numerous cytokines (including IL-6, IL-1b, MIP1β and MIP2) in the periphery, liver and intestinal inflammation and an increase of markers of cellular (LDH and CK) and liver damage (AST and ALT) in the serum. Inhibition of these TNF alone or TNF/zVAD induced manifestations can be shown by PO pre-dosing with selected compounds. For example, mice (8 mice per group) were pre-dosed PO with vehicle or compound 15 minutes before i.v. administration of mouse TNF (30 μg/mouse) alone or in combination with zVAD (0.4 mg/mouse) simultaneously. Temperature loss in the mice was measured by rectal probe. The study was terminated when the control group lost 7 degrees, per our IACUC protocol. All data are shown as means±standard error of the mean. Representative data for the compounds of Examples 20, 32, 71, 78, 85, 108, 109, and 131 expressed over time and at the 2.0, 3.0, and 7.5 hour time points, respectively, are provided in FIGS. 1A-5B. Data for the compounds of Examples 20, 32, 71, 78, 85, 108, 109, and 131 tested in this model are provided in Table A.

TABLE A

| Example No. | Dose (mg/kg) | % Inhibition | Route |
|---|---|---|---|
| 20 | 10 | 92 | PO |
| 32 | 10 | 104 | PO |
| 71 | 10 | 93 | PO |
| 78 | 10 | 110 | PO |
| 78 | 0.01 | 9 | PO |
| 78 | 0.1 | 37 | PO |
| 78 | 1.0 | 99 | PO |
| 78 | 10 | 99 | PO |
| 85 | 3 | 88 | PO |
| 108 | 100 | 94 | PO |
| 108 | 0.1 | −4 | PO |
| 108 | 1.0 | −11 | PO |
| 108 | 10 | 54 | PO |
| 108 | 100 | 81 | PO |
| 109 | 3 | 84 | PO |
| 131 | 10 | 83 | PO |

Rd10 Mouse Model of Human Retinitis Pigmentosa

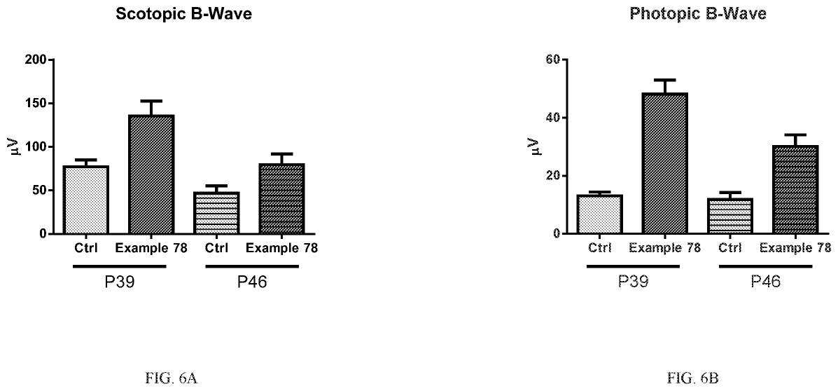

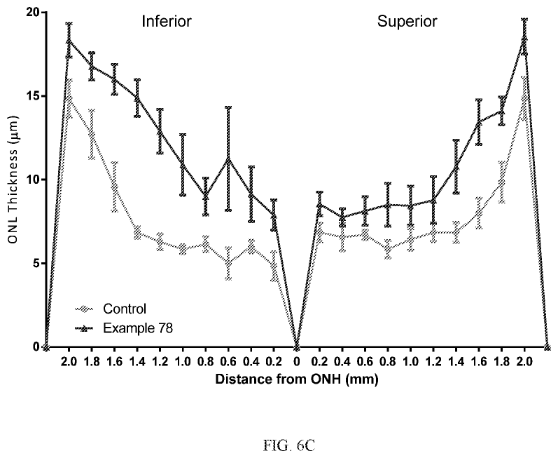

Inhibition of RIP1 has been implicated in protection against the Rd10 mouse model of human retinitis pigmentosa (RP) (Y. Murakami et al., PNAS 109(36):14598-14603 (2012)). Rd10 mice have a mutation, the rod-specific gene that encodes rod cGMP phosphodiesterase β-subunit. Mice were dark reared to P30, at which point they were moved to a 12-hour light/dark cycle to induce retinal degeneration. Mice were pre-dosed with RIP1 inhibitors in food-based dosing on day P28, two days prior to the switch to normal cyclic light, such that mice (15 mice per group) received on average 100 mg/kg/day RIP1 inhibitor in diet or control diet. Electroretinography (ERG) recordings were made at P39 and P46 as a measure of retinal cell function. Retinal cell loss was assessed by measurement of the thickness of the Outer Nuclear Cell (ONL) layers at various distances from the Optic Nerve Head (ONH) in hematoxylin and eosin stained retinal tissue sections collected at P46. Representative data for the compound of Example 78 are provided in FIGS. 6A and 6B.

EAE Mouse Model of Human MS

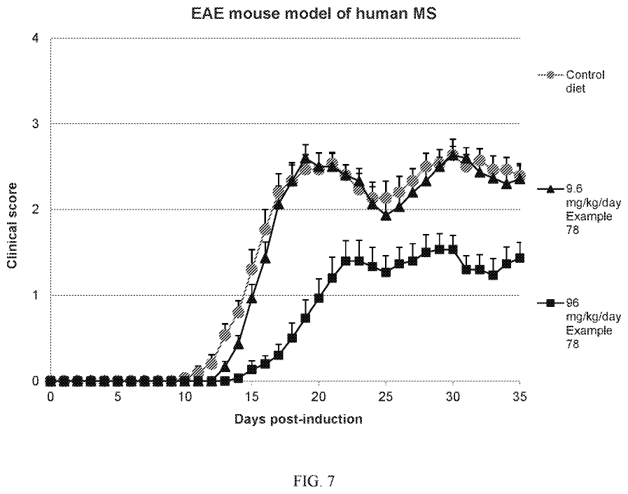

Inhibition of RIP1 has been implicated in protection against the experimental autoimmune encephalomyelitis (EAE) mouse model of human Multiple Sclerosis (MS) (D. Ofengeim et al. Cell Reports 10(11):1836-1849, (2015)). Mice were pre-dosed with RIP1 inhibitors in food-based dosing one day prior to EAE induction, such that mice (15 mice per group) received on average 96 mg/kg/day or 9.6 mg/kg/day of RIP1 inhibitor in diet or control diet. Mice were inoculated with 100 ul of inoculum containing 100 ug of myelin oligodendrocyte protein amino acid 35-55 (MOG₃₅₋₅₅) and 200 ug heat inactivated Mycobacterium tuberculosis in mineral oil. Inoculation was done by giving each mouse two 100 ul injections subcutaneously to the lower and higher aspect of the back, respectively. Intraperitoneal injections of pertussis toxin (4 ug/ml) 100 ul each were given at 2 hours and 24 hours after inoculation. Mice were monitored daily until day 35 post-induction and clinical signs were scored as follows: 0.5 for partial tail weakness, 1.0 for complete tail paralysis (all of tail dragging along), 1.5 for flaccid tail and abnormal gait, 2.0 for flaccid tail and clear weakness of hind legs, 2.5 for partial paralysis in one hindlimb (no movement preserved in affected limb), 3.0 for complete paralysis in both hindlimbs, 4.0 for complete paralysis in hindlimbs and partial weakness in forelimbs, and 5.0 for complete paralysis in both forelimbs and hindlimbs (tetraplegia) or moribund. Representative data for the compound of Example 78 are provided in FIG. 7.

Glucose Homeostasis

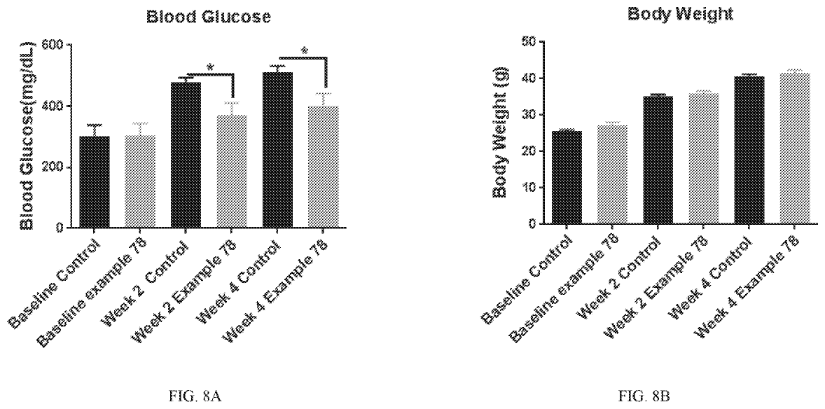

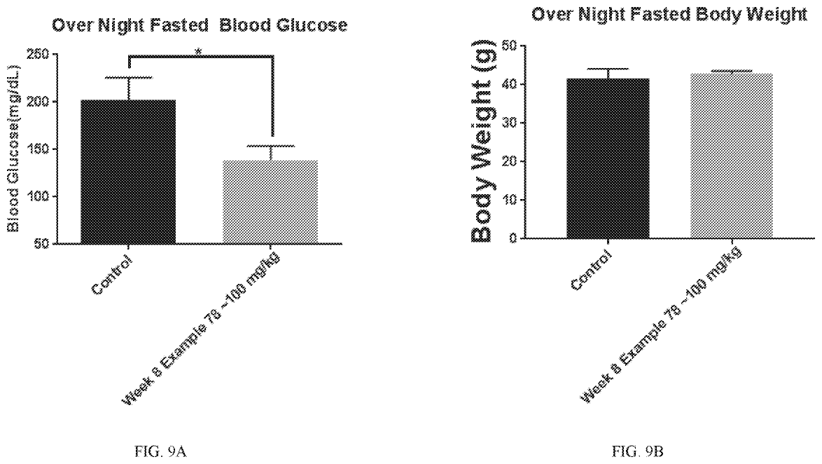

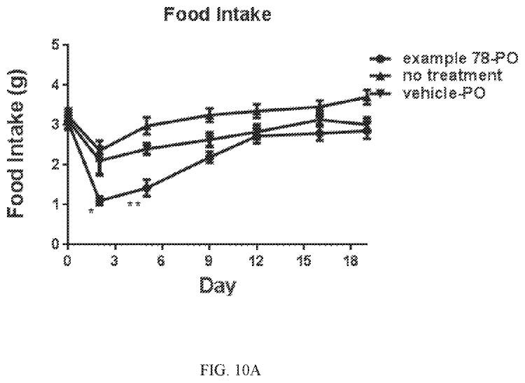

Blocking the actions of TNF at the TNF receptor has been shown to improve glucose homeostasis in animals and humans (Stagakis et al., Arthritis Research & Therapy (2012)). We therefore investigated the ability of our small molecule RIPK1 inhibitor to improve glucose homeostasis in a diabetic animal model. Mice genetically deficient for the leptin receptor (db/db mice; Jackson Labs, BKS.Cg-Dock7m+/+Leprdb/J) are a widely used animal model of insulin resistance and diabetes. In our study, 4-week old male db/db mice were acclimated for a period of one week. At this point, when the mice were 5 weeks of age, the study was started. Mice were then divided into two group (n=10/group) and baseline measurements of non-fasted blood glucoses (using a glucometer) and body weight were taken. After baseline measurements, one group of mice was fed chow containing RIPK1 inhibitor (100 mg/kg/day, n=10) and the other remained on normal chow (n=10). Animals remained on these diets for 6 weeks. Additional measurements of non-fasted blood glucose and body weight were taken 2 and 4 weeks after study start. At week 8, mice were fasted overnight (16 hours) and fasted blood glucose and body weight were measured on the following day. Reduced non-fasted blood glucose at weeks 2 and 4 was observed (FIGS. 8A and 8B). Additionally, fasted blood glucose at week 8 was also significantly lower in db/db mice receiving the RIPK1 inhibitor versus normal chow controls (FIGS. 9A and 9B). These data demonstrate a significant improvement in glucose homeostasis, in db/db mice receiving RIPK1 inhibitor in their diet versus the control diet cohort. Measurements of both non-fasted and fasted body weight showed no difference between groups, suggesting that the improved glycemic control observed in mice receiving RIPK1 inhibitor were not secondary to a loss in body weight. These data therefore demonstrate a direct, beneficial effect of blocking RIPK1 kinase activity to improve glucose homeostasis. Representative data for the compound of Example 78 are provided in FIGS. 8A, 8B, 9A, and 9B.

Obesity

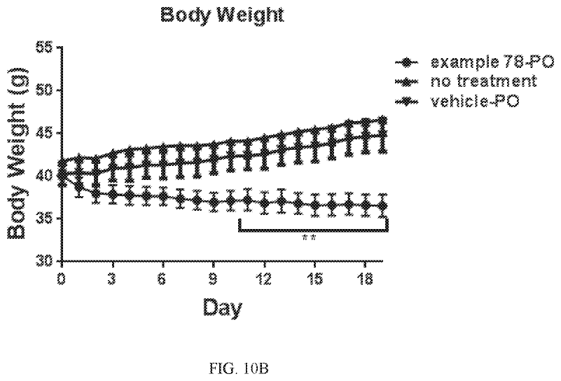

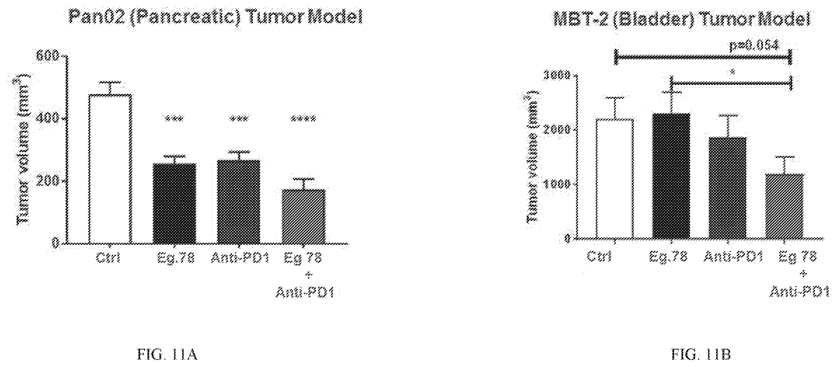

Inflammation is known to be a contributing factor in the pathogenesis of diabetes and obesity (Chen. et. al., International Journal of Endocrinology (2015)). We therefore explored the effect of blocking RIPK1 kinase activity on body weight in mice made obese by chronic feeding of a high fat diet (HFD). Diet-induced obese, male mice C57Bl/6J (Jackson Labs Stock#380050) arrived at 22 weeks of age, with average body weight of 40 g and acclimated for one week and maintained on HFD. Mice were then acclimated to oral gavage with water for 7 days before study start. Thereafter mice received RIPK 1 inhibitor (50 mg/kg, bid, n=9) or vehicle (bid, n=9) for 19 days. Food intake was measured twice weekly and body weight was measured daily throughout the study. Although high fat diet-fed mice dosed with the RIPK1 inhibitor initially reduced their food intake (day 2 and day 5 (FIG. 10A)), after the first week food intake in the two groups was equivalent. However while control animals continued to gain weight on the HFD, the HFD-fed mice PO dosed with the RIPK1 inhibitor did not gain weight and in fact showed a modest decrease in body weight from pre-compound exposure levels (FIG. 10B). By the end of the first week, control animals were significantly heavier than mice given treated with RIPK1 inhibitor and this was maintained for at least 21 days. We demonstrated, therefore, that RIPK1 inhibition has therapeutic potential as a treatment for obesity. Representative data for the compound of Example 78 are provided in FIGS. 10A and 10B.

Subcutaneous Tumor Efficacy

The efficacy of RIP1 inhibition was tested in 12 different murine (6-8 week old) syngeneic subcutaenous tumor models. RIP1 inhibition was tested as a single agent in all models, with anti-PD1 combination arms added to the five of the final models.

TABLE B
Study Design

| | | | Dose | Dosing | |
|---|---|---|---|---|---|
| Group | N | Treatment | (mg/kg) | Route | Schedule |
| 1 | 8 | PBS (saline) | 0 | i.p. | BIW x up to 21 days |
| 2 | 8 | Example 78 | 40 | p.o. | BID x up to 21 days |

Note:
N: animal number
Dosing volume: adjust dosing volume based on body weight (10 μl/g).
Treatment regimen may be changed per BW (body weight) loss.
The interval of BID dosing is 8 hours.

Study endpoints: The major endpoints of the study include the following:
1) Tumor growth inhibition (TGI): TGI % is an indication of antitumor effectiveness, and expressed as: TGI (%)=100×(1−T/C). T and C are the mean tumor volume of the treated and control groups, respectively, on a given day.
2) Tumor and plasma collection at study end for further investigation.

### EXPERIMENTAL METHODS

Cell Culture

The 12 syngenic cell lines were maintained in vitro with different medium (indicated in Table C) at 37° C. in an atmosphere of 5% CO₂ in air. The tumor cells were routinely subcultured twice weekly. The cells in an exponential growth phase were harvested and counted for tumor inoculation.

TABLE C
Medium information

| Cell line (tumor/carcinoma) | Medium |
|---|---|
| MBT2 (bladder) | RPMI1640 + 10% FBS |
| EMT-6 (breast) | DMEM + 10% FBS |
| CT26 (colon) | RPMI1640 + 10% FBS |
| MC38 (colon) | DMEM + 10% FBS |
| H22 (hepatoma) | RPMI1640 + 10% FBS |
| LL/2 (Lewis lung) | DMEM + 10% FBS |
| Renca (renal) | DMEM + 10% FBS |
| A20 (B lymphoma) | RPMI1640 + 10% FBS |
| B16F10 (melanoma) | DMEM + 10% FBS |
| B16BL6 (melanoma) | RPMI1640 + 10% FBS |
| Pan02 (pancreatic) | RPMI1640 + 10% FBS |
| RM-1(prostate) | RPMI1640 + 10% FBS |

Tumor Inoculation

Each mouse was inoculated subcutaneously with tumor cells in 0.1 mL of PBS for tumor development. The treatments were started when the mean tumor size reached approximately 80-120 mm³ (around 100 mm³). The test article (Example 78 or anti-PD1 (anti-mouse PD-1 antibody (clone RPM1-14), BioXcell) administration and the animal numbers in each study group are shown in the experimental design Table B. The date of tumor cell inoculation is denoted as day 0.

### Study Results

TABLE D
Mean tumor growth inhibition (TGI) at model termination

| | | | RIP1i + |
|---|---|---|---|
| MuScreen Model | RIP1i TGI | Anti-PD1 TGI | Anti-PD1 TGI |
| EMT-6 | 21% | | |
| CT-26 | 19% | | |
| H-22 | 19% | | |
| LL/2 | 22% | | |
| Renca | 19% | | |
| A20 | 0.6% | | |
| B16F10 | 20% | | |
| Pan02 | PG21: 46% | PG21: 44% | PG21: 64% |
| MC38 | −16% | 46% | 62% |
| B16BL6 | 16% | −1.25% | 32% |
| RM-1 | −8% | −4% | 4% |
| MBT2 | −4% | 15.3% | 46% |

Sharpin-Deficient Mouse Model of TNF-Dependent Dermatitis

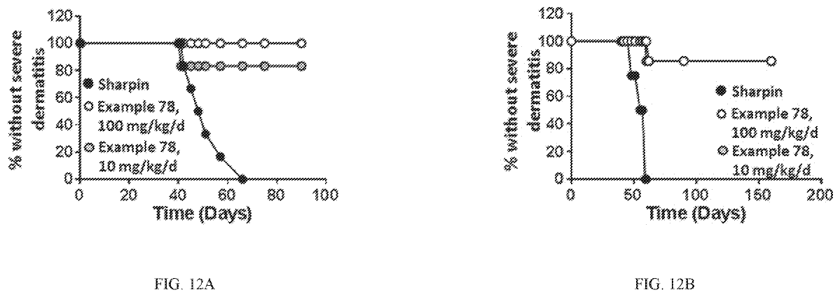

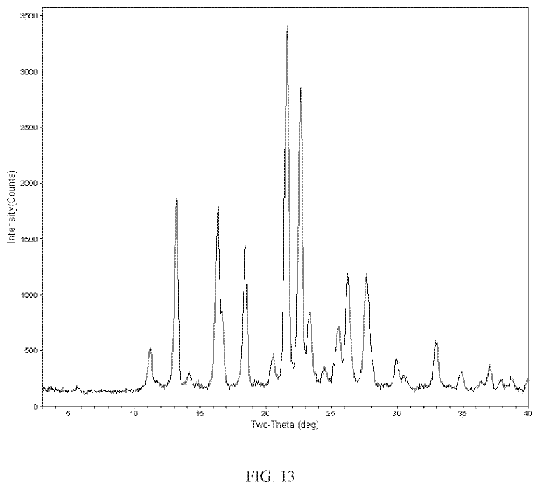

Sharpin-deficient mice (cpdm) develop a spontaneous and severe TNF- and RIPK1-dependent dermatitis and multi-organ immunopathology around 6-8 weeks of age (S. B. Berger et al., Journal of Immunology, 192(12):5476-5480, (2014)). Mice were dosed with RIP1 inhibitors at the time of weaning (3-4 weeks of age) prior to the development of dermatitis lesions or therapeutically after the development of dermatitis lesions (about 6 weeks of age) using a food-based dosing regimen, such that mice (4-7 mice per group) received on average 100 mg/kg/day or 10 mg/kg/day of RIP1 inhibitor in diet or control diet. Mice were observed for signs of proliferative dermatitis by using a dermatitis scoring system based on lesion character and regions affected. The character of the lesion was categorized according the following, in order of increasing severity, 0=none, 1=excoriation only or one small punctuated crust (≤2 mm), 2=multiple, small punctuate crusts or coalescing crust (>2 mm), 3=erosion or ulceration. The regions were identified as: Region 1: the head cranial to the medial pinna attachment and/or lesions affecting the mandible cranial to the sternum, Region 2: inner and outer pinna, dorsal cervical region caudal to the medial pinna attachment, dorsal and ventral thorax, and thoracic limbs, Region 3: any region caudal to the ribcage. A score for the regions affected was categorized per the following: 0=none; 1=Region 2 or 3; 2=Region 2 and 3; 3=Region 1+/−other affected regions. To calculate the dermatitis severity score, the lesion score and regions affected score were summed, divided by 6, and then multiplied by 100. A severity score of 66 was considered severe dermatitis. Pre-dosing with RIP1 inhibitors in food-based dosing resulted in a complete protection from the development of severe dermatitis. Additionally, therapeutic dosing with RIP1 inhibitors in food-based dosing rescued established dermatitis. Representative data for the compound of Example 78 are provided in FIGS. 12A and 12B.

1-32. (canceled)

33. A compound according to Formula (I): 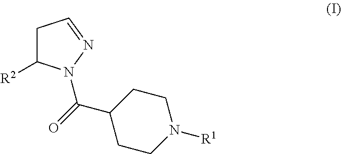
wherein:
R¹ is a substituted or unsubstituted 5-6 membered heteroaryl or 9-10 membered heteroaryl group,
wherein said substituted 5-6 membered heteroaryl or 9-10 membered heteroaryl group is substituted by 1 or 2 substituents independently selected from hydroxyl, cyano, halogen, (C₁-C₄)alkyl, halo(C₁-C₄)alkyl, hydroxy(C₁-C₄)alkyl, (C₂-C₄)alkynyl, optionally substituted (C₁-C₄)alkoxy, optionally substituted 5-6 membered heterocycloalkyl-CO—, fused 5-6 membered heterocycloalkyl, H₂N—, ((C₁-C₄)alkyl)-NH—, ((C₁-C₄)alkyl)((C₁-C₄)alkyl)N—, H₂NCO—, H₂NCO—(C₁-C₄)alkyl-, ((C₁-C₄)alkyl)NHCO—, (hydroxy-(C₁-C₄)alkyl)NHCO—, (C₃-C₆)cycloalkyl-NHCO—, optionally substituted 5-6 membered heterocycloalkyl-NHCO—, ((C₁-C₄)alkyl)((C₁-C₄)alkyl)N—CO—, (C₁-C₄)alkyl-CONH—, ((C₁-C₄)alkyl)((C₁-C₄)alkyl)N—NHCO—, —CO₂H, —CO₂(C₁-C₄)alkyl, (C₁-C₄)alkylthio-, phenyl-(C₁-C₄)alkylthio-, (C₁-C₄)alkyl-SO₂—, phenyl, optionally substituted 5-6 membered heterocycloalkyl, and optionally substituted 5-6 membered heteroaryl group,
wherein said optionally substituted (C₁-C₄)alkoxy is optionally substituted by hydroxyl, —CO₂H, —CONH₂, 5-6 membered heterocycloalkyl, or 5-6 membered heteroaryl; or said optionally substituted 5-6 membered heterocycloalkyl-CO—, optionally substituted 5-6 membered heterocycloalkyl, or optionally substituted 5-6 membered heteroaryl group is optionally substituted by (C₁-C₄)alkyl or oxo; or said optionally substituted 5-6 membered heterocycloalkyl-NHCO— is optionally substituted by (C₁-C₄)alkyl-CO—; and
R² is a substituted or unsubstituted phenyl or 5-6 membered heteroaryl group,
wherein said substituted phenyl or 5-6 membered heteroaryl group is substituted by 1 or 2 substituents independently selected from halogen, (C₁-C₄)alkyl, (C₁-C₄)alkoxy, and cyano;
or a pharmaceutically acceptable salt thereof,
wherein said compound or pharmaceutically acceptable salt thereof is not:
(5-(5-fluoropyridin-3-yl)-4,5-dihydro-1H-pyrazol-1-yl)(1-(5-methylpyrimidin-2-yl)piperidin-4-yl)methanone;
(1-(5-fluoropyridin-2-yl)piperidin-4-yl)(5-phenyl-4,5-dihydro-1H-pyrazol-1-yl)methanone;
(1-(5-methylpyridin-2-yl)piperidin-4-yl)(5-phenyl-4,5-dihydro-1H-pyrazol-1-yl)methanone;
(1-(5-methylpyrimidin-2-yl)piperidin-4-yl)(5-phenyl-4,5-dihydro-1H-pyrazol-1-yl)methanone;
(1-(5-fluoropyrimidin-2-yl)piperidin-4-yl)(5-phenyl-4,5-dihydro-1H-pyrazol-1-yl)methanone;
(5-(5-fluoropyridin-3-yl)-4,5-dihydro-1H-pyrazol-1-yl)(1-(5-fluoropyrimidin-2-yl)piperidin-4-yl)methanone;
(5-(5-fluoropyridin-3-yl)-4,5-dihydro-1H-pyrazol-1-yl)(1-(pyrimidin-2-yl)piperidin-4-yl)methanone;
(1H-indol-2-yl)(5-phenyl-4,5-dihydro-1H-pyrazol-1-yl)methanone;
(5-(pyridin-3-yl)-4,5-dihydro-1H-pyrazol-1-yl)(1-(pyrimidin-2-yl)piperidin-4-yl)methanone;
(5-(6-methylpyridin-3-yl)-4,5-dihydro-1H-pyrazol-1-yl)(1-(pyrimidin-2-yl)piperidin-4-yl)methanone;
(5-(6-methylpyridin-3-yl)-4,5-dihydro-1H-pyrazol-1-yl)(1-(pyridin-2-yl)piperidin-4-yl)methanone;
(1-(pyridin-2-yl)piperidin-4-yl)(5-(pyridin-3-yl)-4,5-dihydro-1H-pyrazol-1-yl)methanone;
(5-(5-methylpyrazin-2-yl)-4,5-dihydro-1H-pyrazol-1-yl)(1-(pyridin-2-yl)piperidin-4-yl)methanone;
(1-(benzo[d]oxazol-2-yl)piperidin-4-yl)(5-(pyridin-3-yl)-4,5-dihydro-1H-pyrazol-1-yl)methanone;
(5-phenyl-4,5-dihydro-1H-pyrazol-1-yl)(1-(pyrimidin-2-yl)piperidin-4-yl)methanone;
(5-phenyl-4,5-dihydro-1H-pyrazol-1-yl)(1-(pyridin-2-yl)piperidin-4-yl)methanone 2,2,2-trifluoroacetate;
(5-phenyl-4,5-dihydro-1H-pyrazol-1-yl)(1-(5-(trifluoromethyl)pyridin-2-yl)piperidin-4-yl)methanone 2,2,2-trifluoroacetate;
(1-(benzo[d]oxazol-2-yl)piperidin-4-yl)(5-phenyl-4,5-dihydro-1H-pyrazol-1-yl)methanone 2,2,2-trifluoroacetate;
(1-(5-fluoropyrimidin-2-yl)piperidin-4-yl)(5-(6-methylpyridin-3-yl)-4,5-dihydro-1H-pyrazol-1-yl)methanone; or
4-(1-(1-(5-fluoropyrimidin-2-yl)piperidine-4-carbonyl)-4,5-dihydro-1H-pyrazol-5-yl)benzonitrile.

34. The compound, or a pharmaceutically acceptable salt thereof, according to claim 33, wherein:
R¹ is a substituted or unsubstituted 5-6 heteroaryl group;
wherein said substituted 5-6 heteroaryl group is substituted by 1 or 2 substituents independently selected from hydroxyl, cyano, halogen, (C₁-C₄)alkyl, halo(C₁-C₄)alkyl, hydroxy(C₁-C₄)alkyl, (C₂-C₄)alkynyl, optionally substituted (C₁-C₄)alkoxy, optionally substituted 5-6 membered heterocycloalkyl-CO—, fused 5-6 membered heterocycloalkyl; H₂N—, ((C₁-C₄)alkyl)-NH—, ((C₁-C₄)alkyl)((C₁-C₄)alkyl)N—, H₂NCO—, H₂NCO—(C₁-C₄)alkyl-, ((C₁-C₄)alkyl)NHCO—, (hydroxy-(C₁-C₄)alkyl)NHCO—, (C₃-C₆)cycloalkyl-NHCO—, optionally substituted 5-6 membered heterocycloalkyl-NHCO—, ((C₁-C₄)alkyl)((C₁-C₄)alkyl)-NCO—, (C₁-C₄)alkyl-CONH—, ((C₁-C₄)alkyl)((C₁-C₄)alkyl)N—NHCO—, —CO₂H, —CO₂(C₁-C₄)alkyl, (C₁-C₄)alkylthio-, phenyl-(C₁-C₄)alkylthio-, (C₁-C₄)alkyl-SO₂—, phenyl, optionally substituted 5-6 membered heterocycloalkyl, and optionally substituted 5-6 membered heteroaryl group,
wherein said optionally substituted (C₁-C₄)alkoxy is optionally substituted by hydroxyl, —CO₂H, —CONH₂, 5-6 membered heterocycloalkyl, or 5-6 membered heteroaryl; or said optionally substituted 5-6 membered heterocycloalkyl-CO—, optionally substituted 5-6 membered heterocycloalkyl, or optionally substituted 5-6 membered heteroaryl group is optionally substituted by (C₁-C₄)alkyl or oxo; or said optionally substituted 5-6 membered heterocycloalkyl-NHCO— is optionally substituted by (C₁-C₄)alkyl-CO—; and
R² is a substituted or unsubstituted phenyl or 5-6 membered heteroaryl group,
wherein said substituted phenyl or 5-6 membered heteroaryl group is substituted by 1 or 2 substituents independently selected from halogen, (C₁-C₄)alkyl, (C₁-C₄)alkoxy, and cyano.

35. The compound, or a pharmaceutically acceptable salt thereof, according to claim 33, wherein:
R¹ is a substituted or unsubstituted 5-6 membered heteroaryl group;
wherein said substituted 5-6 membered heteroaryl group is substituted by 1 or 2 substituents independently selected from cyano, halogen, (C₁-C₄)alkyl, H₂N—, H₂NCO—, and —CO₂H; and
R² is a substituted or unsubstituted phenyl or 5-6 membered heteroaryl group,
wherein said substituted phenyl or 5-6 membered heteroaryl group is substituted by 1 or 2 substituents independently selected from halogen, (C₁-C₄)alkyl, (C₁-C₄)alkoxy, and cyano.

36. The compound, or a pharmaceutically acceptable salt thereof, according to claim 33, having Formula (II):

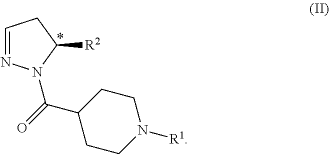

37. The compound, or a pharmaceutically acceptable salt thereof, according to claim 33, wherein R¹ is a substituted or unsubstituted 5-6 membered heteroaryl group, wherein said substituted or unsubstituted 5-6 membered heteroaryl group is a substituted or unsubstituted pyrimidinyl, pyrazinyl, pyridazinyl, or pyridyl,
wherein said substituted pyrimidinyl, pyrazinyl, pyridazinyl, or pyridyl is substituted by 1 or 2 substituents independently selected from hydroxyl, cyano, halogen, (C₁-C₄)alkyl, halo(C₁-C₄)alkyl, hydroxy(C₁-C₄)alkyl, (C₂-C₄)alkynyl, optionally substituted (C₁-C₄)alkoxy, optionally substituted 5-6 membered heterocycloalkyl-CO—, fused 5-6 membered heterocycloalkyl; H₂N—, ((C₁-C₄)alkyl)-NH—, ((C₁-C₄)alkyl)((C₁-C₄)alkyl)N—, H₂NCO—, H₂NCO—(C₁-C₄)alkyl-, ((C₁-C₄)alkyl)NHCO—, (hydroxy-(C₁-C₄)alkyl)NHCO—, (C₃-C₆)cycloalkyl-NHCO—, optionally substituted 5-6 membered heterocycloalkyl-NHCO—, ((C₁-C₄)alkyl)((C₁-C₄)alkyl)-NCO—, (C₁-C₄)alkyl-CONH—, ((C₁-C₄)alkyl)((C₁-C₄)alkyl)N—NHCO—, —CO₂H, —CO₂(C₁-C₄)alkyl, (C₁-C₄)alkylthio-, phenyl-(C₁-C₄)alkylthio-, (C₁-C₄)alkyl-SO₂—, phenyl, optionally substituted 5-6 membered heterocycloalkyl, and optionally substituted 5-6 membered heteroaryl group,
wherein said optionally substituted (C₁-C₄)alkoxy is optionally substituted by hydroxyl, —CO₂H, —CONH₂, 5-6 membered heterocycloalkyl, or 5-6 membered heteroaryl; or said optionally substituted 5-6 membered heterocycloalkyl-CO—, optionally substituted 5-6 membered heterocycloalkyl, or optionally substituted 5-6 membered heteroaryl group is optionally substituted by (C₁-C₄)alkyl or oxo; or said optionally substituted 5-6 membered heterocycloalkyl-NHCO— is optionally substituted by (C₁-C₄)alkyl-CO—.

38. The compound, or a pharmaceutically acceptable salt thereof, according to claim 33, wherein R¹ is a substituted or unsubstituted 5-6 membered heteroaryl group, wherein said substituted or unsubstituted 5-6 membered heteroaryl group is a substituted or unsubstituted pyrimidinyl, pyrazinyl, pyridazinyl, or pyridyl,
wherein said substituted pyrimidinyl, pyrazinyl, pyridazinyl, pyridyl is substituted by 1 or 2 substituents independently selected from cyano, halogen, (C₁-C₄)alkyl, H₂N—, H₂NCO—, and —CO₂H.

39. The compound, or a pharmaceutically acceptable salt thereof, according to claim 33, wherein R¹ is a substituted or unsubstituted 5-6 membered heteroaryl group, wherein said substituted or unsubstituted 5-6 membered heteroaryl group is a substituted or unsubstituted pyrimidinyl,
wherein said substituted pyrimidinyl is substituted by 1 or 2 substituents independently selected from cyano, halogen, (C₁-C₄)alkyl, H₂N—, H₂NCO—, and —CO₂H.

40. The compound, or a pharmaceutically acceptable salt thereof, according to claim 33, wherein R¹ is a substituted or unsubstituted 5-6 membered heteroaryl group, wherein said substituted or unsubstituted 5-6 membered heteroaryl group is a substituted or unsubstituted oxazolyl, oxadiazolyl, thiazolyl, or tetrazolyl,
wherein said substituted oxazolyl, oxadiazolyl, thiazolyl, or tetrazolyl is substituted by 1 or 2 substituents independently selected from hydroxyl, cyano, halogen, (C₁-C₄)alkyl, halo(C₁-C₄)alkyl, hydroxy(C₁-C₄)alkyl, (C₂-C₄)alkynyl, optionally substituted (C₁-C₄)alkoxy, optionally substituted 5-6 membered heterocycloalkyl-CO—, fused 5-6 membered heterocycloalkyl; H₂N—, ((C₁-C₄)alkyl)-NH—, ((C₁-C₄)alkyl)((C₁-C₄)alkyl)N—, H₂NCO—, ((C₁-C₄)alkyl)NHCO—, (hydroxy-(C₁-C₄)alkyl)NHCO—, (C₃-C₆)cycloalkyl-NHCO—, optionally substituted 5-6 membered heterocycloalkyl-NHCO—, ((C₁-C₄)alkyl)((C₁-C₄)alkyl)-NCO—, (C₁-C₄)alkyl-CONH—, ((C₁-C₄)alkyl)((C₁-C₄)alkyl)N—NHCO—, —CO₂H, —CO₂(C₁-C₄)alkyl, (C₁-C₄)alkylthio-, phenyl-(C₁-C₄)alkylthio-, (C₁-C₄)alkyl-SO₂—, phenyl, optionally substituted 5-6 membered heterocycloalkyl, and optionally substituted 5-6 membered heteroaryl group,
wherein said optionally substituted (C₁-C₄)alkoxy is optionally substituted by hydroxyl, —CO₂H, 5-6 membered heterocycloalkyl, or 5-6 membered heteroaryl; or said optionally substituted 5-6 membered heterocycloalkyl-CO—, optionally substituted 5-6 membered heterocycloalkyl, or optionally substituted 5-6 membered heteroaryl group is optionally substituted by (C₁-C₄)alkyl or oxo; or said optionally substituted 5-6 membered heterocycloalkyl-NHCO— is optionally substituted by (C₁-C₄)alkyl-CO—.

41. The compound, or a pharmaceutically acceptable salt thereof, according to claim 33, wherein R¹ is a substituted 5-6 membered heteroaryl group, wherein said substituted 5-6 membered heteroaryl group is a substituted oxazolyl, oxadiazolyl, thiazolyl, or tetrazolyl,
wherein said substituted oxazolyl, oxadiazolyl, thiazolyl, or tetrazolyl is substituted by 1 or 2 substituents independently selected from cyano, (C₁-C₄)alkyl, H₂NCO—, ((C₁-C₄)alkyl)NHCO—, —CO₂(C₁-C₄)alkyl, and phenyl.

42. The compound, or pharmaceutically acceptable salt thereof, according to claim 33, wherein R¹ is a substituted or unsubstituted 5-6 membered heteroaryl group, wherein said substituted or unsubstituted 5-6 membered heteroaryl group is a substituted or unsubstituted oxadiazolyl, wherein said substituted oxadiazolyl is substituted by (C₁-C₄)alkyl.

43. The compound, or pharmaceutically acceptable salt thereof, according to claim 33, wherein R² is a substituted or unsubstituted phenyl or 5-6 membered heteroaryl group, wherein said substituted phenyl or 5-6 membered heteroaryl group is substituted by 1 or 2 substituents independently selected from halogen and cyano.

44. The compound, or pharmaceutically acceptable salt thereof, according to claim 33, wherein R² is a substituted 5-6 membered heteroaryl group, wherein said substituted 5-6 membered heteroaryl group is a substituted pyridyl, wherein said substituted pyridyl is substituted by 1 or 2 fluoro groups.

45. A compound which is (S)-6-(4-(5-(3,5-difluorophenyl)-4,5-dihydro-1H-pyrazole-1-carbonyl)piperidin-1-yl)pyrimidine-4-carbonitrile 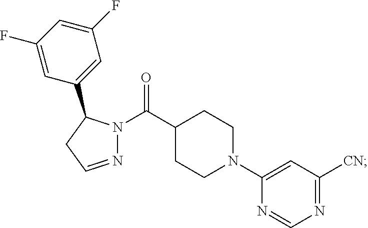 [SMILES: N#Cc1cc(N2CCC(C(=O)N3N=CC[C@H]3c3cc(F)cc(F)c3)CC2)ncn1]
or an acceptable salt thereof.

46. A compound which is (S)-6-(4-(5-(3,5-difluorophenyl)-4,5-dihydro-1H-pyrazole-1-carbonyl)piperidin-1-yl)pyrimidine-4-carbonitrile

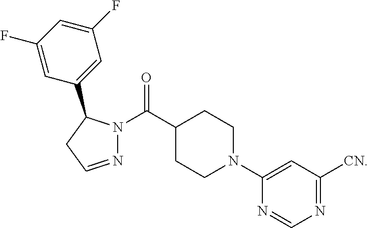 [SMILES: N#Cc1cc(N2CCC(C(=O)N3N=CC[C@H]3c3cc(F)cc(F)c3)CC2)ncn1]

47. A pharmaceutical composition comprising the compound, or pharmaceutically acceptable salt thereof, according to claim 33, and one or more pharmaceutically acceptable excipients.

48. A pharmaceutical composition according to claim 47, which further comprises at least one other therapeutically active agent.

49. A method of treating a RIP1 kinase-mediated disease or disorder in a human in need thereof, wherein the method comprises administering to the human a therapeutically effective amount of the compound, or pharmaceutically acceptable salt thereof, according to claim 33.

50. The method according to claim 49, wherein the disease or disorder is amyotrophic lateral sclerosis.

51. The method according to claim 49, wherein the disease or disorder is ulcerative colitis.

52. The method according to claim 49, wherein the disease or disorder is psoriasis.

53. The method according to claim 49, wherein the disease or disorder is rheumatoid arthritis.

54. The compound according to Formula (II): 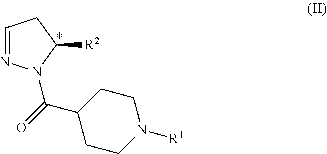
wherein:
R¹ is a substituted or unsubstituted 5-6 membered heteroaryl or 9-10 membered heteroaryl group,
wherein said substituted 5-6 membered heteroaryl or 9-10 membered heteroaryl group is substituted by 1 or 2 substituents independently selected from hydroxyl, cyano, halogen, (C₁-C₄)alkyl, halo(C₁-C₄)alkyl, hydroxy(C₁-C₄)alkyl, (C₂-C₄)alkynyl, optionally substituted (C₁-C₄)alkoxy, optionally substituted 5-6 membered heterocycloalkyl-CO—, fused 5-6 membered heterocycloalkyl, H₂N—, ((C₁-C₄)alkyl)-NH—, ((C₁-C₄)alkyl)((C₁-C₄)alkyl)N—, H₂NCO—, H₂NCO—(C₁-C₄)alkyl-, ((C₁-C₄)alkyl)NHCO—, (hydroxy-(C₁-C₄)alkyl)NHCO—, (C₃-C₆)cycloalkyl-NHCO—, optionally substituted 5-6 membered heterocycloalkyl-NHCO—, ((C₁-C₄)alkyl)((C₁-C₄)alkyl)N—CO—, (C₁-C₄)alkyl-CONH—, ((C₁-C₄)alkyl)((C₁-C₄)alkyl)N—NHCO—, —CO₂H, —CO₂(C₁-C₄)alkyl, (C₁-C₄)alkylthio-, phenyl-(C₁-C₄)alkylthio-, (C₁-C₄)alkyl-SO₂—, phenyl, optionally substituted 5-6 membered heterocycloalkyl, and optionally substituted 5-6 membered heteroaryl group,
wherein said optionally substituted (C₁-C₄)alkoxy is optionally substituted by hydroxyl, —CO₂H, —CONH₂, 5-6 membered heterocycloalkyl, or 5-6 membered heteroaryl; or said optionally substituted 5-6 membered heterocycloalkyl-CO—, optionally substituted 5-6 membered heterocycloalkyl, or optionally substituted 5-6 membered heteroaryl group is optionally substituted by (C₁-C₄)alkyl or oxo;
or said optionally substituted 5-6 membered heterocycloalkyl-NHCO— is optionally substituted by (C₁-C₄)alkyl-CO—; and
R² is a substituted or unsubstituted phenyl or 5-6 membered heteroaryl group,
wherein said substituted phenyl or 5-6 membered heteroaryl group is substituted by 1 or 2 substituents independently selected from halogen, (C₁-C₄)alkyl, (C₁-C₄)alkoxy, and cyano;
or a pharmaceutically acceptable salt thereof,
wherein said compound or pharmaceutically acceptable salt thereof is not:
(S)-(1-(5-fluoropyridin-2-yl)piperidin-4-yl)(5-phenyl-4,5-dihydro-1H-pyrazol-1-yl)methanone;
(S)-(1-(5-fluoropyrimidin-2-yl)piperidin-4-yl)(5-phenyl-4,5-dihydro-1H-pyrazol-1-yl)methanone;
(S)-(5-(5-fluoropyridin-3-yl)-4,5-dihydro-1H-pyrazol-1-yl)(1-(5-fluoropyrimidin-2-yl)piperidin-4-yl)methanone;
(S)-(5-(pyridin-3-yl)-4,5-dihydro-1H-pyrazol-1-yl)(1-(pyrimidin-2-yl)piperidin-4-yl)methanone;
(S)-(1-(pyridin-2-yl)piperidin-4-yl)(5-(pyridin-3-yl)-4,5-dihydro-1H-pyrazol-1-yl)methanone;
(S)-(5-phenyl-4,5-dihydro-1H-pyrazol-1-yl)(1-(pyrimidin-2-yl)piperidin-4-yl)methanone; or
(S)-(5-phenyl-4,5-dihydro-1H-pyrazol-1-yl)(1-(pyridin-2-yl)piperidin-4-yl)methanone.

55. The compound according to Formula (II): 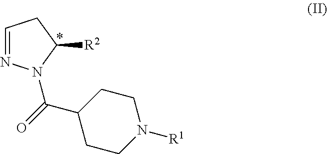
wherein
R¹ is a substituted or unsubstituted 5-6 membered heteroaryl group, wherein said substituted or unsubstituted 5-6 membered heteroaryl group is a substituted or unsubstituted pyrimidinyl or oxadiazolyl,
wherein said substituted pyrimidinyl is substituted by 1 or 2 substituents independently selected from cyano, halogen, (C₁-C₄)alkyl, H₂N—, H₂NCO—, and —CO₂H; or said substituted oxadiazolyl is optionally substituted by (C₁-C₄)alkyl; and
R² is a substituted or unsubstituted phenyl or pyridyl,
wherein said substituted phenyl or pyridyl is substituted by 1 or 2 fluoro groups;
or a pharmaceutically acceptable salt thereof,
wherein said compound or pharmaceutically acceptable salt thereof is not:
(S)-(1-(5-fluoropyrimidin-2-yl)piperidin-4-yl)(5-phenyl-4,5-dihydro-1H-pyrazol-1-yl)methanone,
(S)-(5-(5-fluoropyridin-3-yl)-4,5-dihydro-1H-pyrazol-1-yl)(1-(5-fluoropyrimidin-2-yl)piperidin-4-yl)methanone,
(S)-(5-(pyridin-3-yl)-4,5-dihydro-1H-pyrazol-1-yl)(1-(pyrimidin-2-yl)piperidin-4-yl)methanone, or
(S)-(5-phenyl-4,5-dihydro-1H-pyrazol-1-yl)(1-(pyrimidin-2-yl)piperidin-4-yl)methanone.

56. A pharmaceutical composition comprising the compound, or pharmaceutically acceptable salt thereof, according to claim 54, and one or more pharmaceutically acceptable excipients.

57. A pharmaceutical composition according to claim 56, which further comprises at least one other therapeutically active agent.

58. A method of treating a RIP1 kinase-mediated disease or disorder in a human in need thereof, wherein the method comprises administering to the human a therapeutically effective amount of the compound, or pharmaceutically acceptable salt thereof, according to claim 54.

59. The method according to claim 58, wherein the disease or disorder is amyotrophic lateral sclerosis.

60. The method according to claim 58, wherein the disease or disorder is ulcerative colitis.

61. The method according to claim 58, wherein the disease or disorder is psoriasis.

62. The method according to claim 58, wherein the disease or disorder is rheumatoid arthritis.
